(19) United States
(12) Patent Application Publication
(10) Pub. No.: US 2004/0086863 A1
(43) Pub. Date: May 6, 2004

(54) Ph interacting protein

(76) Inventors: Maria Rozakis- Adcock, Burlington (CA); Janet Farhang-Fallah, Toronto (CA); Alec Cheng, Saint Louis, MO (US)

Correspondence Address: FOLEY AND LARDNER, SUITE 500, 3000 K STREET NW, WASHINGTON, DC, 20007, US

(21) Appl. No.: 10/275,762

(22) PCT Filed: May 10, 2001
(86) PCT No.: PCT/CA01/00673

(51) Int. Cl. C07K 14/705; C12Q 1/68; C07H 21/04

(52) U.S. Cl. 435/6; 435/69.1; 435/320.1; 435/325; 530/350; 536/23.5

(57) ABSTRACT

The invention relates to nucleic acid molecules of a Pleckstrin Homology (PH) Domain-Interacting Protein, proteins encoded by such nucleic acid molecules; and uses of the proteins and nucleic acid molecules in the preparation of therapeutic and diagnostic agents. The proteins, nucleic acids molecules, and agents may be used in the diagnosis, prevention, and treatment of conditions and disorders involving the proteins and nucleic acid molecules including but not limited to cancer, and disorders associated with insullin response.

### FIELD OF THE INVENTION

The invention relates to nucleic acid molecules of a Pleckstrin Homology (PH) Domain-Interacting Protein, proteins encoded by such nucleic acid molecules; and uses of the proteins and nucleic acid molecules in the preparation of therapeutic and diagnostic agents. The proteins, nucleic acids molecules, and agents may be used in the diagnosis, prevention, and treatment of conditions and disorders involving the proteins and nucleic acid molecules including but not limited to cancer, and disorders associated with insulin response.

### BACKGROUND OF THE INVENTION

Upon ligand stimulation of insulin receptors, insulin receptor substrate-1 (“IRS-1”) is rapidly phosphorylated on multiple tyrosine residues which serve as docking sites for the assembly and activation of Src homology 2 (SH2) containing signaling proteins that function in eliciting many insulin-dependent biological responses (1). The N-terminus of IRS-1 contains a PH domain followed by the structurally homologous phosphotyrosine binding (PTB) domain that have been shown to co-operatively contribute in mediating productive receptor/substrate interactions (2). The PTB domain of IRS-1 binds directly to phosphorylated Tyr960 within the NPEY motif in the juxtamembrane region of the activated insulin receptor (IR) (3). However, the exact molecular mechanism by which the PH domain promotes receptor coupling is not known. Previous studies have demonstrated that deletion of the PH domain attenuates IRS-1 phosphorylation and subsequent insulin-mediated mitogenesis (2). Moreover, heterologous PH domains from unrelated proteins fail to restore mitogenic responses to insulin, suggesting that the IRS-1 PH domain is not simply a membrane targeting device but may interact with specific cellular ligands (4).

### SUMMARY OF THE INVENTION

Applicants isolated a novel protein designated “PH-Interacting Protein” or “PHIP” which is a physiological ligand of IRS-1 that links IRS-1 to the insulin receptor. Applicants have established that PHIP is a critical component of insulin-mediatedgene transcription, mitogenesis, glucose transport, and actin remodeling.

In particular, the inventors found that PHIP selectively binds to the pleckstrin homology (PH) domain of IRS-1 in vitro, and stably associates with IRS-1 and IRS-2 in vivo. Overexpression of PHIP enhanced insulin-induced transcriptional responses. By contrast, a dominant-negative mutant of PHIP specifically blocked mitogenic signals elicited by insulin and inhibited insulin-induced IRS-1 tryosine phosphorylation. Furthermore, DN-PHIP prevented insulin remodeling of the actin cytoskeleton in L6 myoblasts, which was accompanied by a profound inhibition of insulin-stimulated GLUT4 membrane translocation. Ectopically expressed PHIP proteins co-segregated with IRS-1 in low-density microsomes (LDM) fractions, and modulated the phosphoserine/threonine content of IRS-1 known to be important in IRS-1/LDM interactions.Applicants are the first to identify a physiological protein ligand of the IRS-1 PH domain, which may enhance coupling of IRS-1 to the IR by regulating the spatial compartmentalization and intracellular routing of IRS-1. The gene encoding PHIP was mapped to chromosome6. The present inventors also found that PHIP associates with STAT (Signal Transducer and Activator of Transcription) transcription factors, in particular STAT3, and it may link STAT transcription factors to the insulin family of receptors. Therefore, PHIP is an adaptor protein that recruits signaling molecules such as IRS-1 and STAT3, to activated receptors that interact with, and phosphorylate the signaling molecules.

Therefore, broadly stated the present invention provides an adaptor protein that recruits proteins of the IRS protein family and STAT transcription factors to receptors that interact with, and phosphorylate the proteins and STAT transcription factors.

The present invention also contemplates an isolated nucleic acid molecule encoding PHIP, including mRNAs, DNAs, cDNAs, genomic DNAs, PNAs, as well as antisense analogs and biologically, diagnostically, prophylactically, clinically or therapeutically useful variants or fragments thereof and compositions comprising same.

The invention also contemplates an isolated PHIP encoded by a nucleic acid molecule of the invention, including a truncation, an analog, an allelic or species variation thereof, a homolog of the protein or a truncation thereof, or an activated (e.g. phosphorylated)PHIP. (PHIP and truncations, analogs, allelic or species variations, homologs thereof, and activated PHIP are collectively referred to herein as “PHI Proteins”). An isolated PHI Protein may be obtained from any species, particularly mammalian, including bovine, ovine, porcine, murine, equine, preferably human, from any source whether natural, synthetic, semi-synthetic,or recombinant. A PHI Protein is characterized by an N-terminal α-helical region predicting a coiled coil structure and a region containing two bromodomains.

In accordance with an aspect of the invention an isolated Pleckstrin Homology domain Interacting Protein (“PHI Protein”) is provided which is capable of forming a stable interaction with a PH domain of insulin receptor substrate-1 (IRS-1), and is characterized by an N-terminal α-helical region predicting a coiled coil structure and a region containing two bromodomains.

The nucleic acid molecules which encode for a mature PHI Protein may include only the coding sequence for the mature polypeptide; the coding sequence for the mature polypeptide and additional coding sequences (e.g. leader or secretory sequences, propolypeptide sequences); the coding sequence for the mature polypeptide (and optionally additional coding sequence) and non-coding sequence, such as introns or non-coding sequence 5′ and/or 3′ of the coding sequence of the mature polypeptide.

Therefore, the term “nucleic acid molecule encoding a PHI Protein” encompasses a nucleic acid molecule which includes only coding sequence for a PHI Protein as well as a nucleic acid molecule which includes additional coding and/or non-coding sequences.

The nucleic acid molecules of the invention may be inserted into an appropriate vector, and the vector may contain the necessary elements for the transcription and translation of an inserted coding sequence. Accordingly, vectors may be constructed which comprise a nucleic acid molecule of the invention, and where appropriate one or more transcription and translation elements linked to the nucleic acid molecule.

In accordance with an aspect of the invention, a vector is provided comprising a DNA molecule with a nucleotide sequence encoding at least one epitope of a PHI Protein, and suitable regulatory sequences to allow expression in a host cell.

A vector can be used to transform host cells to express a PHI Protein. Therefore, the invention further provides host cells containing a vector of the invention. The invention also contemplates transgenic non-human mammals whose germ cells and somatic cells contain a vector comprising a nucleic acid molecule of the invention in particular one that encodes an analog of PHIP, or a truncation of PHIP.

A protein of the invention may be obtained as an isolate from natural cell sources, but it is preferably produced by recombinant procedures. In one aspect the invention provides a method for preparing a PHI Protein utilizing the isolated nucleic acid molecules of the invention. In an embodiment a method for preparing a PHI Protein is provided comprising:

(a) transferring a vector of the invention comprising a nucleic acid sequence encoding a PHI Protein, into a host cell;

(b) selecting transformed host cells from untransformed host cells;

(c) culturing a selected transformed host cell under conditions which allow expression of the PHI Protein; and

(d) isolating the PHI Protein.

The invention further broadly contemplates a recombinant PHI Protein obtained using a method of the invention.

A PHI Protein of the invention may be conjugated with other molecules, such as polypeptides, to prepare fusion polypeptides or chimeric polypeptides. This may be accomplished, for example, by the synthesis of N-terminal or C-terminal fusion polypeptides.

An aspect of the invention provides molecules (e.g. peptides) derived from a binding region of a PHI Protein.

The invention also permits the construction of nucleotide probes that are unique to nucleic acid molecules of the invention and/or to proteins of the invention. Therefore, the invention also relates to a probe comprising a sequence encoding a PHI Protein, or a portion (i.e. fragment) thereof. The probe may be labeled, for example, with a detectable substance and it may be used to select from a mixture of nucleic acid molecules, a nucleic acid molecule of the invention including nucleic acid molecules coding for a polypeptide which displays one or more of the properties of a PHI Protein.

An aspect of the invention provides a complex comprising a PHI Protein or a binding region thereof, and a binding partner. In an embodiment of the invention a complex is provided comprising a PHI Protein or a PH domain binding region, and a PH domain containing protein or a PH domain. The invention also contemplates a complex comprising a PHI Protein or a binding region thereof, in particular an IR binding region, and a receptor that interacts with a protein of the IRS protein family, or a binding region thereof. Still further, the invention contemplates a complex comprising a PHI Protein or a binding region thereof, in particular a STAT binding region, and a STAT transcription factor or a binding region thereof that interacts with a PHI Protein.

The invention further contemplates antibodies having specificity against an epitope of a PHI Protein or complex of the invention. Antibodies may be labeled with a detectable substance and used to detect proteins or complexes of the invention in biological samples, tissues, and cells. Antibodies may have particular use in therapeutic applications, for example to react with tumor cells, and in conjugates and immunotoxins as target selective carriers of various agents which have antitumor effects including chemotherapeutic drugs, toxins, immunological response modifiers, enzymes, and radioisotopes.

In accordance with an aspect of the invention there is provided a method of, and products for, diagnosing and monitoring conditions involving a PHI Protein by determining the presence of nucleic acid molecules, proteins, and complexes of the invention.

The invention provides a method for identifying a substance which binds to a PHI Protein or a binding region thereof (e.g. a PH domain binding region, IR binding region, or STAT binding region), comprising reacting the protein or binding region with at least one substance which potentially can interact or bind with the protein or binding region, under conditions which permit the formation of complexes between the substance and protein or binding region, and detecting binding or recovering complexes. Binding may be detected by assaying for complexes, for free substance, or for non-complexed protein or binding region. The invention also contemplates methods for identifying substances that bind to other intracellular proteins that interact with a PHI Protein or binding region thereof. Methods can also be utilized which identify compounds which bind to phip nucleic acid regulatory sequences (e.g. promoter sequences).

Still further the invention provides a method for evaluating a test compound for its ability to modulate the activity of a PHI Protein of the invention. “Modulate” refers to a change or an alteration in the biological activity of a PHI Protein of the invention. Modulation may be an increase (i.e. promotion) or a decrease (i.e. disruption) in activity, a change in characteristics, or any other change in the biological, functional, or immunological properties of the protein.

For example a substance which reduces or enhances the activity of a PHI Protein may be evaluated. The association or interaction between a PHI Protein and a binding partner may be promoted or enhanced either by increasing production of a PHI Protein, or by increasing expression of a PHI Protein, or by promoting interaction of a PHI Protein and a binding partner (e.g. PH domain containing protein or receptor that interacts with a protein of the IRS protein family) or by prolonging the duration of the association or interaction. The association or interaction between a PHI Protein and a binding partner may be disrupted or reduced by preventing production of a PHI Protein or by preventing expression of a PHI Protein, or by preventing interaction of a PHI Protein and a binding partner or interfering with the interaction. A method may include measuring or detecting various properties including the level of signal transduction and the level of interaction between a PHI Protein or binding region thereof and a binding partner.

In an embodiment, the method comprises reacting a PHI Protein or binding region thereof, with a substance which interacts with or binds to the protein or binding region thereof and a test compound under conditions which permit the formation of complexes between the substance and protein or binding region, and removing and/or detecting complexes.

In other embodiments, the invention provides a method for identifying inhibitors of a PHI Protein interaction, comprising

(a) providing a reaction mixture including a PHI Protein and a binding partner, or at least a portion of each which interact;

(b) contacting the reaction mixture with one or more test compounds;

(c) identifying compounds which inhibit the interaction of the PHI Protein and binding partner.

In certain preferred embodiments, the reaction mixture is a whole cell. In other embodiments, the reaction mixture is a cell lysate or purified protein composition. The subject method can be carried out using libraries of test compounds. Such agents can be proteins, peptides, nucleic acids, carbohydrates, small organic molecules, and natural product extract libraries, such as isolated from animals, plants, fungus and/or microbes.

Still another aspect of the present invention provides a method of conducting a drug discovery business comprising:

(a) providing one or more assay systems for identifying agents by their ability to inhibit or potentiate the interaction of a PHI Protein and binding partner;

(b) conducting therapeutic profiling of agents identified in step (a), or further analogs thereof, for efficacy and toxicity in animals; and

(c) formulating a pharmaceutical composition including one or more agents identified in step (b) as having an acceptable therapeutic profile.

In certain embodiments, the subject method can also include a step of establishing a distribution system for distributing the pharmaceutical composition for sale, and may optionally include establishing a sales group for marketing the pharmaceutical preparation.

Yet another aspect of the invention provides a method of conducting a target discovery business comprising:

(a) providing one or more assay systems for identifying agents by their ability to inhibit or potentiate the interaction of a PHI Protein and binding partner,

(b) (optionally) conducting therapeutic profiling of agents identified in step (a) for efficacy and toxicity in animals; and

(c) licensing, to a third party, the rights for further drug development and/or sales for agents identified in step (a), or analogs thereof.

Compounds which modulate the biological activity of a PHI Protein may also be identified using the methods of the invention by comparing the pattern and level of expression of a nucleic acid molecule or protein of the invention in biological samples, tissues and cells, in the presence, and in the absence of the test compounds.

Methods are also contemplated that identify compounds or substances (e.g. polypeptides) which interact with phip regulatory sequences (e.g. promoter sequences, enhancer sequences, negative modulator sequences).

The disruption or promotion of the interaction between the molecules in complexes of the invention may be useful in therapeutic procedures. Therefore, the invention features a method for treating a subject having a condition characterized by an abnormality in a signal transduction pathway involving an interaction between a PHI Protein or a PH domain binding region, and a PH domain containing protein or a PH domain; an interaction between an IR binding region and a receptor that interacts with a protein of the IRS protein family; or, an interaction between a PHI Protein or a STAT binding region, and a STAT transcription factor or a binding region thereof that interacts with a PHI Protein.

The nucleic acid molecules, proteins, complexes, peptides, and antibodies of the invention, and substances, agents, and compounds identified using the methods of the invention, may be used to modulate the biological activity of a PHI Protein or complex of the invention, or a signal transduction pathway involving a PHI Protein or complex of the invention, and they may be used in the treatment of conditions mediated by a PHI Protein or a signal transduction pathway involving a PHI Protein or complex of the invention. Accordingly, the nucleic acid molecules, proteins, antibodies, complexes of the invention, and substances, agents, and compounds may be formulated into compositions for administration to individuals suffering from one or more of these conditions. In an embodiment of the invention the condition is cancer. In another embodiment of the invention the condition is a disorder associated with an insulin response. Therefore, the present invention also relates to a composition comprising one or more of a protein, antibody, complex, or nucleic acid molecule of the invention, or substance, compound, or agent identified using the methods of the invention, and a pharmaceutically acceptable carrier, excipient or diluent. A method for treating or preventing these conditions is also provided comprising administering to a patient in need thereof a composition of the invention.

The invention also contemplates the use of a nucleic acid molecule, protein, complex, peptide, antibody, substance, agent, or compound of the invention in the preparation of a medicament for the treatment of a condition or disorder mediated by a PHI Protein or a signal transduction pathway involving a PHI Protein or a complex of the invention.

In accordance with a further aspect of the invention, there are provided processes for utilizing proteins, complexes, or nucleic acid molecules described herein, for in vitro purposes related to scientific research, synthesis of DNA and manufacture of vectors.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description. These and other aspects, features, and advantages of the present invention should be apparent to those skilled in the art from the following drawings and detailed description.

### DESCRIPTION OF THE DRAWINGS

The invention will now be described in relation to the drawings in which:

FIG. 1 show the deduced amino-acid sequence and schematic representation of PHIP. (A) Alignment of mouse (m) and human (h) PHIP sequences. (B) There are two bromodomains in PHIP, BD1 (230-345) and BD2 (387-503). The PHIP IRS-1/PH binding region (PBR) (amino acids 8-209) isolated from the yeast clone VP1.32 is underlined.

FIG. 2 are blots showing that PHIP associates with IRS-1 both in vitro and in vivo. (A) PHIP migrates with an apparent molecular mass of 104 kDa. PHIP was immunoprecipitated from multiple myeloma U266 cell lysates and immunoblotted with anti-PHIP antibodies (Abs) (10) (B) Two forms of PHIP (97 and 104 KDa) observed in anti-PHIP immunoprecipitates from cell lysates of U266, human A431 epidermoid carcinoma, Rat-2 and mouse NIH/3T3 fibroblasts. (C) PHIP interacts selectively with the IRS-1 PH domain in vitro. Yeast cell lysates expressing HA-tagged PH domains from either IRS-1, SOS1, ECT-2 or Ras-GAP (GAP) were mixed with immobilized GST-PHIP fusion proteins and complexes were subjected to Western blot analysis with anti-HA Abs (13). (D) Binding of IRS-1 PH domain mutants to PHIP. Left, Immunodetection of HA-tagged IRS-1 PH domain mutants from whole cell lysates (50 μg) of transiently transfected COS-1 cells. PH^{WT} (IRS-1 PH domain residues3-133), PH^{NT} (residues 3-67), PH^{CT} (residues 55-133), PH^{W106A} (Trp106 residue conserved in all PH domains changed to Ala); Right cell lysates (500 μg) expressing the indicated IRS-1 PH domain mutant were mixed with either GST or GST-PHIP (PBR) proteins and processed as in (C). (E) PHIP stably associates with IRS-1 in vivo. Serum deprived NIH/IR cells were either left unstimulated or stimulated with insulin (2 μM) for 5 minutes. Cell lysates were immunoprecipitated with anti-IRS-1^{PCT} (Upstate Biotechnology Inc.,UBI), anti-IRS-1 ^{PH} or anti-PHIP Abs and subjected to western blotting with anti-PHIP or anti-IRS-1^{PCT} Abs as indicated. Anti-IRS-2Abs were used to coimmunoprecipitateIRS-2/PHIP complexes from asynchronized cells. (F) PHIP is not a substrate of the IR. PHIP was immunoprecipitated from untreated and insulin-treated human kidney 293 cell extracts using anti-PHIP Abs directed against the PBR region. Immune complexes were resolved by SDS-PAGE and immunoblotted with anti-phosphotyrosine Abs (anti-pTyr, PY20, New England Biolabs). The blot was stripped and reprobed with either anti-IRS-1^{PCT} or anti-PHIP Abs. A 103 KDa phosphoprotein denoted by an asterisk likely represents STAT3.

FIG. 3 are graphs showing the effect of PHIP on insulin signaling. (A) Human PHIP potentiates transcription of 5×SRE-fos luciferase expression by insulin. COS-1 cells were transiently transfected with increasing amounts of pCGN/hPHIP (6 μg, 9 μg, 12 μg) or empty vector as control (12 μg) together with 3 μg of 5×SRE-fos luciferase reporter construct (5×SRE-LUC). Serum-starved cells were either left untreated or treated with Mek-1 inhibitor (50 μM) for 2 hours. Cells were incubated for 10 hours with or without insulin (0.2 μM) and relative luciferase activity was measured in cell lysates using a dual-light system (Tropix) (16). Results are expressed as the mean+SD of triplicates from a representative experiment (B) IRS-1 PH domain inhibits PHIP-induced SRE-LUC activity. COS cells were cotransfected with pCGN/hPHIP (4 μg) and the indicated amount of pCGN/IRS-1 PH together with 2 μg of 5×SRE-LUC. Cells were insulin treated and processed as in (A). C) IRS-1 PH mediated inhibition of PHIP-stimulated luciferase activity is restored by wild-type IRS-1 in a dose-dependent manner. COS cells were cotransfected with 1 μg of pCGN/hPHIP, 2 μg of 5×SRE-LUC, either 1 μg of pCGN/IRS-1-PH or vector DNA and increasing amounts of pCGN/IRS-1 cDNA as indicated. Cells were then insulin treated and processed as in (A).

FIG. 4 shows blots illustrating the dominant negative PHIP inhibits insulin-induced tyrosine phosphorylation of IRS-1. (A,B) COS-7 cells were transiently transfected with either pCGN/HA-DN-PHIP (DN/PHIP), pCGN/HA-PHIP (PHIP) or empty vector. Cell cultures were treated with or without insulin (0.2 μM) for 5 minutes. Whole cell lysates or anti-IRS-1 immunoprecipitates were subjected to immunoblot analysis with either anti-IRS 1PCT, anti-pTyr or anti-HA Abs as indicated. Anti-IR immunoprecipitates were blotted with anti-pTyr antibodies. The membrane was stripped and reprobed with anti-IR antibodies. (C) Rat-1 fibroblasts were transiently transfected with either pCGN/HA-DN-PHIP or empty vector. Cell cultures were treated with insulin (0.2 μM) for 5 minutes. Cell lysates were precipitated with anti-IRS-1PCT or anti-Shc Abs and were subjected to immunoblot analysis with anti-pTyr Abs. The membrane containing Shc immune complexes was stripped and reprobed with anti-Shc Abs. (D) DN/PHIP inhibits MAPK activity through IRS-1 and not SHC adaptor protein. COS cells were transiently transfected with pCDNA1/HA-p44MAPK and either pCGN/HA-DN-PHIP or empty vector. Cell cultures were treated with or without insulin. Cell lysates were precipitated with anti-HA Abs and subjected to an in-vitro kinase assay with MBP as substrate. The HA-depleted lysates were then precipitated with anti-Shc Abs and subjected to analysis with anti-pTyr Abs.

FIG. 5 shows PHIP oveexpression altos IRS-1 electrophoretic mobility (A) PHIP and IRS-1 are co-localized in the LDM. LDM and cytosolic fractions were prepared from unstimulated and insulin-stimulated COS-7 cells transiently transfected with 20 μg of pCGN/hPHIP (Human PHIP) or empty vector as control. Two hundred microgram of protein from each fraction is resolved by SDS-PAGE and analyzed by immunoblotting using anti-IRS-1^{PCT} antibodies (Abs). Anti-phosphotyrosine(pTyr) and Anti-HA Abs are used to detect insulin-induced tyrosine phosphorylated IRS-1 and ectopically expressed PHIP, respectively. Anti-transferrin receptor Abs are used as the marker for the LDM compartment (B) PHIP regulates IRS-1 subcellular localization by regulating IRS-1 serine/threonine phosphorylation. Western blot analysis using anti-IRS-1^{PCT} Abs were performed on COS-7 cell lysates transiently transfected with empty vector (20 μg), and plasmid expressing HA-tagged hPHIP (5 μg, 10 μg, and 20 μg). Ectopic hPHIP expression was monitored using anti-HA Abs.

FIG. 6 is a schematic representation of PHIP and neuronal differentiation related protein (NDRP). There are two bromodomains in PHIP, BD1 (230-345) and BD2 (387-503). The PHIP/IRS-1 PH binding region (PBR) (amino-acids 5-209) is underlined.

FIG. 7 shows an amino acid sequence alignment of human and mouse neuronal differentiation related protein (NDRP).

FIG. 8 shows a nucleic acid sequence alignment of human and mouse neuronal differentiation related protein (NDRP).

FIG. 9 shows an amino acid sequence alignment of WD-Repeat Protein 9 and PHIP.

FIG. 10 shows a nucleic acid sequence alignment of WD-Repeat Protein 9 and PHIP.

### DETAILED DESCRIPTION OF THE INVENTION

In accordance with the present invention there may be employed conventionalmolecular biology, microbiology, and recombinant DNA techniques within the skill of the art Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

1. Glossary

The term “agonist” of a protein of interest, for example, a PHI Protein, refers to a compound that binds the protein or part thereof and maintains or increases the activity of the protein to which it binds. Agonists may include proteins, nucleic acids, carbohydrates, or any other molecules that bind to a protein, complex, or molecule of the complex (e.g. PHI Protein). Agonists also include a molecule (e.g. peptide) derived from a PHI Protein or binding region thereof (e.g. PH binding domain region, IR binding region, or STAT binding region) but will not include the full length sequence of the wild-type molecule. Peptide mimetics, synthetic molecules with physical structures designed to mimic structural features of particular peptides, may serve as agonists. The stimulation may be direct, or indirect, or by a competitive or non-competitive mechanism.

The term “antagonist”, as used herein, of a protein of interest, for example, a PHI Protein, refers to a compound that binds the protein or part thereof, but does not maintain the activity of the protein to which it binds. Antagonists may include proteins, nucleic acids, carbohydrates, or any other molecules that bind to a protein, complex, or molecule of the complex (e.g. PHI Protein). Antagonists also include a molecule (e.g. peptide) derived from a PHI Protein or binding region thereof (e.g. PH binding domain region, IR binding region, or STAT binding region) but preferably will not include the full length sequence of the wild-type molecule. Peptide mimetics, synthetic molecules with physical structures designed to mimic structural features of particular peptides, may serve as antagonists. The inhibition may be direct, or indirect, or by a competitive or non-competitive mechanism.

“Antibody” includes intact monoclonal or polyclonal molecules, and immunologically active fragments (e.g. a Fab or (Fab)₂ fragment), an antibody heavy chain, humanized antibodies, and antibody light chain, a genetically engineered single chain Fᵥ molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art. Antibodies that bind a protein, complex, or peptide of the invention can be prepared using intact proteins, peptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to proteins or peptides include bovine serum albumin and thyroglobulin, keyhole limpet hemocyanin. The coupled protein or peptide may then be used to immunize the animal (e.g., a mouse, a rat, or a rabbit).

A “binding region” is that portion of a PHI Protein or molecule in a complex of the invention which interacts with or binds directly or indirectly with another molecule (e.g. PH domain or STAT3) or with another molecule in a complex of the invention. The binding domain may be a sequential portion of the molecule i.e. a contiguous sequence of amino acids, or it may be conformational i.e. a combination of non-contiguous sequences of amino acids which when the molecule is in its native state forms a structure that interacts with another molecule in a complex of the invention.

The term “complementary” refers to the natural binding of nucleic acid molecules under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules.

By being “derived from” a binding region is meant any molecular entity which is identical or substantially equivalent to the native binding region of a PHI Protein or a molecule in a complex of the invention. A peptide derived from a specific binding region may encompass the amino acid sequence of a naturally occurring binding site, any portion of that binding site, or other molecular entity that functions to bind to an associated molecule. A peptide derived from such a binding region will interact directly or indirectly with an associated molecule in such a way as to mimic the native binding region. Such peptides may include competitive inhibitors, peptide mimetics, and the like.

“Interaction” or “interacting” means any physical association between proteins, other molecules such as lipids, carbohydrates, nucleotides, and other cell metabolites, which may be covalent or non-covalent (e.g. electrostatic bonds, hydrogen bonds, and Van der Waals bonds). Interactions include interactions between proteins and cellular molecules, including protein-protein interactions, protein-lipid interactions, and others. Certain interacting molecules interact only after one or more of them have been stimulated. For example, a PH domain containing protein may only bind to a ligand if the protein is phosphorylated. Interactions between proteins and other cellular molecules may be direct or indirect. An example of an indirect interaction is the independent production, stimulation, or inhibition of a PHI Protein or binding domain thereof, by a modulator. Various methods known in the art may be used to measure the level of an interaction.

“IR binding region” refers to a binding region of a PHI Protein of the invention that interacts with or binds a receptor that interacts with a protein of the IRS protein family. In preferred embodiments the interaction is specific and a binding region does not interact, or interacts to a lesser extent with molecules that are not such receptors. The K_{d} for an interaction between an IR binding region and a receptor is preferably less than 10 μM, more preferably 1,000 nM, most preferably 500 nM. In embodiments of the invention, an IR binding region may be provided as part of a protein, alone or in isolation from the remainder of the amino acid sequence of the protein, or contained in a lipid vesicle or as a freely soluble small molecule. An example of an IR binding region is the region corresponding to bromodomain BD1 comprising amino acids 230-345 of SEQ. ID. NO. 2 or 5, or the amino acid sequence of SEQ.ID. NO. 15, or bromodomain BD2 comprising amino acids 387-503 of SEQ. ID. NO. 2 or 5, or the amino acid sequence of SEQ.ID. NO. 17.

“IRS protein family” refers to docking proteins that provide an interface between multiple receptor complexes and various signaling proteins with Src homology 2 domains. The proteins are involved in signaling events initiated by several classes of receptors including the insulin receptor, growth factor receptors (e.g. insulin-like growth factor I (IGF-I) receptor, receptors for growth hormone and prolactin), cytokine receptors (e.g. receptors for IL-2, IL-4, IL-9, IL-13, and IL-15, members of the IL-6 receptor family), and interferon receptors (e.g. receptors for IFNα/β and IFNγ). The insulin receptor substrate, IRS-1 is the prototype for this class of molecules. Other members of the family include IRS-2, Gab-1, and p62^{dok}. The proteins contain several common structures including an NH₂-terminal PH domain and/or phosphotyrosine binding (PTB) domain that mediate protein-protein interactions; multiple COOH-terminal tyrosine residues that bind SH2-containing proteins; proline-rich regions to interact with SH3 or WW domains; and serine/threonine-rich regions which regulate intracellular localization/trafficking of IRS proteins likely through protein-protein interactions (M. F. White and L. Yenush, 1998 and references therein). IRS-1 and IRS-2 have a PH domain at the extreme NH₂ terminus, followed immediately by a PTB domain that binds to phosphorylated NPXY motifs. An activated i.e. phosphorylated protein of the IRS protein family may be used for purposes of the invention.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or agonist or antagonist of the invention. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a peptide, or agonist or antagonist of the invention.

A “PH domain” refers to a distinct approximately 100 amino acid region originally identified in pleckstrin but are known to occur in many signaling proteins M. F. White and L. Yenush, 1998 and references therein). The PH domain has a distinct structural module characterized by two anti-parallel β sheets forming a sandwich, with one corner covered by an amphipathicCOOH-terminal α-helix (Lemmon et al, 1996, Cell 85:621-624). PH domains may be identified using sequence alignment techniques and three-dimensional structure comparisons. Preferred PH domains are the PH domains of proteins of the IRS protein family, preferably IRS-1 and IRS-2 PH domains. In embodiments of the invention, a PH domain may be provided as part of a protein, alone or in isolation from the remainder of the amino acid sequence of the protein, or contained in a lipid vesicle or as a freely soluble small molecule.

“PH domain binding region” refers to a binding region of a PHI Protein that interacts with or binds a PH domain. In preferred embodiments the interaction is specific and a binding region does not interact, or interacts to a lesser extent with molecules that are non-PH domains. The K_{d} for an interaction between a PH domain binding region and a PH domain is preferably less than 10 μM, more preferably 1,000 nM, most preferably 500 nM. In embodiments of the invention, a PH domain binding region may be provided as part of a protein, alone or in isolation from the remainder of the amino acid sequence of the protein, or contained in a lipid vesicle or as a freely soluble small molecule. An example of a PH domain binding region is the PH domain binding region corresponding to amino acids 8 to 209 in SEQ. ID. NO. 2, 5, 8, or 10 or the amino acid sequence of SEQ. ID. NO. 12 or 13 (referred to herein as “PH binding region” or “BR”).

A “PH domain containing protein” refers to proteins or peptides, or parts thereof which comprise or consist essentially of a PH domain. In embodiments of the invention, a PH domain containing protein may be provided as part of a protein, alone or in isolation from the remainder of the amino acid sequence of the protein, or contained in a lipid vesicle or as a freely soluble small molecule. Examples of such proteins include proteins of the IRS protein family, preferably IRS-1 and IRS-2.

A “receptor that interacts with a protein of the IRS protein family” refers to receptor tyrosine kinases and cytokine receptors that interact with, and phosphorylate a protein of the IRS protein family. Examples of these receptors include the insulin receptor, growth factor receptors (e.g. insulin-like growth factor I (IGF-I) receptor, receptors for growth hormone and prolactin), cytokine receptors (e.g. receptors for IL-2, IL-4, IL-9, IL-13, and IL-15, members of the IL-6 receptor family), and interferon receptors (e.g. receptors for IFNα/β and IFNγ). Preferably,the invention uses the insulin receptor (“IR”) and insulin-like growth factor I receptor (“IGF-1R”).

The terms “sequence similarity” or “sequence identity” refer to the relationship between two or more amino acid or nucleic acid sequences, determined by comparing the sequences, which relationship is generally known as “homology”. Identity in the art also means the degree of sequence relatedness between amino acid or nucleic acid sequences, as the case may be, as determined by the match between strings of such sequences. Both identity and similarity can be readily calculated (Computational Molecular Biology, Lesk, A. M., ed., Oxford University Press New York, 1988; Biocomputing: Informatics and Genome Projects, Smith, D. W. ed., Academic Press, New York, 1993; Computer Analysis of Sequence Data, Part I Griffin, A. M., and Griffin, H. G. eds. Humana Press, New Jersey, 1994; Sequence Analysis in Molecular Biology, von Heinje, G., Academic Press, New York, 1987; and Sequence Analysis Primer, Gribskov, M. and Devereux, J., eds. M. Stockton Press, New York 1991). While there are a number of existing methods to measure identity and similarity between two amino acid sequences or two nucleic acid sequences, both terms are well known to the skilled artisan (Sequence Analysis in Molecular Biology, von Heinje, G., Academic Press, New York, 1987; Sequence Analysis Primer, Gribskov, M. and Devereux, J., eds. M. Stockton Press, New York, 1991; and Carillo, H., and Lipman, D. SIAM J. Applied Math., 48:1073, 1988). Preferred methods for determining identity are designed to give the largest match between the sequences tested. Methods to determine identity are codified in computer programs. Preferred computer program methods for determining identity and similarity between two sequences include but are not limited to the GCG program package (Devereux, J. et al, Nucleic Acids Research 12(1): 387, 1984), BLASTP, BLASTN, and FASTA (Atschul, S. F. et al., J. Molec. Biol. 215:403, 1990). Identity or similarity may also be determined using the alignment algorithm of Dayhoff et al [Methods in Enzymology 91: 524-545 (1983)].

“Signal transduction pathway” refers to the sequence of events that involves the transmission of a message from an extracellular protein to the cytoplasm through the cell membrane. Signal transduction pathways contemplated herein include pathways involving a PHI Protein or a complex of the invention or an interacting molecule thereof In particular, the pathways are those involving the IRS protein family, in particular IRS-1, or a STAT transcription factor (e.g. STAT3) that regulate cellular processes including the control of glucose metabolism, protein synthesis, and cell survival, growth, and transformation. Such pathways include the MAP kinase pathway leading to c-fos gene expression; IRS-1 regulated IL-4 stimulation of hematopoietic cells; and IRS-1 mediated GH and interferon γ (FNγ) signaling. IRS-1 also mediates pathways dependent on phosphatidylinositol3-linase. In addition, IRS proteins regulate cellular processes through IGR-I/IGF-R signaling pathways which when activated stimulate mitogenesis and cellular transformation, and inhibit apoptosis. The amount and intensity of a given signal in a signal transduction pathway can be measured using conventional methods (See Example 1 herein). For example, the concentration and localization of various proteins and complexes in a signal transduction pathway can be measured, conformational changes that are involved in the transmission of a signal may be observed using circular dichroism and fluorescence studies, and various symptoms of a condition associated with an abnormality in the signal transduction pathway may be detected.

“STAT transcription factor” or “STAT” refers to a member of the family of proteins required for cytokine-mediated signal transduction and immune function (Schindler et al., Ann. Rev. Biochem. 64: 621-651, 1995). Following receptor ligation by cytokines, STAT family members become activated by tyrosine phosphorylation, through the action of Janus family kinase (JAK) members. Activated STAT proteins form homodimeric and heterodimeric complexes that translocate from the cytoplasm to the nucleus where they bind to cis-acting promoter sequences and regulate transcription of a number of genes required for the immune response. Examples of STAT transcriptional factors include but are not limited to STAT1 (α and β), STAT3 (α and β), STAT4, and STAT6, and all isoforms, and homo- and heterodimers thereof, preferably STAT3 (α and β). STAT3 activation is required for IL-6 dependent responses associated with tissue inflammation, and IL-10 responses are associated with Th2 helper cell function (Inoue, M. et al J. Biol Chem. 272: 9550-9555, 1975 and Weber-North et al, J. Biol. Chem. 271: 27954, 1996)

“STAT binding region” refers to a binding region of a PHI Protein that interacts with a STAT transcription factor. In preferred embodiments the interaction is specific and a binding region does not interact, or interacts to a lesser extent with molecules that are non-STAT transcription factors. The K_{d} for an interaction between a PHI Protein and a STAT transcription factor is preferably less than 10 μM, more preferably 1,000 nM, most preferably 500 nM. In embodiments of the invention, a STAT binding region may be provided as part of a protein, alone or in isolation from the remainder of the amino acid sequence of the protein, or contained in a lipid vesicle or as a freely soluble small molecule

2. Nucleic Acid Molecules

As hereinbefore mentioned, the invention provides an isolated nucleic acid molecule comprising or consisting essentially of a sequence encoding a PHI Protein. The term “isolated” refers to a nucleic acid (or protein) removed from its natural environment, purified or separated, or substantially free of cellular material or culture medium when produced by recombinant DNA techniques, or chemical reactants, or other chemicals when chemically synthesized. Preferably, an isolated nucleic acid is at least 60% free, more preferably at least 75% free, and most preferably at least 90% free from other components with which it is naturally associated. The term “nucleicacid” is intended to include modified or unmodified DNA, RNA, including mRNAs, DNAs, cDNAs, and genomic DNAs, or a mixed polymer, and can be either single-stranded, double-stranded or triple-stranded. For example, a nucleic acid sequence may be a single-stranded or double-stranded DNA, DNA that is a mixture of single-and double-stranded regions, or single-, double- and triple-stranded regions, single- and double-stranded RNA, RNA that may be single-stranded, or more typically, double-stranded, or triple-stranded, or a mixture of regions comprising RNA or DNA, or both RNA and DNA. The strands in such regions may be from the same molecule or from different molecules. The DNAs or RNAs may contain one or more modified bases. For example,the DNAs or RNAs may have backbones modified for stability or for other reasons. A nucleic acid sequence includes an oligonucleotide, nucleotide, or polynucleotides. The term “nucleic acid molecule” and in particular DNA or RNA refers only to the primary and secondary structure and it does not limit it to any particular tertiary forms.

In accordance with an aspect of the invention, an isolated nucleic acid molecule is provided of at least 30 nucleotides which hybridizes to one of SEQ ID NO. 1, 4, 7, 9, 11, 14, 16, or 18 through 34 or the complement of one of SEQ ID NO. 1, 4, 7, 9, 11, 14, 16, or 18 through 34 under stringent hybridization conditions.

In an embodiment of the invention an isolated nucleic acid molecule is contemplated which comprises:

(i) a nucleic acid sequence encoding a protein having substantial sequence identity with an amino acid sequence of SEQ. ID. NO. 2, 3, 5, 6, 8, 10, 12, 13, 15, or 17;

(ii) a nucleic acid sequence complementary to (i);

(iii) a nucleic acid sequence differing from any of (i) or (ii) in codon sequences due to the degeneracy of the genetic code;

(iv) a nucleic acid sequence comprising at least 10, preferably at least 15, more preferably at least 18, most preferably at least 20 nucleotides capable of hybridizing to a nucleic acid sequence of one of SEQ. ID. NO. 1, 4, 7, 9, 11, 14, 16, or 18 through 34 or to a degenerate form thereof;

(v) a nucleic acid sequence encoding a truncation, an analog, an allelic or species variation of a protein comprising the amino acid sequence of SEQ. ID. NO. 2, 3, 5, 6, 8, 10, 12, 13, 15, or 17; or

(vi) a fragment, or allelic or species variation of (i), (ii) or (iii)

In a specific embodiment, the isolated nucleic acid molecule comprises:

(i) a nucleic acid sequence having substantial sequence identity or sequence similarity with a nucleic acid sequence of one of SEQ. ID. NO. 1, 4, 7, 9, 11, 14, 16, or 18 through 34;

(ii) nucleic acid sequences comprising the sequence of one of SEQ. ID. NO. 1, 4, 7, 9, 11, 14, 16, or 18 through 34 wherein T can also be U;

(iii) nucleic acid sequences complementary to (i), preferably complementary to the full nucleic acid sequence of one of SEQ. ID. NO. 1, 4, 7, 9, 11, 14, 16, or 18 through 34;

(iv) nucleic acid sequences differing from any of the nucleic acid sequences of (i), (ii), or (iii) in codon sequences due to the degeneracy of the genetic code; or

(v) a fragment, or allelic or species variation of (i), (ii) or (iii).

In a preferred embodiment the isolated nucleic acid comprises a nucleic acid sequence encoded by the amino acid sequence of SEQ. ID. NO. 2, 3, 5, 6, 8, 10, 12, 13, 15, or 17, or comprises the nucleic acid sequence of one of SEQ. ID. NO. 1, 4, 7, 9, 11, 14, 16, or 18 through 34 wherein T can also be U. In another embodiment, the isolated nucleic acid comprises a nucleic acid sequence encoding the amino acid sequence of SEQ. ID. NO. 71, 73, 75 or 77 or comprises the nucleic acid sequence of SEQ. ID. NO. 70, 72, 74 or 76 wherein T can also be U.

Preferably, the nucleic acid molecules of the present invention have substantial sequence identity using the preferred computer programs cited herein, for example greater than 50% nucleic acid identity; preferably greater than 60% nucleic acid identity; and more preferably greater than 65%, 70%, 75%, 80%, or 85% sequence identity, most preferably at least 95%, 96%, 97%, 98%, or 99% sequence identity to the sequence of one of SEQ. ID. NO. 1, 4, 7, 9, 11, 14, 16, or 18 through 34.

Isolated nucleic acids encoding a PHI Protein, or part thereof and comprising a sequence that differs from the nucleic acid sequence of one of SEQ. ID. NO. 1, 4, 7, 9, 11, 14, 16, or 18 through 34, due to degeneracy in the genetic code are also within the scope of the invention. Such nucleic acids encode equivalent proteins. As one example, DNA sequence polymorphisms within a nucleic acid molecule of the invention may result in silent mutations that do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of a PHI Protein. These amino acid polymorphisms are also within the scope of the present invention. In addition, species variations i.e. variations in nucleotide sequence naturally occurring among different species, are within the scope of the invention.

Another aspect of the invention provides a nucleic acid molecule which hybridizes under selective conditions, (e.g. high stringency conditions), to a nucleic acid which comprises a sequence which encodes a PHI Protein, or part thereof. The sequence preferably encodes the amino acid sequence of SEQ. ID. NO. 2, 3, 5, 6, 8, 10, 12, 13, 15, or 17 and comprises at least 10, 15, 18, 20, 25, 30, 35, 40, 45 nucleotides, more typically at least 50 to 200 nucleotides. Selectivity of hybridization occurs with a certain degree of specificity rather than being random. Appropriate stringency conditions which promote DNA hybridization are known to those skilled in the art, or can be found in Current Protocols in Molecular Biology, John Wiley & Sons, N.Y. (1989), 6.3.1-6.3.6. For example,5.0 to 6.0×sodium chloride/sodium citrate (SSC) or 0.5% SDS at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

It will be appreciated that the invention includes nucleic acid molecules encoding a PHI Protein, including truncations of the proteins, allelic and species variants, and analogs of the proteins as described herein. In particular, fragments of a nucleic acid of the invention are contemplated that are a stretch of at least 10, 15, 18, 20, 25, 30, 35, 40, or 45 nucleotides, more typically at least 50 to 200 nucleotides but less than 2 kb. In an embodiment fragments are provided comprising nucleic acid sequences encoding a binding region of a PHI Protein, for example, the PH domain binding region (e.g. SEQ ID NO. 11), or IR binding region (e.g. SEQ ID NO. 14 or 16). It will further be appreciated that variant forms of the nucleic acid molecules of the invention which arise by alternative splicing of an mRNA corresponding to a cDNA of the invention are encompassed by the invention.

An isolated nucleic acid molecule of the invention which comprises DNA can be isolated by preparing a labeled nucleic acid probe based on all or part of the nucleic acid sequence of SEQ. ID. NO. 1, 4, 7, 9, 11, 14, 16, 18, 19, 20, 21, 22, 23, 24, 25, 26, 27, 28, 29, 30, 31, 32, 33, or 34. The labeled nucleic acid probe is used to screen an appropriate DNA library (e.g. a cDNA or genomic DNA library). For example, a cDNA library can be used to isolate a cDNA encoding a PHI Protein, by screening the library with the labeled probe using standard techniques. Alternatively, a genomic DNA library can be similarly screened to isolate a genomic clone encompassing a phip gene. Nucleic acids isolated by screening of a cDNA or genomic DNA library can be sequenced by standard techniques.

An isolated nucleic acid molecule of the invention that is DNA can also be isolated by selectively amplifying a nucleic acid of the invention. “Amplifying” or “amplification” refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.). In particular, it is possible to design synthetic oligonucleotide primers from the nucleotide sequence of SEQ. ID. NO. 1, 4, 7, 9, 11, 14, 16, 18, 19, 20, 21, 22, 23, 24, 25, 26, 27, 28, 29, 30, 31, 32, 33, or 34 for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a PHI Protein, into an appropriate vector which allows for transcription of the cDNA to produce an RNA molecule which encodes a PHI Protein. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

Nucleic acid molecules of the invention may be chemically synthesized using standard techniques. Methods of chemically synthesizing polydeoxynucleotides are known, including but not limited to solid-phase synthesis which like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

The nucleic acid molecules of the invention can be engineered using methods generally known in the art in order to alter PHI Protein encoding sequences for reasons including alterations that modify cloning, processing, or expression of a PHI Protein. The molecules may be engineered using DNA shuffling by random fragmentation and PCR reassembly of gene fragments and synthetic oligonucleotides. Site-directed mutagenesis may be used to introduce mutations, and insert new restriction sites, alter glycosylation patterns, change codon preference, produce splice variants, and the like.

Determination of whether a particular nucleic acid molecule encodes a PHI Protein, can be accomplished by expressing the cDNA in an appropriate host cell by standard techniques, and testing the expressed protein in the methods described herein. A cDNA encoding a PHI Protein, can be sequenced by standard techniques, such as dideoxynucleotide chain termination or Maxam-Gilbert chemical sequencing, to determine the nucleic acid sequence and the predicted amino acid sequence of the encoded protein.

The initiation codon and untranslated sequences of a nucleic acid molecule of the invention may be determined using computer software designed for the purpose, such as PC/Gene (IntelliGenetics Inc., Calif.). The intron-exon structure and the transcription regulatory sequences of a nucleic acid molecule of the invention may be identified by using a nucleic acid molecule of the invention to probe a genomic DNA clone library. (See SEQ. ID. NO. 69 showing the intron/exon structure of human PHIP and NDRP.) Regulatory elements can be identified using standard techniques. The function of the elements can be confirmed by using these elements to express a reporter gene such as the lacZ gene that is operatively linked to the elements. These constructs may be introduced into cultured cells using conventional procedures or into non-human transgenic animal models. In addition to identifying regulatory elements in DNA, such constructs may also be used to identify nuclear polypeptides interacting with the elements, using techniques known in the art.

The invention contemplates nucleic acid molecules comprising a regulatory sequence of a phip gene contained in appropriate vectors. The vectors may contain sequences encoding heterologous polypeptides. “Heterologous polypeptide” refers to a polypeptide not naturally located in the cell, i.e. it is foreign to the cell.

In accordance with another aspect of the invention, the nucleic acid molecules isolated using the methods described herein are mutant phip gene alleles. For example, the mutant alleles may be isolated from individuals either known or proposed to have a genotype that contributes to symptoms of a particular condition or disease (e.g. a disorder associated with insulin response, or cancer). Mutant alleles and mutant allele products may be used in therapeutic and diagnostic methods described herein. For example, a cDNA of a mutant phip gene may be isolated using PCR as described herein, and the DNA sequence of the mutant allele may be compared to the normal allele to ascertain the mutation(s) responsible for the loss or alteration of function of the mutant gene product A genomic library can also be constructed using DNA from an individual suspected of or known to carry a mutant allele, or a cDNA library can be constructed using RNA from tissue known, or suspected to express the mutant allele. A nucleic acid encoding a normal phip gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant phip allele. Gene products from putatively mutant tissue may be expressed and screened, for example using antibodies specific for a PHI Protein as described herein. Library clones identified using the antibodies can be purified and subjected to sequence analysis.

Nucleic acid molecules of the invention also include oligonucleotides and fragments thereof, complementary to strategic sites along a sense PHIP nucleic acid molecule, e.g. antisense oligonucleotides. Antisense oligonucleotides may be two to two hundred nucleotide bases long; more preferably ten to one hundred bases long, most preferably ten to forty bases long. Oligonucleotides are selected from complementary or substantially complementary oligonucleotides to strategic sites along a nucleic acid molecule of the invention (e.g. mRNA sense strand) that inhibit formation of a functional PHI Protein. Any combination or subcombination of antisense nucleic acid molecules that modulate a PHI Protein is suitable for use in the invention. The antisense oligonucleotides may also include nucleotides flanking the complementary or substantially complementary to strategic sites or other sites along a PHIP nucleic acid molecule. The flanking portions are preferably from about five to about fifty bases, preferably five to about twenty bases in length. It is also preferable that the antisense molecules be complementary to a non-conserved region of a PHIP nucleic acid molecule to minimize homology for nucleic acid molecules coding for other genes.

Sense and antisense oligonucleotides of the invention may comprise oligonucleotides having modified sugar-phosphodiester backbones (or other sugar linkages, such as those described in WO91/06629). Such sugar linkages may render the molecules resistant to endogenous nucleases. These oligonucleotides are relatively stable in vivo (i.e. capable of resisting enzymatic degradation) but retain their specificity for binding to target nucleotide sequences. The oligonucleotides may be covalently linked to molecules that increase affinity of the oligonucleotides for a target nucleic acid sequence, such as poly-(L-lysine). Intercalating agents, such as ellipticine, and alkylating agents or metal complexes may be linked to sense or antisense oligonucleotides to modify the binding specificity for a target sequence.

The invention also contemplates ribozymes, enzymatic RNA molecules, that function to inhibit translation of a PHI Protein or one or more molecules of a complex of the invention.

The antisense molecules and ribozymes contemplated within the scope of the invention may be prepared by any method known in the art for the synthesis of nucleic acid molecules. For example, techniques for chemically synthesizing oligonucleotides such as solid phase phosphoramidite chemical synthesis may be used. RNA molecules may also be generated by in vitro and in vivo transcription of DNA sequences encoding a PHI Protein. The DNA sequences may be incorporated into vectors with suitable RNA polymerase promoters including T7 or SP6. In the alternative, cDNA constructs that produce antisense RNA constitutively or inducibly can be introduced into cell lines, cells, or tissues. The RNA molecules can be modified to increase intracellular stability and half-life, for example, by adding flanking sequences at the 5′ and/or 3′ ends of the molecule, or using phosphorothioate or 2′ O-methyl rather than phosphodiesterase linkages within the backbone of the molecule. The molecules can also be modified by inserting nontraditional bases such as inosine, queosine, and wybutosine, or acetyl-, methyl-, thio-, and similarly modified forms of adenine, cytidine, guanine, thymine, and uridine which are not as readily recognized by endogenous endonucleases.

3. PHI Proteins

A PHI Protein is characterized by an N-terminal α-helical region predicting a coiled coil structure and a region containing two bromodomains. Amino acid sequences of PHI Protein comprise a sequence of SEQ.ID.NO. 2, 3, 5, 6, 8, 10, 12, 13, 15, 17, 71, 73, 75 or 77. “Amino acid sequences” refer to an oligopeptide, peptide, polypeptide or protein sequence and to naturally occurring or synthetic molecules.

In an embodiment of the invention an isolated PHI Protein is provided that is encoded by a nucleic acid molecule selected from:

(a) a nucleic acid molecule comprising SEQ ID NO. 1, 4, 7, 9, 11, 14, 16, 18, 19, 20, 21, 22, 23, 24, 25, 26, 27, 28, 29, 30, 31, 32, 33, or 34; and

(b) a nucleic acid molecule encoding a protein comprising SEQ ID NO: 2, 3, 5, 6, 8, 10, 12, 13, 15, or 17;

wherein the protein is capable of forming a stable interaction with a PH domain of insulin receptor substrate-1.

In preferred embodiments of the invention an isolated human PHIP is provided comprising SEQ ID NO. 2, 3, or 8, and a mouse PHIP is provided comprising SEQ ID NO. 5, 6, or 10. The PHIP of SEQ ID NOs. 8 and 10 are long forms of PHIP comprising a fusion of PHIP and neuronal differentiation-related protein (NDRP). The only difference with SEQ ID NOs. 2, 3, 5, and 6 is the N-terminal end which is encoded by different exons. The sequence diverges at amino acid position 4 of the short forms (SEQ.ID.NOs. 2 and 5) in both human and mouse sequences. The long form of PHIP contains N-terminal alternatively spliced sequences.

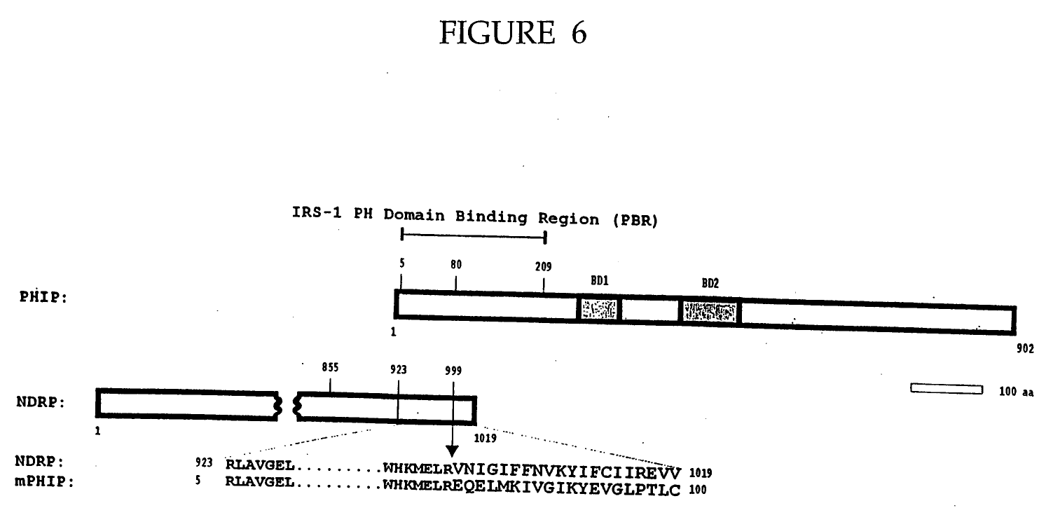

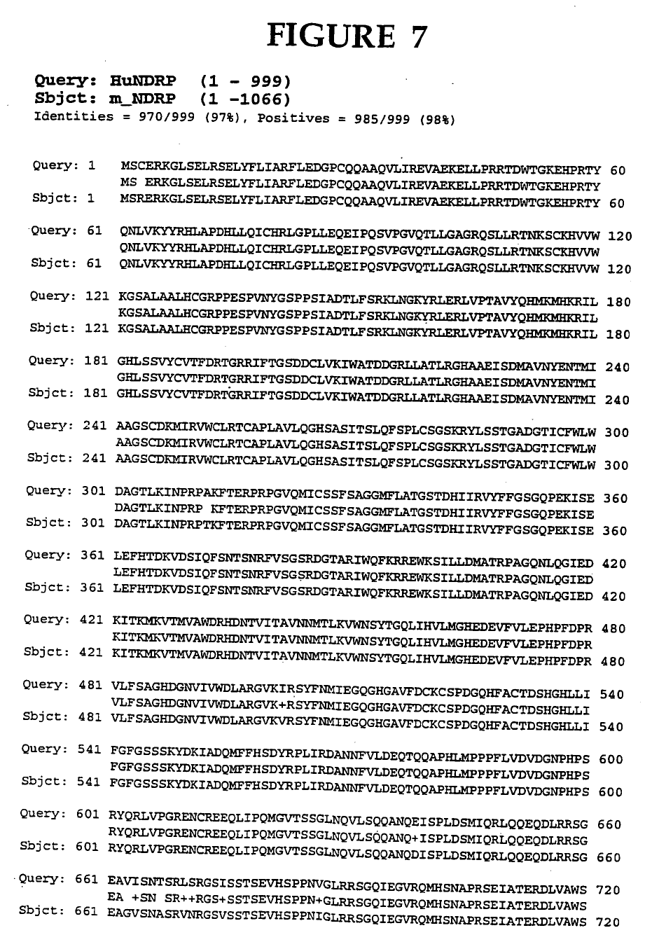

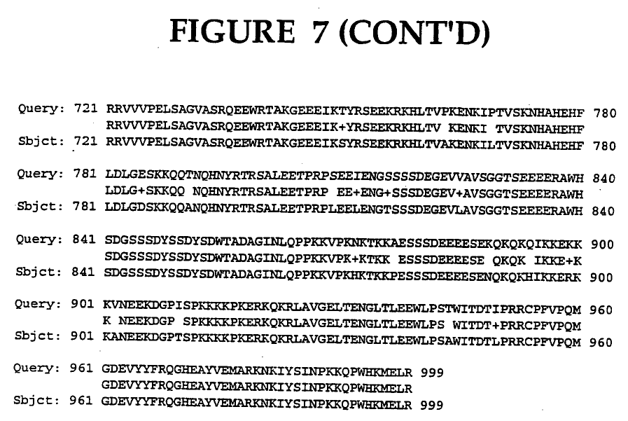

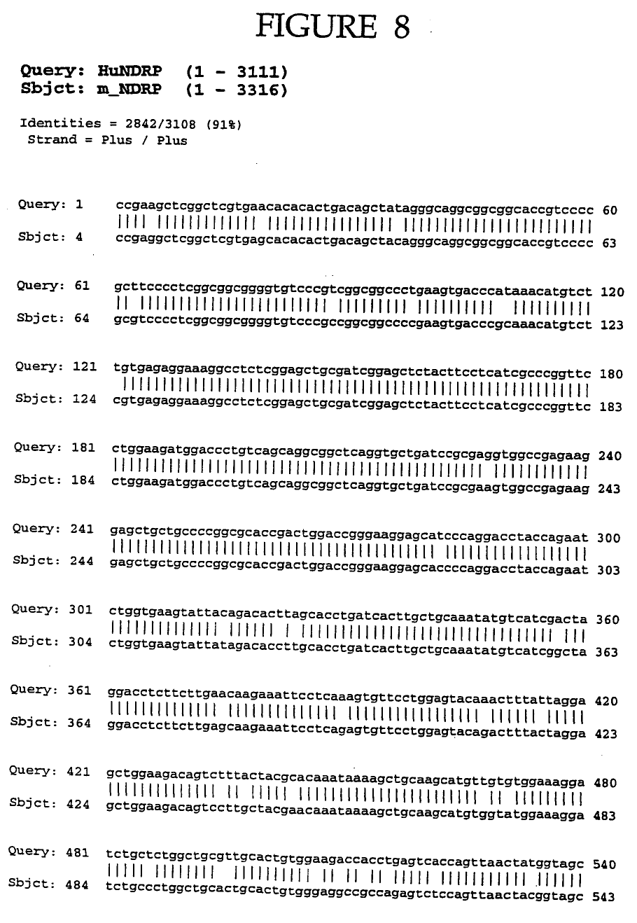

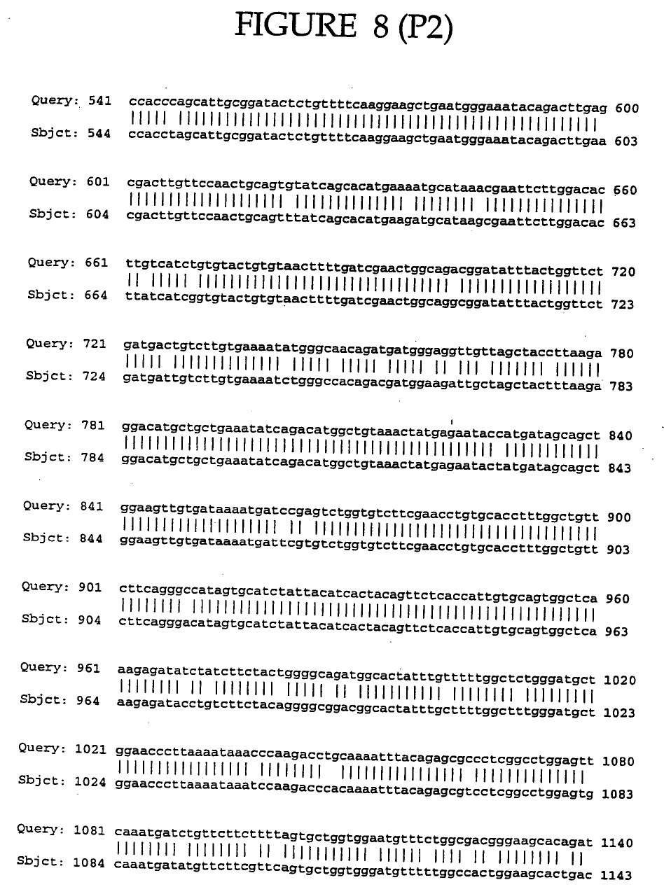

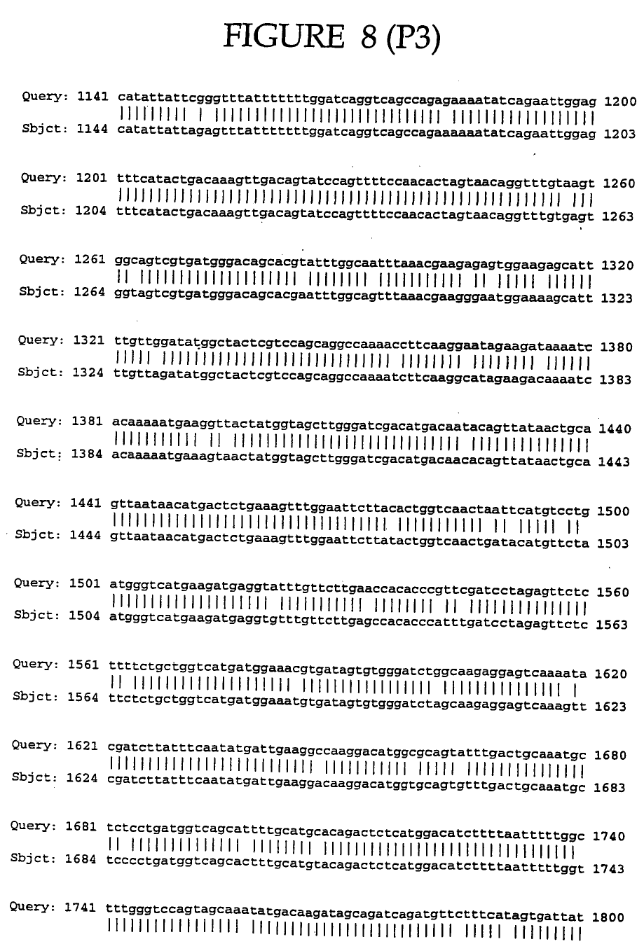

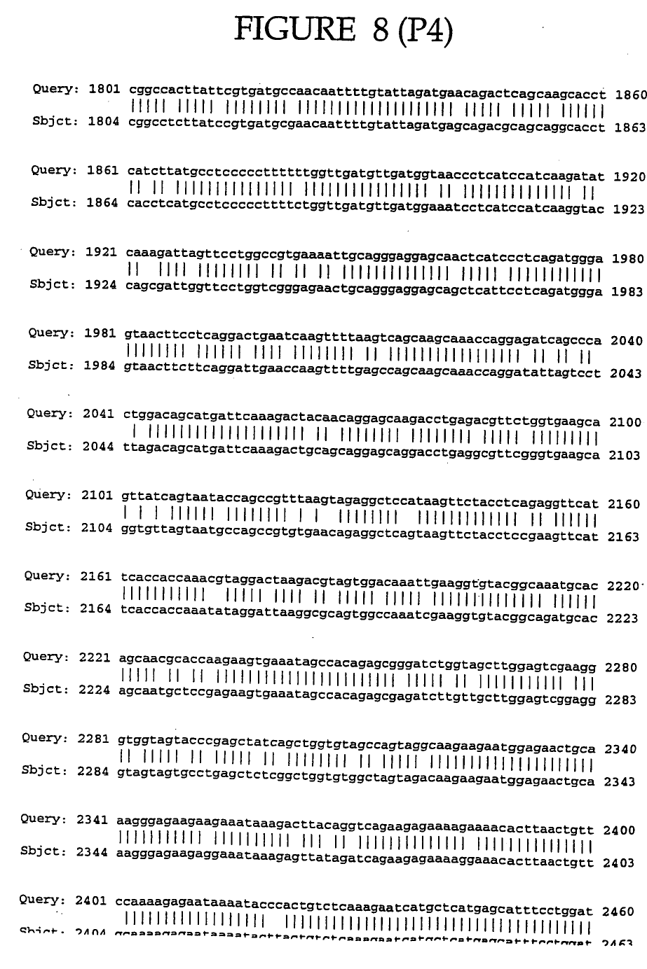

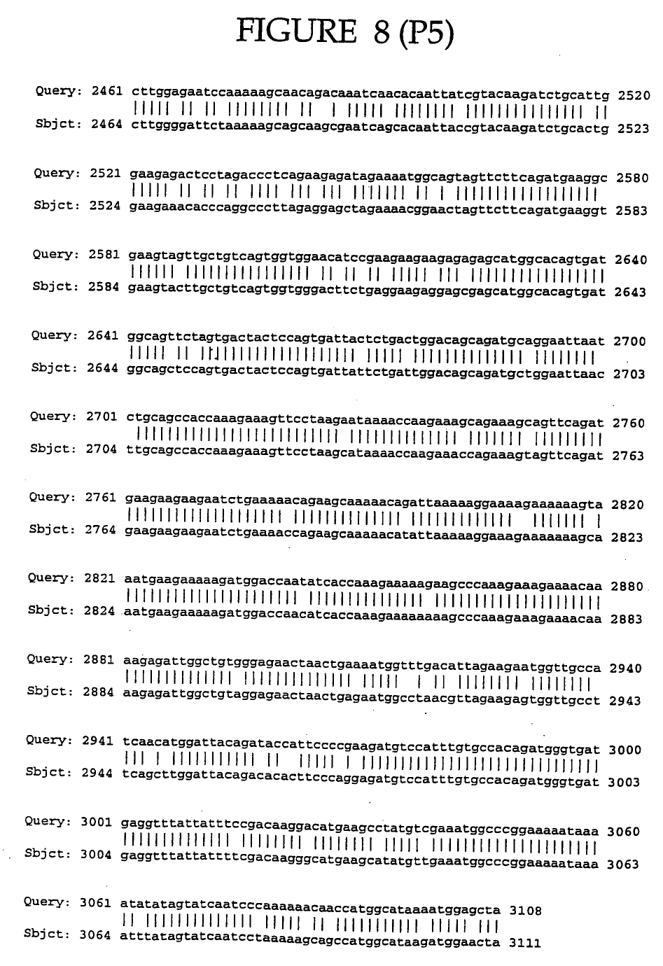

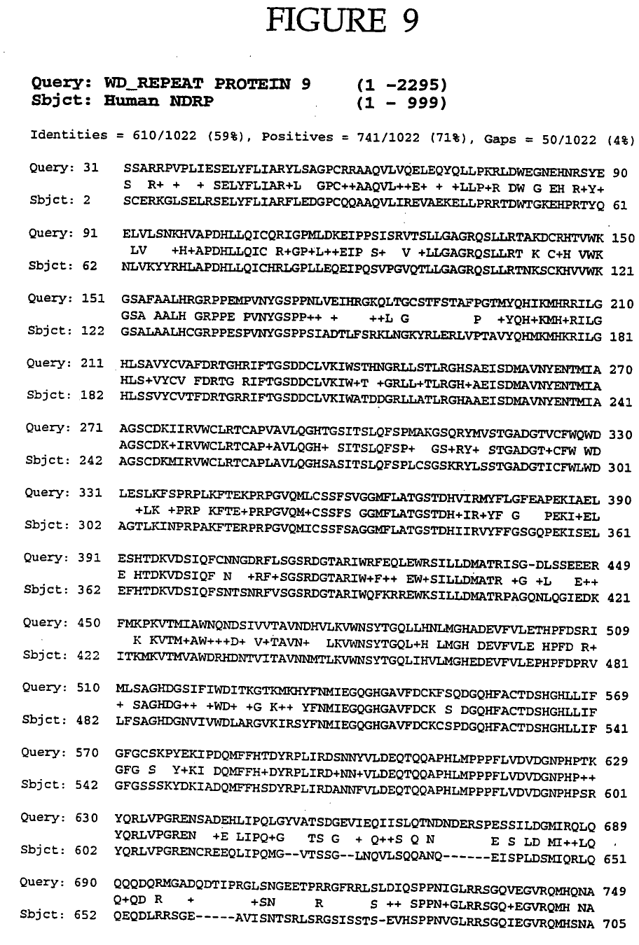

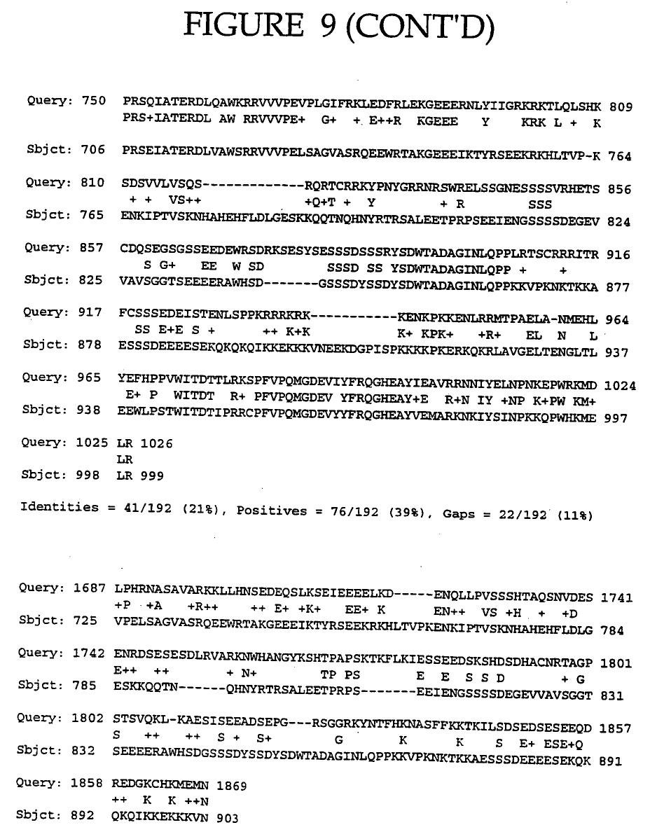

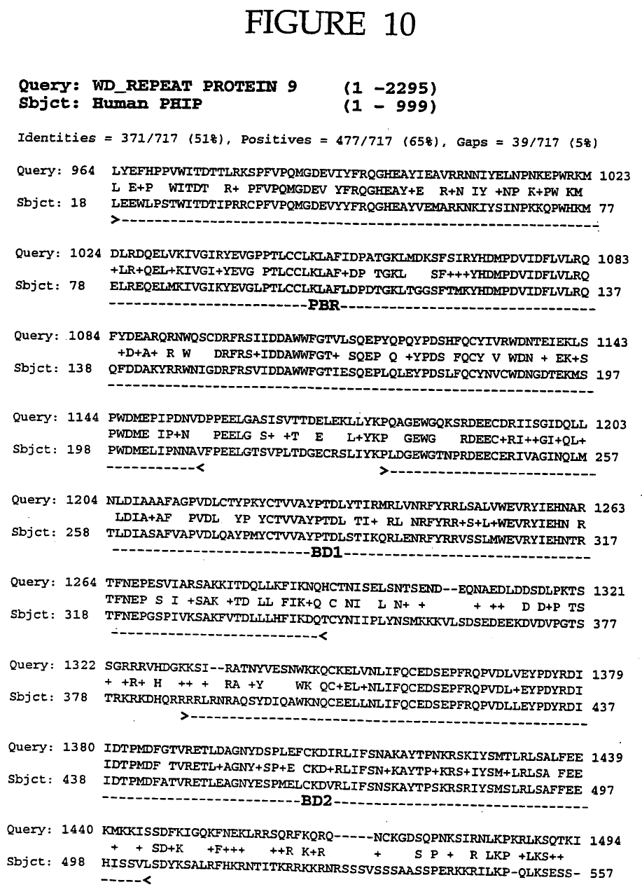

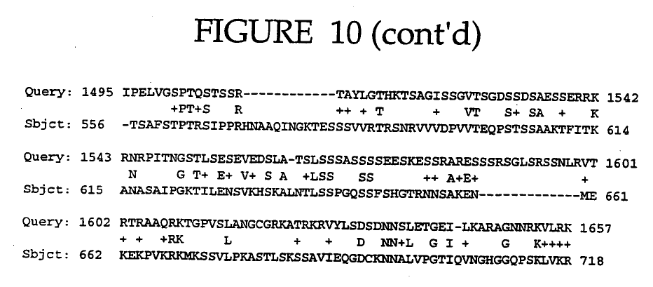

A second member of the PHI Protein family, neuronal differentiation-related protein (NDRP), was identified which is predominantly expressed in developing neurons and may be involved in neuronal regeneration and differentiation. The pre-carboxy terminal region of NDRP is identical to the amino-terminal region of PHIP (residues 5-80). (See FIGS. 6 and 7). This region may correspond to a conserved functional domain in NDRP. FIGS. 7 and 8 show alignments of the amino acid sequences and nucleic acid sequences of human and mouse NDRP, respectively. SEQ. ID. NO. 69 shows the introns and exons of PHIP and NDRP. The sequence shown is the complementary sequence. The introns are shown in black; PHIP exons are shown in blue; NDRP exons are shown in red; and PHIP/NDRP shared exons are shown in pink.

Therefore, the invention also relates to an isolated nucleic acid molecule comprises:

(vi) a nucleic acid sequence having substantial sequence identity or sequence similarity with a nucleic acid sequence of one of SEQ. ID. NO. 35, and 39 through 63;

(vii) nucleic acid sequences comprising the sequence of one of SEQ. ID. NO. 35, and 39 through 63, wherein T can also be U;

(viii) nucleic acid sequences complementary to (i), preferably complementary to the full nucleic acid sequence of one of SEQ. ID. NO. 35, and 39through 63;

(ix) nucleic acid sequences differing from any of the nucleic acid sequences of (i), (ii), or (iii) in codon sequences due to the degeneracy of the genetic code; or

(x) a fragment, or allelic or species variation of (i), (ii) or (iii).

An isolated neuronal differentiation-related protein is also provided that is encoded by:

(a) a nucleic acid molecule comprising one of SEQ ID NO. 35, and 39 through 63; or

(b) a nucleic acid molecule encoding a protein comprising SEQ ID NO: 36.

In preferred embodiments of the invention an isolated human NDRP is provided comprising SEQ ID NO. 36. The invention also includes truncations, analogs, proteins with substantial sequence identity, isoforms and mimetics of the NDRPs disclosed herein.

An ortholog of PHIP has also been identified which is referred to as “WDR9”. The full amino acid sequence for WDR9 is GenBank Accession No. Q9NSI6, and the nucleic acid sequence for WDR9 is spliced from the nucleic acid sequence of GenBank Accession No. AL163279. Partial amino acid sequences for WDR9 are shown in SEQ ID NO. 64 and NO. 65. Amino acid and nucleic acid sequence alignments of WD-Repeat Protein 9 and PHIP are shown in FIGS. 13, and 14, respectively.

In addition to proteins comprising an amino acid sequence of SEQ.ID.NO. 2, 3, 5, 6, 8, 10, 12, 13, 15, or 17, the PHI Proteins of the present invention include truncations of a PHI Protein, analogs of a PHI Protein, and proteins having sequence identity or similarity to a PHI Protein, and truncations thereof as described herein. Truncated proteins may comprise, for example, peptides of between 3 and 275 amino acid residues, ranging in size from a tripeptide to a 275 mer protein. In one aspect of the invention, fragments of a PHI Protein are provided having an amino acid sequence of at least five consecutive amino acids of SEQ.ID. NO.2, 3, 5, 6, 8, 10, 12, 13, 15, or 17 where no amino acid sequence of five or more, six or more, seven or more, or eight or more, consecutive amino acids present in the fragment is present in a polypeptide other than a PHI Protein. In an embodiment of the invention the fragment is a stretch of amino acid residues of at least 12 to 20 contiguous amino acids from particular sequences such as the sequences of SEQ.ID. NO. 2, 3, 5, 6, 8, 10, 12, 13, 15, or 17. The fragments maybe immunogenic and preferably are not immunoreactive with antibodies that are immunoreactive to polypeptides other than a PHI Protein. In an embodiment, the fragments comprise an amino acid sequence of a binding region of a PHI Protein, for example a PH domain binding region (e.g. SEQ ID NO 12 or 13), or an IR binding region (e.g. SEQ ID NO. 15 or 17). (Also see description of peptides herein.)

The proteins of the invention may also include analogs of a PHI Protein, and/or truncations thereof as described herein, which may include, but are not limited to a PHIP Protein, containing one or more amino acid substitutions, insertions, and/or deletions. Amino acid substitutions may be of a conserved or non-conserved nature. Conserved amino acid substitutions involve replacing one or more amino acids of a PHI Protein amino acid sequence with amino acids of similar charge, size, and/or hydrophobicity characteristics. When only conserved substitutions are made the resulting analog is preferably functionally equivalent to a PHI Protein. Non-conserved substitutions involve replacing one or more amino acids of a PHI Protein amino acid sequence with one or more amino acids which possess dissimilar charge, size, and/or hydrophobicity characteristics.

One or more amino acid insertions may be introduced into a PHI Protein. Amino acid insertions may consist of single amino acid residues or sequential amino acids ranging from 2 to 15 amino acids in length.

Deletions may consist of the removal of one or more amino acids, or discrete portions from a PHI Protein sequence. The deleted amino acids may or may not be contiguous. The lower limit length of the resulting analog with a deletion mutation is about 10 amino acids, preferably 20 to 40 amino acids. (Deletion mutants are described in Example 2 and in SEQ ID NOs. 67 and 68.)

An allelic variant at the polypeptide level differs from another polypeptide by only one, or at most, a few amino acid substitutions. A species variation of a PHI Protein of the invention is a variation which is naturally occurring among different species of an organism.

The proteins of the invention include proteins with sequence identity or similarity to a PHI Protein and/or truncations thereof as described herein. Such PHI Proteins may include proteins whose amino acid sequences are comprised of the amino acid sequences of PHIP Protein regions from other species that hybridize under selected hybridization conditions (see discussion of stringent hybridization conditions herein) with a probe used to obtain a PHI Protein. These proteins will generally have the same regions which are characteristic of a PHI Protein. Preferably a protein will have substantial sequence identity for example, about 65%, 70%, 75%, 80%, or 85% identity, preferably 90% identity, more preferably at least 95%, 96%, 97%, 98%, or 99% identity, and most preferably 98% identity with an amino acid sequence of SEQ.ID.NO. 2, 3, 5, 6, 8, 10, 12, 13, 15, or 17. A percent amino acid sequence homology, similarity or identity is calculated as the percentage of aligned amino acids that match the reference sequence using known methods as described herein. For example, a percent amino acid sequence homology or identity is calculated as the percentage of aligned amino acids that match the reference sequence, where the sequence alignment has been determined using the alignment algorithm of Dayhoff et al; Methods in Enzymology 91: 524-545 (1983).

The invention also contemplates isoforms of the proteins of the invention. An isoform contains the same number and kinds of amino acids as a protein of the invention, but the isoform has a different molecular structure. Isoforms contemplated by the present invention preferably have the same properties as a protein of the invention as described herein.

Still further the invention contemplates activated PHI Proteins. For example, a PHI Protein may be tyrosine phosphorylated or serine/threonine phosphorylated.

The invention provides molecules derived from a PHI Protein or binding region thereof. The molecules are preferably peptides derived from a PH domain binding region, an IR binding region, or a STAT binding region. In embodiments of the invention the peptides consist essentially of SEQ ID. NO. 12, 13, 15, or 17. Peptides may also be derived from a binding region of a PH domain containing protein, receptor that interacts with a protein of the IRS protein family, or STAT transcription factor, that interact with or bind directly or indirectly with a PHI Protein binding region.

All of these peptides, as well as molecules substantially homologous, complementary or otherwise functionally or structurally equivalent to these peptides may be used for purposes of the present invention. In addition to a full-length binding region (e.g. PH domain binding region, an IR binding region, or a STAT binding region), truncations of the peptides are contemplated. Truncated peptides may comprise peptides of about 5 to 200 amino acid residues, preferably 5 to 100 amino acid residues, more preferably 5 to 50 amino acid residues.

The invention also relates to novel chimeric proteins comprising at least one PHI Protein or peptide of the invention fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e. are heterologous). A target protein is a protein that is selected for insertion of a PH domain binding region, IR binding region, or STAT binding region, and for example may be a protein that is mutated or over expressed in a disease condition. The targeting domain can be a membrane spanning domain, a membrane binding domain, or a sequence directing the protein to associate with for example vesicles or with the nucleus. The targeting domain can target the chimeric protein to a particular cell type or tissue. For example, the targeting domain can be a cell surface ligand or an antibody against cell surface antigens of a target tissue (e.g. tumor antigens).

Cyclic derivatives of peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The relative flexibility of a cyclic peptide can be determined by molecular dynamics simulations.

Combined with certain formulations, peptides can be effective intracellular agents. However, in order to increase the efficacy of peptides, a fusion peptide can be prepared comprising a second peptide which promotes “transcytosis”, e.g. uptake of the peptide by epithelial cells. To illustrate, a peptide of the invention can be provided as part of a fusion polypeptide with all or a fragment of the N-terminal domain of the HIV protein Tat, e.g. residues 1-72 of Tat or a smaller fragment thereof which can promote transcytosis. In other embodiments, a peptide of the invention can be provided as a fusion polypeptide with all or a portion of an antennapedia protein. To further illustrate, a peptide of the invention can be provided as a chimeric peptide which includes a heterologous peptide sequence (“internalizing peptide”) which drives the translocation of an extracellular form of a peptide sequence across a cell membrane in order to facilitate intracellular localization of the peptide.

Hydrophilic polypeptides may be also be physiologically transported across the membrane barriers by coupling or conjugating the polypeptide to a transportable peptide which is capable of crossing the membrane by receptor-mediated transcytosis. Examples of internalizing peptides of this type can be generated using all or a portion of, e.g. a histone, insulin, transferrin, basic albumin, prolactin and insulin-like growth factor I (IGF-I), insulin-like growth factor II (IGF-II) or other growth factors.

Another class of translocating/internalizing peptides exhibits pH-dependent membrane binding. An example of a pH-dependent membrane-binding internalizing peptide in this regard is aa1-aa2-aa3-EAALA(EALA)4-EALEALAA-amide, which represents a modification of the peptide sequence of Subbarao et al. (Biochemistry 26:2964, 1987).

Internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, internalizing peptides may be modified through attachment of substituents that enhance the alpha-helical character of the internalizing peptide at acidic pH.

Other suitable internalizing peptides within the present invention include hydrophobic domains that are “hidden” at physiological pH, but are exposed in the low pH environment of the target cell endosome. Such internalizing peptides may be modeled after sequences identified in, e.g., Pseudomonas exotoxin A, clathrin, or Diphtheria toxin.

Pore-forming proteins or peptides may also serve as internalizing peptides. Pore-forming proteins or peptides may be obtained or derived from, for example, C9 complement protein, cytolytic T-cell molecules or NK-cell molecules.

Membrane intercalation of an internalizing peptide may be sufficient for translocation of the CPD peptide or peptidomimetic, across cell membranes. However, translocation may be improved by fusing to the internalizing peptide a substrate for intracellular enzymes (i.e., an “accessory peptide”). Suitable accessory peptides include peptides that are kinase substrates, peptides that possess a single positive charge, and peptides that contain sequences which are glycosylated by membrane-bound glycotransferases.

An accessory peptide can be used to enhance interaction of a peptide or peptide mimetic of the invention with a target cell. Examples of suitable accessory peptides for this use include peptides derived from cell adhesion proteins containing the sequence “RGD”, or peptides derived from laminin containing the sequence CDPGYIGSRC.

An internalizing and accessory peptide can each, independently, be added to a peptide or peptide mimetic of the present invention by either chemical cross-linking or in the form of a fusion protein. For fusion proteins, unstructured polypeptide linkers may be included between each of the peptide moieties.

An internalization peptide will generally be sufficient to also direct export of the polypeptide. However, when certain accessory peptides are used, such as an RGD sequence, it may be necessary to include a secretion signal sequence to direct export of the fusion protein from its host cell. A secretion signal sequence may be located at the extreme N-terminus, and is (optionally) flanked by a proteolytic site between the secretion signal and the rest of the fusion protein. In certain instances, it may also be desirable to include a nuclear localization signal as part of a peptide of the invention.

In the generation of fusion polypeptides including a peptide of the invention, it may be necessary to include unstructured linkers in order to ensure proper folding of the various peptide domains. Many synthetic and natural linkers are known in the art and can be adapted for use in the present invention, for example the (Gly₃Ser)₄ linker.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Peptides of the invention may be developed using a biological expression system. The use of such a system allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The invention contemplates peptide mimetics i.e. compounds based on, or derived from, peptides and proteins. Peptide mimetics of the present invention typically can be obtained by structural modification of a known PHI Protein sequence using unnatural amino acids, conformational restraints, isosteric replacement, and the like. The peptide mimetics constitute the continum of structural space between peptides and non-peptide synthetic structures; peptide mimetics of the invention may be useful, therefore, in delineating pharmacophores and in helping to translate peptides into nonpeptide compounds with the activity of the parent PHI peptides.

Moreover, mimetopes of peptides of the invention can be provided. Such peptide mimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. Peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), α-aminoalcohols (Gordon et al. (1985) Biochem Biophys Res Commun126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). (See generally, Session III: Analytic and synthetic methods, in in Peptides: Chemistry and Biology, G. R. Marshall ed., ESCOM Publisher: Leiden, Netherlands, 1988)

In addition to a variety of sidechain replacements which can be carried out to generate peptide mimetics, the present invention specifically contemplates the use of conformationally restrained mimics of peptide secondary structure. Many surrogates have been developed for the amide bond of peptides. Exemplary surrogates for the amide bond include the following groups (i) trans-olefins, (ii) fluoroalkene, (iii) methyleneamino, (iv) phosphonamides, and (v) sulfonamides. Peptide mimietics can also be based on more substantial modifications of the backbone of a PHI peptide. Peptide mimetics which are within this category include (i) retro-inverso analogs, and (ii) N-alkyl glycine analogs (so-called peptoids).

Combinatorial chemistry methods may also be brought to bear, c. f. Verdine et al. PCT publication WO9948897, on the development of new peptide mimetics. For example, a so-called “peptide morphing” strategy may be used that focuses on the random generation of a library of peptide analogs that comprise a wide range of peptide bond substitutes.

Another class of peptide mimetic derivatives include phosphonate derivatives. The synthesis of such phosphonate derivatives can be adapted from methods known by skilled artisans. (See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptide mimetic structures are known in the art and can be readily adapted for use in the present invention. A peptide mimetic of the invention may incorporate a 1-azabicyclo[4.3.0]nonane surrogate (see Kim et al. (1997) J. Org. Chem. 62:2847), an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348. Certain amino acid residues may be replaced with aryl and bi-aryl moieties, e.g., monocyclic or bicyclic aromatic or heteroaromatic nucleus, or a biaromatic, aromatic-heteroaromatic, or biheteroaromatic nucleus.

Peptide mimetics of the invention can be optimized by, e.g., combinatorial synthesis techniques combined with high throughput screening.

The present invention also includes PHI Proteins or peptides of the invention conjugated with a selected protein, or a marker protein (see below) to produce fusion proteins. Additionally, immunogenic portions of a PHI Protein or a peptide of the invention are within the scope of the invention.

A protein or peptide of the invention may be prepared using recombinant DNA methods. Accordingly, the nucleic acid molecules of the present invention having a sequence which encodes a protein or peptide of the invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used. Human artificial chromosomes (HACs) may be used to deliver larger fragments of DNA that can be contained and expressed in a plasmid.

The invention therefore contemplates a recombinant expression vector of the invention containing a nucleic acid molecule of the invention, and the necessary regulatory sequences for the transcription and translation of the inserted protein-sequence. Suitable regulatory sequences may be derived from a variety of sources, including bacterial, fungal, viral, mammalian, or insect genes [For example, see the regulatory sequences described in Goeddel, Gene Expression Technology: Methods in Enzymology 185, Academic Press, San Diego, Calif. (1990)]. Selection of appropriate regulatory sequences is dependent on the host cell chosen as discussed below, and may be readily accomplished by one of ordinary skill in the art. The necessary regulatory sequences may be supplied by the native protein and/or its flanking regions.

The invention further provides a recombinant expression vector comprising a DNA nucleic acid molecule of the invention cloned into the expression vector in an antisense orientation. That is, the DNA molecule is linked to a regulatory sequence in a manner which allows for expression, by transcription of the DNA molecule, of an RNA molecule which is antisense to the nucleic acid sequence of a protein of the invention or a fragment thereof. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The recombinant expression vectors of the invention may also contain a marker gene which facilitates the selection of host cells transformed or transfected with a recombinant molecule of the invention. Examples of marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The markers can be introduced on a separate vector from the nucleic acid of interest.

The recombinant expression vectors may also contain genes that encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinantproteinto allow separation of the recombinant protein from the fusion moiety subsequent to purification of the fusion protein. Typical fusion expression vectors include pET (Novagen) that have a histadine tag, pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

The recombinant expression vectors may be introduced into host cells to produce a transformant host cell. “Transformant host cells” include host cells which have been transformed or transfected with a recombinant expression vector of the invention. The terms “transformed with”, “transfected with”, “transformation” and “transfection” encompass the introduction of a nucleic acid (e.g. a vector) into a cell by one of many standard techniques. Prokaryotic cells can be transformed with a nucleic acid by, for example, electroporation or calcium-chloride mediated transformation. A nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g. cleaves) the protein in a desired fashion. Host systems or cell lines may be selected which have specific and characteristic mechanisms for post-translational processing and modification of proteins. For example, eukaryotic host cells including CHO, VERO, BHK, HeLA, COS, MDCK, 293, 3T3, and W138 may be used. For long-term high-yield stable expression of the protein, cell lines and host systems which stably express the gene product may be engineered.

Host cells and in particular cell lines produced using the methods described herein may be particularly useful in screening and evaluating compounds that modulate the activity of a PHI Protein.

A PHI Protein may be expressed in non-human transgenic animals including but not limited to mice, rats, rabbits, guinea pigs, micro-pigs, goats, sheep, pigs, non-human primates (e.g. baboons, monkeys, and chimpanzees) [see Hammer et al. (Nature 315:680-683, 1985), Palmiter et al. (Science 222:809-814, 1983), Brinster et al. (Proc Natl. Acad. Sci USA 82:44384442, 1985), Palmiter and Brinster (Cell. 41:343-345, 1985) and U.S. Pat. No. 4,736,866)]. Procedures known in the art may be used to introduce a nucleic acid molecule of the invention encoding a PHI Protein into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries the phip gene in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant PHI Protein in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissue may also be evaluated immunocy to chemically using antibodies against a PHI Protein.

Proteins or peptides of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

N-terminal or C-terminal fusion proteins comprising a protein or peptide of the invention conjugated with other molecules, such as proteins, may be prepared by fusing, through recombinant techniques, the N-terminal or C-terminal of a protein or peptide, and the sequence of a selected protein or marker protein with a desired biological function. The resultant fusion proteins contain the protein or peptide fused to the selected protein or marker protein as described herein. Examples of proteins which may be used to prepare fusion proteins include immunoglobulins, glutathione-S-transferase (GST), hemagglutinin (HA), and truncated myc.

4. Complexes of the Invention

A complex of the invention comprises a PHI protein or a binding region thereof, and a binding partner. A binding partner includes a PH domain containing protein, a receptor that interacts with a protein of the IRS protein family, and a STAT transcription factor, or a binding region thereof, that interacts with a PHI Protein or binding region thereof. In aspects of the invention complexes are provided comprising (a) a PHI Protein or a PH domain binding region, and a PH domain containing protein or a PH domain; (b) a PHI Protein or an IR binding region, and a receptor that interacts with a protein of the IRS protein family, or a binding region thereof; or, (c) a PHI Protein or a STAT binding region, and a STAT transcription factor or a binding region thereof that interacts with a PHI Protein. It will be appreciated that the complexes may comprise only the regions of the interacting molecules and such other flanking sequences as are necessary to maintain the activity of the complexes. Under physiological conditions the interacting molecules in a complex are capable of forming a stable, non-covalent interaction with the other molecules in the complex.

5. Antibodies

A PHI Protein, peptide, or complex of the invention can be used to prepare antibodies specific for the protein, peptide or complex. The invention can employ intact monoclonal or polyclonal antibodies, and immunologically active fragments (e.g. a Fab, (Fab)₂ fragment, or Fab expression library fragments and epitope-binding fragments thereof), an antibody heavy chain, and antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared which recognize a distinct epitope in an unconserved region of a PHI Protein. An unconserved region of the protein is one that does not have substantial sequence homology to other proteins. A region from a conserved region such as a well-characterized domain can also be used to prepare an antibody to a conserved region of a PHI Protein. Antibodies having specificity for a PHI Protein may also be raised from fusion proteins created by expressing fusion proteins in bacteria as described herein. In an embodiment, antibodies are prepared which are specific for a binding region of a PH Protein or a molecule in a complex of the invention.

Antibodies may be produced that are capable of specifically recognizing a complex or an epitope thereof, or of specifically recognizing an epitope on either of the interacting molecules of the complex, in particular epitopes that would not be recognized by the antibody when the molecules are present separate and apart from the complex. The antibodies may be capable of interfering with the formation of a complex of the invention and as described below they may be administered for the treatment of disorders involving a molecule capable of forming the complex with an interacting molecule (e.g. PHI Protein or binding region thereof, a PH domain, or PH domain containing protein).

Antibodies specific for a PHI Protein or complex of the invention may be used to detect PHI Protein or the complexes in tissues and to determine their tissue distribution. In vitro and in situ detection methods using the antibodies of the invention may be used to assist in the prognostic and/or diagnostic evaluation of conditions or diseases involving a PHI Protein, a complex of the invention, or a signal transduction pathway, including but not limited to proliferative and/or differentiative disorders associated with a PHI Protein or complex of the invention. Some genetic diseases may include mutations at the binding domain regions of the interacting molecules in the complexes of the invention. Therefore, if a complex of the invention is implicated in a genetic disorder, it may be possible to use PCR to amplify DNA from the binding regions to quickly check if a mutation is contained within one of the domains. Primers can be made corresponding to the flanking regions of the domains and standard sequencing methods can be employed to determine whether a mutation is present. This method does not require prior chromosome mapping of the affected gene and can save time by obviating sequencing the entire gene encoding a defective protein.

6. Applications

The nucleic acid molecules, PHI Proteins, antibodies, peptides, complexes compounds, substances and agents of the invention may be used in the prognostic and diagnostic evaluation of conditions and diseases mediated by a PHI Protein, a complex of the invention or an individual component thereof, or a signal transduction pathway, (e.g. cancer or disorders associated with insulin response), and the identification of subjects with a predisposition to such conditions or diseases (Section 6.1.1 and 6.1.2 below). Methods for detecting nucleic acid molecules and PHI Proteins of the invention, can be used to monitor diseases and conditions by detecting PHI Proteins and nucleic acid molecules encoding PHI Proteins. It would also be apparent to one skilled in the art that the methods described herein may be used to study the developmental expression of PHI Proteins and, accordingly, will provide further insight into the role of PHI Proteins. The applications of the present invention also include methods for the identification of compounds that modulate the biological activity of nucleic acid molecules encoding PHIP, PHI Proteins, peptides, complexes of the invention or components thereof, or mediate signal transduction pathways (e.g. IGF-R signaling pathways) (Section 6.2). The compounds, antibodies etc. may be used for the treatment of diseases and conditions mediated by a PHI Protein, a complex of the invention, or a signal transduction pathway (e.g. cancer or disorders associated with insulin response) (Section 6.3).

6.1 Diagnostic Methods

A variety of methods can be employed for the diagnostic and prognostic evaluation of diseases and conditions mediated by a PHI Protein, a complex of the invention or an individual component thereof, or a signal transduction pathway (e.g. cancer or disorders associated with insulin response), and the identification of subjects with a predisposition to such diseases and conditions. Such methods may, for example, utilize nucleic acid molecules of the invention, and fragments thereof, and antibodies directed against PHI Proteins, including peptide fragments, or complexes of the invention. In particular, the nucleic acids and antibodies may be used, for example, for (1) the detection of the presence of PHIP mutations, or the detection of either over- or under-expression of PHIP mRNA relative to anon-disorder state or the qualitative or quantitative detection of alternatively spliced forms of PHIP transcripts which may correlate with certain conditions or susceptibility toward such conditions; and (2) the detection of either an over- or an under-abundance of PHI Proteins relative to a non-disorder state or the presence of a modified (e.g., less than full length) PHI Protein which correlates with a disorder state, or a progression toward a disorder state.

The methods described herein may be performed by utilizing pre-packaged diagnostic kits comprising at least one nucleic acid molecule or antibody described herein, which may be conveniently used, e.g., in clinical settings, to screen and diagnose patients and to screen and identify those individuals exhibiting a predisposition to developing a disorder.

Nucleic acid-based detection techniques are described, below, in Section 6.1.1. Peptide detection techniques are described, below, in Section 6.1.2. The samples that may be analyzed using the methods of the invention include those which are known or suspected to express phip or contain PHI Proteins. The samples may be derived from a patient or a cell culture, and include but are not limited to biological fluids, tissue extracts, freshly harvested cells, and lysates of cells which have been incubated in cell cultures.

Oligonucleotides or longer fragments derived from any of the nucleic acid molecules of the invention may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505, Heller, R. A. et al. (1997) Proc. Natl. Acad. Sci. 94:2150-2155; and Heller, M. J. et al. (1997) U.S. Pat. No. 5,605,662.)

6.1.1 Methods for Detecting Nucleic Acid Molecules of the Invention

The nucleic acid molecules of the invention allow those skilled in the art to construct nucleotide probes for use in the detection of nucleic acid sequences of the invention in samples. Suitable probes include nucleic acid molecules based on nucleic acid sequences encoding at least 5 sequential amino acids from regions of the PHI Protein, preferably they comprise 15 to 30 nucleotides. A nucleotide probe may be labeled with a detectable substance such as a radioactive label which provides for an adequate signal and has sufficient half-life such as ³²P, ³H, ¹⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acidprobes may be used to detect genes, preferably in human cells, that encode PHI Proteins. The nucleotide probes may also be useful in the diagnosis of cancer, in monitoring the progression of diseases and conditions mediated by a PHI Protein, a complex of the invention, or a signal transduction pathway (e.g. cancer or disorders associated with insulin response); or monitoring a therapeutic treatment.

The probe may be used in hybridization techniques to detect genes that encode PHI Proteins. The technique generally involves contacting and incubating nucleic acids (e.g. recombinant DNA molecules, cloned genes) obtained from a sample from a patient or other cellular source with a probe of the present invention under conditions favorable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules of the invention may involve the amplification of specific gene sequences using an amplification method such as PCR, followed by the analysis of the amplified molecules using techniques known to those skilled in the art. Suitable primers can be routinely designed by one of skill in the art.

Genomic DNA may be used in hybridization or amplification assays of biological samples to detect abnormalities involving phip structure, including point mutations, insertions, deletions, and chromosomal rearrangements. For example, direct sequencing, single stranded conformational polymorphism analyses, heteroduplex analysis, denaturing gradient gel electrophoresis, chemical mismatch cleavage, and oligonucleotide hybridization may be utilized.

Genotyping techniques known to one skilled in the art can be used to type polymorphisms that are in close proximity to the mutations in a phip gene. The polymorphisms may be used to identify individuals in families that are likely to carry mutations. If a polymorphism exhibits linkage disequalibrium with mutations in a phip gene, it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

Hybridization and amplification techniques described herein may be used to assay qualitative and quantitative aspects of phip expression. For example, RNA may be isolated from a cell type or tissue known to express phip and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively spliced transcripts detected in normal individuals relative to those individuals exhibiting symptoms of a disease or condition (e.g. including cancer or a disorder associated with insulin response).

The primers and probes may be used in the above described methods in situ i.e directly on tissue sections (fixed and/or frozen) of patient tissue obtained from biopsies or resections.

6.1.2 Methods for Detecting PHI Proteins

Antibodies specifically reactive with a PHI Protein, or derivatives, such as enzyme conjugates or labeled derivatives, may be used to detect PHI Proteins in various samples (e.g. biological materials). They may be used as diagnostic or prognostic reagents and they may be used to detect abnormalities in the level of PHI Protein expression, or abnormalities in the structure, and/or temporal, tissue, cellular, or subcellular location of a PHI Protein. Antibodies may also be used to screen potentially therapeutic compounds in vitro to determine their effects on diseases and conditions mediated by a PHI Protein, a complex of the invention, or a signal transduction pathway (e.g. cancer or disorders associated with insulin response), and other conditions. In vitro immunoassays may also be used to assess or monitor the efficacy of particular therapies. The antibodies of the invention may also be used in vitro to determine the level of phip expression in cells genetically engineered to produce a PHI Protein.

The antibodies may be used in any known immunoassays which rely on the binding interaction between an antigenic determinant of a PHI Protein and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify PHI Proteins in a sample in order to determine its role in particular cellular events or pathological states, and to diagnose and treat such pathological states.

In particular, the antibodies of the invention may be used in immuno-histochemical analyses, for example, at the cellular and sub-subcellular level, to detect a PH Protein, to localize it to particular cells and tissues, and to specific subcellular locations, and to quantitate the level of expression.

Cytochemical techniques known in the art for localizing antigens using light and electron microscopy may be used to detect a PHI Protein. Generally, an antibody of the invention may be labeled with a detectable substance and a PHI Protein may be localised in tissues and cells based upon the presence of the detectable substance. Examples of detectable substances include, but are not limited to, the following: radioisotopes (e.g., ³ H, ¹⁴C., ³⁵S, ¹²⁵I, ¹³¹I, fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined protein epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a PHI Protein. By way of example, if the antibody having specificity against a PHI Protein is a rabbit IgG antibody, the second antibody may be goat anti-rabbit gamma-globulin labeled with a detectable substance as described herein.

Where a radioactive label is used as a detectable substance, a PHI Protein may be localized by radioautography. The results of radioautography may be quantitated by determining the density of particles in the radioautographs by various optical methods, or by counting the grains.

6.2 Methods for Identifying or Evaluating Substances/Compounds

The methods described herein are designed to screen for substances that modulate the biological activity of a PHI Protein including substances that interact with or bind with a PHI Protein, or interact with or bind with other proteins that interact with a PHI Protein, to compounds that interfere with, or enhance the interaction of a PHI Protein or interacting molecules in a complex, and substances that bind to a PHI Protein or other proteins that interact with a PHI Protein. Methods are also utilized that identify compounds that bind to phip regulatory sequences.

The substances and compounds identified using the methods of the invention include but are not limited to peptides such as soluble peptides including Ig-tailed fusion peptides, members of random peptide libraries and combinatorialchemistry-derived molecular libraries made of D- and/or L-configuration amino acids, polysaccharides, oligosaccharides, monosaccharides, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The substance or compound may be an endogenous physiological compound or it may be a natural or synthetic compound.

Substances can be screened based on their ability to interact with or bind to a PHI Protein or binding region thereof. Therefore, the invention also provides methods for identifying substances which interact with or bind to PHI Proteins. Substances identified using the methods of the invention may be isolated, cloned and sequenced using conventional techniques. A substance that interacts with a protein of the invention may be an agonist or antagonist of the biological or immunological activity of a PHI Protein.

Substances which can interact with or bind to a PHI Protein may be identified by reacting a PHI Protein or a binding region thereof, with a test substance which potentially interacts with or binds to a PHI Protein or binding region, under conditions which permit the formation of substance-PHI Protein or binding region complexes and removing and/or detecting the complexes. The complexes can be detected by assaying for PHI Protein or binding region complexes, for free substance, or for non-complexed PHI Proteins or binding regions. Conditions which permit the formation of substance-PHI Protein or binding region complexes may be selected having regard to factors such as the nature and amounts of the substance and the protein.

The substance-protein or binding region complex, free substance or non-complexed proteins or binding regions may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against PHI Proteins or a binding region thereof, or the substance, or labeled PHI Proteins or binding regions, or a labeled substance may be utilized. The antibodies, proteins, or substances may be labeled with a detectable substance as described above.

A PHI Protein or binding region, or the substance used in the method of the invention may be insolubilized. For example, a PHI Protein, binding region, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein, binding region, or substance may be prepared by reacting the material with a suitable insoluble carrier using known chemical or physical methods, for example, cyanogen bromide coupling.

It is possible to screen for agents that can be tested for their ability to treat a disease or condition characterized by an abnormality in a signal transduction pathway by testing compounds for their ability to affect the interaction between a PHI Protein and a binding partner, wherein the complex formed by such an interaction is part of the signal transduction pathway.

The interaction between a PHI Protein and a binding partner may be promoted or enhanced either by increasing production of a PHI Protein or binding partner, or by increasing expression of a PHI Protein or binding partner, or by promoting interaction of a PHI Protein and a binding partner, or by prolonging the duration of the interaction. The interaction between a PHI Protein and binding partner may be disrupted or reduced by preventing production of a PHI Protein or binding partner, or by preventing expression of a PHI Protein or binding partner, or by preventing interaction of a PHI Protein and binding partner, or interfering with the interaction. A method may also include measuring or detecting various properties including the level of signal transduction and the level of interaction between a PHI Protein and a binding partner. Depending upon the type of interaction present various methods may be used to measure the level of interaction. For example, the strengths of covalent bonds may be measured in terms of the energy required to break a certain number of bonds. Non-covalent interactions may be described as above and also in terms of the distance between the interacting molecules. Indirect interactions may be described in different ways including the number of intermediary agents involved, or the degree of control exercised over the PHI Protein relative to the control exercised over the binding partner.

The invention also contemplates a method for screening by assaying for an agonist or antagonist of the interaction of, or binding of, a PHI Protein or binding region thereof (e.g. PH domain binding region, IR binding region, or STAT binding region) with a substance which interacts with or binds with a PHI Protein or binding region thereof (e.g. binding partners including but not limited to a PH domain containing protein, a PH domain, a receptor that interacts with a protein of the IRS protein family, or STAT transcription factor). The basic method for evaluating if a compound is an agonist or antagonist of the interaction or binding of a PHI Protein or binding region thereof and a substance that binds to the protein, is to prepare a reaction mixture containing the PHI Protein or binding region thereof and the substance under conditions which permit the formation of substance PHI Protein or binding region complexes, in the presence of a test compound. The test compound may be initially added to the mixture, or may be added subsequent to the addition of the PHI Protein or binding region, and substance. Control reaction mixtures without the test compound or with a placebo are also prepared. The formation of complexes is detected and the formation of complexes in the control reaction but not in the reaction mixture, or the formation of more complexes in the control reaction compared to the reaction mixture, indicates that the test compound interferes with the interaction of the PHI Protein or binding region and substance. The reactions may be carried out in the liquid phase or the PHI Protein, binding region, substance, or test compound may be immobilized as described herein. The ability of a compound to modulate the biological activity of a PHI Protein or complex of the invention may be tested by determining the biological effects on cells or organisms using techniques known in the art.

It will be understood that the agonists and antagonists that can be assayed using the methods of the invention may act on one or more binding regions on a PHI Protein or substance including agonist binding sites, competitive antagonist binding sites, non-competitive antagonist binding regions or allosteric sites.

The invention also makes it possible to screen for antagonists that inhibit the effects of an agonist of the interaction of a PHI Protein or binding region thereof, with a substance which is capable of binding to a PHI Protein or binding region thereof. Thus, the invention may be used to assay for a compound that competes for the same binding site of a PHI Protein.

The invention also contemplates methods for identifying compounds that bind to proteins that interact with a PHI Protein. Protein-protein interactions may be identified using conventional methods such as co-immunoprecipitation, crosslinking and co-purification through gradients or chromatographic columns. Methods may also be employed that result in the simultaneous identification of genes which encode proteins interacting with a PHI Protein. These methods include probing expression libraries with labeled PHI Proteins. Additionally, x-ray crystallographic studies may be used as a means of evaluating interactions with substances and PHI Proteins. For example, purified recombinant molecules in a complex of the invention when crystallized in a suitable form are amenable to detection of intra-molecular interactions by x-ray crystallography. Spectroscopy may also be used to detect interactions and in particular, Q-TOF instrumentation may be used. Two-hybrid systems may also be used to detect protein interactions in vivo.

It will be appreciated that fusion proteins may be used in the above-described methods. For example, PHI Proteins fused to a glutathione-S-transferase may be used in the methods.

It will also be appreciated that the complexes of the invention may be reconstituted in vitro using recombinant molecules and the effect of a test substance may be evaluated in the reconstituted system.

The reagents suitable for applying the methods of the invention to evaluate compounds that modulate a PHI Protein may be packaged into convenient kits providing the necessary materials packaged into suitable containers. The kits may also include suitable supports useful in performing the methods of the invention.

Peptides of the invention may be used to identify lead compounds for drug development. The structure of the peptides of the invention can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds that can be tested for predicted properties as related to the target molecule.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess desired activities.

In an aspect of the invention, a method using a PHI Protein, a binding partner, or a binding region of a PHI Protein or binding partner to design small molecule mimetics, agonists, or antagonists is provided comprising determining the three dimensional structure of a PHI Protein, binding partner, or binding region and providing a small molecule or peptide capable of binding to the PHI Protein, binding partner, or binding region. Those skilled in the art will be able to produce small molecules or peptides that mimic the effect of the PHI Protein, binding partner, or binding region and that are capable of easily entering the cell. Once a molecule is identified, the molecule can be assayed for its ability to bind a PHI Protein, binding partner, or binding region, and the strength of the interaction may be optimized by making amino acid deletions, additions, or substitutions or by adding, deleting or substituting a functional group. The additions, deletions, or modifications can be made at random or may be based on knowledge of the size, shape, and three-dimensional structure of the PHI Protein, binding partner, or binding region.

Computer modelling techniques known in the art may also be used to observe the interaction of a PHI Protein, or binding region thereof, or agent, substance or compound identified in accordance with a method of the invention, with an interacting molecule or binding partner (e.g. an IRS protein family member, a receptor that interacts with a protein of the IRS protein family, or STAT transcription factor, or binding region thereof). (For example, Homology Insight II and Discovery available from BioSym/Molecular Simulations, San Diego, Calif., U.S.A. may be used for modelling). If computer modelling indicates a strong interaction, an agent, substance, compound or peptide can be synthesized and tested for its ability to interfere with the binding of a PHI Protein or binding region thereof with an interacting molecule or binding partner.

6.3 Compositions and Treatments

PHI Proteins, peptides, and complexes of the invention, and substances or compounds identified by the methods described herein, antibodies, and antisense nucleic acid molecules of the invention may be used for modulating the biological activity of a PHI Protein, a complex of the invention or individual components of the complex, or a signal transduction pathway, and they may be used in the prognostic and diagnostic evaluation of diseases and conditions mediated by a PHI Protein, a complex of the invention or an individual component of the complex, or a signal transduction pathway.

PHIP potentiates the effects of insulin on gene expression and mitogenesis, transcriptional responses, DNA synthesis, actin remodeling, and glucose transporter translocation. DN PHIP mutants completely block insulin mediated transciptional responses and DNA synthesis. This inhibitory effect of DN PHIP is very specific to the insulin receptor family. Specifically serum stimulated transcriptional and mitogenic responses are refractile to the effects of DN PHIP. Thus, PHIP is a useful target for therapeutic intervention in conditions or disorders associated with insulin response.

Thus, a protein, peptide, or complex of the invention, or substance or compound identified by the methods described herein, antibodies, and antisense nucleic acid molecules of the invention may be administered to a subject to prevent or treat a disorder associated with insulin response. Examples of these disorders include but are not limited to type 2 (non-insulin-dependent) diabetes mellitus, hyperglycemia, myotonic muscular dystrophy, acanthosis, nigricans, retinopathy, nephropathy, artherosclerotic coronary and peripheral arterial disease, and peripheral and autonomic neuropathies.

A protein, peptide, or complex of the invention or a substance or compound identified by the methods described herein, antibodies, and antisense nucleic acid molecules of the invention may be administered to a subject to prevent or treat cancer. Cancers that may be prevented or treated include but are not limited to adenocarcinoma, leukemia, lymphoma, melanoma, myeloma, sarcoma, and teratocarcinoma, and in particular cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus, preferably breast, prostate, colon, and ovarian carcinomas. In particular, cancers that may be prevented or treated in accordance with the invention are tumors dependent on receptors that interact with proteins of the IRS protein family, preferably IGF-1 mediated cancers.

A protein, peptide, or complex of the invention or a substance, agent, or compound identified by the methods described herein, antibodies, and antisense nucleic acid molecules of the invention may also be useful in treating or preventing other conditions including infectious diseases, autoimmune diseases, immune deficiency diseases, and inflammation.

In accordance with one aspect, antibodies which bind a PHI Protein may be used directly as an antagonist or indirectly as a targeting or delivery mechanism for bringing a pharmaceutical agent to cells or tissues which express a PHI Protein. In another aspect, a peptide of the invention, or a vector expressing the complement of a nucleic acid molecule encoding a PHI Protein i.e. antisense oligonucleotide, may be administered to a subject to treat or prevent cancer.

The disruption or promotion of the interaction between the molecules in complexes of the invention is also useful in therapeutic procedures. Therefore, the invention features a method for treating a subject having a condition characterized by an abnormality in a signal transduction pathway involving the interaction of a PHI Protein or a binding region thereof and a binding partner. In embodiments of this method, the interaction involves a PHI Protein or a PH domain binding region and a PH domain containing protein or a PH domain; a PHI Protein or an IR binding region and a receptor that interacts with a protein of the IRS protein family; or, a PHI Protein or a STAT binding region, and a STAT transcription factor or a binding region thereof that interacts with a PHI Protein.

The abnormality may be characterized by an abnormal level of interaction between the interacting molecules in a complex of the invention. An abnormality may be characterized by an excess amount, intensity, or duration of signal or a deficient amount, intensity, or duration of signal. An abnormality in signal transduction may be realized as an abnormality in cell function, viability, or differentiation state. The method involves disrupting or promoting the interaction (or signal) in vivo, or the activity of a complex of the invention. A compound that will be useful for treating a disease or condition characterized by an abnormality in a signal transduction pathway involving a complex of the invention can be identified by testing the ability of the compound to affect (i.e disrupt or promote) the interaction between the molecules in a complex. The compound may promote the interaction by increasing the production of a PHI Protein, or by increasing expression of a PH domain, or by promoting the interaction of the molecules in the complex. The compound may disrupt the interaction by reducing the production of a PHI Protein, preventing expression of a PH domain, or by specifically preventing interaction of the molecules in the complex.

In an embodiment of the invention the PHI Proteins, peptides, and complexes of the invention, and substances, agents, or compounds identified by the methods described herein, antibodies, and antisense nucleic acid molecules of the invention are used to modulate an IGFR signaling pathway. IGF-1 exerts pleiotropic effects on cellular processes through its stimulation of IGFR, a receptor tyrosine kinase. The activated IGF-1/IGFR system displays mitogenic, transforming, and anti-apoptotic properties in various cell types. Dysregulation of IGFR signaling pathways has been found to contribute to the development and metastatic dissemination of breast, colon, pancreatic, prostate, testicular, and ovarian carcinomas. The anti-apoptotic effect of IGF-1R may also mediate decreased sensitivity to chemotherapeutic drugs.

Therefore, the invention provides a method for preventing and treating tumor cell growth and metastasis in a subject comprising administering a PHI Protein, peptide, complex, agent, antibody, substance, or compound of the invention, preferably a peptide of the invention, most preferably a peptide comprising or consisting essentially of a PH domain binding region, in an amount effective to reduce the oncogenic properties of IGFR or reduce or inhibit IGF-1 mediated transformation.

In another aspect of the invention, a vector expressing the complement of a nucleic acid molecule encoding a PHI Protein i.e. antisense oligonucleotide, may be administered to a subject in an amount effective to treat or prevent tumor cell growth and metastasis by reducing the oncogenic properties of IGFR, or reducing or inhibiting IGF-1 mediated transformation.

In yet another aspect of the invention, a method is provided for enhancing the sensitivity of tumor cells to a pro-apoptotic agent in a subject comprising administering an effective amount of a PHI Protein, peptide, complex, or nucleic acid molecule of the invention, preferably a peptide or antisense oligonucleotide of the invention. An effective amount is the amount necessary to reduce the anti-apoptotic effect of IGF-IR against pro-apoptotic agents. Examples of pro-apoptotic agents include taxol, doxorubicin, etoposide, cisplatin, vinblastin, methotrexate, 5′ fluorouracil, camptothecin, mitoxanthone, cytosine arabinoside, cyclophosphamide, and paclitaxel.

A protein of the invention, peptide, complex, substance or compound identified by the methods described herein, antibodies, and antisense nucleic acid molecules of the invention may be administered in combination with other appropriate therapeutic agents (See discussion above re pro-apoptotic agents). The appropriate agents for use in combination therapy can be selected by a person skilled in the art based on conventional pharmaceutical principles. The combination of pharmaceutical agents may act synergistically to effect the treatment and prevention of conditions described herein. Combination therapy may enable one to achieve therapeutic efficacy with lower dosages of each agent thereby reducing potential adverse side effects.

The proteins, substances, antibodies, complexes, peptides, agents, antibodies, and compounds can be administered to a subject either by themselves, or they can be formulated into pharmaceutical compositions for administration to subjects in a biologically compatible form suitable for administration in vivo. By “biologically compatible form suitable for administration in vivo” is meant a form of the active substance to be administered in which any toxic effects are outweighed by the therapeutic effects. Administration of a therapeutically active amount of a pharmaceutical composition of the present invention is defined as an amount effective, at dosages and for periods of time necessary to achieve the desired result For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The pharmaceutical compositions or active agents contained therein may be administered to subjects including humans, and animals (e.g. dogs, cats, cows, sheep, horses, rabbits, and monkeys). Preferably, they are administered to human and veterinary patients.

An active substance may be administered in a convenient manner such as by injection (subcutaneous, intravenous, etc.), oral administration, inhalation, transdermal application, or rectal administration. Depending on the route of administration, an active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

Vectors derived from a retrovirus, adenovirus, herpes or vaccinia virus, papovavirus, adeno-associated virus, of avian, murine, or human origin, or from various bacterial plasmids, may be used to deliver nucleic acid molecules of the invention to a targeted organ, tissue, or cell population. Methods well known to those skilled in the art may be used to construct recombinant vectors which will express nucleic acid molecules of the invention (e.g. nucleic acid molecules encoding PHIP, a PH domain binding region, or antisense nucleic acid molecules). (See, for example, the techniques described in Sambrook et al (supra) and Ausubel et al (supra)).

The nucleic acid molecules comprising full length cDNA sequences and/or their regulatory elements enable a skilled artisan to use sequences encoding a PHI Protein as an investigative tool in sense (Youssoufian H and H F Lodish 1993 Mol Cell Biol 13:98-104) or antisense (Eguchi et al (1991) Annu Rev Biochem 60:631-652) regulation of gene function. Such technology is well known in the art, and sense or antisense oligomers, or larger fragments, can be designed from various locations along the coding or control regions.

Genes encoding a PHI Protein can be turned off by transfecting a cell or tissue with vectors which express high levels of a desired nucleic acid molecule of the invention. Such constructs can inundate cells with untranslatable sense or antisense sequences. Even in the absence of integration into the DNA, such vectors may continue to transcribe RNA molecules until all copies are disabled by endogenous nucleases. Modifications of gene expression can be obtained by designing antisense molecules, DNA, RNA or PNA, to the regulatory regions of a gene encoding a protein of the invention, ie, the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic uses of triplex DNA are reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a protein of the invention.

Specific ribozyme cleavage sites within an RNA target may initially be identified by scanning the target molecule for ribozyme cleavage sites including the following sequences: GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. A vector of the invention may be administered to a subject to correct a genetic condition characterized by a defective or nonexistent PHI Protein or complex of the invention. Cell populations of a subject may also be modified by introducing altered forms of a PHI Protein or binding region thereof, or complex of the invention in order to modulate the activity of the protein or complex. Inhibiting a PHI Protein or complex of the invention within the cells, may decrease, inhibit, or reverse a signal transduction pathway event that leads to a condition or disease. Deletion or missense mutants of a PHI Protein that retain the ability of the PHI Protein to interact with other molecules but cannot retain their function in signal transduction maybe used to inhibit an abnormal, deleterious signal transduction pathway event

The invention contemplates products and methods for performing PHI Protein related gene therapy and gene transfer techniques, including cell lines and transgenic mice (i.e. knock-out) mice for performing such techniques. The selection of transfected lineages, vectors, and targets may be confirmed in mouse models.

For ex vivo therapy, vectors may be introduced into cells obtained from a patient and clonally propagated for autologous transplant into the same patient (See U.S. Pat. Nos. 5,399,493 and 5,437,994). Delivery by transfection and by liposome are well known in the art. Therefore, the invention contemplates a method of administering a nucleic acid molecule of the invention to a subject comprising the steps of removing cells from the animal, transducing the cells with the nucleic acid molecule, and reimplanting the transduced cells into the animal.

The invention also provides a method of administering a nucleic acid molecule of the invention using an in vivo approach comprising the steps of administering directly to the subject the nucleic acid molecule selected from the group of methods consisting of intravenous injection, intramuscular injection, or by catheterization and direct delivery of the nucleic acid molecule. The nucleic acid may encode a human protein or peptide, and the subject to which the nucleic acid is administered may be a human. The nucleic acid may be administered as naked DNA or may be contained in a viral vector. The nucleic acid molecule may be administered in a two-component system comprising administering a packaging cell which produces a viral vector. The packaging cell may be administered to cells in vitro.

The nucleic acid molecules of the invention may also be used in molecular biology techniques that have not yet been developed, provided the new techniques rely on properties of nucleotide sequences that are currently known, including but not limited to such properties as the triplet genetic code and specific base pair interactions.

The invention also provides methods for studying the function of a protein of the invention. Cells, tissues, and non-human animals lacking in expression or partially lacking in expression of a nucleic acid molecule or gene of the invention may be developed using recombinant expression vectors of the invention having specific deletion or insertion mutations in the gene. A recombinant expression vector may be used to inactivate or alter the endogenous gene by homologous recombination, and thereby create a deficient cell, tissue, or animal.

Null alleles may be generated in cells, such as embryonic stem cells by deletion mutation. A recombinant gene may also be engineered to contain an insertion mutation that inactivates the gene. Such a construct may then be introduced into a cell, such as an embryonic stem cell, by a technique such as transfection, electroporation, injection, etc. Cells lacking an intact gene may then be identified, for example by Southern blotting, Northern Blotting, or by assaying for expression of the encoded protein using the methods described herein. Such cells may then be fused to embryonic stem cells to generate transgenic non-human animals deficient in a protein of the invention. Germline transmission of the mutation may be achieved, for example, by aggregating the embryonic stem cells with early stage embryos, such as 8 cell embryos, in vitro transferring the resulting blastocysts into recipient females and; generating germline transmission of the resulting aggregation chimeras. Such a mutant animal may be used to define specific cell populations, developmental patterns and in vivo processes, normally dependent on gene expression.

The invention thus provides a transgenic non-human mammal all of whose germ cells and somatic cells contain a recombinant expression vector that inactivates or alters a gene encoding a PHI Protein. In an embodiment the invention provides a transgenic non-human mammal all of whose germ cells and somatic cells contain a recombinant expression vector that inactivates or alters a gene encoding a PHI Protein resulting in a PHI Protein associated pathology. Further the invention provides a transgenic non-human mammal which doe not express a PHI Protein of the invention. In an embodiment, the invention provides a transgenic non-human mammal which does not express a PHI Protein of the invention resulting in a PHI Protein associated pathology. A PHI Protein associated pathology refers to a phenotype observed for a PHI Protein homozygous or heterozygous mutant.

A transgenic non-human animal includes but is not limited to mouse, rat, rabbit, sheep, hamster, dog, cat goat, and monkey, preferably mouse.

The invention also provides a transgenic non-human animal assay system which provides a model system for testing for an agent that reduces or inhibits a PHI Protein associated pathology, comprising:

(a) administering the agent to a transgenic non-human animal of the invention; and

(b) determining whether said agent reduces or inhibits the pathology (e.g. PHI Protein associated pathology) in the transgenic non-human animal relative to a transgenic non-human animal of step (a) which has not been administered the agent.

The agent may be useful in the treatment and prophylaxis of conditions such as cancer or disorders associated with insulin response as discussed herein. The agents may also be incorporated in a pharmaceutical composition as described herein.

The activity of the proteins, peptides, complexes, substances, agents, compounds, antibodies, nucleic acid molecules, agents, and compositions of the invention may be confirmed in animal experimental model systems. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

The following non-limiting examples are illustrative of the present invention:

### EXAMPLE 1

Materials and Methods:

Antibodies: Anti-PHIP antibodies were raised against bacterial glutathione S-transferase (GST)-PHIP fusion protein (38). Anti-IRS-1^{PCT} (generated against a 16 amino acid pre C-terminal polypeptide sequence) was purchased from Upstate Biotechnology Inc. (UBI). Monoclonal anti-HA (12CA5) and anti-myc (9E10) antibodies were from Babco and Santa Cruz Biotechnology, respectively. Anti-CAT antibodies and mouse antibody to BrdU were purchased from 5 prime-3 prime Inc. and Sigma, respectively. Rhodamine-conjugated phalloidin was obtained from Molecular Probes. Anti transferrin receptor is purchased from Zymed.

Subcellular Fractionation Assay: COS-7 cells growing in 10-cm² dishes (four dishes/condition) were transiently transfected with pCGN plasmid encoding HA-PHIP or empty vector control using calcium phosphate method. Twenty-four hours after transfection, cells were serum starvedfor 12-18 hours and left untreated or treated with 100 nM of insulin for 5 minutes. Cell fractions were then prepared as previously described (27) with slight modifications. All procedures were performed at 0-4° C. Briefly, cells were washed and homogenized in ice-cold Buffer A containing 20 mM Tris-HCl, pH 7.5, 1 mM EDTA, 255 mM sucrose, 1 mM PMSF, 10 mM NaF, 100 μM Na₃VO₄, 1 mM NaPPi, 10 μg/ml aprotinin, and 10 μg/ml leupeptin for twenty strokes with a motor-driven Teflon/glass homogenizer. The homogenate was centrifuged at 16,000×g for 20 minutes. The supernatant was centrifuged at 48,000×g for 1 hour and subsequently at 250,000×g to purify the low-density membrane (LDM) pellet from the high-density membrane (HDM). The final LDM pellet was resuspended in hot 2×SDS sample buffer. The supernatant from 250,000×g centrifugation step was concentrated using a UFV2BGC40 filter apparatus (Millipore Corp.) which had been previously blocked with for 1 hour with 5% Tween 80 and washed extensively with water to remove any traces of the detergent Immunoprecipitation and immunoblotting was carried out (38).

Reporter Gene Assays: COS cells were transiently transfected in triplicate samples with 5×SRE-fos luciferase reporter gene (5×SRE-LUC) and the indicated plasmids. Twenty-four hours after transfection, the cells were serum starved for 16 hours. Serum-starved cells were either left untreated or treated with Mek-1 inhibitor (50 μM, NEB) for 2 hours. Cells were incubated for 10 hours with or without insulin (0.2 μM, Sigma). Luciferase activity was then analysed in cell lysates (Roche) and normalized to protein concentrations.

Microinjection Assays: Rat-1 or NIH/3T3 cells overexpressing insulin receptor (NIH/IR) plated onto gridded glass cover slips and serum starved for 30 hours, were microinjected with the indicated plasmids with or without 5×SRE-CAT reporter gene. For the reporter assay, 2 hours after injection, cells were treated with 0.5 μM insulin or serum (20%) as indicated and incubated for 5 hours before fixation. For the mitogenesis assay, 3 hours after injection, cells were treated with 10 μM BrdU (Roche), followed by addition of either 0.5 μM insulin or 20% serum. Cells were incubated for36 hours before fixation. Anti-CAT and anti-BrdU antibodies were then used to analyse reporter gene expression or DNA synthesis levels, respectively.

GLUT4myc Translocation Assay: L6^{GLUT4myc} stable cell lines were generated as previously described (49-51). Cells growing on cover slips were transfected with the indicated constructs according to the Effectene protocol manual (Qiagen). Fourty-three hours after transfection, cells were deprived of serum in culture medium for three hours and were left either untreated or treated with 100 nM insulin for 20 minutes. Indirect immunofluorescence for expression of cDNA constructs and GLUT4myc translocation was carried out on intact cells as previously described (53). Several representative images of at least three separate experiments were quantified with the use of NIH (National Institute of Health) image software. Raw data for GLUT4myc translocation were expressed as fold stimulation relative to basal levels of surface GLUT4myc in untransfected cells. Statistical analyses were carried out with analysis of variance (Fisher, multiple comparisons).

Actin Labeling: Growing L6^{GLUT4myc} cells on cover slips were left untreated or treated with 100 nM insulin for 10 minutes following serum deprivation. Cells were rinsed with ice-cold PBS (100 mM NaCl, 1 mM CaCl₂, 1 mM MgCl₂, 50 mM NaH₂PO₄/Na₂HPO₄, pH 7.4) before fixing with 3% paraformaldehyde in PBS for 30 min (initiated at 4° C. for 5 minutes and shifted immediately to room temperature). The rest of the procedure was performed at room temperature. The cells were then rinsed once with PBS, and unreacted fixative was quenched with 100 nM glycine in PBS for 10 minutes. Permeabilized cells (0.1% Triton X-100 in PBS for 3 minutes) were washed quickly with PBS and blocked with 5% goat serum in PBS for 10 minutes. To detect filamentous actin, cells were incubated in the dark with Rhodamine-conjugated phalloidin for 1 hour. Rinsed cover slips were then mounted and analyzed with the Leica TCS 4D fluorescence microscope (Leica Mikroscoipe Systeme GmbH, Wetzlar, Germany).

Results:

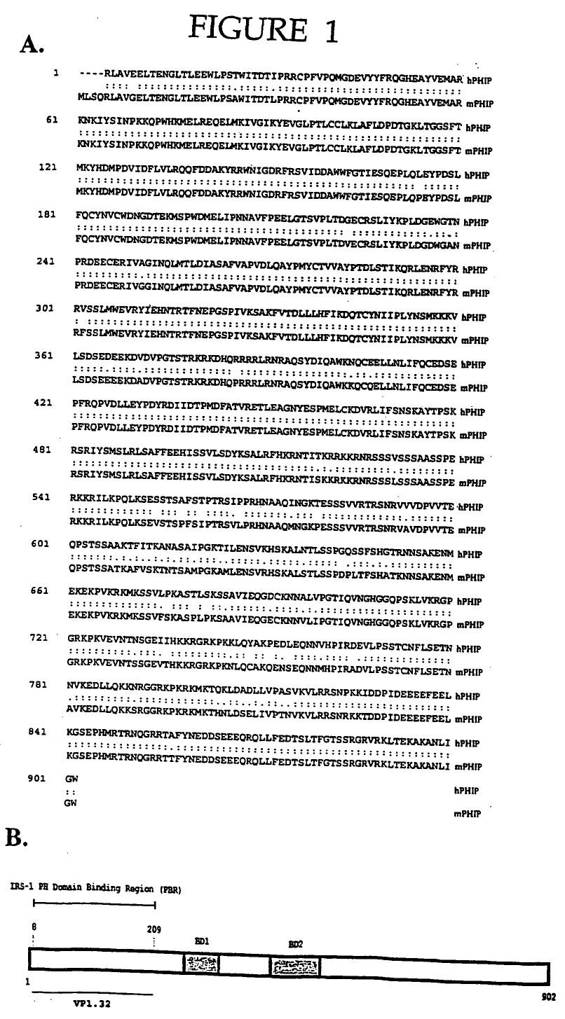

In an attempt to identify functional partners of the IRS-1 PH domain, a yeast two-hybrid screen was used in which the PH domain from rat IRS-1 was used as a bait to screen a murine 10.5 day embryonic cDNA library (5). Sequence analysis of a cDNA clone, VP1.32, which displayed the strongest interaction with the IRS-1 PH domain, revealed an open reading frame of 201 amino acids. VP1.32 was subsequently used to screen human fetal brain and mouse thymus cDNA libraries (7) to obtain the complete coding region of human and mouse PHIP (hPHIP and mPHIP) respectively. The conceptual translation predicts a 902 amino acid (aa) protein of relative molecular weight of 104 kDa (FIG. 1A).

PHI Proteins do not share sequence homology with any known proteins. The IRS-1 PH binding region (PBR) is located at the amino-terminus of the protein (residues 5-209). The only known structural motifs they possess are two bromodomains, BD1 (residues 230 to 345) and BD2 (387 to 503), located in tandem in the center of the molecule (FIG. 1B). Bromodomains are conserved sequences of approximately 100aa that have been proposed to mediate protein-protein interactions (8). A homology search revealed that PHIP BD sequences were most homologous (44% identity, 61% homology) to the bromodomain of mouse CBP (CREB binding protein), a transcriptional coactivator (9). Northern blot analysis of PHIP mRNA from adult mouse tissues detected a transcript size of approximately 7.0 kb whose expression is widespread.

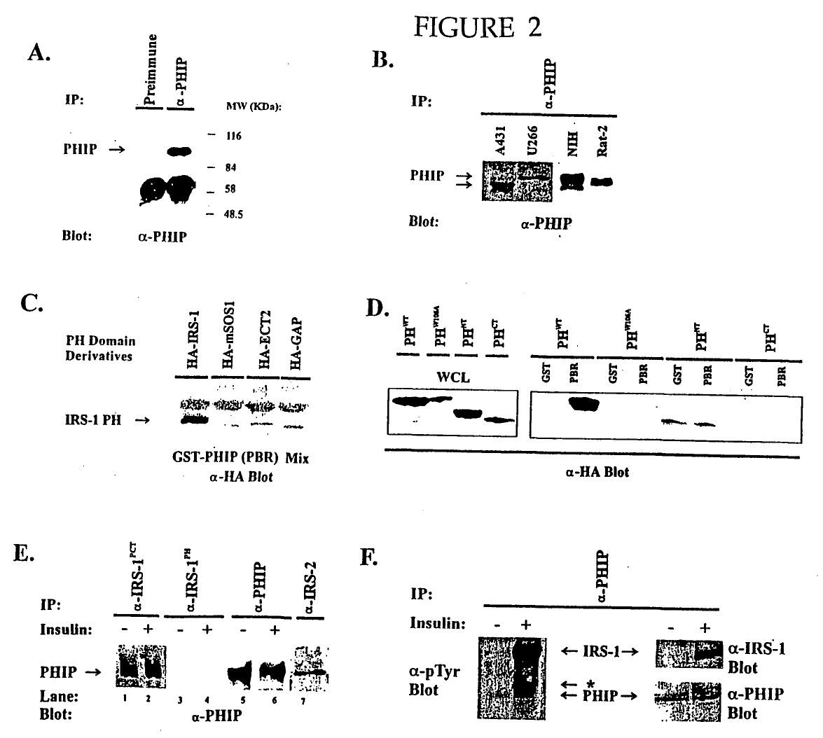

Western blot analysis with antibodies (Abs) raised against a bacterial glutathione S-transferase (GST)-PHIP fusion protein identified a 104 kD protein from U266 cell lysates which was not precipitated by preimmume sera (FIG. 2A). Further analysis of PHIP expression in mammalian cell extracts revealed two forms of PHI Protein, the long 104 kD form and a shorter 97 kD form (FIG. 2B). The 97 kD and 104 kD polypeptides likely result from alternative usage of two putative translation initiation sites (Met1 and Met41, see FIG. 1) as ectopic expression of full-length hPHIP containing both sites produced a doublet in PHIP immunoblots.

To recapitulate the interaction of PHIP with the IRS-1 PH domain in vitro and to assess the specificity of PH domain binding, GST-PHIP, containing residues 8-209 isolated from the yeast clone VP1.32, was used to probe yeast cell lysates expressing hemagglutinin antigen (HA)-tagged derivatives of PH domains from IRS-1, and from unrelated signaling proteins mSos1 (Ras nucleotide exchanger), Ect-2 (Rho/Rac exchanger) and RasGAP (GTPase activating protein) (12). Interacting proteins were analyzed by western blotting with anti-HA Abs (FIG. 2C). Whereas GST-PHIP bound to the IRS-1 PH domain, there was no discernable association with PH domains of other proteins, suggesting that PHIP may function as a specific ligand of the IRS-1 PH domain.

Next, to examine whether a functional PH domain or a smaller motif within the domain is responsible for PHIP binding, we generated three independent mutants of the IRS-1 PH domain that disrupt the PH fold: PH^{NT} encompasses the first half of the IRS-1 PH domain, spanning residues 3-67, PH^{CT} comprises the C-terminal residues 55-133, and PH^{W106A} defines a mutant where the Tryptophan at position 106, a residue conserved in all PH domains, was changed to Ala As expected, all three PH-domain mutants expressed transiently in COS-1 cells did not detectably associate with GST-PHIP, consistent with the notion that an intact PH domain is required for PHIP binding (FIG. 2D).

To investigate the interaction of PHIP and IRS-1 in vivo, lysates from NIH/IR cells (NIH3T3 cells overexpressing the insulin receptor) were immunoprecipitated with anti-IRS-1 Abs directed against the C-terminus of IRS-1. Endogenous PHIP was found to associate with IRS-1 in both unstimulated and insulin-treated cells. (FIG. 2E, lanes 1 and 2). By contrast, when antibodies directed against the IRS-1 PH domain were used in similar co-immunoprecipitation assays, no interaction was detected, confirming that structural determinants within the PH domain of IRS-1 confer binding to PHIP. PHIP was also detected in anti-IRS-2 immunoprecipitates (FIG. 2E, lane 7), consistent with the observation that IRS-1 and IRS-2 PH domains have been shown to be functionally interchangeable in promoting substrate recognition by the IR (4). Thus, PHIP may have a conserved function in recruiting members of the IRS protein family to activated IR complexes. To evaluate the effect of insulin binding on regulating PHIP/IRS-1 PH interactions, antibodies directed against the PHIP PH binding region (PBR) were used, as an indirect score for measuring conformational changes in this region induced upon insulin stimulation. PHIP/IRS-1 immune complexes were observed only in the insulin-treated cells using the PHIP Abs in immunoprecipitation assays (FIG. 2F). These results indicate that although PHIP and IRS-1 proteins are stably associated in cells, contact sites between the PHIP PBR region and the IRS-1 PH domain are regulated by insulin. This raises the possibility that structural changes at the PHIP PBR/IRS-1 PH interface observed upon insulin stimulation, may influence the interactions of the IRS-1 PTB with the phosphorylated insulin receptor. Consistent with this idea, substitution of the IRS-1 PH domain with heterologous PH domains from (adrenergic receptor kinase, and phospholipase C (impairs binding of the tandem PTB domain to phosphorylated NPEY peptides (4).

Whether PHIP functions as a substrate of the IR in vivo was examined, as there are several potential tyrosine phosphorylation sites in the PHIP sequence. Anti-phosphotyrosine immunoblots of PHIP failed to show any discernible IR-regulated phosphorylation of PHIP (FIG. 2F). PHIP however inducibly associated with a prominent 103 kDa phosphoprotein (i.e. STAT3).

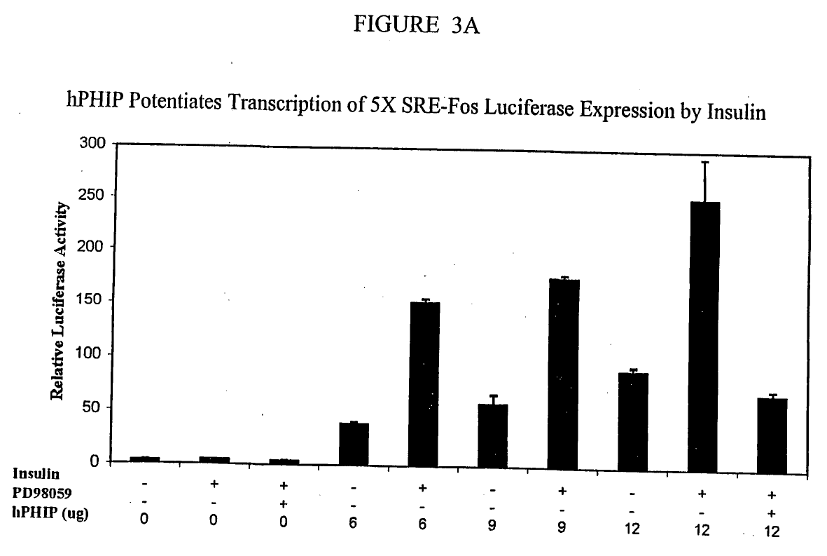

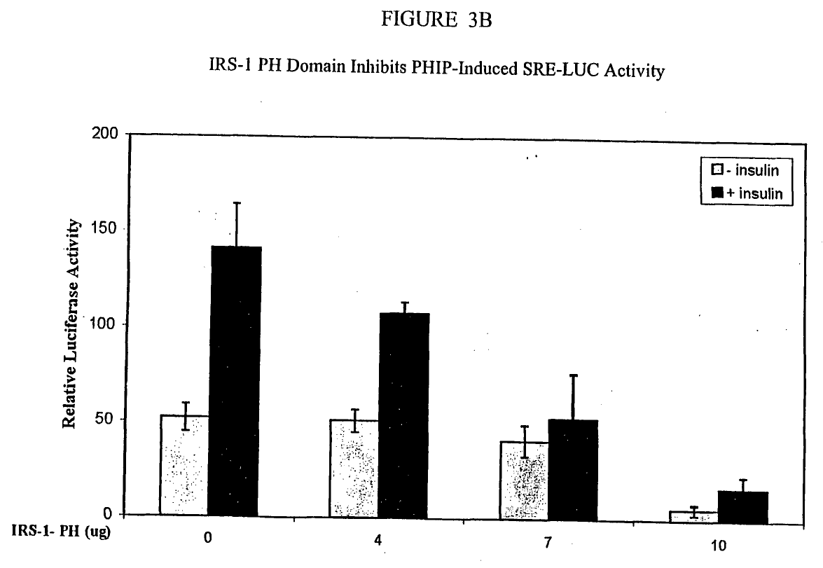

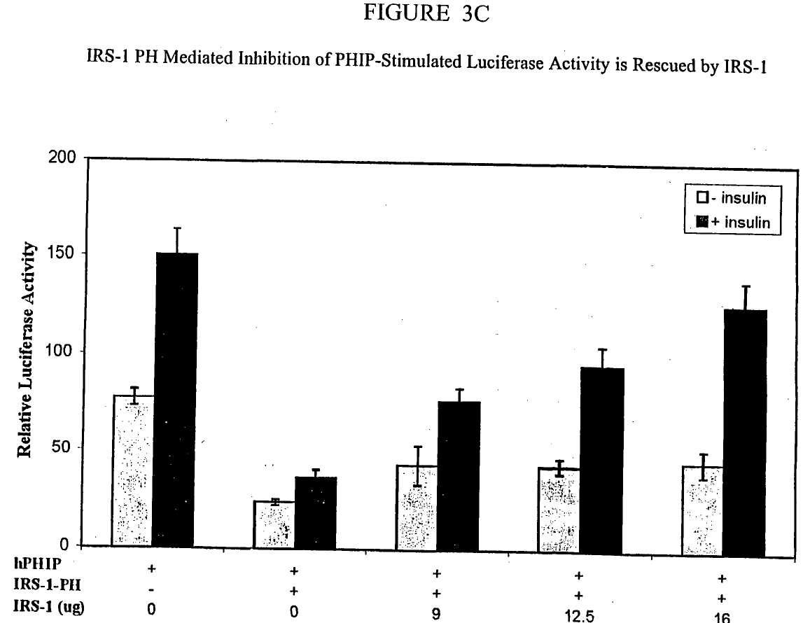

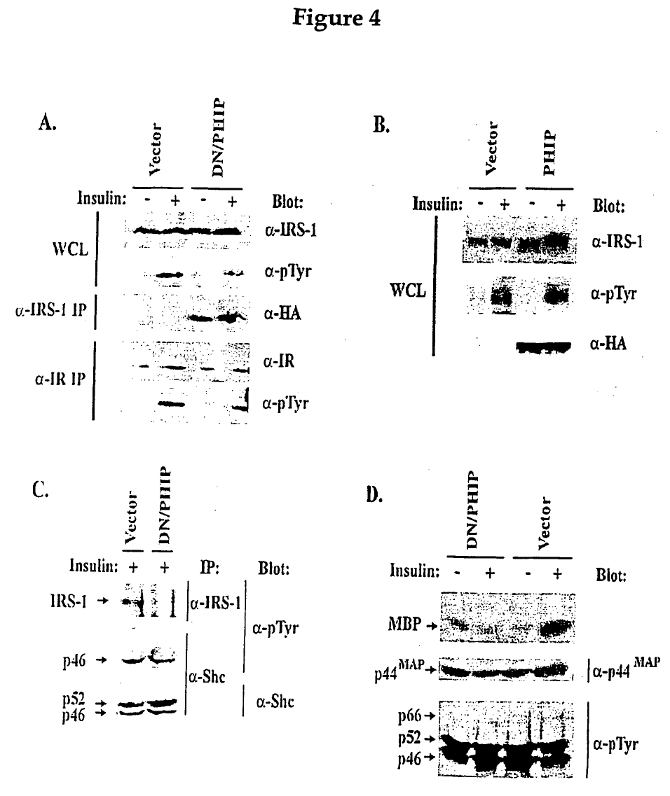

One of the early signaling events initiated by the IR is activation of MAP kinase (14). Moreover, in many cells, IRS-1 has been shown to be an upstream mediator MAP kinase activation during insulin stimulation. To evaluate the effect of PHIP on IRS-1-mediated MAP kinase activation, hemagglutinin antigen (HA)-tagged PHIP constructs were used that encode the IRS-1 PHIP PBR region alone (residues 8-209) which was predicted to function in a dominant inhibitory fashion by competing with the endogenous PHIP for the IRS-1 PH domain. Indeed, ectopically expressed dominant-negative PHIP (DN-PHIP) binds to endogenous IRS-1 in both untreated and insulin-stimulated cell lysates (FIG. 4A, panel 3). COS cells were co-transfected with DN-PHIP and HA-tagged p44^{MAPK} and anti-HA immune complexes from serum starved and insulin-stimulated cell lysates were subjected to an in vitro kinase assay using myelin basic protein (MBP) substrate. As shown in FIG. 4D, insulin-stimulated MAP kinase activation was reduced to basal levels by DN-PHIP expression. As expected, SHC phosphorylation remained retractile to the effects of DN-PHIP, suggesting that in these cells the PHIP/IRS-1 signaling pathway is essential for promoting MAP kinase activation during insulin stimulation. To evaluate the involvement of PHIP in insulin mediated transcriptional responses, its ability to induce transcription from a synthetic reporter, 5×SRE-LUC, which contains five copies of the serum responsive element (SRE) from the human c-fos promoter (15) was tested. COS-1 cells transiently transfected with the 5×SRE-LUC reporter gene and increasing amounts of hPHIP led to a dose-dependent increase in basal levels of transcription in untreated cells which was further enhanced by response to insulin (FIG. 3A). In order to investigate the relative importance of the MAP kinase pathway as a downstream effector of PHIP mediated gene expression, the Mek1 inhibitor, PD98059, was used to block MAP kinase activation (17). The complete sensitivity of ligand-dependent PHIP SRE-LUC transactivation to PD98059, suggests that the MAP kinase cascade is an important component of insulin-stimulated PHIP transcriptional responses.

To determine whether IRS-1 PH binding is required for PHIP's ability to potentiate insulin responses, the effect of overexpressing the N-terminal IRS-1 PH domain (IRS-PH) on PHIP-stimulated SRE-LUC transactivation was evaluated. Increasing expression of IRS-PH progressively blocked the PHIP signal, indicating that PH-domain-directed interaction between PHIP and IRS-1 is required for PHIP-induced gene expression (FIG. 3B). Overexpression of IRS-1 overcame this inhibition in a dose-dependent manner, indicating that the IRS-1 PH domain competes with wild type IRS-1 for PHIP complex formation (FIG. 3C).

To further establish the physiological significance of IRS-1/PHIP interactions for gene expression, HA-tagged DN-PHIP was microinjected into insulin-responsive Rat-1 fibroblasts. Insulin and serum treatment of parental Rat-1 fibroblasts microinjected with the reporter plasmid 5×SRE-CAT (chroramphenicol acetyltransferase) resulted in expression of the CAT protein readily detectable by immunofluorescence staining with anti-CAT Abs. However, cells co-injected with the construct expressing HA-tagged DN-PHIP blocked insulin-but not serum-stimulated CAT expression, indicating that PHIP is a critical component of the signaling pathway used by IR to regulate gene expression. This is consistent with the finding that DN-PHIP has a pronounced inhibitory effect on MAP kinase activation in insulin-treated cells. Co-injection of IRS-1 with DN-PHIP, fully restored SRE-CAT expression further supporting the idea that IRS-1 lies downstream of PHIP in the insulin signaling pathway.

Previous studies have demonstrated that the growth stimulatory effects of insulin are dependent on IRS-1 (19, 45). To examine the role of PHIP in IRS-1 mediated mitogenic signaling, DN-PHIP was microinjected into fibroblasts overexpressing IR (NIH/IR) cells to study its effect on 5-bromodeoxyuridine (BrdU) incorporation into newly synthesized DNA. Whereas the growth stimulatoy effects of serum were not affected by microinjection of DN-PHIP, insulin-induced stimulation of DNA synthesis was markedly attenuated in NIH/IR cells injected with DN-PHIP, consistent with the notion that PHIP/IRS-1PH interactions are essential in promoting the proliferative actions of insulin.

In order to establish the mechanism by which DN-PHIP inhibits insulin-mediated gene expression and DNA synthesis, whether DN-PHIP had the ability to disrupt IRS-1 phosphorylation in response to insulin was examined. Transient expression of DN-PHIP, but not fill length PHIP, significantly impaired IRS-1 tyrosine phosphorylation (>5-fold) in insulin-treated cells. To ascertain whether the reduction in IRS-1 phosphorylation occurred through interference with receptor function, changes were looked for in phosphotyrosine levels of immunoprecipitated IR and Shc, a direct substrate of the activated IR. The results demonstrate that diminution of IRS-1 tyrosine phosphorylation levels was not attributable to inhibition of IR kinase activity in at least two cell backgrounds. Next the association of PHIP with the insulin receptor was examined. Co-immunoprecipitation assays failed to detect PHIP in IR immune complexes.

Similar results have previously been reported for the association of the IR with either IRS-1 or the SHC adaptor, suggesting that IR/effector interactions are weak or transient in nature, and not detected in receptor immune complexes (73-75).

One of the main metabolic effects of insulin action on fat and muscle cells is the regulation of glucose uptake by inducing the redistribution of the glucose transporter, GLUT4, from intracellular compartments to the plasma membrane (44). Activation of the p85/p110 isoform of PI 3-kinase through its recruitment to phosphotyrosine sites on IRS-1 is a necessary component of insulin-stimulated GLUT4 translocation (45, 46). The role of IRS-1 in this process is somewhat controversial, with some studies indicating that IRS-1 tyrosine phosphorylation can be blocked without any effect on GLUT4 transport (47-48). In order to examine whether PHIP/IRS-1 complexes participate in the signal transduction pathway linking the IR to GLUT4 traffic in muscle cells, L6 myoblasts stably expressing a myc-tagged GLUT4 construct (L6GLUT4myc) (49-51) were transiently transfected with either wild-type or dominant-interfering forms of PHIP or IRS-1. Co-expression of green fluorescent protein (GFP) cDNA was used to facilitate recognition of transfected cells. As previously shown, insulin treatment of L6GLUT4myc myoblasts generates a two-fold gain in cell-surface GLUT4myc detected by immunofluorescence labeling of the exofacial myc epitope (52, 53). Ectopic expression of DN-PHIP caused a near complete inhibition of insulin-dependent GLUT4myc membrane translocation (>90%), in a manner identical to that observed with a dominant-negative mutant of the p85 subunit of PI 3-kinase (Δp85) (45, 54). The effect of DN-PHIP was specific for the insulin-stimulated state, as the content of cell surface GLUT4myc in unstimulated cells was not altered by the PHIP mutant. Expression from a plasmid encoding the IRS-1 PH domain also caused a significant reduction in insulin-dependent GLUT4myc translocation, albeit somewhat less robust (60%) than that induced by DN-PHIP. The incomplete inhibition may be accounted for in part by the presence of other IRS proteins that may partially substitute for IRS-1 function. By contrast, neither full-length PHIP nor full-length IRS-1 caused any measurable change in GLUT4myc redistribution. Taken together, these results support the idea that PHIP/IRS-1 complex formation is necessary but not sufficient in promoting the metabolic effects of insulin in muscle cells.

Recent evidence points to the potential participation of the actin microfilament network in promoting not only insulin-dependentredistribution of PI 3-kinase to GLUT4-containing vesicles but also in mobilizing GLUT4 to the cell surface (55-57). In light of the fact that previous reports have demonstrated the requirement of functional IRS-1 for insulin-stimulated actin cytoskeletal rearrangement (47), the role of PHIP in this process was examined. Rhodamine-conjugated phalloidin was used to detect changes in the pattern of filamentous actin in L6GLUT4myc cells ectopically expressing either wild-type PHIP or DN-PHIP. Whereas actin staining in the basal state exhibits a filamentous pattern that runs along the longitudinal axis of the cell, a marked reorganization of actin into dense structures throughout the myoplasm was observed upon insulin stimulation. This effect was dramatically decreased by the expression of DN-PHIP but not by the empty vector or wild-type PHIP. Intriguingly, overexpression of wild-type PHIP appeared to induce remodeling of the actin cytoskeleton even under basal conditions. Taken together, the observations clearly implicate PHIP in the regulation of insulin-dependent processes that promote cytoskeletal remodeling and accompany incorporation of GLUT4 vesicles at the plasma membrane surface of muscle cells.

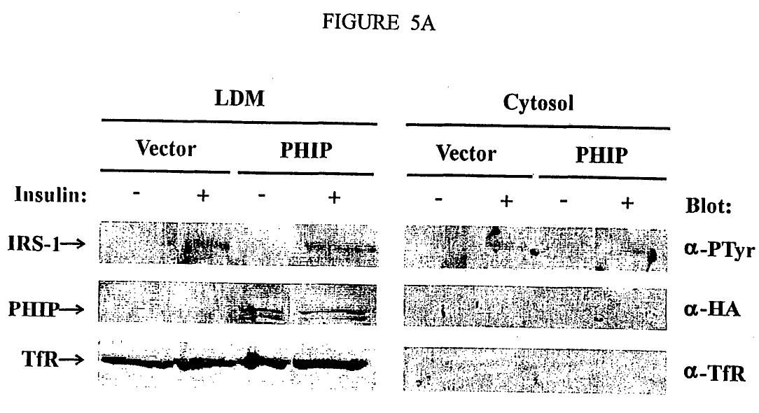

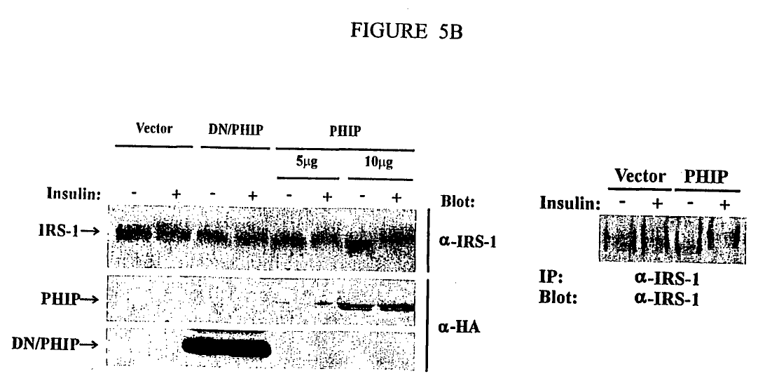

Cellular compartmentalization and intracellular trafficking of IRS-1 are essential in its ability to elicit insulin responses (30). Previous reports have shown that under basal conditions, insulin receptors are predominantly localized at the plasma membrane, while about two-thirds of the IRS-1 molecules associate with the LDM, and one-third are distributed within the cytoplasm (27-30, 58). Biochemical analyses of the LDM from cultured adipocytes indicates that IRS-1 does not associate with membranes in this fraction, but rather with what appears to be an insoluble protein matrix highly enriched in cytoskeletal elements that include actin (57, 59). Given that PHIP stably associates with IRS-1, whether PHIP co-localizes with IRS-1 in the LDM was examined. Immunoblot analysis of endogenous and ectopically expressed IRS-1 in L6 myoblasts failed to reveal strong immunoreactive signals, so a heterologous system was used to examine the cellular distribution of PHIP and IRS-1. Immunofluorescence microscopy of COS-7 cells indicated that PHIP and IRS-1 are immunolocalized in the cytoplasm (data not shown). Moreover, as demonstrated in FIG. 5A, subcellular fractionation of COS-7 cells revealed that tyrosine phosphorylated IRS-1 is distributed between the LDM fraction and the cytosol, consistent with the distribution of IRS-1 previously observed in adipocytes. Significantly, HA-PHIP ectopically expressed in COS-7 cells was found co-localized with IRS-1 primarily in the LDM fraction. (FIG. 5A). Furthermore, insulin treatment did not detectably alter the subcellular location of PHIP from the LDM to the cytosol. Therefore, PHIP may represent the putative IRS-1 binding component that serves to tether IRS-1 proteins, through its association with the IRS-1 PH domain, to cytoskeletal elements in the LDM compartment.

Biochemical studies in 3T3-L1 adipocytes indicate that IRS-1 is preferentially tyrosine phosphorylated in the LDM compartment (27, 58). Furthermore, insulin treatment induces a pronounced retardation in the electrophoretic mobility of IRS-1, due to hyperphosphorylation on serine/threonine (S/T) residues, which triggers the release of IRS-1 from the LDM to the cytosol(27, 28, 58, 60, and 61). This has led to the hypothesis that S/T phosphorylation of IRS-1 modulates IRS-1/LDM interactions. Given that PHIP segregates with IRS-1 in the LDM and is known to regulate IR-mediated IRS-1 tyrosine phosphorylation, the effect of PHIP overexpression on IRS-1 S/T phosphorylation was tested by monitoring the electrophoretic properties of IRS-1 by sodium dodecyl sulfate-polyacrylamide gel electrophoresis (SDS-PAGE). Under basal conditions, increasing amounts of ectopically expressed PHIP induced a dose-dependent increase in the electrophoretic mobility of IRS-1 (FIG. 5B). Given that hypophosphorylated forms of IRS-1 display increased association with LDM fractions (28, 58), the data suggest that PHIP overexpression may modulate a S/T phosphorylation event that enhances sequestration of IRS-1 to the LDM compartment. By contrast, acute insulin stimulation(5 min) of PHIP transfectants, produced a significant retardation in the mobility of IRS-1, consistent with an increase in the phospho-S/T content of IRS-1. This shift is typically observed with prolonged insulin treatment (15-60 min) (27, 58, and 62). Importantly, the amount of tyrosine phosphorylated IRS-1 remained fairly constant if not slightly increased in the highest PHIP expressors, when normalized for protein levels. These findings indicate that PHIP-dependent phosphorylation of IRS-1 S/T residues may elicit a positive regulatory effect on downstream signaling events. A recent study revealed that phosphorylation of serine residues within the PTB domain of IRS-1 by insulin-stimulated PKB, protects IRS-1 proteins from the rapid action of protein tyrosine phosphatases, and enables serine-phosphorylated IRS-1 proteins to maintain their tyrosine-phosphorylated active conformation (63).

Discussion

These results are the first to identify a protein ligand of the IRS-1 PH domain with a clear physiological role in both insulin-mediated mitogenic and metabolic responses. A dominant negative N-terminal truncation mutant of PHIP has been described, DN-PHIP, which potently inhibits insulin-induced transcriptional and proliferative responses. This inhibition is remarkably specific for insulin, as serum induced transactivation and DNA synthesis is unaffected by DN-PHIP. Moreover this inhibition is overcome by co-expression of IRS-1. Taken together, the data indicate that regions of PHIP implicated in interactions with the IRS-1 PH domain can disengage IR from IRS-1 proteins and subsequently decrease sensitivity to growth-promoting responses of insulin.

The role of IRS-1 proteins in insulin action on glucose transport is less clear. Several lines of evidence support the involvement of IRS-1 for GLUT4 externalization For example, expression of anti-sense ribozyme directed against rat IRS-1 significantly reduces GLUT4 translocation to the plasma membrane of rat adipose cells in response to insulin (64). Moreover, mutations of IR Tyr960 which do not alter receptor kinase activity, but are critical for IRS-1 binding and phosphorylation, abolish glucose transport (65-67). However, in contrast to these findings, other reports indicate that microinjection of anti-IRS-1 antibodies or expression of dominant inhibitory PTB domains of IRS-1 are able to block the mitogenic effects of insulin in fibroblasts but not GLUT4 trafficking in cultured adipocytes (47, 68). Interpretation of the results in adipocytes, is confounded by the observation that glucose uptake proceeds unabated in IRS-1 PTB-expressing cells, despite a near complete inhibition of not only IRS-1 tyrosine phosphorylation but of IR kinase activity (68).

In this current study, strong support is provided for the involvement of PHIP/IRS-1 complexes in glucose transporter translocation in muscle cells. The use of PHIP or IRS-1 constructs known to interfere with efficient IR/IRS-1 protein interaction and hence productive signal transduction from IRS-1 to PI 3-kinase, are capable of interfering with insulin-stimulated GLUT4 translocation in L6 myoblasts. Moreover, this inhibition does not coincide with changes in the autophosphorylation status of the IR. The data also indicate that overexpression of either PHIP or IRS-1 alone in muscle cells was not sufficient in promoting transport of GLUT4 to plasma membrane surfaces. This is consistent with other observations indicating that activation of IRS-1-associated signaling effectors such as PI 3-kinase, although necessary, is not sufficient for GLUT4 activation. Notably, growth factors such as PDGF and IL4 can activate PI 3-kinase as efficiently as insulin yet fail to stimulate glucose transport in insulin-sensitive cells (69, 70). One possible explanation is that additional PHIP/IRS-1/PI 3-kinase-independent pathways are required to coordinate GLUT4 intracellular routing. Indeed, recent evidence points to a novel insulin-responsive pathway that recruits flotillin/CAP/CBL complexes to IR-associated lipid rafts in the plasma membrane, an event which is thought to potentiate GLUT4 docking to the cell surface following insulin receptor activation (71).

A commonly held view to account for the specificity of insulin signaling on glucose transport, is that biological specificity is conferred at the level of cellular compartmentalization of signaling intermediates. Indeed, subcellular fractionation studies in 3T3-L1 adipocytes and IR-overexpressing CHO cells, revealed that activated PI 3-kinase complexes are found predominantly in the LDM following insulin treatment , whereas activation of PI 3-kinase in response to PDGF in the same cells, occurs at the plasma membrane (58, 59). Analogously, differences in the pattern of intracellular distribution have been documented among the four members of the IRS protein family (IRS 1-IRS4) and may account for differences in their ability to engage downstream signaling elements which may ultimately contribute to their functional specifity in vivo (28, 29, 72). In support of the idea that subcellular compartmentalization is central to IRS signal transduction, it has been demonstrated that altered trafficking and tight membrane association of CAAX-modified IRS-1 dramatically impairs insulin signaling. Moreover, based on the present studies, colocalization of PHIP with IRS-1 in the LDM compartment may be a key determinant in the selectivity and specificity of PHIP inhibitory action on IR signaling.

The molecular basis for sequestration of IRS-1 to internal low density microsomal fractions remains unclear. One obvious candidate is the IRS-1 PH domain. Previous studies have demonstrated the importance of PH domains in targeting proteins to cellular membranes by binding to phospholipids (33). However, the majority of these interactionsare weak and non-selective, suggesting the presence of specific cellular ligands for PH domain targeting function.

PHIP may serve as a molecular scaffold to sequester IRS-1 to cytoskeletal elements in the LDM. There are several observations that support this. First, the majority of IRS-1 is not anchored to membrane components but rather to an insoluble protein matrix in the LDM. This indicates that IRS-1 must be maintained at this location by specific association with other protein (s). Second, this Triton-insoluble fraction of the LDM contains a significant fraction of the actin cytoskeleton as determined by sedimentation analysis and electron microscopy (57, 59). Third, PHIP is stably associated and cofractionates with IRS-1 in the LDM under basal conditions. Finally, ectopic expression of PHIP can induce filamentous actin reorganization at discrete sites in the myoplasm, implicating PHIP in the spatial control of actin assembly. Taken together these data suggest that PHIP, through direct association with the IRS-1 PH domain may regulate tethering of IRS-1 molecules to the cytoskeletal component in the LDM. Thus PHIP may be important for the preassembly of IRS-1 proteins onto a cytoskeletal scaffold that is in close apposition to IR-enriched lipid rafts, providing a kinetic advantage in IRS-1 substrate recognition following receptor ligation. Moreover, the observation that ectopic expression of PHIP modulates the S/T phosphorylation status of IRS-1 proteins, a mechanism known to regulate the intracellular routing of IRS-1 between the LDM and cytosol, suggests that PHIP may also be involved in temporal desensitization or dampening of insulin signals by terminating access of IRS-1 to the IR. The insulin-regulatable effect of PHIP overexpression on the phospho-S/T content of IRS-1 could be due to the activation of a kinase and/or inhibition of a serine/threonine phosphatase acting on IRS-1.

In conclusion, PHIP represents a novel physiological protein target of the IRS-1 PH domain, that may contribute to IR coupling by regulating the spatial-temporal subcellular localization of IRS-1 protein complexes, which plays a pivotal role in the specificity and selectivity of IRS-1 function.

### EXAMPLE 2

Mutants of DN-PHIP were made in both GST and HIS tagged vectors. The sequences of the mutants are as follows:

| | |
|---|---|
| DN-mPHIP (aa 5-209) | |
| RLAVGELTENGLTLEEWLPSAWITDTLPRRCPFVPQMGDEVYYFRQGHEAYVEMARKNKIYSI | (SEQ ID NO: 66) |
| NPKKQPWHKMLEREQELMKIVGIKYEVGLPTLCCLKLAFLDPDTGKLTGGSFTMKYHDMPDVI | |
| DFLVLRQQFDDAKYRRWNIGDRFRSVIDDAWWFGTIESQEPLQPEYPDSLFQCYNVCWDNGD | |
| TEKMSPWDMELIPNNAV | |
| Mutant DN-mPHIP #1 (aa 5-170) | |
| RLAVGELTENGLTLEEWLPSAWITDTLPRRCPFVPQMGDEVYYFRQGHEAYVEMARKNKIYSI | (SEQ ID NO: 67) |
| NPKKQPWHKMELREQELMKIVGIKYEVGLPTLCCLKLAFLDPDTGKLTGGSFTMKYHDMPDVI | |
| DFLVLRQQFDDAKYRRWNIGDRFRSVIDDAWWFGTIESQE | |
| Mutant DN-mPHIP #2 (aa 19-170) | |
| EEWLPSAWITDTLPRRCPFVPQMGDEVYYFRQGHEAYVEMARKNKIYSINPKKQPWHKMELR | (SEQ ID NO:68) |
| EQELMKIVGIKYEVGLPTLCCLKLAFLDPDTGKLTGGSFTMKYHDMPDVIDFLVLRQQFDDAK | |
| YRRWNIGDRFRSVIDDAWWFGTIESQE | |

The mutants became insoluble when expressed in bacteria. This indicates that these small N- and C-terminal deletions perturb the structural integrity of the PBR protein module.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

### References and Notes

1. M. F. White and L. Yenush, Curr. Top. Microbiol. Immunol. 228, 179 (1998)

2. L. Yenush et al., J. Biol. Chem. 271, 24300 (1996); M. G. Myers, Jr., et al., J. Biol. Chem. 270, 11715 (1995); H. Voliovitch et al., J. Biol. Chem. 270, 18083 (1995)

3. G. Wolf et al., J. Biol. Chem. 270, 27407 (1995); M. J. Eck, S. Dhe-Paganon, T. Trub, R. T. Notle, S. E. Shoelson, Cell 85, 695 (1996)

4. D. J. Burks et al., J. Biol. Chem. 272, 27716 (1997)

5. The PH domain from rat IRS-1 (residues 3-133) was fused to the LexA binding domain within the BTM116 vector and used as a ‘bait’ to screen for interacting clones from a mouse 10.5 day embryonic complementary DNA (cDNA) library fused with the VP16-activation domain (6). A total of 89 positive clones were identified, most of which were represented at least twice, indicating that the screen was saturated. The clone which displayed the strongest interaction with the IRS-1 PH domain, VP1.32 was representative of 18/89 positive clones.

6. R. H. Schiestl, and R. D. Gietz, Genes & Dev. 7, 555 (1993)

7. Molecular Cloning: A laboratory manual (Cold Spring Harbor Laboratory Press, New York, 1989.

8. F. Jeanmougin, J.-M. Wurts, B. Le Douarin, P. Chambon, R. Losson, Trends Biochem. Sci. 22, 151 (1997)

9. J. C. Chrivia et al., Nature 365, 855 (1993)

10. PHIP PBR region (residues 8-209) was subcloned in frame into BamHI/EcoRI sites of the pGEX-3X vector (Invitrogen). Bacterially expressed GST-PHIP fusion proteins were injected into rabbits to raise anti-PHIP antibodies. Rabbit sera were depleted of anti-GST antibodies using a GST affinity column. Immunoprecipitation and immunoblot analysis were performed as previously described (11).

11. M. Rozakis-Adcock, R. Fernley, J. Wade, T. Pawson, D. Bowtell, Nature 363, 83 (1993)

12. M. Trahey, and F. McCormick, Science 238, 542 (1987); D. Bowtell, P. Fu, M. Simon, P. Senior, Proc. Natl. Acad. Sci. 89, 6511 (1992); T. Mild, C. L. Smith, J. E. Long, A. Eva, T. P. Fleming, Nature 362, 462 (1993)

13. L40 yeast cell lysates expressing various HA-tagged PH domains (mSos1 residues 448-577; human RasGAP residues 464-603; mouse Ect-2 residues 495-621) were lysed with acid-washed beads in 1 ml of distilled water containing 0.1 mM PMSF. Clarified cell lysates were incubated with ˜5 μg of GST-PHIP fusion proteins for 90 minutes at 4° C. Bound proteins were resolved by SDS-PAGE and analyzed by immunoblotting with anti-HA abs.

14. K. De Fea and R. A. Roth, J. Biol. Chem. 272, 31400 (1997)

15. R Graham and M. Gilman, Science 251, 189 (1991)

16. Luciferase activity measurements are normalized over the background and protein amount (30 μg/assay).

17. D. R. Alessi, A. Cuenda, P. Cohen, D. T. Dudley, A. R. Saltiel, J. Biol. Chem. 270, 27489 (1995)

18. M. Serrano, E. Gomez-Lomez, R. A. DePinho, D. Beach, D. Bar-Sagi, Science 267, 249 (1995)

19. D. W. Rose, A. R. Saltiel, M. Majumar, S. J. Decker, J. M. Olesfsky, Proc. Natl. Acad. Sci. 91, 797 (1994); L.-M. Wang et al., Science 261, 1591 (1993)

20. COS-7 whole cell lysates (WCL) were prepared by harvesting cells 48 hours after transfection with hot 2×SDS sample buffer. Equal amounts of protein (quantitated using the Bradford assay) were resolved by SDS-PAGE and probed with either anti-IRS-1, anti-Ptyr or anti-HA Abs as indicated. Rat-1 fibroblasts were transfected using GenePorter 2 (Gene Therapy Systems) as per manufacturer's instructions.

21. M. A. Lemmon and K. M. Fergusson, Curr. Top. Microbiol. Immunol. 228, 39 (1998).

22. C. R. Artalejo, M. A. Lemmon, J. Schlessinger, H. C. Palfrey, EMBO J. 16, 1565 (1997); A. D. Ma, L. F. Brass, C. S. Abrams, J. Cell Biol. 136, 1071 (1997)

23. Yenush L., Makati K. J., Smith-Hall J., Ishibashi O., Myers M. G. J. & White M. F. The pleckstrin homology domain is the principal link between the insulin receptor and IRS-1 J Biol Chem 271, 24300-24306 (1996).

24. Voliovitch H., Schindler D. G., Hadari Y. R., Taylor S. I., Accili D. & Zick Y. Tyrosine phosphorylation of insulin receptor substrate-1 in vivo depends upon the presence of its pleckstrin homology region J Biol Chem 270, 18083-18087 (1995).

25. Myers M. G. J., Grammer T. C., Brooks J., et al. The pleckstrin homology domain in insulin receptor substrate-1 sensitizes insulin signaling. J Biol Chem 270, 11715-11718 (1995).

26. White M. F. & Yenush L. The IRS-signaling system: a network of docking proteins that mediate insulin and cytokine action Curr Top Microbiol Immunol 228, 179-208 (1998).

27. Heller-Harrison R. A., Morin M. & Czech M. P. Insulin regulation of membrane-associated insulin receptor substrate 1. J Biol Chem 270, 24442-24450 (1995).

28. Inoue G., Cheatham B., Emkey R. & Kahn C. R. Dynamics of insulin signaling in 3T3-L1 adipocytes. Differential compartmentalization and trafficking of insulin receptor substrate (IRS)-1 and IRS-2 J Biol Chem 273, 11548-11555 (1998).

29. Anai M., Ono H., Funaki M., et al. Different subcellular distribution and regulation of expression of insulin receptor substrate (IRS)-3 from those of IRS-1 and IRS-2 J Biol Chem 273, 29686-29692 (1998).

30. Kriauciunas K. M., Myers M. G. J. & Kahn C. R. Cellular compartmentalization in insulin action: altered signaling by a lipid-modified IRS-1 Mol Cell Biol 20, 6849-6859 (2000).

31. Wolf G., Trub T., Ottinger E., et al. PTB domains of IRS-1 and Shc have distinct but overlapping binding specificities J Biol Chem 270, 27407-27410 (1995).

32. Eck M. J., Dhe-Paganon S., Trub T., Nolte R. T. & Shoelson S. E. Structure of the IRS-1 PTB domain bound to the juxtamembrane region of the insulin receptor Cell 85, 695-705 (1996).

33. Lemmon M. A. & Ferguson K. M. Signal-dependent membrane targeting by pleckstrin homology (PH) domains Biochem J 350, 1-18 (2000).

34. Isakoff S. J., Cardozo T., Andreev J., et al. Identification and analysis of PH domain-containing targets of phosphatidylinositol 3-kinase using a novel in vivo assay in yeast EMBO J 17, 5374-5387 (1998).

35. Pitcher J. A., Touhara K., Payne E. S. & Lefkowitz R. J. Pleckstrin homology domain-mediated membrane association and activation of the beta-adrenergic receptor kinase requires coordinate interaction with G beta gamma subunits and lipid J Biol Chem 270, 11707-11710 (1995).

36. Ravichandran K. S., Zhou M. M., Pratt J. C., et al. Evidence for a requirement for both phospholipid and phosphotyrosine binding via the Shc phosphotyrosine-binding domain in vivo. Mol Cell Biol 17, 5540-5549 (1997).

37. Qian X., Vass W. C., Papageorge A. G., Anborgh P. H. & Lowy D. R. N terminus of Sos1 Ras exchange factor: critical roles for the Db1 and pleckstrin homology domains Mol Cell Biol 18, 771-778 (1998).

38. Farhang-Fallah J., Yin X., Trentin G., Cheng A. M. & Rozakis-Adcock M. Cloning and characterization of PHIP, a novel insulin receptor substrate-1 pleckstrin homology domain interacting protein J Biol Chem 275, 40492-40497 (2000).

39. De Fea K & Roth R. A. Modulation of insulin receptor substrate-1 tyrosine phosphorylation and function by mitogen-activated protein kinase J Biol Chem 272, 31400-31406 (1997).

40. Graham R. & Gilman M. Distinct protein targets for signals acting at the c-fos serum response element Science 251, 189-192 (1991).

41. Alessi D. R., Cuenda A., Cohen P., Dudley D. T. & Saltiel A. R. PD 098059 is a specific inhibitor of the activation of mitogen-activated protein kinase kinase in vitro and in vivo J Biol Chem 270, 27489-27494 (1995).

42. Rose D. W., Saltiel A. R., Majumdar M., Decker S. J. & Olefsky J. M. Insulin receptor substrate 1 is required for insulin-mediated mitogenic signal transduction Proc Natl Acad Sci USA Jan. 18, 1994 91, 797-801

43. Wang L. M., Myers M. G. J., Sun X. J., Aaronson S. A., White M. & Pierce J. H. IRS-1: essential for insulin- and IL4-stimulated mitogenesis in hematopoietic cells Science 261, 1591-1594 (1993).

44. Czech M. P. & Corvera S. Signaling mechanisms that regulate glucose transport J Biol Chem 274, 1865-1868 (1999).

45. Quon M. J., Chen H., Ing B. L., et al. Roles of 1-phosphatidylinositol 3-kinase and ras in regulating translocation of GLUT4 in transfected rat adipose cells Mol Cell Biol 15, 5403-5411 (1995).

46. Sharma P. M., Egawa K., Huang Y., et al. Inhibition of phosphatidylinositol 3-kinase activity by adenovirus-mediated gene transfer and its effect on insulin action J Biol Chem 273, 18528-18537 (1998).

47. Morris A. J., Martin S. S., Haruta T., et al. Evidence for an insulin receptor substrate 1 independent insulin signaling pathway that mediates insulin-responsive glucose transporter (GLUT4) translocation Proc Natl Acad Sci USA 93, 8401-8406 (1996).

48. Sharma P. M., Egawa K., Gustafson T. A., Martin J. L. & Olefsky J. M. Adenovirus-mediated overexpression of IRS-1 interacting domains abolishes insulin-stimulated mitogenesis without affecting glucose transport in 3T3-L1 adipocytes Mol Cell Biol 17, 7386-7397 (1997).

49. Kanai F., Nishioka Y., Hayashi H., Kamohara S., Todaka M. & Ebina Y. Direct demonstration of insulin-induced GLUT4 translocation to the surface of intact cells by insertion of a c-myc epitope into an exofacial GLUT4 domain J Biol Chem 268, 14523-14526 (1993).

50. Kishi K., Muromoto N., Nakaya Y., et al. Bradykinin directly triggers GLUT4 translocation via an insulin-independent pathway Diabetes 47, 550-558 (1998).

51. Mitsumoto Y., Burdett E., Grant A. & Klip A. Differential expression of the GLUT1 and GLUT4 glucose transporter during differentiation of L6 muscle cells Biochem Biophys Res Commum 175, 652-659 (1991).

52. Ueyama A., Yaworsky K. L., Wang Q., Ebina Y. & Klip A. GLUT-4myc ectopic expression in L6 myoblasts generates a GLUT-4-specific pool conferring insulin sensitivity Am J Physiol 277, E572-E578 (1999).

53. Randhawa V. K., Bilan P. J., Khayat Z. A., et al. VAMP2, but not VAMP3/cellubrevin, mediates insulin-dependent incorporation of GLUT4 into the plasma membrane of L6 myoblasts Mol Biol Cell 11, 2403-2417 (2000).

54. Wang Q., Somwar R., Bilan P. J., et al. Protein kinase B/Akt participates in GLUT4 translocation by insulin in L6 myoblasts Mol Cell Biol 19, 4008-4018 (1999).

55. Tsakiridis T., Vranic M. & Klip A. Disassembly of the actin network inhibits insulin-dependent stimulation of glucose transport and prevents recruitment of glucose transporters to the plasma membrane J Biol Chem 269,29934-29942 (1994).

56. Wang Q., Bilan P. J., Tsakiridis T., Hinek A. & Klip A. Actin filaments participate in the relocalization of phosphatidylinositol3-kinase to glucose transporter-containing compartments and in the stimulation of glucose uptake in 3T3-L1 adipocytes Biochem J 331, 917-928 (1998).

57. Khayat Z. A., Tong P., Yaworsky K, Bloch R. J. & Klip A. Insulin-induced actin filament remodeling colocalizes actin with phosphatidylinositol 3-kinase and GLUT4 in L6 myotubes J Cell Sci 113 Pt 2, 279-290 (2000).

58. Clark S. F., Molero J. C. & James D. E. Release of insulin receptor substrate proteins from an intracellular complex coincides with the development of insulin resistance J Biol Chem 275, 3819-3826 (2000).

59. Clark S. F., Martin S., Carozzi A. J., Hill M. M. & James D. E. Intracellular localization of phosphatidylinositide 3-kinase and insulin receptor substrate-1 in adipocytes: potential involvement of a membrane skeleton J Cell Biol 140, 1211-1225 (1998).

60. Kublaoui B., Lee J. & Pilch P. F. Dynamics of signaling during insulin-stimulated endocytosis of its receptor in adipocytes J Biol Chem 270, 59-65 (1995).

61. Ricort J. M., Tanti J. F., Van Obberghen E. & Le Marchand-Brustel Y. Different effects of insulin and platelet-derived growth factor on phosphatidylinositol 3-kinase at the subcellular level in 3T3-L1 adipocytes. A possible explanation for their specific effects on glucose transport Eur J Biochem 239, 17-22 (1996).

62. Haruta T., Uno T., Kawahara J., et al. A rapamycin-sensitive pathway down-regulates insulin signaling via phosphorylation and proteasomal degradation of insulin receptor substrate-1 Mol Endocrinol 14, 783-794 (2000).

63. Paz K, Liu Y. F., Shorer H., et al. Phosphorylation of insulin receptor substrate-1 (IRS-1) by protein kinase B positively regulates IRS-1 function J Biol Chem 274, 28816-28822 (1999).

64. Quon M. J., Butte A. J., Zarnowski M. J., Sesti G., Cushman S. W. & Taylor S. I. Insulin receptor substrate 1 mediates the stimulatory effect of insulin on GLUT4 translocation in transfected rat adipose cells J Biol Chem 269, 27920-27924 (1994).

65. White M. F., Livingston J. N., Backer J. M., et al. Mutation of the insulin receptor at tyrosine 960 inhibits signal transmission but does not affect its tyrosine kinase activity Cell 54, 641-649 (1988).

66. Backer J. M., Kahn C. R., Cahill D. A., Ullrich A. & White M. F. Receptor-mediated internalization of insulin requires a 12-amino acid sequence in the juxtamembrane region of the insulin receptor beta-subunit J Biol Chem 265, 16450-16454 (1990).

67. Kanai F., Ito K., Todaka M., et al. Insulin-stimulated GLUT4 translocation is relevant to the phosphorylation of IRS-1 and the activity of PI3-kinase Biochem Biophys Res Commun 195, 762-768 (1993).

68. Sharma P. M., Egawa K., Gustafson T. A., Martin J. L. & Olefsky J. M. Adenovirus-mediated overexpression of IRS-1 interacting domains abolishes insulin-stimulated mitogenesis without affecting glucose transport in 3T3-L1 adipocytes Mol Cell Biol 17, 7386-7397 (1997).

69. Isakoff S. J., Taha C., Rose E., Marcusohn J., Klip A. & Skohlik E. Y. The inability of phosphatidylinositol 3-kinase activation to stimulate GLUT4 translocation indicates additional signaling pathways are required for insulin-stimulated glucose uptake Proc Natl Acad Sci USA 92, 10247-10251 (1995).

70. Guilherme A. & Czech M. P. Stimulation of IRS-1-associated phosphatidylinositol 3-kinase and Akt/protein kinase B but not glucose transport by beta1-integrin signaling in rat adipocytes J Biol Chem 273, 33119-33122 (1998).

71. Baumann C. A., Ribon V., Kanzaki M., et al. CAP defines a second signalling pathway required for insulin-stimulated glucose trasport Nature 407, 202-207 (2000).

72. Giovannone B., Scaldaferri M. L., Federici M., et al. Insulin receptor substrate (IRS) transduction system: distinct and overlapping signaling potential Diabetes Metab Res Rev 16, 434-441 (2000).

73. Pronk, G. J., McGlade, J., Pelicci G., Pawson, T., and Bos, J. L. (1993) J Biol.Chem 268, 5748-5753.

74. Backer, J. M., Myers, M. G. J., Sun, X. J., Chin, D. J., Shoelson, S. E., Miralpeix, M., and White, M. F. (1993) J Biol.Chem 268, 8204-8212.

75. Yenush, L., Fernandez, R., Myers, M. G. J., Grammer, T. C., Sun, X. J., Blenis, J., Pierce, J. H., Schlessinger, J., and White, M. F. (1996) Mol Cell Bio May 16, 1996 2509-2517.

We claim:

1. An isolated Pleckstrin Homology domain Interacting Protein (“PHI Protein”) that recruits proteins of the IRS protein family and STAT transcription factors to receptors that interact with, and phosphorylate the proteins and STAT transcription factors.

2. An isolated Pleckstrin Homology domain Interacting Protein (“PHI Protein”) according to claim 1 characterized by an N-terminal α-helix region predicting a coiled coil structure and a region containing two bromodomains, which is capable of interacting with a PH domain of insulin receptor substrate-1.

3. An isolated protein as claimed in claim 1 or 2 comprising an amino acid sequence of SEQ.ID.NO. 2, 3, 5, 6, 8, 10, 12, 13, 15, or 17.

4. An isolated nucleic acid molecule comprising at least 30 nucleotides which hybridizes to one of SEQ ID NO. 1, 4, 7, 9, 11, 14, 16, 18, 19, 20, 21, 22, 23, 24, 25, 26, 27, 28, 29, 30, 31, 32, 33, or 34 or the complement of SEQ ID NO. 1, 4, 7, 9, 11, 14, 16, 18, 19, 20, 21, 22, 23, 24, 25, 26, 27, 28, 29, 30, 31, 32, 33, or 34, under stringent hybridization conditions.

5. An isolated nucleic acid molecule which comprises:
(i) a nucleic acid sequence encoding a protein having substantial sequence identity with the amino acid sequence of SEQ. ID. NO. 2, 3, 5, 6, 8, 10, 12, 13, 15, or 17;
(ii) a nucleic acid sequence complementary to (i);
(iii) a nucleic acid sequence differing from any of (i) or (ii) in codon sequences due to the degeneracy of the genetic code;
(iv) a nucleic acid sequence comprising at least 10, preferably at least 15, more preferably at least 18, most preferably at least 20 nucleotides capable of hybridizing to a nucleic acid sequence of SEQ. ID. NO. 1, 4, 7, 9, 11, 14, 16, 18, 19, 20, 21, 22, 23, 24, 25, 26, 27, 28, 29, 30, 31, 32, 33, or 34 or to a degenerate form thereof;
(v) a nucleic acid sequence encoding a truncation, an analog, an allelic or species variation of a protein comprising the amino acid sequence of SEQ. ID. NO. 2, 3, 5, 6, 8, 10, 12, 13, 15, or 17; or
(vi) a fragment, or allelic or species variation of (i), (ii) or (iii).

6. An isolated nucleic acid molecule comprises:
(i) a nucleic acid sequence having substantial sequence identity or sequence similarity with a nucleic acid sequence of SEQ. ID. NO. 1, 4, 7, 9, 11, 14, 16, 18, 19, 20, 21, 22, 23, 24, 25, 26, 27, 28, 29, 30, 31, 32, 33, or 34;
(ii) nucleic acid sequences comprising the sequence of SEQ. ID. NO. 1, 4, 7, 9, 11, 14, 16, 18, 19, 20, 21, 22, 23, 24, 25, 26, 27, 28, 29, 30, 31, 32, 33, or 34 wherein T can also be U;
(iii) nucleic acid sequences complementary to (i), preferably complementary to the full nucleic acid sequence of SEQ. ID. NO. 1, 4, 7, 9, 11, 14, 16, 18, 19, 20, 21, 22, 23, 24, 25, 26, 27, 28, 29, 30, 31, 32, 33, or 34;
(iv) nucleic acid sequences differing from any of the nucleic acid sequences of (i), (ii), or (iii) in codon sequences due to the degeneracy of the genetic code; or
(v) a fragment, or allelic or species variation of (i), (ii) or (iii).

7. An isolated nucleic acid molecule which encodes a protein which binds an antibody of a protein as claimed in claim 1, 2 or 3.

8. A regulatory sequence of an isolated nucleic acid molecule as claimed in any one of claims 4 to 7 fused to a nucleic acid which encodes a heterologous protein.

9. A vector comprising a nucleic acid molecule of any one of claims 4 to 7.

10. A host cell comprising a nucleic acid molecule of any one of claims 4 to 7.

11. An isolated protein as claimed in claim 1, 2 or 3 comprising an amino acid sequence of SEQ. ID. NO. 2,3,5 or 6.

12. An isolated protein as claimed in claim 1 or 2 having at least 65% amino acid sequence identity to an amino acid sequence of SEQ. ID. NO. 2, 3, 5, or 6.

13. A method for preparing a protein as claimed in claim 1 comprising:
(a) transferring a vector as claimed in claim 9 into a host cell;
(b) selecting transformed host cells from untransformed host cells;
(c) culturing a selected transformed host cell under conditions which allow expression of the protein; and
(d) isolating the protein.

14. A protein prepared in accordance with the method of claim 13.

15. A binding region of a protein as claimed in any one of claims 1 to 3, 11, 12 or 14 which is a PH domain binding region, an IR binding region, or a STAT binding region.

16. A complex comprising a protein as claimed in claim 1 to 3, 11, 12 or 14 or a binding region thereof, and a binding partner.

17. A complex as claimed in claim 16 wherein the binding partner is a PH domain containing protein, a PH domain, a receptor that interacts with a protein of the IRS protein family or a binding region thereof that interacts with the PHI Protein, or a STAT transcription factor or a binding region thereof that interacts with the PHI Protein.

18. An antibody having specificity against an epitope of a protein as claimed in any one of claims 1 to 3, 11, 12 or 14.

19. An antibody as claimed in claim 18 labeled with a detectable substance and used to detect the polypeptide in biological samples, tissues, and cells.

20. A probe comprising a sequence encoding a protein as claimed in any one of claims 1 to 3, 11, 12 or 14, or a part thereof.

21. A method of diagnosing and monitoring conditions mediated by a protein as claimed in any one of claims 1 to 3, 11, 12 or 14 by determining the presence of a nucleic acid molecule as claimed in any one of claims 4 to 8 or a polypeptide as claimed in any one of claims 1 to 3, 11, 12 or 14.

22. A method as claimed in claim 21 wherein the condition is associated with an insulin response, or is cancer.

23. A method for identifying a substance which interacts with a protein as claimed in claim 1 to 3, 11, 12 or 14 comprising (a) reacting the protein with at least one substance which potentially can associate with the protein, under conditions which permit the association between the substance and protein, and (b) removing or detecting protein associated with the substance, wherein detection of associated protein and substance indicates the substance associates with the protein.

24. A method for evaluating a compound for its ability to modulate the biological activity of a protein as claimed in claim 1 to 3, 11, 12 or 14 comprising reacting the protein with a substance that interacts with the protein and a test compound under conditions which permit the formation of complexes between the substance and protein, and removing and/or detecting complexes.

25. A method for identifying inhibitors of a PHI Protein interaction, comprising
(a) providing a reaction mixture including a PHI Protein and a binding partner, or at least a portion of each which interact;
(b) contacting the reaction mixture with one or more test compounds; and
(c) identifying compounds which inhibit the interaction of the PHI Protein and binding partner.

26. A method for detecting a nucleic acid molecule encoding a protein comprising an amino acid sequence of SEQ. ID. NO. 2, 3, 5, or 6 in a biological sample comprising the steps of:
(a) hybridizing a nucleic acid molecule of any one of claims 4 to 7 to nucleic acids of the biological sample, thereby forming a hybridization complex; and
(b) detecting the hybridization complex wherein the presence of the hybridization complex correlates with the presence of a nucleic acid molecule encoding the protein in the biological sample.

27. A method as claimed in claim 26 wherein nucleic acids of the biological sample are amplified by the polymerase chain reaction prior to the hybridizing step.

28. A method for treating a condition mediated by a protein as claimed in claim 1 to 3, 11, 12 or 14 comprising administering an effective amount of an antibody as claimed in claim 18 or a substance, compound, or inhibitor identified in accordance with a method claimed in claim 23, 24, or 25.

29. A method as claimed in claim 28 wherein the condition is associated with an insulin response, or is cancer.

30. A composition comprising one or more of a nucleic acid molecule as claimed in any one of claims 4 to 7 or protein claimed in any one of claims 1 to 3, 11, 12 or 14, or a substance or compound identified using a method as claimed in any preceding claim, and a pharmaceutically acceptable carrier, excipient or diluent.

31. Use of one or more of a nucleic acid molecule as claimed in any one of claims 4 to 7 or protein claimed in any one of claims 1 to 3, 11, 12 or 14, or a substance or compound identified using a method as claimed in any preceding claim in the preparation of a medicament for treating a condition mediated by a protein as claimed in claim 1.

32. A transgenic non-human mammal which doe not express a PHI Protein as claimed in claim 1 to 3, 11, 12 or 14 resulting in a PHI Protein associated pathology.

33. A transgenic animal assay system which provides a model system for testing for an agent that reduces or inhibits a PHI Protein associated pathology comprising
(a) administering the agent to a transgenic non-human animal as claimed in claim 32; and
(b) determining whether said agent reduces or inhibits a PHI Protein associated pathology in the transgenic non-human animal relative to a transgenic non-human animal of step (a) which has not been administered the agent.

34. A method of conducting a drug discovery business comprising:
(a) providing one or more assay systems for identifying agents by their ability to inhibit or potentiate the interaction of a protein as claimed in any preceding claim and a binding partner;
(b) conducting therapeutic profiling of agents identified in step (a), or further analogs thereof, for efficacy and toxicity in animals; and
(c) formulating a pharmaceutical composition including one or more agents identified in step (b) as having an acceptable therapeutic profile.

35. A method as claimed in claim 34 further comprising establishing a distribution system for distributing the pharmaceutical composition for sale, and optionally establishing a sales group for marketing the pharmaceutical preparation.

36. A method of conducting a target discovery business comprising:
(a) providing one or more assay systems for identifying agents by their ability to inhibit or potentiate the interaction of a protein as claimed in any preceding claim and a binding partner;
(b) optionally conducting therapeutic profiling of agents identified in step (a) for efficacy and toxicity in animals; and
(c) licensing, to a third party, the rights for further drug development and/or sales for agents identified in step (a), or analogs thereof.

37. An isolated nucleic acid molecule comprises:
(i) a nucleic acid sequence having substantial sequence identity or sequence similarity with a nucleic acid sequence of one of SEQ. ID. NO. 35, and 39 through 63;
(ii) nucleic acid sequences comprising the sequence of one of SEQ. ID. NO. 35, and 39 through 63, wherein T can also be U;
(iii) nucleic acid sequences complementary to (i), preferably complementary to the full nucleic acid sequence of one of SEQ. ID. NO. 35, and 39 through 63;
(iv) nucleic acid sequences differing from any of the nucleic acid sequences of (i), (ii), or (iii) in codon sequences due to the degeneracy of the genetic code; or
(v) a fragment, or allelic or species variation of (i), (ii) or (iii).

38. An isolated neuronal differentiation-related protein encoded by:
(a) a nucleic acid molecule comprising one of SEQ ID NO. 39 through 63; or
(b) a nucleic acid molecule encoding a protein comprising SEQ ID NO: 36.


SEQUENCE LISTING (77 sequences)

SEQ ID NO: 1 - DNA, 2835 bp, Homo sapiens
agattggctgtgggagaactaactgaaaatggtttgacattagaagaatggttgccatcaacatggattacagataccattccccgaagatgtccatttgtgccacagatgggtgatgaggtttattatttccgacaaggacatgaagcctatgtcgaaatggcccggaaaaataaaatatatagtatcaatcccaaaaaacaaccatggcataaaatggagctacgggaacaagaacttatgaaaatagttggcataaagtatgaagtgggattacctaccctttgctgccttaaacttgcttttctagatcctgatactggtaaactgactggtggatcatttaccatgaaataccatgatatgcctgacgtcatagattttctagtcttgagacaacaatttgatgatgcaaaatacaggcgatggaatataggtgaccgcttcaggtctgtcatagatgatgcctggtggtttggaacaatcgaaagccaggaacctcttcaacttgagtaccctgatagtctgtttcaatgctacaatgtttgctgggacaatggagatacagaaaagatgagtccttgggatatggagcttatacctaataatgctgtatttcctgaagaactaggtaccagtgttcctttaactgatggtgagtgcagatcactaatctataaacctcttgatggagaatggggtaccaatcccagggatgaagaatgtgaaagaattgtggcaggaataaaccagttgatgacactagatattgcctcagcatttgtggcccccgtggatctgcaagcctatcccatgtattgcacagtagtggcatatccaacggatctaagtacaattaaacaaagactggaaaacaggttttacaggcgggtttcttccctaatgtgggaagttcgatatatagagcataatacacgaacatttaatgagcctggaagccctattgtgaaatctgctaaattcgtgactgatcttcttctacattttataaaggatcagacttgttataacataattccactttataattcaatgaagaagaaagttttgtctgattctgaggatgaagagaaagatgttgatgtgccaggaacttctactcgaaaaaggaaggaccatcagcgtagaagaagattacgtaatagagcccagtcttacgatattcaagcatggaagaaccagtgtgaagaattgttaaatctcatatttcaatgtgaagattcagagcctttccgtcagccggtagatctccttgaatatccagactacagagacatcattgacactccaatggattttgctaccgttagagaaactttagaggctgggaattatgagtcaccaatggagttatgtaaagatgtcagacttattttcagtaattccaaagcatatacaccaagcaaaagatcaaggatttacagcatgagtttgcgcctgtctgctttctttgaagaacacattagttcagttttatcagattataaatctgctcttcgttttcataaaagaaataccataaccaaaaggaggaagaaaagaaacagaagcagctctgtttccagtagtgctgcatcaagccctgaaaggaaaaaaaggatcttaaaaccccagctaaaatcagaaagctctacctctgcattctctacacctacacgatcaataccgccaagacacaatgctgctcagataaacggtaaaacagaatctagttctgtggttcgaaccagaagcaaccgagtggttgtagatccagttgtcactgagcaaccatctacttcttcagctgcaaagacttttattacaaaagctaatgcatctgcaataccagggaaaacaatactagagaattctgtgaaacattccaaagctttgaatactctttccagtcctggtcaatccagttttagtcatggcactaggaataattctgcaaaagaaaacatggaaaaggaaaagccagtcaaacgtaaaatgaagtcatctgtactcccaaaggcgtccactctttcaaagtcatcagctgtcattgagcaaggagattgtaagaacaacgctcttgtaccaggaaccattcaagtaaatggccatggaggacagccatcaaaacttgtgaagaggggacctggaaggaaacctaaagtagaagttaataccaatagtggtgaaattatacacaagaaaaggggtagaaagcccaaaaagctacagtatgcaaagccagaagatttagagcaaaataatgtgcatcccatcagagatgaagtacttccttcttcaacatgcaattttctttctgaaactaataatgtaaaggaagatttgttacagaaaaagaatcgtggaggtaggaagcccaaaaggaagatgaagacacaaaaattagatgcagatctcctagtccctgcaagtgtcaaagtgttaaggagaagtaacccgaaaaaaatagatgatcctatagatgaggaagaagagtttgaagaactcaaaggctctgaaccccacatgagaactagaaatcaaggtcgaaggacagctttctataatgaggatgactctgaagaggagcaaaggcagctgttgttcgaagacacctctttaacttttggaacttctagtagaggacgagtccgaaagttgactgaaaaagcaaaagctaatttaattggttggtaacttgtaccaaaatattttacttcaaaatctataaagcaggtacagttaaggaataagtaggactaaggcttctgcttccttgctgctgtggtggagtagggaatgttatgatttgatttgcaaaaaaaaaaaaaaaag

SEQ ID NO: 2 - protein, 898 aa, Homo sapiens
RLAVGELTENGLTLEEWLPSTWITDTIPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQLEYPDSLFQCYNVCWDNGDTEKMSPWDMELIPNNAVFPEELGTSVPLTDGECRSLIYKPLDGEWGTNPRDEECERIVAGINQLMTLDIASAFVAPVDLQAYPMYCTVVAYPTDLSTIKQRLENRFYRRVSSLMWEVRYIEHNTRTFNEPGSPIVKSAKFVTDLLLHFIKDQTCYNIIPLYNSMKKKVLSDSEDEEKDVDVPGTSTRKRKDHQRRRRLRNRAQSYDIQAWKNQCEELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTITKRRKKRNRSSSVSSSAASSPERKKRILKPQLKSESSTSAFSTPTRSIPPRHNAAQINGKTESSSVVRTRSNRVVVDPVVTEQPSTSSAAKTFITKANASAIPGKTILENSVKHSKALNTLSSPGQSSFSHGTRNNSAKENMEKEKPVKRKMKSSVLPKASTLSKSSAVIEQGDCKNNALVPGTIQVNGHGGQPSKLVKRGPGRKPKVEVNTNSGEIIHKKRGRKPKKLQYAKPEDLEQNNVHPIRDEVLPSSTCNFLSETNNVKEDLLQKKNRGGRKPKRKMKTQKLDADLLVPASVKVLRRSNPKKIDDPIDEEEEFEELKGSEPHMRTRNQGRRTAFYNEDDSEEEQRQLLFEDTSLTFGTSSRGRVRKLTEKAKANLIGW

SEQ ID NO: 3 - protein, 862 aa, Homo sapiens
MGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQLEYPDSLFQCYNVCWDNGDTEKMSPWDMELIPNNAVFPEELGTSVPLTDGECRSLIYKPLDGEWGTNPRDEECERIVAGINQLMTLDIASAFVAPVDLQAYPMYCTVVAYPTDLSTIKQRLENRFYRRVSSLMWEVRYIEHNTRTFNEPGSPIVKSAKFVTDLLLHFIKDQTCYNIIPLYNSMKKKVLSDSEDEEKDVDVPGTSTRKRKDHQRRRRLRNRAQSYDIQAWKNQCEELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTITKRRKKRNRSSSVSSSAASSPERKKRILKPQLKSESSTSAFSTPTRSIPPRHNAAQINGKTESSSVVRTRSNRVVVDPVVTEQPSTSSAAKTFITKANASAIPGKTILENSVKHSKALNTLSSPGQSSFSHGTRNNSAKENMEKEKPVKRKMKSSVLPKASTLSKSSAVIEQGDCKNNALVPGTIQVNGHGGQPSKLVKRGPGRKPKVEVNTNSGEIIHKKRGRKPKKLQYAKPEDLEQNNVHPIRDEVLPSSTCNFLSETNNVKEDLLQKKNRGGRKPKRKMKTQKLDADLLVPASVKVLRRSNPKKIDDPIDEEEEFEELKGSEPHMRTRNQGRRTAFYNEDDSEEEQRQLLFEDTSLTFGTSSRGRVRKLTEKAKANLIGW

SEQ ID NO: 4 - DNA, 3841 bp, Mus musculus
ctagaagagtttttagttttgtctgttaggatgtcttttgagagttttgtaaagaatatacgttttgcttttgtctctagccctccatcagtgattaggaaaagctgaataactttcgtcacttctgctgcttttctagtaaaaggttttaatactggagagtaaaatttttgcacagatttatttccttgtgtttgaagatagtactaatgctgttgcatgctttctcagagattggctgtaggagaactaactgagaatggcctaacgttagaagagtggttgccttcagcttggattacagacacacttcccaggagatgtccatttgtgccacagatgggtgatgaggtttattattttcgacaagggcatgaagcatatgttgagatggcccggaaaaataaaatttatagtatcaatcctaaaaagcagccatggcataagatggaactaagggaacaagaactaatgaaaattgttggtataaagtatgaagtggggttgcctaccctttgctgccttaaacttgcttttctagatcctgatactggcaaactgaccggtggatcatttaccatgaaataccatgatatgcctgacgtcatagattttctagtcttgagacaacaatttgatgatgcaaagtatagacgatggaatataggtgaccgcttcagatctgtcatagatgatgcctggtggtttggaacaattgaaagtcaagagcctcttcaacctgagtaccctgatagtttgtttcagtgttataatgtatgttgggacaatggagatacagaaaagatgagtccttgggatatggaattaatacctaataatgctgtctttccagaagaactgggtaccagtgttcctttaactgatgttgaatgtaggtcgctaatttataaacctcttgatggagattggggagccaatcccagggatgaagaatgtgaaagaattgttggaggaataaatcagctgatgacactagatattgcgtctgcatttgttgcccctgtggaccttcaagcttatcccatgtattgcactgtggtggcctatccaacggatctaagtacaattaaacaaagactggagaacaggttttacaggcgcttttcatcactaatgtgggaagttcgatatatagaacataatacacgaacattcaatgagccaggaagcccaattgtgaaatctgctaaatttgtgactgatcttctcctgcattttataaaggatcagacttgttataacataattccactttacaactcaatgaagaagaaagttttgtctgactctgaggaagaagagaaagatgctgatgttccagggacttctaccagaaagcgcaaggatcatcaacctagaagaaggttacgcaacagagctcagtcttacgatattcaggcatggaagaaacaatgtcaagaattactgaatctcatatttcaatgtgaagactcagaaccttttcgacagccagtggatcttcttgaatatccagactaccgagacatcattgacactccaatggactttgccactgttagagagactttagaggctgggaattatgagtcacccatggagttatgtaaagatgtcaggctcattttcagtaattctaaagcatacacaccaagcaagagatcaaggatttacagcatgagtttacgcctgtctgctttctttgaagaacatattagttcagttttgtcagattataaatctgctcttcgttttcataaaagaaacaccataagcaagaagaggaagaagcgaaacaggagcagctccctgtccagcagtgctgcctcaagccctgaaaggaaaaaaaggatcttaaaaccccagctaaagtcagaagtatctacctctccattctccatacctacaagatcagtactaccaagacataatgctgcacaaatgaatggtaaaccagaatccagttctgtggttcgaactaggagcaaccgtgtagctgtagatccagttgtcaccgagcagccctctacatcatcagccacaaaagcttttgtttcaaaaactaatacatctgccatgccaggaaaagcaatgctagagaattctgtgagacattccaaagccttgagcacactttccagccctgatccgctcacattcagccatgctacaaagaataattctgcaaaagaaaacatggaaaaggaaaagcctgtcaaacgtaaaatgaagtcttctgtgttttcaaaagcatctccacttccaaagtcagccgcagtcatagagcaaggagagtgtaagaacaatgttcttataccaggaaccattcaagtaaatggccatggaggacaaccatcaaaactcgtgaagagaggacctgggaggaagcccaaggtagaagttaacaccagcagtggtgaagtgacacacaagaaaagaggtagaaagcccaagaatctgcagtgtgcaaagcaggaaaactctgagcaaaataacatgcatcccatcagggctgacgtgcttccttcttcaacatgcaacttcctttctgaaactaatgctgtcaaggaggatttgttacagaaaaagagtcgtggaggcagaaaacccaaaaggaagatgaaaactcacaacctagattcagaactcatagttcctacaaatgttaaagtgttaaggagaagtaaccggaaaaaaacagatgatcctatagatgaggaagaggagtttgaagaactcaaaggctctgagcctcacatgagaactagaaatcagggtcgaaggacaactttctataatgaggatgactccgaggaagaacagagacagctgttgttcgaggacacctccttgacatttggaacttctagtagaggacgagtccgaaagttgactgaaaaagcaaaggctaatttaattggttggtaacttgaagcaaaatattgcattttaaaaaatctgtaacgcaggtacagttaaggagtaagtagaactaaggtctctgcttccttgctgctatgacggattagggaatgttacaatttgacttgggaaaatggacaaaaacacatttagaagataatttacatctttgaatgaaaaaaatctatatacatatatatttcaaatgtttgctatttattgcccttaggtaggttattcggttccacattcatttcatttgctgtttgaaattgaggacctgttataaattctggtttatttatggaagagacagctctgctacactattaagaaacatagtattcctagagataaagtatgttccctcttaaattgagttatttttgaccaagtgaggtacatttttactgatagcagaaggcatgccctaggaagagagatgttacaaagagtagcagtacattaagaatggcttcctctaaagataactttccagttcccaccatttggtatcctgaaaagtgttgtgaactgtaggtgttcaattacagaatatctagaggaagcttttgttttactccatttctgccaaacttaggagaaaaatgtattgatgcaaaggaaacatatccacattggaaaacatttgactgtctaatttttcagaccttgattcttatatcagtcactctatctctgtttattgtgccaaagactgagaatcagtgcagtggaaagcctgtttttgactgtcaggacagcatacacttttcagtactggaaaagctatatattctaaagagcaagttattacaaaattatgctgagttatatcctttttttggtactaaatgtaggaaaataatgcactggtgggtcctttgacagagatatcttagagaaaaaaaaaaaaaag

SEQ ID NO: 5 - protein, 902 aa, Mus musculus
MLSQRLAVGELTENGLTLEEWLPSAWITDTLPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQPEYPDSLFQCYNVCWDNGDTEKMSPWDMELIPNNAVFPEELGTSVPLTDVECRSLIYKPLDGDWGANPRDEECERIVGGINQLMTLDIASAFVAPVDLQAYPMYCTVVAYPTDLSTIKQRLENRFYRRFSSLMWEVRYIEHNTRTFNEPGSPIVKSAKFVTDLLLHFIKDQTCYNIIPLYNSMKKKVLSDSEEEEKDADVPGTSTRKRKDHQPRRRLRNRAQSYDIQAWKKQCQELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTISKKRKKRNRSSSLSSSAASSPERKKRILKPQLKSEVSTSPFSIPTRSVLPRHNAAQMNGKPESSSVVRTRSNRVAVDPVVTEQPSTSSATKAFVSKTNTSAMPGKAMLENSVRHSKALSTLSSPDPLTFSHATKNNSAKENMEKEKPVKRKMKSSVFSKASPLPKSAAVIEQGECKNNVLIPGTIQVNGHGGQPSKLVKRGPGRKPKVEVNTSSGEVTHKKRGRKPKNLQCAKQENSEQNNMHPIRADVLPSSTCNFLSETNAVKEDLLQKKSRGGRKPKRKMKTHNLDSELIVPTNVKVLRRSNRKKTDDPIDEEEEFEELKGSEPHMRTRNQGRRTTFYNEDDSEEEQRQLLFEDTSLTFGTSSRGRVRKLTEKAKANLIGW

SEQ ID NO: 6 - protein, 862 aa, Mus musculus
MGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQPEYPDSLFQCYNVCWDNGDTEKMSPWDMELIPNNAVFPEELGTSVPLTDVECRSLIYKPLDGDWGANPRDEECERIVGGINQLMTLDIASAFVAPVDLQAYPMYCTVVAYPTDLSTIKQRLENRFYRRFSSLMWEVRYIEHNTRTFNEPGSPIVKSAKFVTDLLLHFIKDQTCYNIIPLYNSMKKKVLSDSEEEEKDADVPGTSTRKRKDHQPRRRLRNRAQSYDIQAWKKQCQELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTISKKRKKRNRSSSLSSSAASSPERKKRILKPQLKSEVSTSPFSIPTRSVLPRHNAAQMNGKPESSSVVRTRSNRVAVDPVVTEQPSTSSATKAFVSKTNTSAMPGKAMLENSVRHSKALSTLSSPDPLTFSHATKNNSAKENMEKEKPVKRKMKSSVFSKASPLPKSAAVIEQGECKNNVLIPGTIQVNGHGGQPSKLVKRGPGRKPKVEVNTSSGEVTHKKRGRKPKNLQCAKQENSEQNNMHPIRADVLPSSTCNFLSETNAVKEDLLQKKSRGGRKPKRKMKTHNLDSELIVPTNVKVLRRSNRKKTDDPIDEEEEFEELKGSEPHMRTRNQGRRTTFYNEDDSEEEQRQLLFEDTSLTFGTSSRGRVRKLTEKAKANLIGW

SEQ ID NO: 7 - DNA, 3263 bp, Homo sapiens
cggatcttggagaatccaaaaagcaacagacaaatcaacacaattatcgtacaagatctgcattggaagagactcctagaccctcagaagagatagaaaatggcagtagttcttcagatgaaggcgaagtagttgctgtcagtggtggaacatccgaagaagaagagagagcatggcacagtgatggcagttctagtgactactccagtgattactctgactggacagcagatgcaggaattaatctgcagccaccaaagaaagttcctaagaataaaaccaagaaagcagaaagcagttcagatgaagaagaagaatctgaaaaacagaagcaaaaacagattaaaaagggaaaagaaaaagcaaatgaagaaaaagatggaccaatatcaccaaagaaaaagaagcccaaagaaagaaaacaaaagagattggctgtgggagaactaactgaaaatggtttgacattagaagaatggttgccatcaacatggattacagataccattccccgaagatgtccatttgtgccacagatgggtgatgaggtttattatttccgacaaggacatgaagcctatgtcgaaatggcccggaaaaataaaatatatagtatcaatcccaaaaaacaaccatggcataaaatggagctacgggaacaagaacttatgaaaatagttggcataaagtatgaagtgggattacctaccctttgctgccttaaacttgcttttctagatcctgatactggtaaactgactggtggatcatttaccatgaaataccatgatatgcctgacgtcatagattttctagtcttgagacaacaatttgatgatgcaaaatacaggcgatggaatataggtgaccgcttcaggtctgtcatagatgatgcctggtggtttggaacaatcgaaagccaggaacctcttcaacttgagtaccctgatagtctgtttcaatgctacaatgtttgctgggacaatggagatacagaaaagatgagtccttgggatatggagcttatacctaataatgctgtatttcctgaagaactaggtaccagtgttcctttaactgatggtgagtgcagatcactaatctataaacctcttgatggagaatggggtaccaatcccagggatgaagaatgtgaaagaattgtggcaggaataaaccagttgatgacactagatattgcctcagcatttgtggcccccgtggatctgcaagcctatcccatgtattgcacagtagtggcatatccaacggatctaagtacaattaaacaaagactggaaaacaggttttacaggcgggtttcttccctaatgtgggaagttcgatatatagagcataatacacgaacatttaatgagcctggaagccctattgtgaaatctgctaaattcgtgactgatcttcttctacattttataaaggatcagacttgttataacataattccactttataattcaatgaagaagaaagttttgtctgattctgaggatgaagagaaagatgttgatgtgccaggaacttctactcgaaaaaggaaggaccatcagcgtagaagaagattacgtaatagagcccagtcttacgatattcaagcatggaagaaccagtgtgaagaattgttaaatctcatatttcaatgtgaagattcagagcctttccgtcagccggtagatctccttgaatatccagactacagagacatcattgacactccaatggattttgctaccgttagagaaactttagaggctgggaattatgagtcaccaatggagttatgtaaagatgtcagacttattttcagtaattccaaagcatatacaccaagcaaaagatcaaggatttacagcatgagtttgcgcctgtctgctttctttgaagaacacattagttcagttttatcagattataaatctgctcttcgttttcataaaagaaataccataaccaaaaggaggaagaaaagaaacagaagcagctctgtttccagtagtgctgcatcaagccctgaaaggaaaaaaaggatcttaaaaccccagctaaaatcagaaagctctacctctgcattctctacacctacacgatcaataccgccaagacacaatgctgctcagataaacggtaaaacagaatctagttctgtggttcgaaccagaagcaaccgagtggttgtagatccagttgtcactgagcaaccatctacttcttcagctgcaaagacttttattacaaaagctaatgcatctgcaataccagggaaaacaatactagagaattctgtgaaacattccaaagctttgaatactctttccagtcctggtcaatccagttttagtcatggcactaggaataattctgcaaaagaaaacatggaaaaggaaaagccagtcaaacgtaaaatgaagtcatctgtactcccaaaggcgtccactctttcaaagtcatcagctgtcattgagcaaggagattgtaagaacaacgctcttgtaccaggaaccattcaagtaaatggccatggaggacagccatcaaaacttgtgaagaggggacctggaaggaaacctaaagtagaagttaataccaatagtggtgaaattatacacaagaaaaggggtagaaagcccaaaaagctacagtatgcaaagccagaagatttagagcaaaataatgtgcatcccatcagagatgaagtacttccttcttcaacatgcaattttctttctgaaactaataatgtaaaggaagatttgttacagaaaaagaatcgtggaggtaggaagcccaaaaggaagatgaagacacaaaaattagatgcagatctcctagtccctgcaagtgtcaaagtgttaaggagaagtaacccgaaaaaaatagatgatcctatagatgaggaagaagagtttgaagaactcaaaggctctgaaccccacatgagaactagaaatcaaggtcgaaggacagctttctataatgaggatgactctgaagaggagcaaaggcagctgttgttcgaagacacctctttaacttttggaacttctagtagaggacgagtccgaaagttgactgaaaaagcaaaagctaatttaattggttggtaacttgtaccaaaatattttacttcaaaatctataaagcaggtacagttaaggaataagtaggactaaggcttctgcttccttgctgctgtggtggagtagggaatgttatgatttgatttgcaaaaaaaaaaaaaaaag

SEQ ID NO: 8 - protein, 1041 aa, Homo sapiens
LDLGESKKQQTNQHNYRTRSALEETPRPSEEIENGSSSSDEGEVVAVSGGTSEEEERAWHSDGSSSDYSSDYSDWTADAGINLQPPKKVPKNKTKKAESSSDEEEESEKQKQKQIKKEKKKVNEEKDGPISPKKKKPKERKQKRLAVGELTENGLTLEEWLPSTWITDTIPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQLEYPDSLFQCYNVCWDNGDTEKMSPWDMELIPNNAVFPEELGTSVPLTDGECRSLIYKPLDGEWGTNPRDEECERIVAGINQLMTLDIASAFVAPVDLQAYPMYCTVVAYPTDLSTIKQRLENRFYRRVSSLMWEVRYIEHNTRTFNEPGSPIVKSAKFVTDLLLHFIKDQTCYNIIPLYNSMKKKVLSDSEDEEKDVDVPGTSTRKRKDHQRRRRLRNRAQSYDIQAWKNQCEELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTITKRRKKRNRSSSVSSSAASSPERKKRILKPQLKSESSTSAFSTPTRSIPPRHNAAQINGKTESSSVVRTRSNRVVVDPVVTEQPSTSSAAKTFITKANASAIPGKTILENSVKHSKALNTLSSPGQSSFSHGTRNNSAKENMEKEKPVKRKMKSSVLPKASTLSKSSAVIEQGDCKNNALVPGTIQVNGHGGQPSKLVKRGPGRKPKVEVNTNSGEIIHKKRGRKPKKLQYAKPEDLEQNNVHPIRDEVLPSSTCNFLSETNNVKEDLLQKKNRGGRKPKRKMKTQKLDADLLVPASVKVLRRSNPKKIDDPIDEEEEFEELKGSEPHMRTRNQGRRTAFYNEDDSEEEQRQLLFEDTSLTFGTSSRGRVRKLTEKAKANLIGW

SEQ ID NO: 9 - DNA, 3819 bp, Mus musculus
ggacagcagatgctggaattaacttgcagccaccaaagcccgttcctcctaagcataaaaccaagaaaccagaaagtagttcagatgaagaagaagaatctgaaaaccagaagcaaaaacatattaaaaaggaaagaaaaaaagcaaatgaagaaaaagatggaccaacatcaccaaagaaaaaaaagcccaaagaaagaaaacaaaagagattggctgtaggagaactaactgagaatggcctaacgttagaagagtggttgccttcagcttggattacagacacacttcccaggagatgtccatttgtgccacagatgggtgatgaggtttattattttcgacaagggcatgaagcatatgttgagatggcccggaaaaataaaatttatagtatcaatcctaaaaagcagccatggcataagatggaactaagggaacaagaactaatgaaaattgttggtataaagtatgaagtggggttgcctaccctttgctgccttaaacttgcttttctagatcctgatactggcaaactgaccggtggatcatttaccatgaaataccatgatatgcctgacgtcatagattttctagtcttgagacaacaatttgatgatgcaaagtatagacgatggaatataggtgaccgcttcagatctgtcatagatgatgcctggtggtttggaacaattgaaagtcaagagcctcttcaacctgagtaccctgatagtttgtttcagtgttataatgtatgttgggacaatggagatacagaaaagatgagtccttgggatatggaattaatacctaataatgctgtctttccagaagaactgggtaccagtgttcctttaactgatgttgaatgtaggtcgctaatttataaacctcttgatggagattggggagccaatcccagggatgaagaatgtgaaagaattgttggaggaataaatcagctgatgacactagatattgcgtctgcatttgttgcccctgtggaccttcaagcttatcccatgtattgcactgtggtggcctatccaacggatctaagtacaattaaacaaagactggagaacaggttttacaggcgcttttcatcactaatgtgggaagttcgatatatagaacataatacacgaacattcaatgagccaggaagcccaattgtgaaatctgctaaatttgtgactgatcttctcctgcattttataaaggatcagacttgttataacataattccactttacaactcaatgaagaagaaagttttgtctgactctgaggaagaagagaaagatgctgatgttccagggacttctaccagaaagcgcaaggatcatcaacctagaagaaggttacgcaacagagctcagtcttacgatattcaggcatggaagaaacaatgtcaagaattactgaatctcatatttcaatgtgaagactcagaaccttttcgacagccagtggatcttcttgaatatccagactaccgagacatcattgacactccaatggactttgccactgttagagagactttagaggctgggaattatgagtcacccatggagttatgtaaagatgtcaggctcattttcagtaattctaaagcatacacaccaagcaagagatcaaggatttacagcatgagtttacgcctgtctgctttctttgaagaacatattagttcagttttgtcagattataaatctgctcttcgttttcataaaagaaacaccataagcaagaagaggaagaagcgaaacaggagcagctccctgtccagcagtgctgcctcaagccctgaaaggaaaaaaaggatcttaaaaccccagctaaagtcagaagtatctacctctccattctccatacctacaagatcagtactaccaagacataatgctgcacaaatgaatggtaaaccagaatccagttctgtggttcgaactaggagcaaccgtgtagctgtagatccagttgtcaccgagcagccctctacatcatcagccacaaaagcttttgtttcaaaaactaatacatctgccatgccaggaaaagcaatgctagagaattctgtgagacattccaaagccttgagcacactttccagccctgatccgctcacattcagccatgctacaaagaataattctgcaaaagaaaacatggaaaaggaaaagcctgtcaaacgtaaaatgaagtcttctgtgttttcaaaagcatctccacttccaaagtcagccgcagtcatagagcaaggagagtgtaagaacaatgttcttataccaggaaccattcaagtaaatggccatggaggacaaccatcaaaactcgtgaagagaggacctgggaggaagcccaaggtagaagttaacaccagcagtggtgaagtgacacacaagaaaagaggtagaaagcccaagaatctgcagtgtgcaaagcaggaaaactctgagcaaaataacatgcatcccatcagggctgacgtgcttccttcttcaacatgcaacttcctttctgaaactaatgctgtcaaggaggatttgttacagaaaaagagtcgtggaggcagaaaacccaaaaggaagatgaaaactcacaacctagattcagaactcatagttcctacaaatgttaaagtgttaaggagaagtaaccggaaaaaaacagatgatcctatagatgaggaagaggagtttgaagaactcaaaggctctgagcctcacatgagaactagaaatcagggtcgaaggacaactttctataatgaggatgactccgaggaagaacagagacagctgttgttcgaggacacctccttgacatttggaacttctagtagaggacgagtccgaaagttgactgaaaaagcaaaggctaatttaattggttggtaacttgaagcaaaatattgcattttaaaaaatctgtaacgcaggtacagttaaggagtaagtagaactaaggtctctgcttccttgctgctatgacggattagggaatgttacaatttgacttgggaaaatggacaaaaacacatttagaagataatttacatctttgaatgaaaaaaatctatatacatatatatttcaaatgtttgctatttattgcccttaggtaggttattcggttccacattcatttcatttgctgtttgaaattgaggacctgttataaattctggtttatttatggaagagacagctctgctacactattaagaaacatagtattcctagagataaagtatgttccctcttaaattgagttatttttgaccaagtgaggtacatttttactgatagcagaaggcatgccctaggaagagagatgttacaaagagtagcagtacattaagaatggcttcctctaaagataactttccagttcccaccatttggtatcctgaaaagtgttgtgaactgtaggtgttcaattacagaatatctagaggaagcttttgttttactccatttctgccaaacttaggagaaaaatgtattgatgcaaaggaaacatatccacattggaaaacatttgactgtctaatttttcagaccttgattcttatatcagtcactctatctctgtttattgtgccaaagactgagaatcagtgcagtggaaagcctgtttttgactgtcaggacagcatacacttttcagtactggaaaagctatatattctaaagagcaagttattacaaaattatgctgagttatatcctttttttggtactaaatgtaggaaaataatgcactggtgggtcctttgacagagatatcttagagaaaaaaaaaaaaaag

SEQ ID NO: 10 - protein, 967 aa, Mus musculus
WTADAGINLQPPKKVPKHKTKKPESSSDEEEESENQKQKHIKKERKKANEEKDGPTSPKKKKPKERKQKRLAVGELTENGLTLEEWLPSAWITDTLPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQPEYPDSLFQCYNVCWDNGDTEKMSPWDMELIPNNAVFPEELGTSVPLTDVECRSLIYKPLDGDWGANPRDEECERIVGGINQLMTLDIASAFVAPVDLQAYPMYCTVVAYPTDLSTIKQRLENRFYRRFSSLMWEVRYIEHNTRTFNEPGSPIVKSAKFVTDLLLHFIKDQTCYNIIPLYNSMKKKVLSDSEEEEKDADVPGTSTRKRKDHQPRRRLRNRAQSYDIQAWKKQCQELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTISKKRKKRNRSSSLSSSAASSPERKKRILKPQLKSEVSTSPFSIPTRSVLPRHNAAQMNGKPESSSVVRTRSNRVAVDPVVTEQPSTSSATKAFVSKTNTSAMPGKAMLENSVRHSKALSTLSSPDPLTFSHATKNNSAKENMEKEKPVKRKMKSSVFSKASPLPKSAAVIEQGECKNNVLIPGTIQVNGHGGQPSKLVKRGPGRKPKVEVNTSSGEVTHKKRGRKPKNLQCAKQENSEQNNMHPIRADVLPSSTCNFLSETNAVKEDLLQKKSRGGRKPKRKMKTHNLDSELIVPTNVKVLRRSNRKKTDDPIDEEEEFEELKGSEPHMRTRNQGRRTTFYNEDDSEEEQRQLLFEDTSLTFGTSSRGRVRKLTEKAKANLIGW

SEQ ID NO: 11 - DNA, 615 bp, Homo sapiens
agattggctgtgggagaactaactgaaaatggtttgacattagaagaatggttgccatcaacatggattacagataccattccccgaagatgtccatttgtgccacagatgggtgatgaggtttattatttccgacaaggacatgaagcctatgtcgaaatggcccggaaaaataaaatatatagtatcaatcccaaaaaacaaccatggcataaaatggagctacgggaacaagaacttatgaaaatagttggcataaagtatgaagtgggattacctaccctttgctgccttaaacttgcttttctagatcctgatactggtaaactgactggtggatcatttaccatgaaataccatgatatgcctgacgtcatagattttctagtcttgagacaacaatttgatgatgcaaaatacaggcgatggaatataggtgaccgcttcaggtctgtcatagatgatgcctggtggtttggaacaatcgaaagccaggaacctcttcaacttgagtaccctgatagtctgtttcaatgctacaatgtttgctgggacaatggagatacagaaaagatgagtccttgggatatggagcttatacctaataatgctgta

SEQ ID NO: 12 - protein, 205 aa, Homo sapiens
RLAVGELTENGLTLEEWLPSTWITDTIPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQLEYPDSLFQCYNVCWDNGDTEKMSPWDMELIPNNAV

SEQ ID NO: 13 - protein, 205 aa, Mus musculus
RLAVGELTENGLTLEEWLPSAWITDTLPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQPEYPDSLFQCYNVCWDNGDTEKMSPWDMELIPNNAV

SEQ ID NO: 14 - DNA, 345 bp, Homo sapiens
aaacctcttgatggagaatggggtaccaatcccagggatgaagaatgtgaaagaattgtggcaggaataaaccagttgatgacactagatattgcctcagcatttgtggcccccgtggatctgcaagcctatcccatgtattgcacagtagtggcatatccaacggatctaagtacaattaaacaaagactggaaaacaggttttacaggcgggtttcttccctaatgtgggaagttcgatatatagagcataatacacgaacatttaatgagcctggaagccctattgtgaaatctgctaaattcgtgactgatcttcttctacattttataaaggatcagact

SEQ ID NO: 15 - protein, 115 aa, Homo sapiens
KPLDGEWGTNPRDEECERIVAGINQLMTLDIASAFVAPVDLQAYPMYCTVVAYPTDLSTIKQRLENRFYRRVSSLMWEVRYIEHNTRTFNEPGSPIVKSAKFVTDLLLHFIKDQT

SEQ ID NO: 16 - DNA, 351 bp, Homo sapiens
agaagaagattacgtaatagagcccagtcttacgatattcaagcatggaagaaccagtgtgaagaattgttaaatctcatatttcaatgtgaagattcagagcctttccgtcagccggtagatctccttgaatatccagactacagagacatcattgacactccaatggattttgctaccgttagagaaactttagaggctgggaattatgagtcaccaatggagttatgtaaagatgtcagacttattttcagtaattccaaagcatatacaccaagcaaaagatcaaggatttacagcatgagtttgcgcctgtctgctttctttgaagaacacattagttcagtttta

SEQ ID NO: 17 - protein, 117 aa, Homo sapiens
RRRLRNRAQSYDIQAWKNQCEELLNLIFQCEDSEPFRQPVDLLEYPDYRDIIDTPMDFATVRETLEAGNYESPMELCKDVRLIFSNSKAYTPSKRSRIYSMSLRLSAFFEEHISSVL

SEQ ID NO: 18 - DNA, 120 bp, Homo sapiens
agattggctgtgggagaactaactgaaaatggtttgacattagaagaatggttgccatcaacatggattacagataccattccccgaagatgtccatttgtgccacagatgggtgatgag

SEQ ID NO: 19 - DNA, 108 bp, Homo sapiens
gtttattatttccgacaaggacatgaagcctatgtcgaaatggcccggaaaaataaaatatatagtatcaatcccaaaaaacaaccatggcataaaatggagctacgg

SEQ ID NO: 20 - DNA, 125 bp, Homo sapiens
gaacaagaacttatgaaaatagttggcataaagtatgaagtgggattacctaccctttgctgccttaaacttgcttttctagatcctgatactggtaaactgactggtggatcatttaccatgaa

SEQ ID NO: 21 - DNA, 83 bp, Homo sapiens
ataccatgatatgcctgacgtcatagattttctagtcttgagacaacaatttgatgatgcaaaatacaggcgatggaatatag

SEQ ID NO: 22 - DNA, 112 bp, Homo sapiens
gtgaccgcttcaggtctgtcatagatgatgcctggtggtttggaacaatcgaaagccaggaacctcttcaacttgagtaccctgatagtctgtttcaatgctacaatgtttg

SEQ ID NO: 23 - DNA, 62 bp, Homo sapiens
ctgggacaatggagatacagaaaagatgagtccttgggatatggagcttatacctaataatg

SEQ ID NO: 24 - DNA, 156 bp, Homo sapiens
ctgtatttcctgaagaactaggtaccagtgttcctttaactgatggtgagtgcagatcactaatctataaacctcttgatggagaatggggtaccaatcccagggatgaagaatgtgaaagaattgtggcaggaataaaccagttgatgacactag

SEQ ID NO: 25 - DNA, 121 bp, Homo sapiens
atattgcctcagcatttgtggcccccgtggatctgcaagcctatcccatgtattgcacagtagtggcatatccaacggatctaagtacaattaaacaaagactggaaaacaggttttacag

SEQ ID NO: 26 - DNA, 126 bp, Homo sapiens
gcgggtttcttccctaatgtgggaagttcgatatatagagcataatacacgaacatttaatgagcctggaagccctattgtgaaatctgctaaattcgtgactgatcttcttctacattttataaa

SEQ ID NO: 27 - DNA, 70 bp, Homo sapiens
ggatcagacttgttataacataattccactttataattcaatgaagaagaaagttttgtctgattctgag

SEQ ID NO: 28 - DNA, 51 bp, Homo sapiens
gatgaagagaaagatgttgatgtgccaggaacttctactcgaaaaaggaag

SEQ ID NO: 29 - DNA, 150 bp, Homo sapiens
gaccatcagcgtagaagaagattacgtaatagagcccagtcttacgatattcaagcatggaagaaccagtgtgaagaattgttaaatctcatatttcaatgtgaagattcagagcctttccgtcagccggtagatctccttgaatatcca

SEQ ID NO: 30 - DNA, 153 bp, Homo sapiens
gactacagagacatcattgacactccaatggattttgctaccgttagagaaactttagaggctgggaattatgagtcaccaatggagttatgtaaagatgtcagacttattttcagtaattccaaagcatatacaccaagcaaaagatcaagg

SEQ ID NO: 31 - DNA, 164 bp, Homo sapiens
atttacagcatgagtttgcgcctgtctgctttctttgaagaacacattagttcagttttatcagattataaatctgctcttcgttttcataaaagaaataccataaccaaaaggaggaagaaaagaaacagaagcagctctgtttccagtagtgctgcatcaag

SEQ ID NO: 32 - DNA, 260 bp, Homo sapiens
ccctgaaaggaaaaaaaggatcttaaaaccccagctaaaatcagaaagctctacctctgcattctctacacctacacgatcaataccgccaagacacaatgctgctcagataaacggtaaaacagaatctagttctgtggttcgaaccagaagcaaccgagtggttgtagatccagttgtcactgagcaaccatctacttcttcagctgcaaagacttttattacaaaagctaatgcatctgcaataccagggaaaacaa

SEQ ID NO: 33 - DNA, 198 bp, Homo sapiens
tactagagaattctgtgaaacattccaaagctttgaatactctttccagtcctggtcaatccagttttagtcatggcactaggaataattctgcaaaagaaaacatggaaaaggaaaagccagtcaaacgtaaaatgaagtcatctgtactcccaaaggcgtccactctttcaaagtcatcagctgtcattgagcaag

SEQ ID NO: 34 - DNA, 776 bp, Homo sapiens
gagattgtaagaacaacgctcttgtaccaggaaccattcaagtaaatggccatggaggacagccatcaaaacttgtgaagaggggacctggaaggaaacctaaagtagaagttaataccaatagtggtgaaattatacacaagaaaaggggtagaaagcccaaaaagctacagtatgcaaagccagaagatttagagcaaaataatgtgcatcccatcagagatgaagtacttccttcttcaacatgcaattttctttctgaaactaataatgtaaaggaagatttgttacagaaaaagaatcgtggaggtaggaagcccaaaaggaagatgaagacacaaaaattagatgcagatctcctagtccctgcaagtgtcaaagtgttaaggagaagtaacccgaaaaaaatagatgatcctatagatgaggaagaagagtttgaagaactcaaaggctctgaaccccacatgagaactagaaatcaaggtcgaaggacagctttctataatgaggatgactctgaagaggagcaaaggcagctgttgttcgaagacacctctttaacttttggaacttctagtagaggacgagtccgaaagttgactgaaaaagcaaaagctaatttaattggttggtaacttgtaccaaaatattttacttcaaaatctataaagcaggtacagttaaggaataagtaggactaaggcttctgcttccttgctgctgtggtggagtagggaatgttatgatttgatttgcaaaaaaaaaaaaaaaag

SEQ ID NO: 35 - DNA, 3123 bp, Homo sapiens
ccgaagctcggctcgtgaacacacactgacagctatagggcaggcggcggcaccgtccccgcttcccctcggcggcggggtgtcccgtcggcggccctgaagtgacccataaacatgtcttgtgagaggaaaggcctctcggagctgcgatcggagctctacttcctcatcgcccggttcctggaagatggaccctgtcagcaggcggctcaggtgctgatccgcgaggtggccgagaaggagctgctgccccggcgcaccgactggaccgggaaggagcatcccaggacctaccagaatctggtgaagtattacagacacttagcacctgatcacttgctgcaaatatgtcatcgactaggacctcttcttgaacaagaaattcctcaaagtgttcctggagtacaaactttattaggagctggaagacagtctttactacgcacaaataaaagctgcaagcatgttgtgtggaaaggatctgctctggctgcgttgcactgtggaagaccacctgagtcaccagttaactatggtagcccacccagcattgcggatactctgttttcaaggaagctgaatgggaaatacagacttgagcgacttgttccaactgcagtgtatcagcacatgaaaatgcataaacgaattcttggacacttgtcatctgtgtactgtgtaacttttgatcgaactggcagacggatatttactggttctgatgactgtcttgtgaaaatatgggcaacagatgatgggaggttgttagctaccttaagaggacatgctgctgaaatatcagacatggctgtaaactatgagaataccatgatagcagctggaagttgtgataaaatgatccgagtctggtgtcttcgaacctgtgcacctttggctgttcttcagggccatagtgcatctattacatcactacagttctcaccattgtgcagtggctcaaagagatatctatcttctactggggcagatggcactatttgtttttggctctgggatgctggaacccttaaaataaacccaagacctgcaaaatttacagagcgccctcggcctggagttcaaatgatctgttcttcttttagtgctggtggaatgtttctggcgacgggaagcacagatcatattattcgggtttatttttttggatcaggtcagccagagaaaatatcagaattggagtttcatactgacaaagttgacagtatccagttttccaacactagtaacaggtttgtaagtggcagtcgtgatgggacagcacgtatttggcaatttaaacgaagagagtggaagagcattttgttggatatggctactcgtccagcaggccaaaaccttcaaggaatagaagataaaatcacaaaaatgaaggttactatggtagcttgggatcgacatgacaatacagttataactgcagttaataacatgactctgaaagtttggaattcttacactggtcaactaattcatgtcctgatgggtcatgaagatgaggtatttgttcttgaaccacacccgttcgatcctagagttctcttttctgctggtcatgatggaaacgtgatagtgtgggatctggcaagaggagtcaaaatacgatcttatttcaatatgattgaaggccaaggacatggcgcagtatttgactgcaaatgctctcctgatggtcagcattttgcatgcacagactctcatggacatcttttaatttttggctttgggtccagtagcaaatatgacaagatagcagatcagatgttctttcatagtgattatcggccacttattcgtgatgccaacaattttgtattagatgaacagactcagcaagcacctcatcttatgcctcccccttttttggttgatgttgatggtaaccctcatccatcaagatatcaaagattagttcctggccgtgaaaattgcagggaggagcaactcatccctcagatgggagtaacttcctcaggactgaatcaagttttaagtcagcaagcaaaccaggagatcagcccactggacagcatgattcaaagactacaacaggagcaagacctgagacgttctggtgaagcagttatcagtaataccagccgtttaagtagaggctccataagttctacctcagaggttcattcaccaccaaacgtaggactaagacgtagtggacaaattgaaggtgtacggcaaatgcacagcaacgcaccaagaagtgaaatagccacagagcgggatctggtagcttggagtcgaagggtggtagtacccgagctatcagctggtgtagccagtaggcaagaagaatggagaactgcaaagggagaagaagaaataaagacttacaggtcagaagagaaaagaaaacacttaactgttccaaaagagaataaaatacccactgtctcaaagaatcatgctcatgagcatttcctggatcttggagaatccaaaaagcaacagacaaatcaacacaattatcgtacaagatctgcattggaagagactcctagaccctcagaagagatagaaaatggcagtagttcttcagatgaaggcgaagtagttgctgtcagtggtggaacatccgaagaagaagagagagcatggcacagtgatggcagttctagtgactactccagtgattactctgactggacagcagatgcaggaattaatctgcagccaccaaagaaagttcctaagaataaaaccaagaaagcagaaagcagttcagatgaagaagaagaatctgaaaaacagaagcaaaaacagattaaaaaggaaaagaaaaaagtaaatgaagaaaaagatggaccaatatcaccaaagaaaaagaagcccaaagaaagaaaacaaaagagattggctgtgggagaactaactgaaaatggtttgacattagaagaatggttgccatcaacatggattacagataccattccccgaagatgtccatttgtgccacagatgggtgatgaggtttattatttccgacaaggacatgaagcctatgtcgaaatggcccggaaaaataaaatatatagtatcaatcccaaaaaacaaccatggcataaaatggagctacgggtatgacattga

SEQ ID NO: 36 - protein, 1000 aa, Homo sapiens
MSCERKGLSELRSELYFLIARFLEDGPCQQAAQVLIREVAEKELLPRRTDWTGKEHPRTYQNLVKYYRHLAPDHLLQICHRLGPLLEQEIPQSVPGVQTLLGAGRQSLLRTNKSCKHVVWKGSALAALHCGRPPESPVNYGSPPSIADTLFSRKLNGKYRLERLVPTAVYQHMKMHKRILGHLSSVYCVTFDRTGRRIFTGSDDCLVKIWATDDGRLLATLRGHAAEISDMAVNYENTMIAAGSCDKMIRVWCLRTCAPLAVLQGHSASITSLQFSPLCSGSKRYLSSTGADGTICFWLWDAGTLKINPRPAKFTERPRPGVQMICSSFSAGGMFLATGSTDHIIRVYFFGSGQPEKISELEFHTDKVDSIQFSNTSNRFVSGSRDGTARIWQFKRREWKSILLDMATRPAGQNLQGIEDKITKMKVTMVAWDRHDNTVITAVNNMTLKVWNSYTGQLIHVLMGHEDEVFVLEPHPFDPRVLFSAGHDGNVIVWDLARGVKIRSYFNMIEGQGHGAVFDCKCSPDGQHFACTDSHGHLLIFGFGSSSKYDKIADQMFFHSDYRPLIRDANNFVLDEQTQQAPHLMPPPFLVDVDGNPHPSRYQRLVPGRENCREEQLIPQMGVTSSGLNQVLSQQANQEISPLDSMIQRLQQEQDLRRSGEAVISNTSRLSRGSISSTSEVHSPPNVGLRRSGQIEGVRQMHSNAPRSEIATERDLVAWSRRVVVPELSAGVASRQEEWRTAKGEEEIKTYRSEEKRKHLTVPKENKIPTVSKNHAHEHFLDLGESKKQQTNQHNYRTRSALEETPRPSEEIENGSSSSDEGEVVAVSGGTSEEEERAWHSDGSSSDYSSDYSDWTADAGINLQPPKKVPKNKTKKAESSSDEEEESEKQKQKQIKKEKKKVNEEKDGPISPKKKKPKERKQKRLAVGELTENGLTLEEWLPSTWITDTIPRRCPFVPQMGDEVYYFRQGHEAYVEMARKNKIYSINPKKQPWHKMELRV

SEQ ID NO: 37 - DNA, 3316 bp, Mus musculus
gagccgaggctcggctcgtgagcacacactgacagctacagggcaggcggcggcaccgtccccgcgtcccctcggcggcggggtgtcccgccggcggccccgaagtgacccgcaaacatgtctcgtgagaggaaaggcctctcggagctgcgatcggagctctacttcctcatcgcccggttcctggaagatggaccctgtcagcaggcggctcaggtgctgatccgcgaagtggccgagaaggagctgctgccccggcgcaccgactggaccgggaaggagcaccccaggacctaccagaatctggtgaagtattatagacaccttgcacctgatcacttgctgcaaatatgtcatcggctaggacctcttcttgagcaagaaattcctcagagtgttcctggagtacagactttactaggagctggaagacagtccttgctacgaacaaataaaagctgcaagcatgtggtatggaaaggatctgccctggctgcactgcactgtgggaggccgccagagtctccagttaactacggtagcccacctagcattgcggatactctgttttcaaggaagctgaatgggaaatacagacttgaacgacttgttccaactgcagtttatcagcacatgaagatgcataagcgaattcttggacacttatcatcggtgtactgtgtaacttttgatcgaactggcaggcggatatttactggttctgatgattgtcttgtgaaaatctgggccacagacgatggaagattgctagctactttaagaggacatgctgctgaaatatcagacatggctgtaaactatgagaatactatgatagcagctggaagttgtgataaaatgattcgtgtctggtgtcttcgaacctgtgcacctttggctgttcttcagggacatagtgcatctattacatcactacagttctcaccattgtgcagtggctcaaagagatacctgtcttctacaggggcggacggcactatttgcttttggctttgggatgctggaacccttaaaataaatccaagacccacaaaatttacagagcgtcctcggcctggagtgcaaatgatatgttcttcgttcagtgctggtgggatgtttttggccactggaagcactgaccatattattagagtttatttttttggatcaggtcagccagaaaaaatatcagaattggagtttcatactgacaaagttgacagtatccagttttccaacactagtaacaggtttgtgagtggtagtcgtgatgggacagcacgaatttggcagtttaaacgaagggaatggaaaagcattttgttagatatggctactcgtccagcaggccaaaatcttcaaggcatagaagacaaaatcacaaaaatgaaagtaactatggtagcttgggatcgacatgacaacacagttataactgcagttaataacatgactctgaaagtttggaattcttatactggtcaactgatacatgttctaatgggtcatgaagatgaggtgtttgttcttgagccacacccatttgatcctagagttctcttctctgctggtcatgatggaaatgtgatagtgtgggatctagcaagaggagtcaaagttcgatcttatttcaatatgattgaaggacaaggacatggtgcagtgtttgactgcaaatgctcccctgatggtcagcactttgcatgtacagactctcatggacatcttttaatttttggttttgggtccagtagcaagtatgacaagatagcagatcagatgttttttcacagtgattatcggcctcttatccgtgatgcgaacaattttgtattagatgagcagacgcagcaggcacctcacctcatgcctcccccttttctggttgatgttgatggaaatcctcatccatcaaggtaccagcgattggttcctggtcgggagaactgcagggaggagcagctcattcctcagatgggagtaacttcttcaggattgaaccaagttttgagccagcaagcaaaccaggatattagtcctttagacagcatgattcaaagactgcagcaggagcaggacctgaggcgttcgggtgaagcaggtgttagtaatgccagccgtgtgaacagaggctcagtaagttctacctccgaagttcattcaccaccaaatataggattaaggcgcagtggccaaatcgaaggtgtacggcagatgcacagcaatgctccgagaagtgaaatagccacagagcgagatcttgttgcttggagtcggagggtagtagtgcctgagctctcggctggtgtggctagtagacaagaagaatggagaactgcaaagggagaagaggaaataaagagttatagatcagaagagaaaaggaaacacttaactgttgcaaaagagaataaaatacttactgtctcaaagaatcatgctcatgagcatttcctggatcttggggattctaaaaagcagcaagcgaatcagcacaattaccgtacaagatctgcactggaagaaacacccaggcccttagaggagctagaaaacggaactagttcttcagatgaaggtgaagtacttgctgtcagtggtgggacttctgaggaagaggagcgagcatggcacagtgatggcagctccagtgactactccagtgattattctgattggacagcagatgctggaattaacttgcagccaccaaagaaagttcctaagcataaaaccaagaaaccagaaagtagttcagatgaagaagaagaatctgaaaaccagaagcaaaaacatattaaaaaggaaagaaaaaaagcaaatgaagaaaaagatggaccaacatcaccaaagaaaaaaaagcccaaagaaagaaaacaaaagagattggctgtaggagaactaactgagaatggcctaacgttagaagagtggttgccttcagcttggattacagacacacttcccaggagatgtccatttgtgccacagatgggtgatgaggtttattattttcgacaagggcatgaagcatatgttgaaatggcccggaaaaataaaatttatagtatcaatcctaaaaagcagccatggcataagatggaactaagggtaaatattggcatattttttaatgtaaaatatattttttgcattattagagaagttgtgtgaaggtttactctttgactgtaagaaactggggctggggaataagagttcagaaggtaatatgcttccatgaaagtgtaaagattgccgggcagtggtggcgcacacctttagtcccagcacttgggaggcagaggcaggc

SEQ ID NO: 38 - protein, 1066 aa, Mus musculus
MSRERKGLSELRSELYFLIARFLEDGPCQQAAQVLIREVAEKELLPRRTDWTGKEHPRTYQNLVKYYRHLAPDHLLQICHRLGPLLEQEIPQSVPGVQTLLGAGRQSLLRTNKSCKHVVWKGSALAALHCGRPPESPVNYGSPPSIADTLFSRKLNGKYRLERLVPTAVYQHMKMHKRILGHLSSVYCVTFDRTGRRIFTGSDDCLVKIWATDDGRLLATLRGHAAEISDMAVNYENTMIAAGSCDKMIRVWCLRTCAPLAVLQGHSASITSLQFSPLCSGSKRYLSSTGADGTICFWLWDAGTLKINPRPTKFTERPRPGVQMICSSFSAGGMFLATGSTDHIIRVYFFGSGQPEKISELEFHTDKVDSIQFSNTSNRFVSGSRDGTARIWQFKRREWKSILLDMATRPAGQNLQGIEDKITKMKVTMVAWDRHDNTVITAVNNMTLKVWNSYTGQLIHVLMGHEDEVFVLEPHPFDPRVLFSAGHDGNVIVWDLARGVKVRSYFNMIEGQGHGAVFDCKCSPDGQHFACTDSHGHLLIFGFGSSSKYDKIADQMFFHSDYRPLIRDANNFVLDEQTQQAPHLMPPPFLVDVDGNPHPSRYQRLVPGRENCREEQLIPQMGVTSSGLNQVLSQQANQDISPLDSMIQRLQQEQDLRRSGEAGVSNASRVNRGSVSSTSEVHSPPNIGLRRSGQIEGVRQMHSNAPRSEIATERDLVAWSRRVVVPELSAGVASRQEEWRTAKGEEEIKSYRSEEKRKHLTVAKENKILTVSKNHAHEHFLDLGDSKKQQANQHNYRTRSALEETPRPLEELENGTSSSDEGEVLAVSGGTSEEEERAWHSDGSSSDYSSDYSDWTADAGINLQPPKKVPKHKTKKPESSSDEEEESENQKQKHIKKERKKANEEKDGPTSPKKKKPKERKQKRLAVGELTENGLTLEEWLPSAWITDTLPRRCPFVPQMGDEVYYFRQGHEAYVEMARKNKIYSINPKKQPWHKMELRVNIGIFFNVKYIFCIIREVVRFTLLETGAGEEFRRYASMKVRLPGSGGAHLSQHLGGRGRMSRERKG

SEQ ID NO: 39 - DNA, 142 bp, Homo sapiens
ccgaagctcggctcgtgaacacacactgacagctatagggcaggcggcggcaccgtccccgcttcccctcggcggcggggtgtcccgtcggcggccctgaagtgacccataaacatgtcttgtgagaggaaaggcctctcgg

SEQ ID NO: 40 - DNA, 71 bp, Homo sapiens
agctgcgatcggagctctacttcctcatcgcccggttcctggaagatggaccctgtcagcaggcggctcag

SEQ ID NO: 41 - DNA, 30 bp, Homo sapiens
gtgctgatccgcgaggtggccgagaaggag

SEQ ID NO: 42 - DNA, 60 bp, Homo sapiens
ctgctgccccggcgcaccgactggaccgggaaggagcatcccaggacctaccagaatctg

SEQ ID NO: 43 - DNA, 151 bp, Homo sapiens
gtgaagtattacagacacttagcacctgatcacttgctgcaaatatgtcatcgactaggacctcttcttgaacaagaaattcctcaaagtgttcctggagtacaaactttattaggagctggaagacagtctttactacgcacaaataaaa

SEQ ID NO: 44 - DNA, 99 bp, Homo sapiens
gctgcaagcatgttgtgtggaaaggatctgctctggctgcgttgcactgtggaagaccacctgagtcaccagttaactatggtagcccacccagcattg

SEQ ID NO: 45 - DNA, 161 bp, Homo sapiens
cggatactctgttttcaaggaagctgaatgggaaatacagacttgagcgacttgttccaactgcagtgtatcagcacatgaaaatgcataaacgaattcttggacacttgtcatctgtgtactgtgtaacttttgatcgaactggcagacggatatttact

SEQ ID NO: 46 - DNA, 222 bp, Homo sapiens
ggttctgatgactgtcttgtgaaaatatgggcaacagatgatgggaggttgttagctaccttaagaggacatgctgctgaaatatcagacatggctgtaaactatgagaataccatgatagcagctggaagttgtgataaaatgatccgagtctggtgtcttcgaacctgtgcacctttggctgttcttcagggccatagtgcatctattacatcactacag

SEQ ID NO: 47 - DNA, 101 bp, Homo sapiens
ttctcaccattgtgcagtggctcaaagagatatctatcttctactggggcagatggcactatttgtttttggctctgggatgctggaacccttaaaataaa

SEQ ID NO: 48 - DNA, 71 bp, Homo sapiens
cccaagacctgcaaaatttacagagcgccctcggcctggagttcaaatgatctgttcttcttttagtgctg

SEQ ID NO: 49 - DNA, 101 bp, Homo sapiens
gtggaatgtttctggcgacgggaagcacagatcatattattcgggtttatttttttggatcaggtcagccagagaaaatatcagaattggagtttcatact

SEQ ID NO: 50 - DNA, 41 bp, Homo sapiens
gacaaagttgacagtatccagttttccaacactagtaacag

SEQ ID NO: 51 - DNA, 99 bp, Homo sapiens
gtttgtaagtggcagtcgtgatgggacagcacgtatttggcaatttaaacgaagagagtggaagagcattttgttggatatggctactcgtccagcagg

SEQ ID NO: 52 - DNA, 154 bp, Homo sapiens
ccaaaaccttcaaggaatagaagataaaatcacaaaaatgaaggttactatggtagcttgggatcgacatgacaatacagttataactgcagttaataacatgactctgaaagtttggaattcttacactggtcaactaattcatgtcctgatg

SEQ ID NO: 53 - DNA, 135 bp, Homo sapiens
ggtcatgaagatgaggtatttgttcttgaaccacacccgttcgatcctagagttctcttttctgctggtcatgatggaaacgtgatagtgtgggatctggcaagaggagtcaaaatacgatcttatttcaatatg

SEQ ID NO: 54 - DNA, 129 bp, Homo sapiens
attgaaggccaaggacatggcgcagtatttgactgcaaatgctctcctgatggtcagcattttgcatgcacagactctcatggacatcttttaatttttggctttgggtccagtagcaaatatgacaag

SEQ ID NO: 55 - DNA, 226 bp, Homo sapiens
atagcagatcagatgttctttcatagtgattatcggccacttattcgtgatgccaacaattttgtattagatgaacagactcagcaagcacctcatcttatgcctcccccttttttggttgatgttgatggtaaccctcatccatcaagatatcaaagattagttcctggccgtgaaaattgcagggaggagcaactcatccctcagatgggagtaacttcctcag

SEQ ID NO: 56 - DNA, 138 bp, Homo sapiens
gactgaatcaagttttaagtcagcaagcaaaccaggagatcagcccactggacagcatgattcaaagactacaacaggagcaagacctgagacgttctggtgaagcagttatcagtaataccagccgtttaagtagag

SEQ ID NO: 57 - DNA, 184 bp, Homo sapiens
gctccataagttctacctcagaggttcattcaccaccaaacgtaggactaagacgtagtggacaaattgaaggtgtacggcaaatgcacagcaacgcaccaagaagtgaaatagccacagagcgggatctggtagcttggagtcgaagggtggtagtacccgagctatcagctggtgtagccag

SEQ ID NO: 58 - DNA, 118 bp, Homo sapiens
taggcaagaagaatggagaactgcaaagggagaagaagaaataaagacttacaggtcagaagagaaaagaaaacacttaactgttccaaaagagaataaaatacccactgtctcaaag

SEQ ID NO: 59 - DNA, 141 bp, Homo sapiens
aatcatgctcatgagcatttcctggatcttggagaatccaaaaagcaacagacaaatcaacacaattatcgtacaagatctgcattggaagagactcctagaccctcagaagagatagaaaatggcagtagttcttcagat

SEQ ID NO: 60 - DNA, 77 bp, Homo sapiens
gaaggcgaagtagttgctgtcagtggtggaacatccgaagaagaagagagagcatggcacagtgatggcagttctag

SEQ ID NO: 61 - DNA, 232 bp, Homo sapiens
tgactactccagtgattactctgactggacagcagatgcaggaattaatctgcagccaccaaagaaagttcctaagaataaaaccaagaaagcagaaagcagttcagatgaagaagaagaatctgaaaaacagaagcaaaaacagattaaaaaggaaaagaaaaaagtaaatgaagaaaaagatggaccaatatcaccaaagaaaaagaagcccaaagaaagaaaacaaaag

SEQ ID NO: 62 - DNA, 120 bp, Homo sapiens; identical to SEQ ID NO: 18

SEQ ID NO: 63 - DNA, 108 bp, Homo sapiens; identical to SEQ ID NO: 19

SEQ ID NO: 64 - protein, 996 aa, Homo sapiens
SSARRPVPLIESELYFLIARYLSAGPCRRAAQVLVQELEQYQLLPKRLDWEGNEHNRSYEELVLSNKHVAPDHLLQICQRIGPMLDKEIPPSISRVTSLLGAGRQSLLRTAKDCRHTVWKGSAFAALHRGRPPEMPVNYGSPPNLVEIHRGKQLTGCSTFSTAFPGTMYQHIKMHRRILGHLSAVYCVAFDRTGHRIFTGSDDCLVKIWSTHNGRLLSTLRGHSAEISDMAVNYENTMIAAGSCDKIIRVWCLRTCAPVAVLQGHTGSITSLQFSPMAKGSQRYMVSTGADGTVCFWQWDLESLKFSPRPLKFTEKPRPGVQMLCSSFSVGGMFLATGSTDHVIRMYFLGFEAPEKIAELESHTDKVDSIQFCNNGDRFLSGSRDGTARIWRFEQLEWRSILLDMATRISGDLSSEEERFMKPKVTMIAWNQNDSIVVTAVNDHVLKVWNSYTGQLLHNLMGHADEVFVLETHPFDSRIMLSAGHDGSIFIWDITKGTKMKHYFNMIEGQGHGAVFDCKFSQDGQHFACTDSHGHLLIFGFGCSKPYEKIPDQMFFHTDYRPLIRDSNNYVLDEQTQQAPHLMPPPFLVDVDGNPHPTKYQRLVPGRENSADEHLIPQLGYVATSDGEVIEQIISLQTNDNDERSPESSILDGMIRQLQQQQDQRMGADQDTIPRGLSNGEETPRRGFRRLSLDIQSPPNIGLRRSGQVEGVRQMHQNAPRSQIATERDLQAWKRRVVVPEVPLGIFRKLEDFRLEKGEEERNLYIIGRKRKTLQLSHKSDSVVLVSQSRQRTCRRKYPNYGRRNRSWRELSSGNESSSSVRHETSCDQSEGSGSSEEDEWRSDRKSESYSESSSDSSSRYSDWTADAGINLQPPLRTSCRRRITRFCSSSEDEISTENLSPPKRRRKRKKENKPKKENLRRMTPAELANMEHLYEFHPPVWITDTTLRKSPFVPQMGDEVIYFRQGHEAYIEAVRRNNIYELNPNKEPWRKMDLR

SEQ ID NO: 65 - protein, 183 aa, Homo sapiens
LPHRNASAVARKKLLHNSEDEQSLKSEIEEEELKDENQLLPVSSSHTAQSNVDESENRDSESESDLRVARKNWHANGYKSHTPAPSKTKFLKIESSEEDSKSHDSDHACNRTAGPSTSVQKLKAESISEEADSEPGRSGGRKYNTFHKNASFFKKTKILSDSEDSESEEQDREDGKCHKMEMN

SEQ ID NO: 66 - protein, 205 aa, Mus musculus; identical to SEQ ID NO: 13

SEQ ID NO: 67 - protein, 166 aa, Artificial Sequence
  mutant DN-mPHIP #1
RLAVGELTENGLTLEEWLPSAWITDTLPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQE

SEQ ID NO: 68 - protein, 152 aa, Artificial Sequence
  mutant DN-mPHIP #2
EEWLPSAWITDTLPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQE

SEQ ID NO: 69 - DNA, 140040 bp, Homo sapiens
  misc_feature (1)..(189): introns
gaattcattatagatcaatttctttcgtttcaaacagtgaatgaaatgaatgtgaaaatgcataacctatctaagggcaataaatagcaaacatttaaaatatatatgtatatatttatatatatatatatattcatttaaagaagtgaagtgtctcgtaagtttgtttttttttttttttttttttttgcaaatcaaatcataacattccctactccaccacagcagcaaggaagcagaagccttagttctacttattccttaactgtacctgctttatagattttgaagtaaaatattttggtacaagttaccaaccaattaaattagcttttgctttttcagtcaactttcggactcgtcctctactagaagttccaaaagttaaagaggtgtcttcgaacaacagctgcctttgctcctcttcagagtcatcctcattatagaaagctgtccttcgaccttgatttctagttctcatgtggggttcagagcctttgagttcttcaaactcttcttcctcatctataggatcatctatcttttttcggttacttctccttaacactttgacacttgcagggactaggagatctgcatctaatttttgtgtcttcatcttccttttgggcttcctacctccacgattctttttctgtaacaaatcttcctttacattattagtttcagaaagaaaattgcatgttgaagaaggaagtacttcatctctgatgggatgcacattattttgctctaaatcttctggctttgcatactgtagctttttgggctttctaccccttttcttgtgtataatttcaccactattggtattaacttctactttaggtttccttccaggtcccctcttcacaagttttgatggctgtcctccatggccatttacttgaatggttcctggtacaagagcgttgttcttacaatctcctaaaagggaacaacagtacacttaatatatggagtttctttttttgtttgactctcaaacttgtcagtaaggccccattggtatacttatatgtaatgacataatccaaattattttattaaaatgagaaaaaagaacctagaaaacactaatagttcaatgatcttatttattttctaattaaaagagacagattcttaacgatctcatgagggaggtaaagctcatagaaaataagtgacttatctaagttaacattgtgattgagtataaagctgggatgactaaaggtttcttattcctaacttagaaataagttactcttggtcaaaaactttgttcttagaactgtttaaagaggatttaaaaacaactaaatggcagttttcacaggctttgaaaagtcctatctccttgtgtaataaatggcaaatgactataatcctaggaaatactgattatatatatatatcaatcaaaatcactatgtgtgccaccattatcatgattaagatcccactgtaacaaactccatgataaaaggccattatgcttcataaaacaggagagaaaatctggcaatcaaattctaaacctgaaggatctggagatgatgaattacctttctgttgtagaaaaaaatgctataacttagataaaggaaaaatatgccacaggaactaccagtaactaactctctttccctccaaatttctgacatgtttttatttgatatggtaggtgatttgcaatgctctatttttgaggaaattcatagatggaaactgcttttaaagagaatacatcttcataacagcatttttggtcacaggttggaactgtactttgtaaataagaaaatcatggttggtcgggtgcagtggctcacgattataatcccagcactctgggaggccaaagtgggcggatcacctgaggtcaggagttcgagaccagcctggccaacatggcgaaaccccgtctctactaaaagtacaaaaagtagctgggcgtggtggtgggtgcctgtaatcccagctactcagggggctgaggcaggagaatcctttgaacccaggaggcggaggttgcagtgagctgagatcacgccactgcactccagcctgggcgacaagagggagactccatctcaaaaacaaaacaaaacaaaacaaaacaacatggttaagagacttaccaaaggtcagagccaagtacagacagaaaatgcaaagcttttaattcctgacccccatagtgaaatgactctctttagattagtggttgggaaaaaatgtgggtgtggacataaagtagttaagtatttcctatggagcaactgacatttaaactgcccagggattctgtcaatctcttttgttttacattatgactacaagggttctaaatatccagtacatgtacctactacagaagataggctttaaggactacatgaatcccttgtaatggtcccaaattttgtatggatatgtttatgtacatttttctgagacagggactataggactcatcaactttataaagcaatatataatgtaaaaaggttaagaatgaatgcagctttcttaaaaaggaattcagaagttttttaaaaaagtttaaaaaccactgatattgaacggtgttgtcctgggcacatgaaaagttatgcagcttaggaactaaatttttatttaaatttcaatttaaataccaaagcagtatacattttaaaatactgattaaatactgaaataacattttggatataatgggataaataaaatattattaaaattaactttacctgtttttactgaacatggtgagtacaaaatttaaaattacatatatggcttgtattatattccactagacaacactgcttgattctaaatagttaatttaggtgtcattatttgtaataaaacactgttaatgttaactaatagataatgatatttctatacagtaccagtactcgagtatttgtaatactcaaaaattaaaaatcaaacaggttatgataccagacctccgctatcataatgctagaaccaattcatcatatatatttgatttctctaggattcatgaataaaaagaagcaaggccaatatactattcaaactctaaattcagttcagaaagggggcagattattaaaaatgtgaaacactcatatgcaaagcattttggttattcaaacacttattatcttctgtataatgggcattaaaaatgagtaaacacattaagctcagattctttggagatacagacatgtgaaaatgaataatatgatcaacattataagtaccaccaaaggtaatgaacagggttttctgggacataaagatggaagtgcttggctgggcgtggtggctcacatctgaaatcacaatactttgggaggccgagtggggtggatcaccagaggccagaagtttgagaccagcctggtcaaaatggtgaaatcctgtctatatcaaaaatacaaaatcagccaggtgtgatggcacacacctataattccagctacttgggaggctgaggcagaagaattgcttgaaccggcaaggcagaggttgcagtgaatggaaatcaggccattgcacttcagcctcggtgacagagcaagatcctgtctttttttaaaaaaaaaaaaaaaaaaaaaaaggaagtgcttgattctatctaaagaagccaggagaagacttcctaaagaagacgatattttaagtgagacgtgaaaggcaacagacaattaaactaggagggaaaaaaagacattcccccaaaaggagaaaagaacaaagactcaggaacttctaagtgtttaggatgactgggatacaaaagagagaaagaaggtaaaagaacctggaatgttaggcaagagccaagtaataaagagtcttgtgtaacaggcaaaaaatttaaaatgtttccatatatgatttgaaggcaaggaagtgttttctctgtgtgtacgtacacacatccacatgtgctagagagaaataaaaagatcgctttggctgcaatatgagagagggactggttaagaaagagttgagaactgaggcaggaagaccagttaggaaactaggaaaatagtccaagcaagaaattatgtaggccttgaaataatgtcatggaggtgagaatggagaggagagaatagatttaagagatgttatggagggagaaacaacaaaaacaaaaagctgttgaacagattcagttgctgaagagaaggctaggatgactccctgattttaagtttacacgggtagatcccaatgccattaacaaaaataagatttcagtagagaaattaaattttgagagaggtttctgaagacaacaatgaagaaatgtcttagacacactttgaaagtcatgatgcaaaatgcttattattgggctgtctgctgccaagaagccatattattttaacatgtcacatggcatattttattatttaccttcttcatctttcaaacataaagactttacaataaaaacctggaggtgaaagaacttgaagtgtaacagtaaggtgtcaaaagttgtattctacagttgtagacaaccccaatgaattattatttagtaaaagtcagtctagaaaaataagtagttttgtgatccaataattacttaaacatttttctagaaaagtgaagaatgctacattgggttaactataccctatttaatttaaactttgaagatttatttcttttttttttttttcttttgagacagggtctcattctgtttaccaggatggagtgcagtggcacaataatagctcattgcagtaaatttatcaactaatacagatgtgtgacttttaagtgggcaacctgaaaagtggatataaatgctgattccaacaaaagcattatttataataaggatctactgtatcttgaaagatacaagtaataccttaccttgctcaatgacagctgatgactttgaaagagtggacgcctttgggagtacagatgacttcattttacgtttgactggcttttccttttccatgttttcttttgcagaattattcctagtgccatgactaaaactggattgaccaggactggaaagagtattcaaagctttggaatgtttcacagaattctctagtactaaaacatacaaacaaaatttaaaaattaagagttattgaacctaaagataagaaaaaaggttaacctgaattatttgaattagccaagacaacaaaacctgaaggatgcttaaagctttcttaggaaagctactttctaataggaaaaaggcgtatccaactagaaactcttaatagtttcagcccttttagaagctgtcccatcatttcaaaatttcgaaggcaagtcttggcaaattgctagctagtgtgggtactgtgatttaaattcaggtagtttagatcagagttgccatttttaagcattagtctataatgacctaaacctcaatttaattcttcttattaaaaacttttttttaaaataggaaattaataaagaaggcaaaaacaacagtgtctgctaggaattactaaaactcagtatattgcatttggcaaagtaaaagcttaaattaagaaaatcatcatatacatttcaatttagaaagtgagtcttacttgttttccctggtattgcagatgcattagcttttgtaataaaagtctttgcagctgaagaagtagatggttgctcagtgacaactggatctacaaccactcggttgcttctggttcgaaccacagaactagattctgttttaccgtttatctgagcagcattgtgtcttggcggtattgatcgtgtaggtgtagagaatgcagaggtagagctttctgattttagctggggttttaagatcctttttttcctttcagggctgtaaataaaatagtattgtcagtcactcttatagctctatgtgaacgaataaaacagtttataatatttttggattcaatatttgtactattatgaaatatgttaaaatatgagatttatagtggatttcatatgattgtgagcctttgaaagtgaatatttagtgaaggatcgctgtaaatgctaaagttatatgacggaaagcatgatgccatcactatcctaaaaatgctgttttactgtatagatttagcagtttgaatttaagcacttacactagtatagctttagttaaaagattaaaaatcctccacatcataggaacttgcatgtcaaattatcattctgcaatatagggaatagtaaaggaagtattaaaaaacaccaagttctatcatttagatgaaagttatagatcagctagtggtatttaaaagaaattaaataccttgatgcagcactactggaaacagagctgcttctgtttcttttcttcctccttttggttatggtatttcttttatgaaaacgaagagcagatttataatctgataaaactgaactaatgtgttcttcaaagaaagcagacaggcgcaaactcatgctgtaaatctgtgagggaaaaaaaaaagtgttcaaccattccttggaggaaaatacctttgttcagtaaatactgtaatgtaaatatttttccagtaaaaaatatttagaatttaattattgttttttacatccctttttcctaatcttttgatgaaaaggtaaactgaagcattttaacaattatgtatttttgtgtttagaacagaaatcttccaagttttgagattcttaaagaaaagtccgactctaaattcaaatggctcatacagacaaaacttattgtcaactttattacactgaaactatcccaaatgtttgaacctgttttctatctaggactagcatctattctttctcatttcgttgctatatagcactcctttgtgatgtcatgtctggtcagagtgttaaattatatttttacttatttgtaaaaatcttcgcaaaaatgctccacaaggcagataatagctagaaaactcaaggccagatggctctggtgcataccaggacaatttgcatcaaccgcactacttcaagaaaagtaaccattcccagacatcaaagataacatcaatgttatttcatacaaggagctgagtagaaaggtataatttctttttccagtaggacaacattaagaatgtaacagaaagttaactttgacctaaattttaagtaaagcaacatttagtcatttaacacactcctctaacttaatctagtcataaaagaaaataatgtaattatatacccttgatcttttgcttggtgtatatgctttggaattactgaaaataagtctgacatctttacataactccattggtgactcataattcccagcctctaaagtttctctaacggtagcaaaatccattggagtgtcaatgatgtctctgtagtcctaggagagggaaaacaggtggtgttatgattattactacacaaagcatcacttctcagtgcagggattcgcacaggatttttatgatgactgcaagtcctagaactcttaataatcactcctgttccccttatcaagagtcccttttttctaataattcttatttatttcatactccccccccttatactgcaatcaacaataattttcttattcaagaacacagaagttattaatttttcactggagacttgggagatggagttgtattggaaaagggaaagtaaaagagtaaggaaaaagcccagctctacaaccgaaagtttgaaagaaaaactcaaaactttatactacttataaattctaaaggtctgactcattaaaacacaactgtaactttaaggaaataaaaacaatggaagtatgccagcatcccatttatgcagacacctaagttctagtaatctcaacttcagtactaaaattgggagttttgctttgcagtaataaagaattacgaatgtaaatagttgtcacaaagtctatgcatgtcacctgagatgtctacctagtcaatagagtataaaattaggtaacagattggaaccaataaaaacacacacgtgaaacaggaagagcaacagaaaattatcataatatggtatataattctaaaattatcagaatatgctatttttttttagcagggacaaagagtattgtaccccccctttttgggagacacagtcttgctgctgcccaggttagagtgcagttggtgccatcaaagctcactgcatccttggcctcccagactcaagcaaccttcccacctcagcctctcaagtagcggggactacaggcaggcgctaccacacccagctaatttttataatttttgtagagacgggtcttagcatgttgctgagactggtctcaaactcctgggcttaaatgacctgcccgtcttgacctcccaaagtgctgggattataggcattagccaccacacctggcctgcagcttttcaacagtccctcagtatgcgactatattttttgaagtgtaacaacttgactttgcaccatcaagtttaaattatgatcaaatacgtctgaccatgaagaaggtgtcctataaggtaggattactgcctttacaaatttttatttcttcctttccaatagttatgccttttatttccttttcttgccttattgcattggctagaatttccagtactacattgaatagcagtggtgagagtgaacatttttcagtcattcctaattcttagggggaaagcactcagtctgtcaccagtaaacatgatattagctgtagatgtactttttatagatgtactttatcaagttgtggaagttttcctttgttcccggttttcttaggggttttataatgaattaatgtctcacttcttcagattctgcatttgtctatttgccatctattcacaggccaatgatgatctggtacctggggggccttacagacctgggaaaagattgccccttcctgggcagtcttagtgaggggttccactgagaacatgtctttcatatacataccaatgaatcccaagtataaagccacaatcagctccttttctcactctcacacactaagccagtatttccctgttttaaatcatctcagagctgggaccagacaactagatacctgtgccccagggcccactggaattattcaaactagccaataataagctgttaactgtgacctgccttgcatttcctgcagaaaccccaataaaggatttctaagcttttccctggttttggtctctcctacccaaccaaaacctagcacttcccctgtggccctgtgtggcatgtggtaagccccgacttttctgggactcttttttacttttttttttttgttgttaatgagatagggtctcactctattgccaggctagagttcagtggtatcatcttggctcactgcaatgtctacctcccaggctcaagcaatcctcccacctcagcctcattagtagcttgaactataggtgcacgccactgcacccggttaatttttgtattttttgtaaagacggggttttgccatattgctcagactggtctcaaactcctgagctcaagtgatccacctaccttggcctcccaaagtgctgggattacaggtgtgagccaccatgcttggcctgggactcgagtataataaactttttccttccaagccttgttttcatttcctcctgtgaccgcactgactttaccataaccaaaatacacattcacagaacaaatgggtgtgaaattttgtcaaatgttctttctggattacttgatataatcatgagatttttcttcattagcctattaatatgatggattacactgactggtttttgaatactgaaccatccttgtatctctggaataaacagcacttggtcatggtataaaatcattttttaatatattcctgaattctatttgctgttatttcgttaaaggtttttcttcttttctactcttattgtctggttttgagatcaggggaacactggtcttcatagagtgagttgggaattttgagtttttctatcttctggaagagattgtgtagaatttgtgttaattctttaaatgtttggttgaattctccagtgaagccatccaggactagacatttgttttttgaaaacttataatcacaaattaaatttccttaatagggttactgagttatttgcttcatactgggtgagttgtggtagtttatactttgaatatcggtctatttcatgtaagttatcaaatttatatatgtagaattctttgtagtattacttatttttactttattatccttctggtatttgcagggtctacagtgatatgctctatatcatctctgatattaacaatctgtcttctctctttataagctgtgtaaatcttaacagaggcttgtcaattctgttgatcttctcaaagaacccagctttcaatttcatagattttctttattgtttttcttttttgagtttcactgatttcagctctttattatttccttttgttggcttacctttgggtgattttactcttctttctctaggttcttgaggagtgagcttcgattattgatttgaaacttctccttttctgctgtactctttagtacattttagtattagaaatttccctgcattgctttaactgcatcctacaaattttgatatactgaatttgttttaattgagttcaatgcattttttaaattcccatgagatttgtttgatccatagattatttagaggtgggctctttcgttaacaagtccttggagattttacattattggctttacaaactttgtggatttgggcaaaatataaaaatgattttttaaaatgttttgcaatattttggttaatgtaaattttatttatgtagaatacttaatttttcttggctcatatcttgggcttacaccatgtagtatagtacataatagctgctcaatacaattctgttgaataaatgaacgttgtagaatattaagcccattcatttccattaaaaatttaatttttaacatcttgctttgaatatttgattaaactcaaaatgtgaaccaatattttcatataaaagatgaaatatgaagtgcatgatctgccttaaatattccactaaaggatgatacagttaattctgaattataaaaagtagattatccgaagttttctttttctcttctgtgacagtaattaacaaaacaacaaacctccatcatggagtactgcaagaggcaagagattacatttttcttatttctactactttttgttgcctaacacgtttagctggtgggacaggttctaagtatttgctaaatattgttctcattattttgaacatgtaaaagatgactgcattcttatatatttcccttttaagtttgaaaagtgaactactttctttatataaaaattcatttgcctattgtctcagaatatcacatataactggtgcactggacataagggatacgggttcccattgtggctttgtctataaatagctacaaataaatagctataaatagttttgtctataaatcttagagtgatgagtatcagttcaacatcagtaaagtgagtggcttggagcagtctcaggtctcctccattttttctgtatgactgtttcaatattttctttttgactttcaattgttgagtttttttcactcttattctaggtaaagattcttttcttgtatatcccgatcaggaatcatagcttctcaaatgtttaccaattctagaaaattcttggccatcgggcatggtggcttatgcctgtaatcccagcactttggggaggctgaggcaggcagatcacaaggtcaagagattgagaccatcctggccaacatggtgaaaccccacctctactaaaaatatacaaattagttgggcgtggtggcatgcgcctataatcccagctactcgtgaggccgaggcaggagaatcgcttgaatccaggaggcagagtttgtagtgagccgagattgagccactgcactccagcctggagacagagtgagactctgtcccaaaaaaaaaagaaaaaaaaaaaagaaaaaaaaaaaggaaaaaaaaattcttggccattagctcctcaaatattgctcctctcccattctgtctattctcttccactgaaatttttgttagacatattttggaccttctctttctatctaccactacctcttaccctctccttcacactcttaatctctttatcattctgtgtgggattctatagaatttgcttagatctttacacttactatctctttagtctgtttttaaaactgtccagtaagtttaatttcagtaattatacatttcatttccaggattctatttagttgattttaatatgtcttcactcttttcctctcaaataacatattttttccctatgtttcttattctttcatttattcctttaaccaaaactatctatcaaagtttaactcaacagcatttcttttttcttggtggtacatataacaacagtaatcttacaaggaatgtcatctctcttttttttaatgaagtacagtacttcagaataatctattactcaaattttcagggagagggtactaatattttcatttgttgtttctgttatcttattatggtagatcactcccttatataggtgttgttttttttttaaatcattagttcattcagttaaggattaacattttttccataatggatttctacacaagggtggtgcaaatttggattctaagtccatgtatagtgtaagtttaggaaaatttctcctctctgacactagaaccactgggggaaacattctttgttgtgaaaggaattattcaattcttcttttcattcagggtacagttcttcaatatttctggtttagggttgggtttcagctccaaattcctttttcaccactgcccaaggactcaattatctctgtatagtgttaatacttgtgcctctagaataaaaacattgtcttatttctatctcttcttttctgtgcaaagcccagaatacaaacgcttaaaacaatgaataaactgcaacttatttttcaaaagaatacatagctgagcttgcaagaaccaaagcgaaatccataagttgtgaaaacacagagagaaatgaaagccagaacattatagcatcagctcagtcccaggttttttgaaaggtgaggttctaattagctcaatttatcacgccgctggaattaaagatttctcttccacatttaacattctatgtttctggcattttaaatgacatgaaaaaagtcattttctgatatttatctgttgatgaaatttctttattttcatcattgtaagttagaacaaaaattagcccggctaatttttgtacttttagtagagacgggattttaccatgttggtcaggctggtcttgaactcctggcctcaggtgatccgcctgccttggcttcccaaagtgctgggattacaggtgtaagacaccacgcccgacccctgaactatataacatttaattactttttaaagggatgagaaatcactctacattaaatttagattgctatgattgcacgccaaaataaatacttaaatcatgtttacttagctctttttaccatgtattccataaagattacacattggcataacctaaatatatacaataatgtcaccttacatttgtacacagtgcttcacatttaaaactattttttgtttgcttttgagactcagtctcttgctctgtcgcccaggctggagtacggcagtgggatctcggctcactgcaagctccacctcccgggttcacgccattctcctgcctcagcctcccaagtagctgggactacaggcacccgcccacacgcctggctaatttttttttttgtattttaagtagagacgggatttcaccgtgttagccaggatggtctcgatctcctgacctcgtgatctgcctgcctcggcctcccaaagtgctgggattactggcatgagccaccgtgcccagcctaaaaactatttttatatattctctttacatctccataatcctgtaaggacgtaggcattattctttttttctagataattgccataataaattcatggaatcagtgtagggaagacaaaaaaagaaaaaaaaaattcagatgagaaaactaagggacttgctcaaagctgcacaactagtaggaacagaataacccaattcttacagtgtcttcattcagggctccttccattttaccacactattcaaaatttggattctctatgtagccaaatggataatgagaacatgtataaaataataaagaaataaactataatcataaaaagtaactaaaatagccaactgtcatgtaaaaggtatgtagcaaactgacaggtaaagaaaatattttcaaaaatacttactggatattcaaggagatctaccggctgacggaaaggctctgaatcttcacattgaaatatgagatttaacaattcttcacactgtttcttccatgcttgaatatcgtaagactgggctctattacgtaatcttcttctaggctgatggtcctgtgataaaagtgttcaaatatattaataaaagagcacttacacaataaaatttgtacttttaatgtagtcttagataattgggtaatatacaataattcaaacaaaagaaaatattcaccaagttctaaaaaacatacattttgtaattgaaactaatttgaaatacttaatgtcttttaaaatgctaagagtaaaaaaataaagaaagctcttaatacattttaattcatataaagtacttctgctaaaactaaaactatattaccttcctttttcgagtagaagttcctggcacatcagcatctttctcttcatcctttgaggcaagaatttaccagattcataaaacattttagatgtcattatactttatagttgattaactagcaattatttcctttacacactggaacacctgtaatgtatatgctggggcactttattgactcattaaaaaggttccccccattaaaaaattttttttaactataagaagaatattctactgccagttgttttttttttttaaattaactacactagacaaaaaataatgttcacaacagcttttacctgaaaactacaatatgtaaatttttttatatagagaatatcaatatggtaataataatgaaatattacatacctcagaatcagacaaaactttcttcttcattgaattataaagtggaattatgttataacaagtctgatccctacataacaaggaaatgttaacatgtaagattagaaccatgataatttttttccttaaaaatttgttcgtaaaaccatattttaaggtaaaagttgaagctgaaggcttgctttcttctccattggcttactccaataatttatgcacacacatttaaccctgacccctccactctatgtagagctttcagtgtggcctcactatatcattacaccaaacccaagtctcatctcccagtctttgcttaggtatctgctgtgcttcttccattcctcagtcttcaaacagctaatacatttcttgtcttccaactctttcttttatttttaaatgtatttcctaaaattttgttcttaacgcatcttgacactgtaccttttgcttcacatacttgtggtttatctgtgtaaatgaattccaaaattcctgcaatttgtcctaacccttctcctgagcattctaccaacacctaaaattccacgtctaaacttaatagtgactccccagatatccttgttcctttttctatttttctttaacaacatatcattcttacagtaatgggaatcttggtttttcattattctttccctttcctcctccttataaacccaattagtggtcaagggctgtgaactctatcctcaagtatgtctgggcatctgtccctttctcactttcataaattaagccctcatcaactcttagttggtcaactgtagcagtcaatctgctatttatgctttttgtctcttctctgtcaattaaattagacactgcagtcaaattaacatttttaaggcatagtgtaaaacatgttattctcatgttaatatactttcaacagccccttgctctcagagcttatattttagattcatattcaaagccacccacgatgtggccccaactcagatttatagcactgtatctctactgtgacttctccaatttatattacccttaattaaaacttcctacctcacgctgctccttatccctggaatggctttcttttcatctaacatttccagaatctatcagtatctacaccttgtatacaatgtcttaacagacttctcctcccctgtatctgaatccccatcatcacagctaaaagtaatcctattcttatctaaacttttatacaattctcttactacttgtcacagttttccttatattatactttcttacagatttatctatcctatgaaactgtattaaaaggatccatcacattactttgtatgtatcaattgcttgaaattttgccaaataactcaattaaaaagtataataatcaaaatttcaaggaatacttaatatcttgaagactatctgctaaaaaaagtatttttaaacaaactatacacatctaaaaaaatgccatggatttatttttagaaatatacaatacaaatgctttaagtatttcatgaatctgacttcaaagacatttcaaagtagccgtttgaaagaaatacatttcacagactttcaaatgtattaacaattttcatctaaattatttcactgaaatatgaatatactatctcatgtagttctactgattctctttgaaaaaacagatacacatacacatagttataacacttataaaaaattacagacataagagtcttcagaggataatgcttaattactaatttcaataaggaaaacaaaagcatagcaataatagcccccaaaccattggaaagcaatagatttcttagagaataaggtagagaaagggcacaaactttaccttattaattatgtttggtgttttcttatactgaaatgacctgcattcctcagttaaaacacattaatcaaaaaggagctcaaagattatgtccattaatgagaatgaagcagggatgtttattaaaaaaaaaaaaaaaactgaataatcctgaatttttcattatgtaaaaatgaaagctgataacagctaagtaagcttttaaaatgctgttactacttctcaaccaggaaaaaaaaattcaatacaaataatgacatggaatcacagcagtctttgtacaaaatatagaattcatttctctgccttcaacttaggaggctcaattcattatatgattgcataaaatccttaagataaggaagggaaagtacttctgccttaataaatagtgcttatcactcttgttatgggatcaatgaggaagtaaacttgactttgaagaagaatcatgaaagttaaattcagtctcctgctggactatttaaatacttgttaatatacttgacaggggcaatatactgttaggatgaaaaattctcaaatcagatggcagacactcatttaccgtgcaaccttatacatgttaaccactataggccacagtttcctcaattcaaaattccagataattatctcttccacctgtaagattgttatttgggttagaagagttaatgtaggtaaaacattacatgttaaataaatttttactattattattgtctaacagttagattgagaaaataatctttttttaaacaattttaaccttaaaacacaatggtaatacgatttttatgatttcattcttattattagccaatgaactgtttcttctgaaacccaggatcaaaccagagacctttagatcttcagtctaatgctctcccagctgagctattttggctactcttaaatgtttcttctttacaaacagtatgttttctattttaagaggaactgtagtgccattaattattaaaactatcataattacatatgaaaagataacttactttataaaatgtagaagaagatcagtcacgaatttagcagatttcacaatagggcttccaggctcattaaatgttcgtgtattatgctctatatatcgaacttcccacattagggaagaaacccgccttaaaaaaacaaaatatagaagttttaacttccttatatttagaaatatgtgtacatcatttaaaaccaagacattccaacttttcaacttcagtctaaaccaactgtaaaaaccattggtcttataaagtcattttcaaagcagcataactgcatttgtgttaggggaaaaaaagaggggcaaccataactacgtatttgcatacaagatgtctggaatggaacacaccatattaacaaaggcctctttttgggagggagaacgtctatatggggagtggcaggagagtagaaaggggagagttttaagttttggctttatgtatttttgtcatgtgtgctgtcattttttggtaataaagaaccctcacttctgaacaaaaaggaaacaagtaattttaatctaattatcttactggtaatcaaatgacatatacaaatgagaagttaactgacagccaccttatgagatacaaaatcataaaaatatagcatgctagtttaccaagaaacctaactaaatgagaattattttctgaacacttaattgacaatgctaaaataaaatctggagttttacaattttatttctgaaagtaaataaaaatccaaggacaacttttgagaatattatctaatatgtggcctgacttaaaataataaagaaaacacttagaaaatcttactgattgtgaacagaaatacaatcatatggaataacactgtatctaattgtggacatagaaacataaagaaaaactgtgcatttcaaatagattcacaaggctcattctgataacagaatcacagatatcttcagtgtatcatatagaaaactgtgtgtaaaataaagtattagattaataccagcagggcaaactgacagtaatagtttaacaagagattgaactagaagtttcacgaaagaaaaacaaactgtaagaagtctaacaccaatgagtgaaggaagaagcaaaaacctacttacattgtattgaatgtaatacattgaagtcatcattgtattgaataagaacataacttaggtttataacagagtttattatcaggttggaaaacaggcaatttctaattcatgtaagtattgtctttcaaatgtttttttcctaaattggctacaaaactagggtaatgccaaaagcctatttaaaatataatgtatcttgaaatacagatgttcctcaactaacgatggtgttacatcctgataaacccactgtaaattcaaaataccattaagtcaaaaatgcatgcgatatacttaacctagcaaatattttacctcagccaagcctacctcaaatgtgctcagaacactgacattagcctatggttgggcaacatcatatggcaatcaactgtacaatacactgtacagtattggtttaacctcatgatcacgtggctgactgggagctactgggagctgtagctcactgacatcgctcagcatcatgaaagagtacatcacaagcccagaaaaagatgaaaattcaaaatttgaagtatggtttctactgaattcatatccctttcatatcactgtaaagctgaaaaatctaagtcaaaccattgtaagtcaaaccttatgtatagtatactttagcaattatcatgttgagcaatatgtgagatatttacaacaatattggaagacatcagcagcttatatttctagttgcagtccaacaattagtgtatgtatacaaatagttctttccttctcatccacccatgtcttgtttcatctctgaagcactaggtttaatttccaatctttagcaatttaaggggtcaagggagaaagaggaatatagttaggaattccttttttttttttttttctcttaaacttccaacacctgatattgaaaaagacttgaagaatgctttgagggtgggatggttgggaaccacatagcagggaggacttctccttatctctacgctttttgacaaatatcaaggaagcaacagcaagtacacctaagaagatcagaaaaatctttagaaactaaagtctaatatattagttttctttattccagtggttctcaatcaaggacaattatgctccccagaagacatgtgacaatgtctgaacacatttttggttttcacaactagggggctgctactggcatctagtgggtacaggcacaggatagcccctcacaatgagatacagaaataaaatttaagtcataaaaagaaccaaaggcacattttatatagaaaaatattaatacaaaatatgaataaagcttctctatgttaaaaagaagaataacggaaaggacttcagtaataaataactggtcacaaaaacttttaatgcaatgttacacaaattaaattgttggactgctaagcaaaggtcatatgataaaaattaaaactaaaaacaggattccattatttaaaaactataaacatattttttgacaaaacattttaggtaataataaagcctactgacgattaaagacattcatcaaaattacctagataatgcaaattaattgaaattatgcctgggccatttaactctaattctttttcatgctaaactacaactatgaaaaactgagtattttcaaatttcagtgttataagtaatgataagctgaatacaggcaaaataagaacaaaatacataatacgaatacaaaatttttattatatattatattaaaaatcaatgaacaaatattttaatgttatctaggagaaaatgaaataccttgccttgctttataaaatacaatataaagacagtgaatatcaaatcatgtcagcctctaggaaaaactgtatctaggatctaggaattgtattttaagtgtctaaagaccagcagcatcgacattactgggacttgttagaaatgcagaatctcaggtcccactccaaatctactgaataagattctacattttaacataatccccataatcttatgtgcattcctatatacaaaaagttgagaaacactgctaaagatcagtaaatgggtggaaagatcaccagctttcctttgatagtagctacatcaaatggttctaacctgtaaaacctgttttccagtctttgtttaattgtacttagatccgttggatatgccactactgtgcaatacatgggataggcttgcagatccacgggggccacaaatgctgaggcaatatctaaaataaatagataagtttgtaaatttatttttgtatgcctaaaataattcaagaagaattctgcttgaattaagacttaaaaagtcctaatctacataatcattagtctgccactgtcttttcataaacaatatagtgtagctgacataaagagtctcactttttattatgtctacatttacactgccaaattctaggggtattgttttattcataggttcaccgggagaaagaaacaaattatatatacatatatcagacctaataagtacacatacattgccgtaattgacagttggccattcctgaatctatatttggcaaagccattctattattaacactgtaacatactgcattcaaatctaacctttgactttctcaaacgtatccctttatctcaactttacattattctgtgaaatattagacaagtagagaaaaggaaacagacccagaagttttgattcagtgatactgacccaatacacaccgtaaagttgtacagcaaaaactataactagttaagatcactcccaattttttgctcattcgcatataatacttaatatcctaaaatattgctaaaattatttaaggtagtatttataaggctattcctataaagtgttggcattttataaaatacttcagatttgaatattcctcaatctccgtgtccatccagctcttcttactcatgttaatttctctctagactctttgcagctgattctttattgagagagtgggttgctacaaaccaccacataatctagttacttcagaagcccagaatttagataatcaagttttgtggtcactgttttcttttaacaaggcagagcaattaatataccctctcctctccccttaagaagatcctcttttgtgtgtgtatattaagttgggggagaccagtacaagctacccatataattataactcagctttcaatcctcctcctccaattcatatcatgtcagcctgaatatgtcaagtgttttaaattgggttgtggaggacccagttttttcagagatgcctctggcacttctaggaggcccttattctaaaattcagctaacataacctaatttataactgttttaaatagttaagtcctgtgttaagaccacattcaaaaagagattccacttaaaatgtctgaaaccactgacttaggatattgtgaaaaaaaatttttgttggagaataacagtatttttccattactttgtgttctgccagttttttctatactcgcgtgttgctttacttacctagtgtcatcaactggtttattcctgccacaattctttcacattcttcatccctgggattggtaccccattctccatcaagaggtttatagattagtgatctgcactcaccatcagttaaaggaacactggtacctagttcttcaggaaatacagctgaaatagaaaagcagatcattgcaaatacatggtaacttattagtattcaggttagctttagaatgtaaaaataacagtcacaaaattaaagtatattttgtatagatttgtaaatatactctttattttaacaaaggaaagtatgttttaagggtcactaaaatttaaattaatttttaaatgatactataagtaattctctaaataattacttctccaaaattatacctgaaaatctgctctgtaatcaagtacatgtgcagaaatcatttctataaaatatgcaaatttcaaagttttctgaatcacttcataattgccatgtttactttgataaagtatacacaaggtaaaactgaactaaagtgacattttctagaaatacttcaatcaaagcttcaatttttgaatgtaggaacagagagaattatgaaaactgacaaatgatgcttatgcttattactcataaattatgaaggtatgctttctgcatgcttgaatctttaacagttttagctgagagaatcactagagggttgcggtaacccaaaatctagaaacgtgctaggtaatttttcctttagactaagttttggcagatacactctagaaaatacgccactgtttgtgtacaatcaaaattctcatcacaaactacgattaaactctataggttcgtatgaatgtgtatccaaatagaacaacaacagtaaccacccttcaatatatttaggtaggaaacaaaacagtgaatcagtatcacttattcatttaaaaaatatccaaggcattataacaccaactggctgcattgctatcatattcacaacagttcaatgtgagttcaaattcaaactttctttttaataaatgagagaaacaaaacaaaaacataagccatgtaacatggttaccaattgatttaagatattttataattttaaacagctctaatttagcagtgagataaaaaaaaatatattattggcattaaattttcaaagtgataattcctgcagagaactaatcttagctaatcagtatgacaattttctcatttctgaggtctacgagactgggttattttctccaacagttattttttctctgactttggattacaataactactgagcaactcaattaataaaagattatttctactatgttagaaattagatgttttcctttttgttttttaaagatcttatttatttttatctgctaagagatgtaatatattattattatttttgagatggagtctcgctctgttgcccaggctggagcgcagtggtgcaatctcggctcactgccacctccgcctcccaggttcaagtaattttcctgcctcaaccttctgagtagctgggactacaggcacacgccaccacgccaggctaatttttgtatttttagttgagacggggtttcaccatgttggtcaggctgctctccaacacctgacctcgtgatccacctgcctcagcctctcaaagtgctgggattacaggtgtgagccaccactcctgccctgtaatatattattttttaaaaatcaaacaatatagaataacgtaaaagaatttatgaagtctttataatgctactctataaataatcattattaaaatctcgacaatatccttctaatccctttttggcttatgcaattgtgcatgtgtgttgtattttttacaaacaaacaaaaatgggcaatgaagtggaaagaaaatataatctccaggctttggtcccaacgtccttttctcagtgcaaggaagatgtcatactcactgcctaaggctaattattaaatcctgaatgtgtcaggccatatgcataatgacagttatattatcattattaattacaactatatcttcattgagctcttatatgtgtcaggctctacaataagcactttacacacatgatgctatttaatcttcaaagtagccctataaggaaggtattagctttgacggtttctaaggccgagtactaaaaagttggggtgtgaggctttatggaacttgccaagatcacataaaaaatgacaagtcaggatatgaactgatgtccgtctcactcaaaagcatgacctcttaactattatgttacactttaaacactctgctaaagttacaaaagtgtctctgcctcccaaatgcacactttcttgggtgaatagtaattaataaaacaatttcatgttttgctgtaataaattaatttcaatcaattccaagtaggcaagagttatatctatcttcttcactgctgaatctcccctacttcaaggtgtacaataataaatatttgccaaatgaatgttttctatttgatactcacaactgtaagtggtagcatgttagcaaatcttaaataatttcttaagaaattaactccataatggagaaacaaaaagcctaacacacacttaccattattaggtataagctccatatcccaaggactcatcttttctgtatctccattgtcccagcttaaagaaagaaaaatcattatattaaaaaatctaaattattgtatcacaattttaataaaatcaattatcaaaataattgcttctgtgtttaaaagaagtctctttatctcttaatagatggaaaaaaaaattcaaagcaagcctaggtgaactaaaatacaacaaatatttccttaccaaacattgtagcattgaaacagactatcagggtactcaagttgaagaggttcctggctttcgattgttccaaaccaccaggcatcatctatgacagacctgaagcggtcacctggccaagaacaaaaactaactcatcattctgaaatgcatggctgctgtcactgctttttcctaacgttaacctttaagtacctaaactgcctgtatgatttcagaagacaaaaagtgaaccacaaactccaaaaataagtaagtacaatcagcaataccaagagaaaaaaggaatttagtaagcatacttgaagtgtgacttaacagttttcaattctattttttatatttcattaaggtatacagaaattcacttgttttaggcatttttaccaatctagcatttgaaattcatcattaacactatacccaaacttttcactgaaataaaattataattgcggcaagttccactcaacaattacttagtcttttaatttcttactttctgtaagcaagtttccccaaccaacaatcaatcaagactccacgctaaaaacaacaaacaacataaaatccaacctgtcttccttcatctcaatcacccttaatactcactcactctccctttcctgtaaaaggaaacaaaaaagaaacaaaaataaaacaactattctttttaaaacagaggacactccttgtgtctatttccttatccaattgctgtcgtcgtattacttaacctgctttttcccaaagacctgaacaagctataaatgctatggctttttcttatctaaatattctcatgttccttctgtgtcataagaaaactgtaagtcacttacttcctttatgcaatgttttcctgttcctcagctatcacagtacttgagttttctcgtggctagcataggctagagcctgaaagacttgggttcaaataacgtatctgtataactttgaatatattaattatttttgaacttcattttccttgtctataaaatagaaatggtgatgtctacctcctggtagtttttgactaatgagttctattaaagcactccacattttgacacacagttaagttaaaatgaattaagttagcaattaatctgaatcagttttatttttaaactcaaagagaaaagtagttatgttctcattttcttaccaaaaaggatttaaaagtttataaaaacatatagcatgaaatgaattaaaaattagaaaaacaaggtaaaagaatatgataaaaaagaaaattggagccaggagaaaagttaatagaacacaactgcatgctgttgacagttgttcctcagatgcatactagtgacacttaacaaaaagctactatatagtttaataagaagcattcttgacaccaccactacctcacactaaacataaagtttcaaagagctgtatcttatgaccctacacagactgattattatgctaaagaggaaggactgagtaatgtgtgcctaatcataacgaacaatatctgtagaataacaagaaagttaagcaaagaacctggactactactgaagtccaccattgagagtaagccttaatacatttccgaggaggggctgaagtaaaaattactaatgtaattttaaatggcagtcggtgggtgaatgcatatatcctcagatataaaagattaattagatactaactttagagaaatattaacccataaattagaataaaattgtaagatccaaatgagaaaattatgatgctcattcattttaattctctgtgatttgtccaatgttagccacattactttgccaaggttatgagctcacttctggaatattgctgcactttgatctctattatttgttccgcaatttattggcaaatgcaactccttaaaaaataaaatttatcggccgggcgcggtggctcatgcctgtaatcctagcactttgggaggctgaggcgggcggatcacaaggtcaggagatcaagaccatcctggctaacgcggtgaaaccctgtctctactaaaaatacaaaaaaaaaattagccaggcgtggtggcaggagcctgtagtcccagctaatcgggaggctgaggtagaatggtgtgaacctgggaggtggaacttgcagtgagccaagatcacggcactgcactccagcctgggtgacagagcgagactccatctcaaaaataaaataaaatacaatttatcatatcaagtaatgtatgtgaaaaatttaaacaatcagatgtaccaaaggctgatagccaaaaccaagaaaaatgttttcatctattttacctactacttcttgacttacagatttcttcactcatcaatttttgacagtaagtatcagagttgattcttgaagacatgggttttaactgaccaggtctacttatacacagatttttccaataaacagatttggccctctgtattggcagattctgcatcagcaaccaaatgcagattgaaaatacagtattagtgggatgtgaaatccatgaatatggaagggccaacttttcacatcggggggttccgtaggatcaattctggaacctatgtatgcaaagattttggtatccatggaggtcctggaagtaattccctgtggatactaagggacaactataacttcaatacaactgtgcataaaaagtatgtgtattatatttaatccatattcaatttttaatcatgactgtgtaaatactgcttgctcctaagcaaaacagcatataattccttccttatataattttgttttccctaaaattaataattgcttcatttttttaatgcttggttttcagtgaatttacaattaaatcttcccacaatctctaacagctcaagctgtaaaaaacattcttcaatgtaattttccacaaagacaaacttgttagataagttatgtgttccaattttttccccctgaagactttcctcttgaaggagatttggacctgctaggtagctgctatcctgaggcttctactgaatattatttgggattccttttaacttttcctgttctggacttcctgtttcctgaggggattccattcttttccctttcttggttactcccttatttgatgaaacacatctttcaaaacttctagggaaatggaacatgagatgtaaattttctttctaacttccatgtccataattttgatagcttccaaattttcctaatttctccttacagagcacatacaagtttttaacaaaggacaaaccaccatgtcaatgcgttctagtggcactgaaggacagactggtatatcaattgatgtgctttttcaaaccatacataccgtgtacacatcataccatgacacacactggtcctatatgaaatttttaagagaactactctcataagtggcacatcatctatatgtaaattaatgcaccttaagtgcctgaaaacttttacagattttagtttctttgagacttgtttatgatactaattttaaagattataataggattaaccaataaaagaaaaatgtcagtttagctttagtcccagtacaaacttatacatcttgttaagcttcttttggcttcaaatttaaattgttattaatatttttatacaaaaatttaacttaacatgtaaaacatgaaaataaagtcaaatgtaacagaaaaaatgttttaaatttcaacatcgactgttttcattcctaaacaaaacttaacaatacctctgacatagtatcttactttgctattaacattcctacaaatagcaaaaagatttctctgataatctttttctcaattatgaaaaatagtaaatcacttactaagaaaaaaaaaccacatcaaacatcgatagcttctaaataaattacccacctatattccatcgcctgtattttgcatcatcaaattgttgtctcaagactaggaaatctataacgtcaggcatatcatggtatctaattacaaacagaaacaaattgattaggtcacatacctaaaaatacctaaaagatgttcaaatgtatctgaatcttgaaaaacaatctacaatactaagaaaaccattgcctctttcaacagtccttacttcatggtaaatgatccgccagtcagtttaccagtatcaggatctagaaaagcaagtttaaggcagcaaagggtaggtaatcccacttcatactttatgccaactattttcataagttcttgttcctgagagagacagagaatataaggaaccatctttacaaaataaaccacaaaatagactgctaaacattgttgagaaaaaacttcttggtttaaatcttgatctggatggtgatgactgtgtacatgtaaaaattcactgagctctgtattaagatttgtgcactttatagtatgcaagttacaatgaaatttttaaaacgtaaaaaaggaaaaaaaaataaggagaactttactaaagtatagttactaaagtaagttttctatcaccattccaaagacctattggtaactgaactactgatgacaaatctagaaatacgcatcaaatttacgaatacaaagcttactttagacttattacctaattttcactataactaaattttgtacccaacccataaactgcattggagtatataaattcagtcaaatcttgtgtttctctggatgaaaattaaacacctcctactctctaggactgcttcaattaaacaaggtatcttcatgatgcagtttcttattgtgttaatagtaacctttgatacaattttctccaaattccataattgtatttttggggctattaaataataaatcaatgtcatacccgtagctccattttatgccatggttgttttttgggattgatactatatattttatttttccgggccatttcgacataggcttcatgtccttgtcggaaataataaacctaaaaaataaagtcataatcttacaacctggatgtgtttcctttaatccaatatacagggtatcagctgaaattgcaaagagaaaatctaatgccttttcagctaatagaaattctaatattttctccctcctcctttctctcaagttgtcttaacatcactaaatcataaccaaagtgttctaaataacattactttgaatacacacttataagttaagatgaaaagttattttctggtttcaccattttattcttaaaatcaaaataagttaatctatgttgcataataacgttgactaataagtttattgtccacttttgttgttaacttttttagcaagtagtttgtacatatgcagaaaacattgaaatgaaaaatgtactttcacatatattttaatccttacacaaatcctggtaagacagcagggcatacatttatccacatttcatgaagatcaaatttaagtgactcctgactactgtcttggagaactagtacttgaacacagcagtctgacacccactagtagaagaacgtgttttaaaatgctaagtttttagtaacattttgggatagccactatcccatactttcattctattcattaataccattattgcggggaggggccaagatggccgaataggaacagctccggtctacagctcccagcctgaacgatgcagaagacgggtgatttctgcatttccatctgaggtaacgggttcatctcactagggagtgccagacagtgggcgcaggtcagtgggtgcgcgcaccgtgtgcgagccgaagtagggtgaggcattgcctcactcaggaagcacagggagtcagggagttccctttcctaatcaaagaaaggggtgacggacggcacctggaaaatcgggtcactcccacccgaatactgcgcttttccaatgggcttaaaaaacggcgcaccacgaaattatatcccgcacctggctcggagggtcctaccccacggagtctcgctgattgctagcacagcagtctgaggtcaaactgcaaggcggcagcgaggctgggggaggggcgcccaccattgcccaggcttgcttaggtaaacaaagcagccgggaagctcgaactgggtggagcccaccacagctcaaggaggcctgcctgcctctgtaggctccacctctgggggcagggcacagacaaacaaaaagacagcagtaacctctgcagacttaaatgtccctgtctgacagctttgaagagagcagtggttctcccagtacgcagctggagatatgagaacgggcagactgcctcctcaagtgggtccctgacccctgacccctgagcagcctaactgggaggcaccctccagcaggggcacactgacacctcacactgcagggtactccaacagacctgcagctgagggtcctgtctgttagaaggaaaactaacaaacagaaaggacatccacatcaaaaacccatctgtacatcaccatcatcaaagacaaaaagtagataaaaccacaaagatggggaaaaaacagaacagaaaaactggaaactctaaaaatcagagcacctctcctcctccaaaggaacacagctcctcaccagcaatggaacaaagctggacgcagaatgactttgatgagctgagagaagaaggcttcagacgatcaaattactctgagctacaggaggacattcaaaccaaaggcaaagaagttgaaaactttgaaaaaaatttagaagaatgtgtaactagaataatcaatacagagaagtgcttaaaggagctgatagagctgaaaaccaaggctcgagaactatgtgaagaatgcagaagcctcaggagccgatgcgatgaactggaagaaagggtatcagcaatggaagatgaaatgaatgaaatgaagcgagaagggaagtttagagaaaaaaacaataaaaagaaatgagcaaagcctccaagaaatataggactatgtgaaaagaccaaatctacgtctgattggtgtacctgaaagtgatggggagaatggaaccaagttggaaaacactgcaggatattatccacgagaatttccccaatctagcaaggcaggccaacgttgagattcaggaaatacagagaacgccataaagatactcctcgagaagagcaactccaagacacataattgtcagattcaccaaagttgaaatgaaggaaaaaatgttaagggcagccagagagaaaggtcgggttacgctcaaagggaagcccattagactaacagcggatctctcagcagaaactctacaagccagaagagagtgggggccaatattcaacattcttaaagaaaagaattttcaacccagaatttcatatccagccaaagtaagcttcataagtgaaggagaaataaaatactttacagacaagcaaatgctgagagattttgtcaccaccaggcctgccctacaagagctcctgaaggaagcgctaaacatggaaaggaacaaccggtaccagccgctgcaaaaacatgccaaattgtaaagaccatcgagactaggaagaaactgcatcaactaatgagcaaaataaccagctaacatcataatgacaggatcaaattcacacataacaatattaactttaaatgtcaatgggctaaattctccaattaaaagacacagactggcaaattggataaacagtcaagacccatcagtgtgctgtattcaggaaacccatctcacctgcagagacacacataggctcaaaataaaaggatggaggaagatgtaccaagcaaatggaaaacaaaaaaagacaggggttgcaatcctagtctgataaaacagactttaaaccaacaaagatcaaaagagacaaagaaggccattacataatggtaaaggggtcaattcaacaagaagagctaactatcctaaatatatatgcacccaatacaggagcacccagattcataaagcaagtcttgagtgacctacaaagagacttagactcccacacattaataatgggagactttaacaccccactgtcaacattagacagatcaacgagacagaaagttaacaaggatacccaggaattgaactcagctctgcaccaagcagacctaatagacttctgcagaactctccacctcaaatcaacagaatatacatttttttcagcaccacaccacacctattccaaaattgaccacatagttggaagtaaagctctcctcagcaaatgtaaaagaacagaaattataacaaactgtctctcagaccacagtgcaatcaaactagaactcaggattaagaaactcactcaaaaccactcaactacatggaaactgaacaacctgctcctgaatgactactgggtacataatgaaatgaaggcagaaataaagatgttctttgaaaccaacgagaacaaagacacaacataccagaatctctgggacgcattcaaagcagtgtgtagagggaaatttatagcactaaatgcccacaagagtaagcaggaaagatccaaaattgacaccctaacatcacaattaaaagaactagaaaagcaagagcaaacacattcaaaagctagcagaaggcaagaaataactaagatcagagcagaactgaaggaaatagagacacaaaaaacccttcaaaaaattaatgaatccaggagctggttttttgaaagatcaacaaaatcgatagaccgccagcaagactaataaaagaaaaaaagaagaatcaaatagatgcaataaaaaatgataaaggggatatcaccaccgatcccacagaaatacaaactaccatcagagaatactacaaacacttctacgcaaataaactagaaaatctagaagaaatggataaattccttgacacatacactctcccaagactaaaccaggaagaagttgaatctctgaatagaccaataacaagatctgaaattgtggcaataatcaacagcttaccaaccaaaaagagtccaggatcagatggattcacagccgaattctaccagaggtacaaggaggaactggtaccattccttctgaaactattccaatcaatagaaaaagagggaatcccccctaactcattttatgaggccagcatcattctgataccaaagctgggcagagacacaaccaaaaaagataattttagaccaatatccttgatgaacattgatgcaaaaatcctcaataaaatactggcaaaccgaatccagcagcacatcaaaaagcttatccaccatgaacaagtgggcttcatccctgggatgcaaggctggttcaatatacgcaaatcaataaatgtaatccagcatataaacagagccaaagacaaaaaccacatgattatctcaatagatgcagaaaaggcctttgacaaaaatcaacaacctttcatgctaaaaactctcaataaattaggtattgatgggacatatttcaaaataatcagagctatctatgacaaacccacagccaatatcatactgaatgggcaaaaactggaagcattccctttgaaaactggcacaagacagggatgctctctctcaccactcctattcaacatagtgatggaagttctggccagggcaattaggcaggagaaggaaagaaagggtattcaattaggaaaagaggaagtcaaattgtccctgtttgcagatgacatgattgtgtatctagaaaaccccactgtctcagcccaaaatctccttaagctgataagcaacttcagcaaagtctcaggatacaaaatcaatgtgcaaaaatcacaagcattcctatacaccaacaacggacaaacagagagccaaatcatgagtgaactcccattcacaattgcttcaaagagaataaaatacctaggaatccaacttacaagggatgtgaaggacctcttcaaggagaactacaaaccactgctcaaggaaataaaagaggatacaaacaaatggaagaacattccatgctcatgggtaggaagaatcaatattgtgaaaatggccatactgcccaaagtaatttacagattcaatgccatccccatcaagctaccaatgcctttcttcacagaattggaaaaaactactttaaagttcatatggaaccaaaaaagggcccgcattgccaagtcaatcctaagcaaaaagaacaaagctggaggcatcacactacctgacttcaaactatactacaaggctacagtaaccaaaacagcatggtactggtaccaaaacagagatatagaccaatggaacagaacagagccctcagaaataacgccgcatatctacaaccatctgatctttgacaaacctgagaaaaacaagcaatgggaaaaggattccctatttaataaatggtgctgggaaaactggctagccatatgtagaaagctgaaactggatcccttccttacaccttatacaaaaattaattcaagatggattaaagacttaaacgttagacctaaaaccataaaaaccctagaagaaaacctaggcattaccattcaggacataggcatgggcaaggacttcatgtctaaaacaacaaaagcaatggcaaccaaagccaaaattgacaaatgggatctaattaaactaaagagcttctgcacagcaaaagaaactaccatcagagtgaacaggcaacctacaaaatgggagaaaattttcgcaatctactcatctgacaaagggctaatatccagaatctacaatgaactcaaacaaatttacaagaaaaaaacaaacaaccacatcaaaaagtgggcgaaggacatgaacagacacttctcaaaagaagacatttatgcagccaaaaaacacatgaaaaaatgctcaccatcactgcccatcagagaaatgcaaatcaaaaccacaatgagataccatctcacaccagttagaatggcaatcattaaaaagtcaggaaacaacaggtgctggagaggatgtggagaaataggaacacttttacactgttggtgggactgtaaactaattcaaccattgtggaagtcagtgtggcgattcctcagggatctagaactagaaataccatttgacccagccatcccattactgggtatatacccaaaggactataaatcatgctgctataaagacacatgcacacgtatgtttattgtggcattattcacaatagcaaagacttggaaccaacccaaatgtccaacaatgatagactggattaagaaaatgtggcacatatacaccatggaatactatgcagctataaaaaatgatgagttcatgtcctttgtagggacatggatgaaattggaaaccatcattctcagtaaactattgcaagaacaaaaaaccaaacaccgcatattctcactcataggtgggaattgaacaatgagatcacatggacacaggaaggggaacatcacactctggggactgttgtggggtgggggacgggggagggatagcattgggagatatacctaatgctagatgacgagttagtgggtgcagcacaccagcatggcacatgtatacgtatgtaactaacctgcacaatgtacacatgtaccctaaaacttaaattaaaaaaataccattattgctccagttgtctggaactttaaatgatggaatagtgctgaataatgaagaccttcaagcaccagcagctaaaaagacaacctaaccattttgaaaggaaatcattcaacaaggaattacaaacttttctggccatgttgaaataatgtattaaatataatttcacatcttaatgctacaggatgttaagatctatcactgtactatatcacaacaatgtggtaatgttctgtgctcttcaagtcgtatgaggcaagtaggatttttaaataaattctttcagactgttctttagaatcactctagcattaaaaagtttttgttttcttttttttcttaaagtgtcaattaccactttatactgtaaattagtgtgctttgctaaagaagcctctattttaattcccaatttatttatggtcaatctgttggacaccagataacactgcctcttaaaatttgtgtacaaaacaaggagacattaaccttgagtaaaaaatgttgagaccagttcttacattttcattctaaattcacaagtacatattctaaaacaatggtctcatcctcagtttcttaaaaattaaatagtatttcttattctaagattatagattttttaaagatctaatgaactgtgaacactgtgggtaaacaaaacactaacactttttgaaataaaattaaatgaaaacaaattgctaaatttcatttttcctttaacatgttatacattaagccttaagataaatgaatatattttaatttaaaaagaaaaagaaagctggcaataaaaacctcttactaaaacatacatttctttttaaaacactaacaaaattttcattcttataatattcagatttacagcagcttcaaagaagattattagactttcactcctaattgtactttctccatacatagatacaaatatctagaatgagatactttacaaaactgcttctattttccagaagtctatttcaagagctgttaaaaaatgtttaaaactatgtgaggaaaaatggataatttaaaacttttaaagtacctcatcacccatctgtggcacaaatggacatcttcggggaatggtatctgtaatccatgttgatggcaaccattcttctaatgtcaaaccattttcagttagttctcccacagccaatctctgacaaaatttaagtaataattgttaagtaataatcaaaaaagcattaatccacaaatctactctttaaaataactataaagataattaaataaaaggggaagggcaccattgtacaatattatatacagttggccctgtgtatccatgggttccacatccatgcattcaaccaaccttgactgaaaatatttggaaaaaaaattccacaaagttccaacaggcaaaacttgaatttgctacattctgaatactatgttgaatccacacaaataaaatgatgtgtaggcactgtagtaggtattataaataatctagaaatgacttaaagtatataggagactgtgtgtaggtcacatgcaaatactatatgccattttttataagagacttgaacatccctggattttgttatctgcaggggtcttgggaccaatcctccgtgtctactgagagatgactatcctttaagcaacaaaacacttattctagaaaaaatggcagcagtggcaacagagttattcaatctctccaaatcactgtctacaaagagaactaactagaagcaaaacccaaaaatccatagccaacactcacaacaaggtaacaagatatccccacaaaccccaaagtacaagctctgtgtattctcctttcctttcaaaatgctatcaattttataaacattccatataacaaaccaagacagcctagagtttttttttaatcctagttatagtaagttacaactaaataaggtattctcataggagttgagtagtgcaacatgtagaaagctaattatttccataagctggacattacacttctacacagcatgagaaactatgcctctgagaaagttccttaactttgctggtcacccacaagtggccacaatggtcttgatgttgttaccttagactcaggaaaaaaatgaactttctaagaacatttgaaacctaatatttttacaagtaaaaaaagttatgcaattgattaaagtcttttgtgaatcacacgtaaaacattaaaaatgattgtacactaagactgctacattttacttgtttttttaaaaacaaggtagtgtaattatcagtataaaataatacttgtttactaaaagaagcaatgccataacatgatatcagagaacactacttgcaataggtaatactactacttcccaactgtagtagttgtcattttcctctttttcctattagccacagccacactgagtgtttctcagtcaaacatatcaagagcattaccctggagagttagggtaaaggtctttggaatttactgtacgtgagatgggctcgttacagaattttaggcagaagcatagtacgatctcacttatattttaaaaggatcactctggatgcatcaaaacaaggatcagcaaatcatgaccctcagggcaagtctggcctgccacaattttcataaatttttatcggaacacagccatacccatttatgtattgtctataaatgctattatggcagagtagctggccttctgtagaaaaaatctgtcaacctatgctttaaagaatagaccctagggagaaacaaggagaaacaggaagacacatgagatgctaccacagtaatataaatgagagttcagggtgattcacaccaatgtgatggcagtggagtggtgaaaaacagtaaagtgctgaatatacttataatccattagataattaattccctgatggactggatgtggaatatgagagaaaaagaggaatcaaagatatctccagggtttctggtataaacaactaagagagtcgtcatattactgagataaagagggctggggtacagcgggtttgaggaaaaagcttggtaaataagttttgtaggtgttggatgtgaggagtaaaatgatatccaaacagtaatttgatatatacacagttatcaaataaagtagccattatgttatgcactgagtatatcacagagatcccacaacccaggaacttccactgtgctttattcagagcagctgctatcagttttgtatactgaggagctaaaagtttgtttgaaaaaggtttcctttgactaataaaaaggaaaagaaagacagaaaagtttgaaaatcataattctagcctcaatatggactattaattgctaggcaaggatttctccccataaggaatttatctatgttcaatggggaagctaacaacttttacatcaagacaggtaagttgtatattaaataagaataatcatatgtatgactgaaagactttgggcatcaccaaaaatcattatgaggacatatcttattccccaataattcctgaggaacttagaatgtttggttgaggaagatttctgtcacttattaattataaccattaaggggttaagaatgcattgagtattctttaacatttctagctccatgatttgaggtttttttaaatatggaaagtataaataatttctttcctttccaaccatgggaatgcttctaggcgcagctcaaatcttatttcctctaggaaatctcatctgaaatcccaacataagatgaccctgtttaagaatggaaatacttttatgtttatttgagataactagaggactagggaataagaacaagtccatctactgttacatcactgctaggcatgcgaacaaaatttaaactaaagggaataccctccagtatcctaaccttaccatatttaaagtgacccacataattctattggcagagtcatcctatcacaatactttgttgcaacaacacagtgaaattaaaacctgccaaatgttaacaatagatctatatgtgccagtgcatgaaggagcccatggtattagtttgaggaaaattatcaaattaaggggaccagatgaagcaaactgtactcttgggatgaaggaactgttctcacagtaacttatcactgcaaacctcaaaagttcatcaaaactgtcctgagatgaaaaagaatatttacctaattagccagacacatggctaaatcagttttctttaaccaatctgtctactacatacataaaataacaaatcagactacatttatcatcactgacaaggtagaaaagaaaatgtgtgggtaaatgtgtggcattctctttggtaaagcttccccaaacaaaagcaaaggagacaacaagaaaaacagcagctgtggaatccaaattaaagtaactcataattataaagctgaaacaagaactcaacaaatgtagataaaataaacacttaaaacagatatattcttatactttttgaaagctctgtgggaataaagaccctacggtctcctactatttaattctacaaacgacaatttatacttaaatggcagaggttacaaaaaaatcttccttttaaccaaatactgcttcccatttacagtcttttgggaagcagaaaacatgcccctcgtgttgtatttactttagaacaaaaattaaacaggtctcattttcattcctatgttaaagaatttcaactccataagagctgacagatattcttcctataatatttcttacgttctctgctttctctcaaattgtccacagacctaattgtggtaatcaaatcaatactgctactgaccaaaggactgaaaacttatagctcacacactgtagattaatgaaggaatgttacaagaccatcttaccccactttattcgccttggggtcaactgtaacaagttgtgtcatttctactctgtgtgatgttcctcacagactttgtttattttcagcaataacaattaataaggatggttaaacataaaggcaattcatagattaatacactatatgttttgtatcattatgtatttcacataaaaaaatcccaccaaactgtcaagaaaaatctgatctttttaaggttaaatgttcaatgtttagaatatggcaattttgtaagtaaatacttttacatgttaaatatctaaaaactcgaataaactaaatatatttaggtaaacttttttactactgtgtatttctgagttttttctattattatttgctgatacacttcaacaatgtttgtgatcaattgtacttcaagatgtgtgctttaaactgtttctctagaaaacaccaaatttgtaatgttaaaaaccaaaccttttgttttctttctttgggcttctttttctttggtgatattggtccatctttttcttcatttacttttttcttttcctttttaatctgtttttgcttctgtttttcagattcttcttcttcatctgaactgctttctgctttcttggttttattcttaggaactttctttggtggctgcagattaattcctgcatctgctgtccagtcagagtaatcactggagtagtcactagaaggagagaagggattattagataacacacaagataaaattttaagacttgttttataaaaacgaaaagaaagattataataaagagaaatgacagaacaaatacttaaactacaaaaaaacttccctaattttcaactcatcttcaaaagtttcttgaaattctataggctttacttaaatatcgcaaaaaaactcccccaaaacctgaagatacataaagtcacaactttaagtgaaaaaacaaatttaagtaccttccaagattttaagaaaaatactgacttctgagtgttgcatatgttacaaattctgtgagcatataaaaataagagggggtcacttttctcagttctattgacagcaaaaagataaccctggagagcacagaaggtgtcaacattaaggagatatggggattccttttttaaggggaggtgggagttccagacaggggaactcttatggtccagtactagtgctctcagggtagctggatattgaactggcagagtcagaagtgttcctacccaggggaaagactgtgtttgcaaactttactctgcatgtaaagtaacaaaatttcatttagataaaattattctacctacctacaccctacacgttaggtgtatcttatgcttcaaagcatgccatgattcagaaaaatacaggccttactatattttgtatactgcttccaatcatgtttgtcatttctttttagtttaccttcattgacaaacgatttactgaaccaatcatgttgcaaaataaagaatctagttgtattatcttctatattaattttagtatgtgagcatatttacttattttgtacaattttatgaacataaatgtcttaaaggaacctaagttatactatgttaaaagcaataattagctttcccagatttaatgatatatgacctcatgcctctaaaaatactgctgttaacattaacactgaaaaagcagatttttctggagctttattccattgtcactaaccctccccactacccctctccactgtccaaatgttctttataagcacgtacctaaatctctgattatcctaatcatcccagataagaatcaaataattaagatgttctctagcatgatttagtgatttcactatgaaatttaaaatgatttttaagaacttacactattttcagctatgctattctaaaataggttgtcttcaacttatgagcattttatttatactagctccagccatggccactgtaaatggtacctgccaaatcacacagttatcactctctggagtacttggctcttacagaagatcttctgctgtcagcataaacataattacaaactccagagtccagcattctttacaaccgtcctcaactgctaaaaccaccactactcttttaatacaggaactcctatgtcatctcagggtttgaaatactctgattctgctgtcgagccaaaagaaattcaatttttccttcattgacctgttttgaatacagaaaactactagatataaccacacacatttgcacatcattaccagaaatcactactgtgggaataggagagcccttgattgtacaactgaggaactcttacagcctctacttcatactttgtctcgtctattttaagagccactaaggtgggggataagtgtagcagctaatcataattgccatccccaaaggctatactctctttttgctattaagaccttgcaaagttagccaaggtcataagctctgcaacaatgtgaatcgccagtataaggagttgaaggaatcctcactggtctggtcaaataaggtaggcataacaagaaaaaggagagcttattcctcttatcattagtattccataaattccactatataagatagttaactcaggaggcttgcattgctttcttaacttcatacttttcaaaaccagtaatgaaactggtttgcaattcaacattataacggtattcagaagaaacaatactaagatgataaagttaaaagcatcattttgcagatctagttgcaatcaccaaaaaattattttctatagagaacatatatcagaaaatctacatttcatacaacttcaaaaactctctgaagaactttgaacttacagagactttgaaacgtgttgctggttaaaaaaaaaaacacctttctaaagactttatataacatttggaaaaataaaaagcattcatttacctagaactgccatcactgtgccatgctctctcttcttcttcggatgttccaccactgacagcaactacttcgccttcctaagatatgttgaatacatgtcttattgcataattttataaaataacattttatgattacagaaaatatcagtgatatcttataatatcagtcatattgggatatttaaaatttgatttaaattagttgcaaagggtgttgtggctcacgcctgtaatcccaacactttgagaggtcaaggtgggcagatcacttaaggttaggggttcgagaccagcctggccaacgtggcgacaccctgtctctactaaaacacacaaaaaattagctgggaatggtggcagatgcctgtaatcccagctacttgggagactgaggcacggaattgcttgaacccaggaggcggaggttgccgtgagctgagatcgcaccactacactccagagactgtctcaaaaataataataataaaataataaaagagtcagctgcttctctttaaaaaattagttgcaaaatacagcttttacctttttttcacaaataaatgagaatatgtaaatgtatataaagacataatactaatattggattcagagcaatcctacagattaccattgtaaaagcctcataacgtatctagataaaatgataacaaattccatatttacatttgaaattaatttttacttcttgagcctttttaaaacactaagctttctcttctataggtgctttgagaaggcacatttatgttttatttattataggcacatttatgttttacttattaaaacagctgtcccatataaaaaaggatgttccacttctggtcttattttatctaaatggtaaaggattaaaaaatacttaaacactctgtttctctttggtaaatattcatgagtaaacaaagaatattatctgtgaaagcattttcttcataaaattgggttttttgatggcaaaaatgttgcatgtcttcctcacttaaataaactttatgctccaataacaataacacctcatatttattaagcttttactgtatgttacaaactattctaagcactttgtatgtatcaccccatttgggcttgacaatgcactattggtgatagagagttacttaaataaacaaactaaagctttaaaaatttgagaattttgcccaaggcggcacaactaataagcaacaaaacttgatttaagcccaggtcactttagtccaaaccctggacaacattctgtaagaatggtgttacttacttgctgggcatattcagataacttttaaagaggacaatggggtggattcttttaatatttaaataaaataatatacttgggacaaaagttttcccattttgatgttttaatgaagaatgtgaattatatgtctattccacagttatgaaaactaagcaaaaatgaaaaaatcacatttgcatattttattaacaaaaagcatattttattaacaaatttaatcactgctcgatataacaaatctaagcaagtgatatgatttttcccacattttgtgtgttacttccttaaaggggggaaaaagaaacagaattattcctacattccaaatacaaaatactaaagtacaaaacatctgaaccaaaaaaatcagaattctttccaattaattagtatactgtgcccaaagtgatactttgttctcaatggactcttctagaaatatattctaaatttctaaaaggatgttaaagcataaataaattagtttgaagacacttgaaggacttgttgtaacagatgtgtttctggaaatacagttcttaagtttctaataaacagaatattttaaatattatatagaaacttaatatttagaggaaactctttcaaaaataaagagacattttataagataaataattagcccacatgtattttatgttttgtatgtctatccaaattaccatgtgtcaaattcatataatcagtatatttctctttttttgccagaatgaatatatcattttgacctggctatatgaggacccaaagctattggagactaacttaaattgtggttagaatactctcagtttgagagttaatgttgccctggataaagagaatatatttagagaattctcatatatgcctagcatttcaaagcacaacatggcttcaaaagtgtatagtagtcaatatctaaaatgattacatctctaacctgtaaagaaggaaggtgatgggaaaggttgagaagaaaagaggaatggagatggtttaactgcttaataagattggtttatgattgcaaattgattgccttcactgaaataatatataatttagtaaggacagtaatatttactgaaataatgacgcatgaggaatatttaaaaattacactgagaaagctgtaaactggtacagagattaaaaactaacaaaaattgtcaaatacttattgatttgttgttatacttacactacactacagcttcatggtaccacttaaaaagcttggctttcaagtaaaacaaacttgcatttaatccctgctctacctcttaataggtgactgtatagtctaaatatctttgagcctcagaatccttatctataaaatgagaataattaattcacgagtttattttgtggatttgaaattatgtgtatgtgtgccatgcctagaaatagatttttagaaaataaaagctgtatcataagaccccaaaatgccatatttaagctacatctacaaacatatcaataaatgcgaagaatgaaactcatatttaaaaaaataagtaaaaatgacatctaatttatgaatgtgctgatatcttacatctgaagaactactgccattttctatctcttctgagggtctaggagtctcttccaatgcagatcttgtacgataattgtgttgatttgtctgttgctttttggattctccaagatccaggaaatgctcatgagcatgattctagaaaaaaataaattaaatttattcacagattgtttaaagagcaggatttttagtttaaaagttaaaattttttaattaaaaaaattcaagctgggcatggtggcgcatgcctatagtcccagctacttgggaagattgcttgagcctaggagttcaaggctgcagggagggatgattgcaccactgcactacagcctgggtgacaaagtgagacaatttatcctaacatacaacaacaataaaaacatttgttcatatttaagatatcacttaaatggatgaggttatagtaccaactcaaatattttaacattcatcaagttaaaggcttatcatggttatgctgatatttacttgctgattgattagttacaaaaatgctgttttttttttttctaattaatgacagacataaggattcatgataaaagactaaaccaaacactgaactcttctgtatctacatcaaacctatagaaatgatgtaagaaaaaggataatgggctatgaggcatatgtaataggcagaggcaaattggaaaccaaaatggggatcataaagctaccctgccactatgaagataagatgccatgtggctggcttatttttcttctgctggaataaatgtgagaagtcttctgcagatgagaatacaaccaataaacactaaaaaactgataaagtacaacacagtgatgaagacaaactcaataaacattaaaatttatacctaaaaaacacaaaaaacttaataaagctatctcaaaagtgaatacaaaggccagccatggtagctcatgcctgtaatcccagtactttgggaggccaatgtgggagaatcctgctttggggtcaggagtttaagactggccagggcaatggcaataccctgtctctacaaagttaaaaaataaaatttaaagttagccaggcatggtggtataaacctatagtcccagctatttgggatgataaggcaagaggatcacttgaggcagtccaaggctgcagtcagctaggactgtcccaacgcactctggcctgggtaacacaatgagactctgtctccccaccaaccaaacaaaaaagagaatcacatccatgaaataaatgcagggtcttgtcttagaaaaaaacaaagactcaattagacatccttgaaatcgaaaataaccaattaaatttgggaaaaataaatgatagccagctgaccacataaataataactggttattccagtttttgcgaccacagacgctaaaaatatgtgagtgtgcatgcacacacatacatatatgtatacatatacatatcattccttttatttcttaaaaagtcctcaaagtactactgaattaagagataagcacatcttttaattttaatagatattaacatattccatccataaaggtagtagcaagcaatacaaactctcatcatcattatataagtttatctcagcaaaggatgcaaaaaaggtacgctttccacttaccatgaaaaagccaaccaatcaatcaagcaaaaaacaagattatttcagaaagaaattttcctttattatactaacaggaattctagcattgtgaggtcctactgatcccagggtatgatacactcagtaaagaaaaaaggactacttgagtattttgaagagatgagtaataaaacagaaaagtgaattctagagaagaagactgatttaggagttttaaaaaactcaaatgaaaaaaaggaaggaacacgaagctggtgaagaaggccagttatttatttttcctttgtttcaataattgctttagatagtgacttggacctaaaaagaagtttcacataggcttcctcaatttcagactctaaggctctgaagctttcaagctaagatgtttttctccaaaataaaagaaaagaaaagaaaaaaaggaaaaagcgatatttgtgattccttcacactcaggaagtacgtatctctctcaattaggccatgaccaattgaaatctactgggtgcaacagtttttccagagtaggatgacagaaaagccaataagtcaaaactattagggacaatctacctctcttaatgaagaaaatgagaaatattatctatagcagcattagctgacttgattatctagaataatgaatagatgcaagacaccacaaaaacacatagaaaaacataacaaaatgctatttttagactgtacaaagatggcacacaagattatgaagagctaaagaaagttcttgatgaggcttcagtgtaatttattagaatttcatgagtatgtaagaattggcactttgggaaagggtatgctacaaagcagaaatggaattaaaaattttaaatagtaaacaatagataatccagagataaccaagatttactatgttaatttttatcattaacctgtttataataccatgttaaattacaaaatggagccttaaaatggtcactatacttaagaagcaaatattaaacatcaaaataattaatatgtacctttgagacagtgggtattttattctcttttggaacagttaagtgttttcttttctcttctgacctgtaagtctttatttcttcttctccctttgcagttctccattcttcttgcctactgaaagacaaaagccatatgcattaatctagaattttacacataactttcctccaaaaggaatgttaggtatcaggaaaggaacgctactcctgatgttttattttaagatggaagcatgtgataaagatttctaagtttaatttcataggttgtgggctcaggacatccggcattaaatgactgaaataatggaaagacaaactctgtctaccccaacttgatactactggttcctgctatggtctctggcaaaccaagtattagattctgggaatgtggtgattcctagggtaatttttccaagagtgagaaattggatttttttttttttttttaggatagggcctatgctttgataatcttctgttccatatagatagctggaatctctttaaaaattcaatacaacagaccctgaggttacacggaaagccctgaaagaactacactctttccagagacagggaggtcaggtttcaccgactgaaaaaactcaaaccatcaaggtgaaactgaacaacacaaattgaggaacactaattttaaactttaatacaaaaaatgaactcatgagaagataattttattttttatttcaatatcttacaactttaaagtttttgaaatgcaagggtatttgaaatttaattctcccttaagattttataataagaaggtagttaatactaaaacacagtgcaactatatactgttgatatttctttactagatgatattaatgttatcatgttacttaacggcacaatctccattgtggctggccataattctgaatggaaaataaaatgagcaaatttaaatcttgtgaaaagaacaggcttggatttaatttttttgttcttaattttttttttttaaattaaactttttactgcaataattatagattaacatacagttgtaagaaataacagcgattatttgtatcctttatccagttcccccaaatagtaactttttttttttttttttgagatggagtctcgctgtcgcccaggctggagtgcagtggcatgatctcggctcactgcaggctctgccccccggggttcacaccattctcctgcctcagcctcccgagtagctgggactacatgcgtccgccacctcgcccggctaattttttgtatttttagtagagacgggggtccactgtgctagccaggatggtctcgatctcctgacctcgtgatcccccccacctcggcctcccaaagtgctgggattacaggtgtgagccaccgcgccggcccaaaatggtaacattttgcaaaactttgtatcctttatctaattcccccaaatggtaacattttgtaaaacatggtacaacatcagaaggaggatgctgacatttatacaatccactgatcttactgagacttccccagttttacttctattagtttctattagtttgtgtgtgtgtgtacacaggcatacattgttttactgtgctttgcagatagtgcatttttttttttcacaagctgcaagtttgtggaaccctgcaatgagcaaatgccatttttcaaccacatatgctaacttcgtgtctctgtgtcacattttggtaattcttaaaatatttctggctttttcactaccattatatctattatggagatctgttgtcagtaattttttatgcttctattgtagttgttttgaagcaccatgaacctcacctatatagaacaatgaacttaactgacaaatgttacgtgtgttctgactgctccactaactagccattgccccatcttctccctctcctcaggcctcactattccctgagacagaacaatattaaagttaggccaattaaaaaccctaactgatccagtgaaaacatctctcactttaaatcaaaggtagcaacgattaaactctgtgataaaggcatgtcaaaatctgagacaggctgaaagctatgcctcttgtgcccaacaaccacgttttcaatgaaaaggaaaagctcttgaaggaagttaaatatgctactccagtgaacacaggaatgatatgaaagtgaagcaggcttgttgccgatacagaaatagtttttgcggtctggatagaagattaaaccagctacaacattcccttaagccaaagcctaatccagagcaaggctctaactctattctcttctatgaaggttggaagaggggaaaaagctgcagaagaaaagttggaagctagcagaggttggttcatgaggcctaagaactacctgtgtaacataaaagtgtagggtgaagcagcaagtgctgatgaagtagctgcagcaatttatccagaataactagctaagatcactgaagacagtagctacattaaacaacagactttcaatgtagtaacagatcaaacagccatctaaatggaagaagatatcatctggactttcacagctagagagaagtaagggcttggcttccaagcttcaaagggcagtctaaatcttttcttaggggctaatacagctagtgacttcaagttgaagctaatgctcatttatcattccaaaaatcctagggcacttaagaattatgataaatatactcatcttgtgctccataaatgaaacaacaaagtctagatggcagcacatctatttatagcatggtttactgaatatttttagcccactgttgagacctactactcggaaaacatgattgctttcaaaatattactgttcattgacaatgcccctaatcacccaagagctctgatgcagatatacaaggtgattagtgttctcttcatgcctgctaacagaacatccattctgcagcctatgggtcaaggaataatttctattttcaagtctttttatttaagaaattgcatttcataaggctatagctgcactagttattctgctaacagatctgggcaaaatacattgaaaaccttctagaaaggattcaccattgtagatgccattaagaacatttatgattcatgggaagaggaaaagtatcactgttaacaggagtttgaaagaagctgattccaaccctcatagatgattccgaaggctgaagacatcagtggaagaagttactgtagatgtaatggaaacggcaagagaactataattagaagcggcaactaaagatgtgactgaattgctgaagttgcataataaaactttaaatgaatgaggagtggctcatgaatgaacaaagaggtttcttgagggaggctactctagtgaatatgctttgaacattgctgaaatgttaacaaagattgtagaaaactatatgaacttggccaggcgcggtggctcatgcctgtaatctcagcactttgggaggctgaagtgggcagatcacaaggtcaggagatcaagaccatcctggctaacacagtgaaaccccatctctactaaaactacaaaaaattatccaggcatggtggcatgcgcctgtagtcccagctactgggaaggctgaggcaggggaattgcttgaaactgggaggtggatgttgcggtgagccgagatcacgccactgcactccagcctgggcaacaaagtaagactctgtctcaaaaagaaaagaaaagaaaattacatgaacctaataaagcaatggcagggtttgagaggactgaccccagttttgaaataagttccacacagaaatatttcatgagaggagtcaaatcaatgtggcaagctttattattgtcttattttaagaaattgccacagccactacaagcttcagcaacctccactctgatcagccagcagccatcaacatcgaggcaagacccttcaccagcaagaagagtataatcactgaaggttcagatggttgttagcgtttttcagcagtaaagaattttcaaattaggtatgtagtttttttcagacataatcatattgttcaattaacagattatagaatagtataaccttaacttttatacgcacttggaaacaaacaaacaaaatcatgtgagctgctttactgcactggtctgaaaccaaacctgcaatatccccaaagtatgcctgtatcttagcaacctaaatctgttctccattcctatacccttgtcatttcaagaatatttcataaatggaatcatacaggatgtactttgaaattatttttctccactcagcataattccctcaagattcatacccattgtatcaatatgctgttcctttttattgcacagtagtagtccatggtgtgaatataccattgtttaaccatctacccactaaaatacatctgggtatctttctggcttttgggtattacaaataaagctgctatacatttatgttcaggtttttgtgtgaacatgagttttcatttctttaggataaattcccaatagtgcaactgctgggtcatatgacaggcagttccatgtttaggattttaggaaagtgcaaaactgtttcccaaaatggctctatcattttacattcccactagcaatgcatgagtaattcagttttctctacatcctctctagcatttggtgctgtcacttattttttattttaaccattctgagaagaatgcagtgatatttcactgtggtttcaacttgtatttccctagtggttaatgacattgattatcttttcatgtgcttatttgtcatctatagatcctctttggtaaatgtctgttcatgtcttttgcccattctccggttggattctgttgtttactattgagttatgagaattatttctatgttacttagccccctgttgggtatgtcattggattccattttaattaatggatgaggctgacccatttcagagagcctttttaaaaggaaactttagactacccactggagagattcttaggaagattcccataggatgagtacaaagttttagagacaaagctccaggaagcccaaagaaagaatatctgttaaagttatggccacagtcttgcttgaccataggccaatgaatagttaagcccaatgataaaggaataaaaggatgaagaatatttgaagagaaataaatcttcctcactcctcaggttcccttccatgtgcaggagcctcaacctacaactagcaaccttatctcctgactcattcctctccagaggaggagtaaattagtcaactgatatgctctggaagaaaaacccagcttagcacagcccagccttatgccattgtgtgcaattatacatttggccctgcatcttaaagaagcaaaccacatgtcctgtcccacaaggggagaaaaactgtggcccactgctatctgtgtctggtgaatattactttttgttactgatatggtttttgtggaatattactttttgttactgatatgggttttatttcacgaaataaaaagtgatagtaacaaacagtgataggctgatttactatgtcttattctccatgttatttttcaattatttcagaattggtgttgaaaaaatgggcattatttatttgacccctttcaaaccttaacattagaattaaaaagtaaacaagaatctaactaaaaatatgccatgtgagttaattaatttatgacggataactgggcatatttgtcataagaaagtgtaaatgtatacctttggtgtgtatttaattctaaatcctaacataaattcaaagtatgtccaatcaaaaagcataatctatatgaacattaagaccaaaattttaattatgtaattactatcttctgagttgaagaactagacaatcttaaattcaaattcacattttgaccttcatacttagaacacacttcatacacagaaatcagccttacttaagttcatataggcctcattcatgagtatttcatgactgaagatcaaaaggaactggatataaacttccctcatggcatggtctaagagaatagaattttattaaatcaatacttttaaagtcataaatgtatacttttaaatataaatattttaatatatataaaatctttttgtattttccaatttattttttttttgcatctgaatatcatgtgaatggctggctaaatccattaaaaaattaaataattacaacaaactttataaaatgtattttaagatatagttaaacaggacaaaccaagactgaaatatgctactcaataaaacctcctatttggtcaatccaaatttatgattttcctgttgaccttaacacgtatcattcttatcatttacataaatcattctagaaataaatatattttagaaacttaatttttctacagtcatgaatagtttatactgcccttccagaaaaatttcagaggaattacagagctgaaaataacagctgtgaatgctgttaactccaaaatgaacccaaggaactatggtacataaaaattcacaacttcccttacctggctacaccagctgatagctcgggtactaccacccttcgactccaagctaccagatcccgctctgtggctatttcacttcttggtgcgttgctgtgcatttgccgtacaccttcaatttgtccactacgtcttagtcctacgtttggtggtgaatgaacctctgaggtagaacttatggagcctaagtgaaaaagttactatattaagttctacttagagatatttctccattagtttataacagaaaaaagagataaaacactatcttccataagaaacttcatattgtggcaaaataattaaattaccatatcaggaactaaaccacaggcaaaagtggtttaatgaaaacaaaacatggttattcagttgattagataagtcaatgaatcataactatagactattaagccccaaggatacaaaatcattactttaaaaaaatgctaagtatttttgagaaaacttcataagaagcaaactagacaaacccaagagtattaatggttcataaatttgttttgactcttaaagtttaataatatacaacaataagggtgatgagatgttcaagaatgtggctttgactattctacatgtttactatagaaaatgtggaagacatacacatttaaaaactcattatctctaacttgaaatctcattatgaaccattttcttaggtcattaaatattcttcaaaaccatgatcattttggcaacctaatatttcattttacggatgtaccataatttttttaaaccacttaacactgtgggagatttaggctgtttcaaatttttaaatatttcacttaaaatagaatacctgcttacctctacttaaacggctggtattactgataactgcttcaccagaacgtctcaggtcttgctcctgttgtagtctttgaatcatgctgtccagtgggctgatctcctggtttgcttgctgacttaaaacttgattcagtcctatacaataccacacaaaatattacgaatattttagctactattcaaaccattttactcaccaacctcacttatgagttcagaattaaacataaatgttgcttgggtaaaagacttaaatgatcaactataaatgatgggagttaaaaaaaaaaaggaaagaaaacatctgtccatcttagtttatcaaaaataacgatgaggatggttgctaacttttactaagtgtttatcatgactcagatactcataatccgtatggtaactccattaggtatgtataaacaactccactttatagatgagaaaaccaaaataactgttcgaagtcacatatctaataagtggcaatagtaaggttttccacttagatcatggtttcttttaaaatgacacatctgattacccattttcttaaaaaaaatgtttactgcctcatttattcaatacattattgactgtctactatatgttaggaactacgccaggtactgagatctagtggtaaataagggacatgaagatccctactctttagggagctgacatactgatggggaaagtaaacaataacccaggagttaatacagagtatgaaagagaggtgttgaaataaagaaggtgatggtgggtcagaaaaatcagagaagtcctctcggacaaggtagcatttgcgctgagatttaaattagatagccacataaagatctagataaggaacacttcaggtagaacgcaagaggtacaaacgctgtgaagcagggagaaacttgtattttaatccatccaagttttccagcattaattcttacttgtcatagaggaccacttgacttctgcccttctaaaaagtaaacaccttcatgcactgaaacttttgaacacaatattcccactataaagataaacatgacaatctcttaacttaccattccagcaccaactaaaatggcaattccatgacatttcctctgtgaaatctttccaattacttctgaaagaattacctgctaccctctttcacttccttaaacaacccatacatatctaagtattcagaccaatataaaacataaatgaagtccaaacccattagttcctcaaaagcatgaactgtctcacttaccttatctaaatctagtacaatgctaggcattaaaaaatattcactcttgctgaataaatttttaaaaggtatattataaaatatatgaaaaatattgccatgagtataaagaagggagcaattagtatcagccatacaagtatatgagaagatctgatcagctcagaattttagggtgggcttaaatggtaagaaaagagagcaagaatattctgaaatgaagtagtagtaagaaaggtaaaaggaaaaagaaattacatgtattacatgcataatttcatttataaaagaaaactaaaattcaataggataaaataacttgtttaaggtcaccaaagtagaagtagtaaaagcagacagaagtaaaagttcaaatgtcttatacttctttcctggtttgctgttggaaaaccatgttgcaatactgatgaataatctgatatagctcaagtttacaactgaaaaaaacacatttaaagtatgttgtatggtgccaatacaacaagctaatttattattataataactcaaatctatttttccctaactctgagagatttcccagaataaattttattatctggatttgcaaataaaaagctaaggttttttttgaaaagaattttgcttttgttctgtttttgtattaaccaattttagactcaataagtctaaaatatgacaaaaaaagatttttactctgaattctgagaatcagaacactgaaaaattttgggactcattacaaagccagtattattgctatggattctcttatggataccaacaactggtatctatttcatttttaagattatgcctattttatatggaagaaagaataatggactgagaatcagagctgaggttagagtccctgtgattctatgacacattaccattaaattttgaattctctcaacctgcaggactggaatatcaaagatacaggattaaatattgtttatgaaagccctttataaattggtaaatggatttaaaaagtaataataataattaattagcactgatactttaataaatgagtcttttccttatttccctgtgtctgaaaccctaaagtagctatctattttgaggcttgggaacaatctgattttgccaattcctctccgagagactgaagacatttctcttaatttcagtcctatgaccagaacttctctaatactgaaacttatctaatctgagtctgagtatctgtccatacttctcgaatactacttattctttgagttcatgctgtgtccacgtactgagacacattaatctcacaaactccagataagtccactggactgcactactctaggagtagcagcaggaatgattcctctaatgcttcttctcaccctccattctaagtggacgtgtctaattccaagaggagccccttctatccagtatgtccatctttattgcaacttcatgctaaatcctttaagaaaaataagatgcacgtttgaggttgattttttctgtgctccttacagaatctaatttcattatttaaaagtcactcaacacaaaagctacttagaagcttttgtcgattgaagtctagaacttaaaatattttcataaatatttttctagtctaaaaatatagtagaagtattcataatgacaaaactggtttaaccttctttacagaacctttccttatttttacttaatacactagtgctgcatttcttgtcaaaagagggaaagcagtttgtagactttgactccattttaactctcatttaattcttcaacactccattatacttcactaaaacagctctcaacactttccatgtcaatcctcttataaacctttaaaagttggtaactttttaaaacatcttcaatgtgaacaggcaatctacaatctctctcacatcatgctattattccctttagtaacattcatcacaatttgaactatgtatttgtttattctaatgaactgtgtttattaatctactaacccattccatgaaagcaaggatcatgtttgtttaatccactactgaatagccatggcttagcaggtgtcagagacagaagagattatcaataaatgttgctgagtaagtaaattaattcacttgtttcacaccaaatacaggaactaccttattgatccctaggggttacaaatggataataaatcaaaattattactgtaaatcgcccatgttccataattaacttgtaatccttaagcatgatcaaagacctgtataaaagtataaaacatacttttacttcttatccacttaacaactgcaaacaaagttttactcaattagcaattttaacagttcggtgggaagaagaaaatttgattgtctaagaaaatgagcacctacctgaggaagttactcccatctgagggatgagttgctcctccctgcaattttcacggccaggaactaatctttgatatcttgatggatgagggttaccatcaacatcaaccaaaaaagggggaggcataagatgaggtgcttgctgagtctgttcatctaatacaaaattgttggcatcacgaataagtggccgataatcactatgaaagaacatctgatctgctatctgcaaaaagaaaagtccataaaagactggaaaaataaaaatgtatcattgtagaaaaaaagtctacatcatttctaatagaaagcaaacacctgaatacgaataatggtattaagcaacaatttttaaaatattattttcctgaattaattgtaaataggtattttaattttctaccttaagtattatgatcagaaaagtagctgctttaaattttctctgaaacaagtaagggattatacacaggagtcccctcttatttgcaattttgctttccatggtttcagttacctgtggtcaacctgactccaaaaatgctgttccagaaataaacaactcataagtttaactgtgcactgttccaaaatgcataatgaaatcacgtgccatctcacttgggacacaaatcatccctttgtccagcatatccacattgtctagatatgctacccacccattactgtataggaaaaacacagtgtgaatagggtttggtactttccaaggttttagacatccatttggagtcatggaacatattccctgtggataagaggaggctactgtgtagaaaagctaccactaaaaataagctattcaatattcaattttaaattaaatggtaacatcaaatctagtcataaatcattcaacagtatttttacttccacagagctaccatgctttatcttaaatccttaggtatacagtaagtatgatttgtgtaatattactataaaaatcacataagattttctaagggttaataagcattttctctaaatgttttgagatgatataaaattattaatataaactcaaccaggacataaattttattatataaaatttcactggttaatttgcaattttttaaactaaattataaactactcaatatttactatcataaccttttataccattccaccacaagtaagcaatttaattcttttagtcactaatcttaaagttggggtctttaactgctgagcaaagtggcagggcatatgcctgtagtgccacctgcagttatttagcaggctgaaatgagaggattgtttgagcccacgaattcaaagccagtctaggcaacatagtgagacatttcacccctactctgtaaaaaaaagccccccaaaaactggacaggatctttaataaaaaacattaactacaatcaggtaatataatacatttttaagagccacatgaaagccacagatttgtaccctagataagtacgtattatctatattaaaaaaacattttacataatttcagagtcccactatggataacatcttccatctccagtttatataccccaagttaagaattccaatctagattgccccaggaggctaagagttatttttttcctgaaatatgcagattttcaaataataagccctattaaagtgagatttaactgctgtcttcaccttcctttccgaaactaagtctccagactacttaacacaagattcttcgaattttatcctgaatgtaaagatgctgattctcacccactccaaaaacatacaacccctaaacatgcattaaaaaaaaataaggacatgatatatagtcatcatactctaccttgtcatatttgctactggacccaaagccaaaaattaaaagatgtccatgagagtctgtgcatgcaaaatgctgaccatcaggagagcatttgcagtcaaatactgcgccatgtccttggccttcaatctgaaatatatttaaccaacagtaagaaaactaccattaaaatgaaaagaattggtcactattattacaataattttaagataaaagcatacatcctaatgaagtgaagattaagtaaaacaagcattgtacagtaataaaatttatgaatattcatgaaaagtgccagattaatagccttgaaaaataaagtattctatccacagaccaaacacaattcaggaaataaaagaataccaactcccccacccatagaattctgcttcatgttttaaactcagatttaacacataattatgtcttacactccatctatctccgttcaatcatgccctttctgcaaaagctgacatgtagcaactgtacggattttttggtaattcttggaagtagtagccagagatcattgctcaaatgacactagattttattatttttggtaataaattccctggtagtctgactcgtcttttaaatggtgaatttcaaaggttaaagaaaaggttttttttacctgataattgttttctgttttccagttccattaatccagaacgaatgggtttcctccctgcaacctgtcaatcaaacagaggtggccaatcaccactctgaattttttgctctcagtgcttcttcagactatgttccagttgaaaacttctttagcagtgttctgaaagaggtaaaaattctaccctatctaaagcacatctagggactgaatttcagaaacaactaaaatagggagttgactccctaacaaataacaactaaggatgattttcaacaaatgctgttggatttctggattagcagaatcggaacacacacacattatcaggtaagaaatacctcctttctgttccggtatccactgatctagaacaaattgagctttattagtaatatcccaggaaatgcttggaaaagagaaaggtagtaactttctttctttagtattaaaaatgtggcataaggatggatgtacagttcaccaaatgcagaagttaaaattcagaagaagaataactgatttctcaaggaaggtttgcattttggtaaaaagagaaaatatttctataaaagaaaggactttacctaagaagttaatacaataccacaaatgggactgcctcaaaaagaaagctagctttttcctttggcttgctaaaaagaagaaatgtgtgcaacagtgcttattaatgctcttttggatggttacaataatagaaaagaaagaataactttttaaaaaatgtaaccatattagataaaactaggaaaattatctaatgtttaatatacacaaaaacttagaaaagaaaggaaatgaatcataagtgaaacaggattatatctcgagtgtgaagaaagttggaggatgataagataggagaagaaggtaactgattccaagtctactttaaaacaattcaaaaacaagaaagaatataactatttctccagattaccataagggcaccaggagccatacagctaggcatttgccaaaaatgaaagactataagtacaaattcaggattcaagtctttatttttcagtgttttcctaggcaaataaaaaaaaaagttacatgaactgttataaataagcaaccacatgaacaaagtacacctctaaatagacttttatatcaaaaactaaaaattaggggattgaaactgccttagccatgtgttgttaaatgatttttttttaactcagttcactcaaaatttcacagaagccaagagagagaacaaaaaagcaactactttataaatctactctaataaatgtttccagaagtataattacaagtctaagattacaatttgaagtagagtggagacttgaaagtagtccaatttagcaatttcaaaggaaatctgataaatgttcctaagcatggtatccttcatgtgttgtttaaacaaacattttttctttttgggggtgagggttgcggggcaagtaggactgatcaacccttgaccctattatttatcaatgttgccacatttacagttagtagatctctgaaataatcttggggacagttgaagcttataaagctctaaaagagcaaagaaaaaatagcaatcatatttaagatgcctgtgtgtcctatataacacatttcattgtgaatatggcaagacagtattaattttcttggtataaggcatctgtttaactccaaagtgacttttatatggagaaaatgaaagtatatttcaatcatatcagaaaaaagaaaaggatattatttggattaaccatttgtttactaaaggaggcattaaaagaatctgctttactcatgaaccagttagaaaaggtgcctctaacttcatcaattaaaagaccaactctctatttattaatagatcctcagacaataaacacccatatctataaactgcagactaggttttccagaccaggcttccaaacagttgatgatataaaacaggaaaatattttactttctctatattaactaaaaatagcctaactggttttaaaatgtatggtacgattaagtaagccaatcaaaagaaaagaattttatctttttaaacaagggtcaaagtatttatgagtaagaattctcaaagacaaaattttaaatgaaggcactatttgaatattcacatctactagaaagcagtaaggtttatcttcaaaaacgaaaagaaaataccctctctcaccaaatgaaaggtatataagcctatcataaaattaaatgcactgcgtatcaagaaaatgtgtcaacataaaatttaatactacatatagcttatgctagctagcacttactgcagttgtagtaaaataattagaaatagagtgaaactaataagtaatgagaaattatcaaaataagtgcattttagatgaactattcctctaataaaatcaagtatgcttgttatgcattcttttggatatatagaaataaagaccacataaagctcatagacattaaatatcaataaggtttagctgagataatctatgagacagtatttaccagtaactgtgaaaacttcaaaaagaataagaggagtaaaaagaaaataaaagtatattcagcaattattgtattttgttttatttttaaaaggaggagatgggaggatcagatgtgttaaaataatgactcccttatttgaaaattctcatactgactaaagaattctataaatactaccaataaatgagtagtataaacttgttaggcatttagagatttatactaaactttaaagaaattaaatgatacaaaaacttatgagctaagagctctgatgaggactcattaaggaaagaatactaataccttttttgagggaaggtactatacacaactaacataattttcctgaagcagaaagatgaatgattagacagaggaatgaggtcgaagacccaagacatctctccatcagtaataggtaaatcacctaatctctgtggaatgatggagataaatgatcactagaatccagttctaaaatcctaccatctgaggttctgaaaggtatgttgaaaaaaactggacaaatctggagatgaagtatattaaaagcagaatgcatactaaaattcaggatctcaattatatcaatcatgaagaatatacagagagtgaatatgagaagtatatgcttctagaaaaccttaacacaaagtaggaaggttaaaaaatatgggctatcttaggcagaaccatcctcttctagagttatttcaattctattagcagggtcagtatgtcttgttcttttttttttttttttttttttaagcacaccgttcattagaagaaagcatcttacctaggaaatactccaaaattttaaattatgtatgcaacttttaaaataccctaaaataatcttatgaaatggactcatataccaagaatgaaaagaggtgataaatggaatttatgctaagaataacccttaagaagtccttccttatgtattaaaaaaacttttagattagagcttgccaacctagggaaaaatatgtaaactagatacaaaaaagactcagatgtatatttgaaataagtgttggatcctggtcaacatggtgaagccctgtctctactaaaaacacaaaaattagctgggtgtggtggcgctcgcatgtagtcccagctacttgggaggctgaggcaggcaggaaatcacctgaacccgggaggcggaggttgcagtgagatgagattgtgccaggaggcggaggttgcagtgagctgagattgtgccactgcactccagcctggtgacagagcaagactccgtctaaaaaaaaaaaaaaatccaaattccaacagttcaggtgttatcaaattactttaaaatagttattgcatggctgttttaatcttgaaaattctttaacttatgccaacataaaaaaggaaactgctggacctgacttgataaaaatcagtagatcagattattacataaaatgaaaaaaaaattattatataaaagtgattctgaaaaatcagctctagattttcatcaaaagaaaaatattaccaaaataaatatcttcaaatttaacatcattttgtacccatattatgatttctctggggagagcaatactattgattaggccttccttgaggcttattttcttttggtctttttggcatattagcatggtgtgtcttccttgacacaccctcttaaggattgtgaccccttttccattctgctaaatatatgagtatttcccaaagtttacttctaagccttctgcacttccttctcctgtctccacgagaaagtaaactatatactataagaaacacttcaacactttcttctttttactcctagcccctctaagtagtctatctccaagtgccagctggccatttccacataggtagttcaacgcaataaacattattacaaatgaactgaataaagaagtcagttctcccttatgtctttcatatttccactaataaaaccattgttctcaaggtcacccgggcttaacactctataaacccatttattaaatctttcctccctgtcatcctatagcccaaatcctaatatagtcacaaaacaccaagtcatttatgtatttttttctttacaaatttcctaccaactacccctataatatttcatgactaattaaagtagttgtcctcacacttattcaatttcatacctgaaattgtactactggcaaccaaactatttttctcttagcttctcgaccatcctataaaataatttactaaagcccccacaaggttcataggtatttatgcctatgagatcatttgaagtcactgacagttcatctcaatttgtttttcgtcattatttccaaaatctactgcaatcaagcttcctaaatatctaaatttctatgaacatgtcttgacacttagctttttataatgttcctcttgtttataaaattcattctctttcttactgactcgattcctatttatctttcaaggcatagtttcaattccttctcctcaacaaaacgtctccaatcgtccaccctgacaatgatctctacatcttaagatacagcaactgtcttttctcatttgtcatgctactgtttgaaattatttatcaatatttatcccttaaggtatattttgtatattttgtctccccaacttaactgtaggctgactaaaaagaccacgtcttattctcccttgtggtcctcatattttgtgcttaacacaaaagaaaacactcaaatatttgttaaaatgttttcatctgcatgtttaaattctgtataatttcatataccttctcatataactatcaaatctcaaatacccttgtgatagcaagtcatggactatgtcaaagaattactacataaaagtaatttaccacatataatgcagtgtgagaagctggagagacaaactcacattcatggcaacagattaacatgccttttgtaccaagatatatatatataagagagatataatctaaagaatcttaaaacctgaaagtgataattactaaagtgtatggtaagaagaccgaaagtacttccttacccaaagaagctgaaacatagactggaagcatcagagagtccttttaacacagagagtataaatataggacattatcttgttagatgatgataggaagaaagaaggaaggaaagaagggaggaagggagggagggagaaagggagggttgggtgagggagaaaagaaaagaaagagaaagagagagactcttctcctgattaataagagataacacaatatgagctgtacctattttgagcttctctttctccctttcctagatacatacagctacaattatctaaaactaaagtacaggtcacactgagaacatgttaacatcagaagaagtatgcatgtacaaaaattctgggtggtagattggcagcctctgctcgtttggaacgttgcaaggagaaatatattgttctggattagtggacactggaactttttttttttaaaaaaatctcttaagtaaaagaaaggttaagagaacaattataaaaataaggaaaactttaatcaaataaaaacttatttggaatttacccatgaaacaacaaagtaaagcaaaaatcaaattcagtaaaactgctttctattaagagacatactatactctttgattaaaatgaaaaccagacaggaggcaacaaactacagctttgagtaatgacagaaatagaaagatatggaaagagagatagagacacagctagggctatagagaaaagaaagagtaagagaaataaagcaaaaccgccagaaacatgaggaaagctactaaaaacatggggtctacaaattcaactccaagcatctcttatttactatttaatattcaaatggcctagtactaagaaatgtgaaaagtctctatcttttcaaattaatttaatattcatttagttaaactcgtagttaaaacttagctgtccggtgctaatttaatggggaataaaagaccataaaacaatttatatttaggaacatttaaggttataattaacttctaaacctggcgacctctttcacagaaggccctcagcttcagtcctgagagttgcacacattttcaagctatttctgggaattatttatctgccttttagcatttaatgggagtatagagcctttagagtttagaacaactctcatcaaaacaaagctattctgatgtttacctcctgccaatgccaaacaaatgtgggcttactaagttatacccaactattatagtttggaatattcttaatatacactacttgcttcagtaaaatatccaaatatatactacatttcctctgaatactcaagttatgtaaggactgttcagttgattcgtaaagaaataaaagtactgaaggcctagaatgtagtttgtttgtttttaaagaataaagttgtctcataatattttctacaaaattctctttggtttcttctcctgttcacttaaaaaagaaaaacaacaacaacaaaaagaaccacaaaggctttcccaataagtgcttttaaaagtttttagttaaagatgagacaacagaaagggtagggggagtacaagctacatatactgtctattccatttcatgccctatgttagcctcttttaaaacatcatctcacgtgtcatatacttcttataagtaacaaaaacaaacccagcacccactccccaactgcttttatcattgagatcctctattaggaggaaagttagcagtaaaaacaagaaaaataaacccctcagtttctctggagaatactacttgaaagttgagaatccatttataagatttcagaatgaagtaaattatttaaacataaaagaactaaatagctttatctcaattcccaatctcaaactctttaatttgctgacaaatttagatggtcccaaaataaagcacaagaaatttttaaaagtataagtcatggcttgatacagaaaaaaattagaatacttattacaatgatgactatcagtgcaatattaaaatattaatgttttataatcttatatttaaaaattattaaaatgtaattactatgtatcaaacaggcatttgaaagttcaccttttcacttgaaaggctttttaacataacaggatttttggctatttctaaaatttcaaaaaaagaatttacatttccataattacacaaaaaatgcagtaaaatgctgatgaatacaaaatactaaattatatgttacatgatttccattatcttttgcaaaggtataaatttccaatggaaaattcaattattattcaaaaagcaggagaaatattaaagtattcttaaaatatacttgataaaaaccagtatttaagaaatttgtactaaaactgttattctaaaggtatagtctacattccttattttctagctgtaggtggaatggtgagtttacttatctgttttataaacttcagttttaacagtcacatgaaatattatttaatcttaaaaatacttcacataactttcaccatttctagtcaaaaaaggagtattccaccagaattcttcatcctctaatagaccaaagcactatatatgactagacccttcacatggtgctcaaaaaatatttactaaactgaacttgtgattactactacaacttaacattagggattaaatttgtatgcaatcaagtatcgtggtattttagtaactgaaaaacttattgattagctacagagagccaaatagctataattatagccaaaactcaacattcatgatagcaagcagtgagaacgcaggccctccctcgaattgtttctctttattttcttaatagcaatgctggatgctttatcttccatttgcccataaataaaacaagcaatgaaaagaacaaaagagtgaagagcaaaaagaattagggcaattagataactcataaaagacagacaggaaaaaaaatcaagttaaagagtaagatgtcaaaagatccactcagatttattaccattatgaaaacatttcttcatagacatatcactaactgagtattgttaaaagttagctatgcagtaacattgacaaaagctcaaaaagccaaccatgacaagatttgagtacaaccagagtcatgggtttatgctccaagtgcccgcataatagctgtgtgaactcagtaaattggggcaaagcactttatctctgtaatgtacagtttctccattcctaagaccaagaataataaaatctatcttgatcatcttacaaggttttcatgagacccaaaggaggtaaaatatgtgggagcattttggaaaccattaaacatcatacaaaaaattaaaggtagtatctttattttaatgagatgaagggtggctcactttctgttttttagcttttttggtttatgttttgctcactgtctgcaattctatgaatactatccaattcaacttattacatgtatttgtttcttctgctctttagagttttcctatctcttatccataaaaagaaccagaaaaatatcctcatctaacactcttatttaacaataaacaatttttaaggcagtaatgtaaccaatccctcagctaatttttaaaaatatacaatatattatggctgacttctactcctggttacattactaatttttaatggtctaagagcaaatcacttaactctcaaggtgctgcagtttttgttatctactgaaaaggtagaatattagtgtgaccaacttacctgataagggaaaattcacctttatataactgaaaagttaaagcggtattcaattatgggagaaaagtttccctccaaacactctaccaatataataatgtcctttggaaatacaaaactactaaatgaagccactagtatatatatagctcacatattattttttttaagctataaagacagattgagaaatgactaatttccttattcaacagatattccaaaaaggagcaaatcagaaacacaaggatagaaaagcagaaaatattttttactagcatatttacaggtggctttttaaaaaaatctcaatacaatcacaaaggaaatccatccatcactaacaagtgcacaccaaataattaacactgttttctagaaatagagggttttacaaaccttatttcttacctaatgtattctaaggcacagccttaaagatagctaaagctatttccctcactaaaaaatctgctattatatctgcttactcacgacatagaaataactttactctgattatcaatcaagcatagcatcacattcgtgtaattttttcacaaaccatgtttcacaactgttttgtgaaataatttatttggcaaaataattctgaccacatgtactaattgtattgttttatggttcattactaaagatttctataattggttttttaaaaaaatttaaacatgctgaaatagtggaaactgttttttcttttgttctttgttgaaaggtatctctaatatacagaaagtagacatttaaaaaatatgactacacaaactgcagtagttgaggagaccttaatacttcatacagtaaatagaaacactgctcggtaagttgtatgtgatatattaaaacattgtaattcaaatacttggccaattatgttaacatctaagaaacaaaatgtgaagagaagagtataaactcaaatatttaatatactaccaattgattaaaagcaagaaatgcttgattctttggccttaattttaaaatcagtgtacttgagtaaaattctattgtgctagaagactattaaacaagtacaataatacgagtatttatttataatttcttcacatggttttccaagtattttttcttctctatattgtatcttcatacttgtgaatttccaaagtttcactgctaaaactgataaaactgtatcagttatcacaatgtacaggcactgtaatatgcacaattaattttcttttaaattcagcatgtcaataaaagtgtggaataaatcattctttattgatgggaatttaaagtcaaaataatgaaccaatttttaaatggatttcctttgtgacatgcagagtacctttgtcaaaaagctcccaaatctttagtaggtataaaatgaagagaatgataattaccatattgaaataagatcgtattttgactcctcttgccagatcccacactatcacgtttccatcatgaccagcagaaaagagaactctaggatcgaacgggtgtggttcaagaacaaatacctcatcttcatgaccctgaaatatttttgaagtgtcaaagaatcatagatattaaaaaagaaagaaaaacaaacataatgaaataccaatatggcactatttctagaaatggagttttaagaagtattaaattggcaaaaatcttcaaatttgtcatgtatatataataatgaaactgagctaatatcattgtccagggcttggtggatggatcttttcatattaaaaaaacagcaaatatctactatatatttccaagccaaacctgtttaactgcactgttaaaattattcccaatcactcacaaagccgttaccccagcaagcaagagtttttagctaacataaaagactttattcctcaatgagaatgtttcattctatagtcagcttcagttatatcaaataaagattagaattttttctcaccatcaggacatgaattagttgaccagtgtaagaattccaaactttcagagtcatgttattaactgcagttataactgtattgtcatgtcgatcccaagctaccatagtaaccttcatttttgtgattttatcttctattccttgaaggttttggctgaaagtgaggaagtgttttacactgactattaaaatactgacacaacaaaaaccaaaacatataatctataattagtatttatattcactaacataataactatcacttaacagtaactgagaagtttgaatcaacatggaatgatggtaaaaatccctgcactgagtcaggaaatccaggatttccagaattctagcacttctaagccattagtaagatgatcttgcataaatatcactgctataactctatctagatccaggaattctgtgccatgctgctcactctccatcttttactgttgctgtcatcccctgctgactgttctgctttcccatccatctgagatgactacacacaaatacaagtggagaaaaatattaaaaccaaaataacagctacaacacgcagtttaatttatcttggcttgaagaaaccattggacataaaatattgtccctctgtgataaacgaaacataatatgttctggtttctgaaaaaaatatttaaagttaaaacttttcttacagtacttttatcatctccttaaaaaaattaatgcaatgtcctgaaatatgccaatgaatcatttcataaaaatcaacactgctcatctccaattgtacctctagttatcgcagaaaagtagtaattttaacatgatatataattttataggctgagagtcaccaacctgcactataacatactttaaaaaatcaatatatacataatatttcaaatttgacctcttaccctgctggacgagtagccatatccaacaaaatgctcttccactctcttcgtttaaattgccaaatacgtgctgtcccatcacgactgccacttacaaacctgtattaaaaggaatccgatcccccaaagaaaaatcatacatgctttaccaaaatgcacttttcccagagatcttgtttaaaaagacagcattcatctttatttatgcagcattctaataacttctgaaactttatgggctttttagaattttatatgcaaacattccaattttcatggctaggtcacaaaataacattttcaaaagtgattcaaggtcactatgtactaccctagaaataaaatcgacattttccaaggaaaaaacaatgattttcttctcaatattaaagaatctgacttattctactcaaggtattagaagtagctttatttccttttataacaagaacatgggaaaatttatacaatatcatcaataaacaggtctgccttaaatattatactgtgatacattttcaatctataccaaaagcttctctacattttgaaacatatctggaatatatagggtgatttaaaaagcagtaattactaaaagtgtccaagtatactaacattattacaaataagagaaagacgtgcctatgcatgtgaaggctatattcacttttaagtataactggtcaagatcttttaggtaggtgattatttcctatacagcttcaaataaaatctgatgactaaaattgtctttatttcataattttaaaattagaattaaattagacaaatattttacctgttactagtgttggaaaactggatactgtcaactttgtcctatatataaacaaataaacaaaaaagtgggtgctgaatataaactcttggactcacataaattatactcatctaattcttttcaccaatacttacagtatgaaactccaattctgatattttctctggctgacctgatccaaaaaaataaacccgaataatatgatctgtgcttcccgtcgccagaaacattccacctatgaagaataacagcaattgttaagaagtaaaacataacttcaaaatctctgaaaattagataataaaatgtaagcaaaattacagtatatactgaattaatattttactccaaaaccattcacatttatcctaaatatataacacatctaactggaatttaagaaagaagtttatgaatatatctgactagaatgaatttttcaatatttatagaagtaaagctttgctcaaaaaataatttcactaagtgaatatataaccataccatgttaaaattatcagtcaccttcactagaaaatatttctctcgcaattctgctgaaatttttctgttgtctcatttcccatgtgtcaaaatcttatcaaacttttaaggcccaactccctcagtaacttttctttacaaactttgcaattaaatgaggctgctctcttcagaactcccctaagactttgttttgtagcatattcattctaccttgcattacagttatttgtatatgcagtgtactcaagagtaacactcttccacagtattgaaaacaatacctgtatataataaaatatttaagtacttttgctaaatgatctacccactaaccccttaaaaaaatcaaattgtccttgaaaaaggagtaaaaattcagagtattctggatgcatgtatgatgcctatatttgtatgactactacaaggaatactgaatccatggcagtggcactgaacatctagaagttagaaaatgaacatgtttggatattagtatggcaaagacagactcacttcattagtttgctatcccttatctcaggtaatactcctatccacaattataaaatgagcggaaaaagtaaaactgaaaataaaggtaggaggaacaggtattagacactatttggatctactcatgtttcatttaattttcttatcaatttactacaaataaccagattttttttataacttgtttaaaaataccctaacatccattcaaaatgctgctgcataaacacaaatctgaattggaatcttagcactgctatacaatcactttttaaagtgcaaataagaacaatatgtagcgaattaactgataaagatgtacaaatatgaatcaaatttattttacttaactatagaataccttcaaaatccatgaaaacataaaccagatttaaaataccattcttacaatgaaacaactatttaaacattcattctttaacagggtcgattttgaaactatttattctctcctactagaacattatagtcttcttaaagaaaaacagtcatgtgattatataaactaaactcttgcataaatgaaatatttctaagttagtttataataattctcagttacttattagctctggcatatgtataagaacatgattgataatacaacagtaaatattttcctaaatattacacactccactataaggcttctaaatgaacaactttaagtcgaaaattagaatgagggaaacttaccagcactaaaagaagaacagatcatttgaactccaggccgagggcgctctgtaaattttgcaggtcttgggctgtaatacaaaaaataaagaattaaaaatatcctaaaggaaccagtagcagcagaaataatctgtttcaaaaaataatcccagaaggacaaaattaagaagcaacagatggctcccttcctaaaaacaacttagaaatcattatgtgtcataaatcagaagatcttgtagaaattctagatatagattttgtaggagctccttattacacaacaatacgtacatggaacaattccaaattcactgtcacaccagacatgcagttactagttacacttacttctaatcaaatttaacatgttctcagtttttcatatagaatagcaacgtacaaacatataaggaactaagctattcgcaaacatgaatacattagcaaaataggtgctgtctgtgccttatgtataccatcaataactgaactttttcagtattttacattaattaagcttttcccttctttgacctactaatgtgataaaacatgtcttttaagaccccaaaaagtagggatattacatttaacctagtgaaaatctgaagatactttgactcttacgtcaactacaatgaatgctcattcaaaatagcagtctacagaaaacaggttacacacagctgtatttacattaattgcctaactgtattacagattacatattttatatcaatactactgatattaaatgtttaatgttacagtcaaccaattagagaaaatgaagatttttatcatgacaccagctctaatacatttaacaatgtgtatgtaatgttccaatatactgattatatttgaagccctacttactgtacattttggcatagttcttctacactatttgatgaaatgcaaaaataattagagcttaagcctatataactttcacaatatataacaaatttagagcagttttagttttgtgatcatttactggaaaaaagtatatacataaaatatttctgagctataggttggtgcaaaaataattgcggttttgcttttttttaaaaaaaagctttttaccattaaaataatggcaaaaactgcaattatttttgcaccaacctaatatatattctgagcaaagagaattatcttttttactgatacagaatgcaacaaaatgttaagaatttaaaaaataagtttgtaaatagttttacattagtatttacagcaaattctattaatattcacaggctctaatgtaacagatgagcagaacaaatctcatttagagagacagatattagaacattcttaaaacctaaacatttattcagagcaaaattaactgtaatttaagtaaattaatctgaattatgaaggcaactaaatgcattgctttcattactaccttatggattatagctctagattttttttaatttttggtacatctgctcacataagttccaagcaaccatttacctgaaactcattacaaaaatatgcaaatagtcctataaactaccatttttaaaaggtttttattttagaaaggaaatcagtatattgaagggatatctgcactcccatgttcgctacaacactgttcacaatagcgaagatttgtgggttttttggtttttgagacggagtctcgttctgtggcccaggctagagtgcaatggcacaatctcagctcactgcaacctctgcctcccaggttcaagtgattctcctgcctcaacttaaaattttataagttaaattacagccaaatgacaaaagcaatgaaattatattttaaagtattaaattagtgtgacaatgtaagtaattatgtgtttgtttacttgtttaggtttaaagcaaatcagtaaggttagtttaatggaaaacacacacacatagatgctttggaacctgatggaccatcatttgagtctttgtcattgctaatgttacttatttttagacacttctctttacacactggtgaattattttgattaaccaataaatttaataaagcactacaagttacttttttattggagacagaggctcactctgtcacccagggtgaaatgcagtgacgctatctcagctcactgcaacctctgcctcccaggttcaagtgattctcatgcctcagcctcccaagtagctgggattacaggtgtgcaccaccatgcccggctaattcttatatttttagtagagacaggagttttaccatgttggccaggctggtctcaaactcctgacctcaggtcatctgcctgccttggcctcccaaagtgctgggatcacaggcgtgagccaacacgccccgccacaatagtgaagatttggaaggaaccacagtgtccaacaacagatgaacggataaagaaaatgtggtacttatacacaatggagtactattcagccataaaaaaagaatgaaatcctgtcatttctaacaacacagatggaactggaggttattatgctaagtgaaataaggcaggcacagaaagacaaacatcacatgttctcacttattttggggatctaaaaatcaaaacagttgaattcatgagatagtagaggatggctattataggctgggaaaggtagtgggaggaagaggagggaggtggggatggttaatgagtacaaaactaatagaaagaatgaataaggcctagtatttgatagcacaacagggtaactataatcaatagtagttatacatttttaaataactaaaggagtgtaattggataatttgtaatacaaaggataaatgcttaaagggatggagaccccctttaacaccatgtgattattatgcattgcatgtctgtatcaaaagatctcatgtaccccataaatatatacacctactacatacccacaaaaactaaaattaaaaaataaaaagattttatatttttaaagggaaaaaacaagtagctacccataatttgtttttagatgcattatttgaggaaacatttttaaaaagggccttgggccgagttcagtttctaggtctatcacttatcaagagtgcgaccttaggccaagttaacatttctgtacctcagtatcctcatctgtaaaacaggggtaaaacggaacctatttcagagttgctgggagaattaaatgagtgtgatacatgtaaagtgcttagtacaatgtccaatatgctcaataaatattagtatttttattaggttcaacaagttctagccaatccttcaatgactaactgccacttagtttggcacagtggttaaaaggggtttctgacattatacctctagtagtatttaaatcctggctccagtaccacctgctaacaatgtaacctgctgtgccccaggtttttcccttatctgccccagagataataactgtacctttctcaaagggttgttatagggattgagataacaaatgtgaaatgcttagtactagcttggcagactaagcgcctaataatcacaaataaaaatttgtaatcatcatattatatgcatattttaggattcctagtctctttacacctaagtctaaatatacttggacagcttcctcctacccagagacctctggagctagcttatggttcacttagccacttagactacccatttaagaaacagcatctttgctcgtgagttggtaatacacacatacaagtgaatttataaagatatttgagttcccaaagttgaattgattcattcaactaatgcagatgcaggatttctaaagtcatttcccccagcagaatatacaaaagcattatagctaaatacaatttttgcctttgattattaattaaatcctatgtgacataaacagtataaatctatatcctgccaaatttttggcagttttcaactatgtgtaaacacataaagaaaataggtgttccaaggcttatatctaaagagcaatggattgttcttgtttttgtgtttttaataagacaggatcttggccctgtcgcagaggctacagtacagtggtgagatcacagctcacttcagcctttaactcctgggctcaagcaagcctctcacctcagacccctgagtagctgggactataagtgtgtaccaccatgtctggcgtttgttgttgttgttgtttgtttgtttgaatttctgtagagacaagatcttgctctgttacttaggctggtctcaaagttctgaactcaagtgatcctccttccttgacctcccaaagtcctgggattagataagaatgagccactgtgcccagccagagtactcattcttatgcctgaactctgaatttaaaaattttaagggacaagaataggaaagaatataggaataggaaagaatattacttataaatacctagaaaaaactttgaagtccaaaaataaaaaaattactaagttgtatataacaactctattgaacataatgcaagctattaaaatacatataaatatctatggtaaaatattaagaaaacaaaattatatatatattcctaattatatctatataaaaacattcatggagaaaaaatactgtattagggtagtggtttatatgtgattctacataaaggttctgaaaaaatcatttatatggacaagcttacttctcaagcatccagaaacatgaaatgttattgtacttagcaataaaatcctcaagaagcacaaataaggtgtgagtttaattctgtaaaacattttctgttcctatcccaatttgaacattgctaatcactttttcttctctaaaacaataagacaggaaaagagaaaggtatccccatcaggtccatgaggaggttaaaaaacagtagcaacaattaacaattaactattgctactgtccatatacatcagtaaaatatttcaacttttatctatctacagaaagactttaaatacgagggatgcaactgaagtgaagtcaacttgctttgtccaaagaaccatgttttaaatcacaatcttttttcaaatgaagtagttttgttactcgagctaccatggcccccaagctgccataagaaccactctacaagaatgttcatatacatgaagttaaagaagcatgtgttgcattacaaacaattatctaaacactactgtttttaaaataacaaaggcatacatatattattttattaaataactcaacttgggttgctaatttatacatagcagtcagagataattactgatatataccttctaatctgaatgactttccaccccgagtggcagaaatggccatttcaacactgtgaaatcaactgaataatcaattgaatacactactttcttgttcaaagactatccatggagcaaatacactatttcctctccccactacatccacttaaaagatatggtatagaggctgggcacggtggctcatgcctgtaatcccaacactttgggaggccgaggtgggcggatcacgaggtcaggagatggagaccatcctggctaacacggtgaaaccctgtctctactaaaaatacaaaaaacttatacgggagtggtggcgggcgcctgtagtcccagctactcgggaggctgaggcaggagaatggtgtgaacccgggaggcagagcttgcagtgagctgagatcgcgacactgcaatccagcctgggcgacacagtgagactccgtctcaaaaataaataaataaataaataaataaaagatatggtatagaaagcatcaaagggcagagaagtgctctagtcctggccttgccaatttttaaacatagttttaactatgggaaagtcatttaaccatttcagtgcccttaatccaaagataatactatccagccaacttgttttgataaaccgaagtattaatatgggcgaccgcacaaatgcaaaatgttattatggggagggaggggaatacatctatctaccttgatgcagtttagtgaaacttcaatgattctgtctccctacattttcctagatctaaaataaaatctaaagtttatagattcagtagcatcaataattaaaattattctaaagaacagcattagaaattcttaagattaagttctgagcatcaaaagcagctattaaaactatgcagcacatagaaaggagtggtaataaaacaggtaaatgctgaaggaaagagctaggattaggataaagagaaaaaaaatgtgaacatgagaaactttctttgaaacataaaaaaaggggaggaataaaaataaaacaggttagtaaagagccaaaagaggatttctattatttactcaaggagaaaaagtaaatgtattcctattgtcgactactttatacttttgcaatttcactcattaaactaaacacatttaatctatgaaataaaatagaaactgactttattttaagggttccagcatcccagagccaaaaacaaatagtgccatctgccccagtagaagatagatatctctttgagccactgcacaatggtgagaactgaaaagacaatcacagaaaaaaaatctttacaagagtgacacagtcaaaataaaatctactttttgccatacaaatagcaactaacaacaacagttagaaaatggcaagaatttaccaaggttatgttatttaaagtccatatatttataaagaaagcagacatactcctgtcttcattttagttggccttatatactggattataaaggtgattataaaagtaacttcttaaaatttaataaccaaaagtgacttcattaaatttactttacattataacaacaacaacaacaacaatgtatagggattaagacaattacctgtagtgatgtaatagatgcactatggccctgaagaacagccaaaggtgcacaggttcgaagacaccagactcggatcattttatcacaacttccagctgctatcatggtattctcatagtttacagccatgtctgatatttcagcagcatgtcctcttaaggtagctaacaacctcccatcatctgttgcccatattttcacaagacagtcatcagaacccttaaagtaagaatggatattaatagaattaaccccataaattttaattcaaaatcttaacactgataaatctacctgttctgtccacttctgaacaagtatatttttaaataccaaaaagtgttaaatacttgtgttagcttacacaaagctctttattaaaccacttaaaaagagcacttgtgtactcaccagcaaataagacaagtgggataagataattaatatttacctttggttccctatctactatcaaagtaccctcaatgtggatttctgtaaaagaaattgaacttctgaaataaaaaaaaaaaaatcatagctgcaaaacaaatgcaagctacaatggtgactaatattatctattttgttttgtaatacaaaactaaaagtaagcttgtttggggctttttctctcaggaagctgtgagtttcctatcactgatcttcagctaaaaacatgacattatctaaagccagttatcagaaaaaaattaatctcatctgtatgaagtcaataaaaatacataattacttgtttactctgccatagtagtgtaagtccagaaagaaattgtaaaggatatggagtttcctatgaatatctatatttacaaatgaacattcccattttatatagccaaaatagagatagaacattcagactctatttttattttttattataataattttaaatatataagaaagtagaaagaatagtatcgttaatccccatatatctacttcccagattcaaaacttaatatttttgccacattttctagtctttaacacaaggttaaaagagaaaacacacacttttgcaaaattactgaatattttacaggatacagttttactaagggttcatgttagaatgcttaacgaccttccaatctaattcttaaagaaacaccttcatatctgacattagaaagaactcagaggacctatggaggcatataattcagacaactttctgcatcatagtgacaataaatataacatataaatcatcatactgacaataaatataacatattaatgtttccaaacagagtatgttaaatgctgtatcttaaatcagactctgccaatgatacctaaaacaccccccaattaacgataaaaccagttcctctgattaagctttggagtaaaataaatgggttactaaccactataaagaccagtaaaacttaactttggtcaactatccatattgttgtctagtattcttatcacatacccttaacctcacctcaggctcttcatctataaaatacggaggttaaaatggatggcctttaaagttccctaaacctttaaaattaagcgattcatgcattcatttatccctttgtcttgtgactatgtgacaaccactaaagatataagaatgagatataaagacacagtattcttgtccttgtgaagcaagacagaagaatgtaaatgtgataacgtcctatgtattatataggacatataaacacttatatgtcctaagtgtttaaccactgttaatcagcagagattcaaacaaggaaacagtctactttttctgagagatgcagaggtacggggtggaaaggaacccagaagtggtgatgcttgaatagagtctcacgcaagaagaggcaatgtaatgtagcttagaggacagccttcagggtcaaatgcatgactttggacatgttacatacacttcttgtgcctcagatttctcacctatgcgtacctcataggattgtcacaaaactaaatgagggtgaagagatgagacagtgtctgacacatggttacagctttatcttctaacactgcttttgctggtgacagtattcatattcttattttaataatatcattattattattttaaaaagagcattccataatgtgactataaagtatggtcaaaagcatgaaaatgtgaaacaatgtgagtagttcatagaactgcaagcagcctgcaaggtaatttagagtgtgaggtgaaggcataaggatgaaaaagtaaaaaaaatttgtgaaaggccttttacggtttactaagaaaagtggtgtgtaatgagggtcactgaagatatcaaacaggtgagtatcaggatcatacttgtgttttctaaagatcatcctggcatcagtgtgaattaggctaagacagaaaggagaccagctgaaagttgttaaaatagtatggtcaggagagaataagtggtaacacaaattattatagcaaaaaagaataatcaaagacatggttataacagctgtttggggaataaaagggataaggagcacatcactgattatctgttggaagtgaaggaagagctgtagcagactatgactcccagaaagctggtctatacgcacatcggaaacacattagggatttgctgagtaaaaaaaatgctttagggcattcattcaaattaagttctctacatttcacaaattgaatcaacatattcactacatttggttatcttcccaaaactgaagcaattttggttctcacctgcattcagtacacaaagaattttaagtacactacagtaggtagaccatataacaaaagtaaaatcatgacatcatgttatgcttcacaatactgatacaattcatatcattcttatgaatctttgaataagagtgtgttttacattccactataaagatgcttcacatatttttcatgttaacaaataaaaacaccagtcttttgaccaaaatgtcagttttaatgaaaggagcaatggtaatctgtgacctaaaattaacctccagtgactttcaccaattaaaatgtaacaggaagtcctactatattcctactgggtttatcatttagttatcttaccactttagtattgcttgattaaatttgctctttttagacaagtgctgaaaacaaacaaaaatgcatatgcttccctctgagtgcatattatctcaattaacctttcttttcttccatcaaattgccagagagagaaatttttgaccatcctttcacaaaaatctctccattatcctcttccatgacccacagaagtttgctgcccctacccctaattctacccctcaggactcccggaagattttccaacagaactgcaagcattcttaagcaatttctatctcatatatcatcgcttgtgataattaatttaactttatggaaatttgaaacaaaagataatctgagcatgaattcaatgaaactcttttaagatgactatacaatatacaagtactcaaaaataattgactagaagaactgcagaggaaaaattaaatgtattgggaaaaaatgtttaaagcactataaatgtgttttattttattatttatacatttccttatttactttgagacagtcttgctctgttaccctggctggagtacagtggcgtgatcatggctcactgtgacctccacctcccaggttcaagtggttctcatgcctcagcctcctgagtagctgggattacagatgtgcaccactacacccagttaatttctgtatctttactagagatggggtttcgccctggtgaccaggctggtctcgaactcctggcctcaagtaatccacccaccttggcctcccaacgtgctgggattacaggctataaatgtgttttaaataaatgaggaagaatgaattaaaaatcgataaatatgattattttaaaaaagaccaaaatgtctaacataatttgaacggatacactctcttttccataagcctacctctagttccacgaatgttactaagatcaataagccaaagagtaagatattatagtcttttgaccaaagaaaaataaaatgttaaaaccaagttatggatattaaaaataatgttacgtaaatggtgaaaaggggcaatgacataagatatacctcttctaaggtgtatgaaagaaaaggaagtagggagagatcatgtaacctcagcaaaaacaaaacaaaacaaaatctgaggattaaaagtgagagggagagaacaacaagcgaatgaactaaaaaagtgaagaaagtttggaaatgcagtggaataaaagcagtaagaaaggtggaaaaattctgcaagcaacaattaaagacctgctaaatttaaatagcatgatgttagaaatacctcaactgacatagttttttcagcaaagctccaatactcaagggaaaactaagtagtcatttcttttcagtaacatctcaatgttgctggggattgctgctcgggctaggaattggcaaagtaagaaaacttgaaagtacaaagtgtaagtgaaaataagtgattatgctggaaatgttttacctaagaatgataattgagttttaaatgcctgttaagagtttgtatttaacctgctgaggtagtcactagacaatttgtaagcagacaagacatggtcactatagtattttagccagatcctgctaatgtttatgtgagtaatagatctgaagctacaagaagaggtttaggttagagatagagatctgggtgttatcagtttataccatagtagtcaaaacaatgatagcagatgagattacgaagagagaccacttagtgtagatttttatctggaaaactatgaacaggttttaagaaatccatggggtgggtacaaaatttaaaatcttttttataattcattttaaaatcaaattattgtgctattgtttaatcacaaagataagagagtagtaaatcacttgtttcatttttctttcttgtttttttttgtttgtttgtttgtttgttttgagacgaagtcttcctctgtcgcccaggctggagtattgtagtgcgatctcggctcactgcaacctccgcagcccaggttcaagagattctcctgcctcagcctccagaatagctggaattacaggcgtccgccaccacacccagctaatttttatatttttagtagaggcagggtttcaccatgttggtcaggctggtttcaaactcctgacctcaagtgatccatctgcctcagcctcctaaagtgctgggattataggcatgagccatcacaccaagcctcatttttcaataatgtaaaatggttataattactgcgaaagagtgctcattaatattatcatttgtttatatcaaacttagcataagctggaaaaatctcaagcaaatctgtatagcccttagcttatttaaatcccaaaacaaaatgagacaccaaatttacagggtttctttttaagtcaagacaatcttgtcatcaaaggatgaagccaaggaagctaaaagagtacatctctatacttgaaaacacaacagcatagatattatttatgagaaagtgtgttgagaaagggtgggaattaaacaaaatttacatttttccaatcctaacaatttggcttcaagtactctaaaattagcttagtctactgccacacctgaaaaaaacacacatattatgataaagaaatgtgccttaaaaacagtcaacacacttctgcactttaggatgaaggaagaaaacagcatcagatatttactttgtaaccactgttatttcttctagtacttttacagtatggaggtaatggtaccaattacttttcctttcagcatgcagctggatttcttactataagcaattagtatttttttctgtatatccaaaaaaagttctgattttgtaaatccctttaaaaacttcaacattcttcaaaataaaaagtttcagaggcaagactcaaataaaaacaaatatagtcatacttcctagataccgcaggctcagttccagaccactgcaattaagcaagtattccaacgaagcaaaccacacaaattttttggtttccaagtgcatgtaagagttacgtttacattatgctgcagtgtattaagtgtgcaatagtattatgtctttaaaaaatgggcatacattaacttaaaaatactttattgctaaaaaatgctaattatctgaaacttcagcaagtagtaatatttttgctggtggagggctttgccttgatgctgacagctgttggctgatcagggtggagttgctgaaggttggggtagctgtgacaacttcttaaaataagacaacaatgaagtctgctgcatccattggactcttcctttcacaagagatttctctgcagcatggaatgctatctgacagcattttacccacagtagaacttatttcaaaattggagtcagccctctcaaatcaagccactgctttaccaactgagttcatgtaatagtccacatcctttgttttcatttcaacaatgttcacagcatcttgaccaagagtaaattccatctcaagaaactactttctttgctcatccataagaagcaactcctcccccatttaagtttaatcatgagattgcagcaattcagtcagaccttcaggctccactactaattttagttatcttgctattgcagttaacttcctccactgaagtcttgaacccctgaaagtcatccacaggggatgcaatcaacttcttccaaactcctgttaatgttgacattctgacctcctcccaagaatcacgaatgttcttaatggcatctagaatggcgataataatcgattctcaattacaggtgaaacaggaggttttcgatgtactttgcccagatccatcaaatgaattactatccatgacagctacagccatatgaaatgtatttcttaaataagacttgaaaatcagaattactccttgatccacaggctgcagaataaatgtattgtcagcaagcataaaaacaacactaaccaccttgtatatttccatcagggttcctgggtgaccaggcatcttgttaatgaacagtaatatcttgaaaggaatctttttttttttctgagcagtaggtatcaacagtgggcttaaaatattcagtaaaccacgctataaaaagatatgctgtcatccagactttgtagttccctttacagagcacaagcaaagtagtttagcataattcttatggtcctagaatttaaaaaatggtaaatggacactggtttcaacttcaagtcactagctgcatcagccctgaacaaaagagtcagcctgctttttgaagctttgaagccaggcattgacttctctctaattatgaaagtcctagatgatgtatttttccaatacacagctgtttcacctgtaattgagaatctattgcttagtgtagcaactttcttcaatgatcttagctaggtcttttggataatttgcggcaactacttcattagcacttgttgcttcacgttgcacttttatgttatggagatggcttttttccttaaacctcgtaagccaacctctgctagctccaacttttcttatgtagcttcctcatctctcagccttcacagaattaagagtcagggtcttgctttggattagcctttggcttaaaagagtattgtggctagttttgatctcctatagagaccacttaaactttctctatatcagcaatgagctctttaactttctcatcatttgtgtgctcactggagtagcacgtttaatttccttcaagaacttttgctttacattcacaacttggctaactcttttaacatgcattcctcactcgccttaatcttttctaacttttgaattaaagtgagagacctgagactcttcctctcacttgaacactaagaggccattgtagggttattaattggattaatttcaataggcaggcccaaggagagaaaaatggggaagggccagttggtggagcaatcagaacacatgcaacattcattaagttcgccataagggtgcaggtcatggcaccctaaaaagacttacaataggaacatcagagattatagatcaccataacagttataataataatgaaaaagcttgaaatattgtgagaagtatcgaaatgtgaaagagacaagacttgagcatatgttgttagaaaaatgatgctgacagacttgctttactcagggttttcacaaatatacaatttgtaaaaaatacagtatttgcaaaatgcaataaaggcacaatgaaacagggtacgtctgtattagcatttttcataaagcctaggcagtgtctagtaacacatttgactttaatgttctcatgaagaaaagttccacaggtctttatgacgtggctttctactgttaggcactttggtattaaaattatcctcaaaatccagaaaaaaatggcctacgtactgccatgaaaacttcaaacaacttcagacacagggccatgaatcacttcaattcgatgcagaaaccaaacagcacctaaagtctatgcccccaaattttaataatttaatgagtttccagaggttaagcttcaaaaggcctaattgaactatttatttatttaaagaaaagctaagtttcagaacaacttgatagagctctttctggtatggcttatttacagatactctgactacataaatgaaatacaggcctttctatgcaaggccaagaagtcaatttaggcccagatgttgcaaaactatgaagtagacaattagagaggacaattctgttcagtaataaagtaatttacaggaagcagcataaatgacaaggaatggttgaattctctaagtgaaatcatgccccaaagagttaaagaaatcaacgactaacattgattaacactgaatgactaatattctttgagtgtgcgggatggcaactaagaaacaacttgtccaaacactgaaactccctctacttatgagatagaactggctgaaatcagttggaaccaagatggccaactggagtctgcacagaacaagcttgctgacatcatagcctgactatctaccacatttcatactaactaccctagaatttgcacatgtgacccatgaggtatcataatgagttaactgtgcatgcccagggacattccagacctcccctttccttccaccaaacacctactaatctcagaattcacccctactgaacctgtaataaaaatactgccttgaaaccagcatgaggagacagatttgagcttgacccctgagtcttcttgggagttgactttcaatataaagcttttcttttctcaaaaacccagtgtcatagtattggcttctagtacactgggcagcaagccccctctgctcaataacacaagcagaaaactgtacacattgggaaacagtttacttctgttcagataacttgagaaaccttaaaattaaaatattgacctatgtacctaaaagagaggcataaattatacaaagattactactttgacatgaaaataaaagaaattatgtgattttttaactaaaaatatcttagagaatttggcattccttgaaaacctactgttatctggcagagtcaacaaggagaattttaatttctcttgaggctactttacagcttttgagtcagagatctcatctcttattgccattagaataagcagtagaaatgaatgccaaaaatgttgtctgtattgtcatatttactacaatttcattttcctattcaagctaaaaagtaacctgcttttctagcaaaactaaaaattgcgtacaatttaaatttggttcaatttttttctaggtcatttattttctttcttaccaatctatcagacctgtgtttcatttcccttacaaatcaacctaaacctcagggtcaaacatttcaacacatgtctgatttcattctcgacccttattcatctataccaccaatgaccaacccggtgtcaatctaaccatcacttgctctgatactgctaccaggatgccaagaaacattacggtaaggaatataagcatactaattccacaacactacgaattcatgaatctcctatttactgggtaggctaagcattatcagcaatcatttttcctgtctctattcaatactcttctattgccaagctttatcagtaatttctagaatccataaacaagactctcgccagacagaacatttcatattgaaaagtagaaactgttaattgtggaccaaataactacctttctaaaaagtccacactgctattgtatacatcccacctccttaaatatcattacatatcaataatctcctctcgtatcttcaaatttgctatcttagtagtttcttcctctcaatccagttttcctacaatttactgtcctctaaatgaccatactctgcctcttttccattattatcaaacatgctaaatgctcactattgcttctaaacaacctcctatgcacttaaaataattttgaatttcaaacgtacaaaagtttcaagaacagtacaaatgtttcccatatctccattaccacctttcttccctccctctctctccatataacatacacacataactccattaccattaatcttttttctgaaccatttaaaagcaacgctgggccaggcgcggtggctcacgccagtaatcccagcactttggaggccgaggcgggtagatcatgaggtcaggagatcgagaccatcctggctaacatggtgaaaccctgtctctactaaaaatacaaaaaattagccaggcgtggtggtgggtgcctgtaatgccagctactcgggaggctgaggcagaagaatggcatgaacctgggaggtggagcttgcaagtgagttgagatggtgccactgcactccagcctaggtgacagagcaagactccgtctcaaaaaaaaaaaaaaaaaaaaaaaaagcaaggctgctgacttgataccccattacctctaaatatcgcagtgtatatttttctaaaacaaggatatttccctatgtaagttgggaaatcaatactgataacactaccaataacaatactggtaacatccaaatctacagggcctattcaaattttgtcaactgttttgtcaacaatgttctcttttcctttttggcccacaaacccatatacactatatttaactgacatgtctcatcaggctccttcaatttggaatactttctcaggctttctctttcatgtcctcagcaggttataaagaatacaggccttacaatctgcaggatgacccaaaacctaggtctgtcaatgtttcttcatgaccagatccaggtcatattatcttttacagtaatcccacaaacaagaagctgtgtttttctcagcgcatcacttctgagaacacaacgtcaacacatcccagagctagtgaagttaacatggttacattagtatttccaaggtttttccccataaatttgcaatgtttgctctttaattgattagtatctttgggggacatattgcaagatcttcaaactaagatcttctactggtctttatctgctaattaaaaaataataataataatcagccacatgaatactttggagaaggggatcccaggcaaagtcctaatacaaagacttcaagccacaaatgggctttccaagtctgaagtacaagagatcagtgtaaccgaagtacagtagcagagaggaacttatcttacagatatttgtatgtatttcccctaacagatgctaagttttctgcaagaatggacattagtcatttttatatctcaaattgcttgattcattcatcatttgtggtatgcctcctgtatacatcagatactctgcaaggcactacagatctaaaaaataacaacaaaggcaaagacaaagctcacaacttaataggaaaacagacatctgtcatggcaatgtaataggaaaatacaatgtattagaagcacaaagttaagcggcagtactccacattcagaatacagactatcatatcttttaattgtcaagtttttgaaagtttagtttatattaagtgtattcagtttttcaattcccattccctcttcactgcactgcaatttcattcctatccccaatgctaaaacagaacattttttggtcttagtctgttggagctgctataacaaaataccttagacacttgttaatttgtaaacagaaagttattactcacagttctggaggctgggaaggccaaggccaaagtgccagtagatgtggtgtctggtaaaggctctctttgtgtgtcaaagatagtgccttctagctgtatctttacatggctcccctgagcctcttttgtaagggcactaatcccattcacaagggctccattcttgaggcctcatctcctaaagccctcacctcttaatgctaccacattggggattaattaggtttcaacatattaattttgaggggacagaaacattcagaccatagcagtcatcaattaccttgacaaacccaaaggatgtttttcagtactaatcttactttaggtttctgcctttcatgaaattctcatctcctggcttctaagacaccactcttcttttttttcccctagcctctcaggcagcttctctgtctcaattattgactcttcttttgtctgcttttttaaaagctgaagtttcagccttcttacattagatacatacaagataatgtattccattttccaagctgaatgattcctaaactaaatcttctcacctaaatctgagttcccactgcctactgggcatttctacttgactttccacatagatatctcaaagtcaatacgtttcaccttcacaaacttctccccttaaattcctaccacagtaaatgacaggactttctaaatcacagaagtgaaaaaatatgtcatcctatactcttccatctcactccctacatacaaatcagtctctcgagtcttacaaatcctatttttaatctgtcaattccatcccactgtgactgtttaatccctgatttcttttatagatcactgccagaaactttttgccaatttctctgtatatagagttagtttgaatccatcttctacaataatgcaaaagggttcaatgaaaagaaaaattctcctcactcttctaaccacatcaatcattagctctccattgccttcagaaaagaaacccactatttagcaggtcacaaaagtatcttgattttgtaccacaccaatatcgctagttctttatgatgagccatatttattctatgccatatccattaatacacaattgcatgttacattctcccaaagtctgtaattgcctttccctaagtctgcaatatccaattcgactcctaaatttaccaagctgttacttctctagtaaatttcccttaccatctcctaccacaaggttggattaggtatctttatctcctatggtatctcagtaccttgtacaccttctgtcaaggttttatcacattatatcaatattgtttgtttaccatctgtgaactctccaagaacaaatactactttcaattctatatcccaactgcttaaaacagtggctggttcataaaactctgaagttcattaaaggaatgcataaactcattttctttattataccatattaattagaatcagagagacaatttatgtttctgaaaaggggggaaaactctgctttttatatggcgttccatgtacttttgagtgccttagttgtgaaaattcattaactctgcttttctccgttaaatgtcacttaaggaaatgattttaaaaccaagtaaaaaacattaaaaggctaaaagagaattagtgaacaaaatctgacttggcaattatgctatttccctccttgggtttttctcattaaaataattgggaaagcacccattcttaaaatactgtcatacaaaataatgatacattttcctaatacagaatttcattatcaattacaatgatttcctttttaattcttgtataccatttataaataagattttatttggataaaaaataaaagataaaatttacttaaatctataagtagcagtaggaaaaacctaatgactgctttctattttgttcagtactaattatatgcattatttcatgtaatcccacaaaaatcctatgtggttggtactattatcatctactcccctctctctttaggtgatgagaaaactggagattaaagagatgaggtaatctgtcaaagtttcactagtagaagtggtaaagctgtgactaaaagccctctgatgtcaaagctgatgcttttaaccacagtactgtatgcctccagctgtgcctgttcagaaaggactcaagagaatcccttggaaaaagctttcaaatatatatacacaaatatcttagaaataaatctgcaaggtcttaaaataccaattatataaaaaggaaatactggttgatccattaccaaattgttacctccaaaaataataacagtatgttctctcacaggagtgtttcactggtcaatcatgatctactatcttaaaggctgattctatctattttcaagactgatttccataggactagttagcgtctagtctgtgcctagtgaaatgcaaaaaacactcagcacccactttattaatgagcaatatgaatagtgaacatatgtgtaccctaccaccacttgaagtgaaaataataaaaatacaagaatttttcaaaaaaatagtgccctcatatcttcgttatttcttattgtaaggtaacattctgaaatctgtaactccaaaccaccagtaaaaaattacaaatgagactgaatttagcaaaacaaattctatcacattcttaaaaaataaacatctttagactttggtaagaccatataaaatagtacagtgctacttttcttctcttaattgatgtgctttcaactaaagaaataaccaacaagcagcttcctcttcgcatattattcttgttctctaaatcacatgcccttaaaagaaagaatcaaatgtctagaaaaggatagcaatttttttctgtacagagctggataaatattttaggctttgcaggccatatgttctcagtcaaaactactcaactctgctgttgtagtgcacaggcaaccatagacaatatataaacaactgaacatgcctgtgtttcaataaaattgcatttataagaacaagtgacaaactgggtttgagacgcaggcagcagcatgctgaaccctggttcaaaaagctcttccaagtctgtacctaccacactcatgagatagcaaaaagcacactatttcactgcattctccctaaaaaaattccaggagattatatgtctaattaatatcaaaacatgtaaaatgcttcataaaatatgataaacaaatgcttacaatccctatcatttttaaagaaagaattcctacaaggttctttacaatggcataactttatactgacctactggcacaatatagtgctcctttttcattattttaatttattactgtttttgaaagaattctttcaaacattagaaaaatcaaatttacttaaggtttttgagaggtgaatttgaatatacccatattaaacttgaatggctaaattaattttcgattactatttgagagcaaattactactgtaggtatgtcaggcacttcagcaaatatagagatgcctattttccactctgaaaataatacttgatacaagagaacgtaaaaagggaaaatactgatataagaagtgatgtgcaaatgcctgaggtagattagagccaagggaaaagaaaagtataaaaatgcattcctacatcctgctatttagctgttttacagatgactgggtgtatggatagaggagaagtagtaggtataaataggtctcagtttacaagcaaatttactaaaaagaggaatctataattgtcataactacgtaaaattcacagctgctctcttcaaagacaggaaaatttccatttaacttccacttcaaattttcttatttcaaaagaaattaaaaaccttgtgaatgaatgcataccttcagcccacagggtagtgtttaataataaatatcatcataatagtgtagtattatgcttaatgaatgtagatgttaaggcacctgaaaatcaaatatttccaaaagtaattttctcacttaaaataaagctcaaaagctttgcttttctctattcaacaggttacaagaaacaataacaaataaacaaacccaaagaggctctataaacaaaacatcagatattttgaagaatgaactgttaagaataacaggtaataagagtattagatatgctcagaattttttagcttttttaaaatcactattttaagggaaatttctcatagacaagcaagtgattttctacagataatataaaaaggtatattcaataatctcatacaattataaaaaggcacatttaataatctctcaacatacttagatgtccttagttcaaaattaaaattattttatgccattttgcaaaatgtcaaactgtgtatttgatatatgttgagaaccatacttattcatgatgtacaaccatataataactgtgactgtgctgcaacatgtcatttagaaactttctgaatttggataaagtccaaatttaactaaactcttctgttagagtaagtgaaaccacctgaatttccggtttcctattaaaagaaaaaaaagcaaggttttacttcaagttcacctataagcaatatttcctcaattacatatatgaatataaataatactttagcaattacttacagtaaatatccgtctgccagttcgatcaaaagttacacagtacacagatgacaagtgtccaagaattcgtttatgcattttcatgtgctgatacactgcagttggaacaagtcgctcaagtctgtatttcccattcagcttccttgaaaacagagtatccgctaccaagaaaaagaaggaaaataaatgtaatctggaaattaattttcttacatgatcaccttttaagaattcacatactccaatttgtcatgtgcaggtaaaaataaagaagctttctgatatatatggcttctagttaaaagtctttaaagtaatgaataaaaacattgtttcacctgaaataagtcaggcactatcattctcactttataacttaatttgtaagttaaatgacctgtccaaaaatcacaaagtaaggcatgaagctaggattaaagctcagatttatttactctctggctagtgctctttaaaaacctaaagcatttatatgttatttccttaaaagctgtctatgaaatagtttttcttacaaaggcacttaaaactggaacccagtgtacttttcataaatcagtaacacttgaacactcgaaatctgacatgcagaatgatatttaaaaacatctttataacaagtgaagataaaggaatacgtcatttgcattattaaaaaataataattaaactgggaatcttgccaaacacctgtataatgattccttctctggaatctattagctctcccttagttctccctttcaactcattcattctaatcattattcaagatctgactgaagtttatcttctgtcccaaagcttgatacattgactccagctgaaaatgtcctcttccatctaaattactactgtacttattttctatactggtaacttatggacaaagaaggtgctcaataaatatatgttgactgatctgcaggcacattattaacctacagatgatcttctaatacaggcttttttttttttttctaacagtgactgccatctacattgggtaattagcactagggtttctcggtcgaatttagccctaaagaaaactaaatatatatacaaaatactacttagccaaggtacagagcccagtaattatgccctaaagttgataaaacataaatatattggttgtattatgaagaatctcagtattgcttatattattcacatccaataaatgtttggctcacactatatttccaaccactccacactcccgctgcccccaccccaaacccccaaaaaatctttgggcctggtgaggagatatatagcctttacaggttcctaagcagtaatatttcaaagaataattacactatgcttatatgttctttcatcacagcaataattttatattctgatacagtatttctcttgctgttagcatgtaagttctatgcaacaatctaccccaacacttgggaatttctaaaacaaggtttgacagttgtcaattagcatatttaggcttaagttggttatataccaatttaacagagactcaataagtttcaatcatagcttagaccccagacacattttcttcacgcccaggaatggcctggtaaaagacatcctccaatgctttggctccaaacttatttaatgcaagaatcacacaaaaaccatagacatcaattttcctgccaaatgagaattaaaatcatcttatccaaacaccaaggatcactcacatctcagactcagctgtctgttgtaagaaaataagtgatgacatgccacaaaaacattgatagtattaccaaggtgtactttaattccctctatccaaaatttccaaaatgtcgaattttaggaaactaacagtgttcaggtgtgaaaatatcattgttggaaataatatatcaagacccatttctcgatgattaagcattagtatataggtaagaatttttaagataaatttgttataaagaccatctaaaaatcggagatgataaagtattttataagcaaaaactacttctcttaaaagaaaatgttactgcttcttaaacacaggttttactgaatctttgacctaaactgggattaaatctattttcattttggaagccaattgaaaaaaaagaataaccttttcaaagttactttacagtcaaattttcaagcaacattttccagaatcacattaggtgaaacatatttatagctaaaactatattccacactaccctttgtaatgcttagctaccaattaactattggctatctatactatgactatattctgaaagaaaaggtacttagcagagtcctggctctcaaacattgacaaacttgtgttaacagcactaaaaaataagacatacagaaagaacatacgtagattcccaggtaataaggtggtggttcaaactttctaactataggatttaaaggagaaaatgtaagtatagttcagtagttgtacaactgaagaggttgaatttgaggaaggcttgaccatatcaaataggtagataatttttataaagatagaaggatgagctaggactcacattcgatgaactcccatttttttataataccatttggaaaatgtctatctctcctatcagatcttaagtaccttaaagacaaaaattctcttatatttctcaaacttgagtatagagccttttaaattaaaaaagtatataagtaaatcttccttaataataagatatacaagatggttcaatttaatttatagttttatggcagaagggcaaatggcaattattcactttctaaagaaattaataagagaccaattaatttgagtaaaaaggaaatgtctcttggattattggaagtccttaaattttattaagctagaacatcaatttttaaaatgagcatggttaaacagataaggggacacaaaaggataaattctttatatgacattataatttaaccttaaatcaagaagcatatatgactttcatttaaaagtacatttattttcataagtatagtgatagaaactattcttaaataaagcttcccaaagcaaatctgttcttttttcacttggcaaattcttttttatcctctaggatcagttcaaatgcaacctcttctaggaagatttcaaaagcatcctaactggttgcactacaacactgctcctctgttcaaaactgcaatgctcttcatttcattcagaataaacaccaacttcctgatacatcagcctaagacggccctgcagaaacttatctcccattatctctaaccttacctgctacttctcgccttgacctgctccagccactccaatctcctgttcttccaacacaccaggtgtgctccaaacttagggtctttgcactggctgttccctcttcatgaagtctttttccacatatggctaattcccttataccttttcaagtcttttctcagatgtctgttaccttctgaataaagcctaacctaaccagcccactgaaaatagcaacacagcccctgccatcacccataacttctgatccccttttattcagctttatatttgctcttcatctatcacttattaccagacctatttaattatttattatattcattgcttatttcctgcctgctcctactacaagataacaaacttcacatgggcaggattttattttgttctctgattagcctaagagctgacacaggttggtgaattaacaaataaattacatattaacaaaaataattaggtttagtgggtacagggactcacacctgtaatcccagcactttgggaggctgaggtgggtggattacttgaggtcaggagttcgagaccagcctggccaacatggtgaaaccccatctctactaacaatacaaaaattagctggtgtggcggtaggtgcctgtaatcccagctatgtgggaggctgaggcagaagaacagcttgaaccccggagacggaggttgccataagttgagatcatgccactgcactccagcctggggaacaaagcaagactccatctcaaacaacaacaaattaggtttaagaattaaaaaaaaaaaaaaaaaggaagatttatctcacagattaaaatattcaaaatatctctaaatagtgcttcattttaactgccctgctaaatgaatttaattgggaaataaggggagaacgtattcacttaattttctgaatatagaggataaatgaaataaaaattccagaaatcactgttatccatttgaataaagtctgaagtaaaaaaggagcaaaatactgaagcatgtcatttgcagcaaatcattcagaacagcctttgaaataaagtatatgtgctcaagtctacaaagccaattagtagagatcaacaaaaggcccacaacttcttaaacattagatgtgactatgcgcatattcagcccttgggttctcatccattacttctttaggtgctaggataataagtcaaattcccccataagtcacttcttacttcacacctagttatttttcgagaactgatttacttatccaatcataatactaatgcatattcaatttagaaaagaacataaatgaaagaaaaacccataattctattgtctatagcaatcacttttaaaatttcgcaaaggtttacctcaaaaacagcattttaacagctatgttgtattccttaatgaaataagaggttttaagtctgacagacctgtgttcaaatttgtcactatggagactttaagcaagttactcgttctaaactttaatttctccagttacattattagagaaacatttccatttaccacaatatacctggctttcatattatgctgttctttgacaccccacaatttcacaaattttacacagtcaattatattaatatcttctactgctttaatatgaagttctcctactcagcccaacagtacaataaacatctacttacattttcttttacttttttgtagtttagatttctcacttaactctttaatccatctacaattataatactcattatgaggtctctaaagattattttttcttcctcaagtaactatttgttccagcctttccatttactaaataattcttttctattaaattttgattacattattgtatatatggttatatatacatatgtattttttttttttctgtgctgggccactgacatgactgacagtctatttatatgccaataccaaaattttaatcttacaatcaatgatgttttaatatttatttgggtattcctctcataagtccacgtaaaaatgttgtatctttattatttgttaattttgaaaataaacagaaagttacaaaattttgttggaaatgtaaatttaactttactaccaaactgacatctttctatctagaacatggtgctttcttcctgttgttgggcccaaattttcaatgcagatgattttttaaaaagataaacataataaagttacctcattttctctcactacatcatttgaaccaagttcacaaagaaagaaaaaggtagctgccataaaagagtatctgtaataaccttagtaaatacatttttgaaggcactagaaaaatacatgataaaaaaaaccctgcaaataagtactatagcagaaataccattacctccctacaaaatgtttagacttttttctccttttgcaaagatctttgtaaaatgaacaagcacacatgataaagctgcaataaattacccaagatcaaaattaaccatggttaaaaaagatgacttggaaaaaaatgaaaatgactatgaattaacaaaatacaaaggttagtgttttttgttattattgttttctaactgttaataacaatataatatgctatataatacctactccagtgtaggaaagctgttccctcttaatcagaaatggaggaccacaaaaacagtgcttacaacttctgccaactcatgaaagcagagccctgctggcagcctaatgaaatgcaaggaaaagcatgtagctgtagattctaaaacctggagaaccaatttttatacagtagaagaattaagtgaggaggcacaagaatatgtaactcggaaggtatctcagtaaaatttgggccttttctgctgcagaattgggggtactcagacaagacgcatcagtattttatgagaaggttttcattaattttacttaattcattttttatcctcttttgactagttttactttttttttttttgagacagagtctcactctgtcgtacaggctggagtatactggcacaatctctgcagcctccgcctcctgggttcaagcgattctcatgcctcagcctcctgagtagctgggattacaggtgtgcaccaccatgcctaatttttcttgtatttttagtagagacggggtttcaccatgttggtcaggctggtctcgaactcccagcctcaagtgatctgcctgccttaacttcccaaagtgctgggattacaggcgtgagccaccatggctggtcttgactagttttattctgtgattctaattaaagaaaacacttggaaggaaagctcccaggttttctgtaaataaaatgcaaaagtaattataatttataattaacaactacagaaatgattcctaaattaaaatataaaagggagtaacttctaaataatcagtaacaggtttcattttaatctccaccatctgtattaataaaggctttggctttctacaaatacgattaataactatcactgtaaaacaacagtttggaactccatgacactaaaattgagtaacttaagagtacatgaaaacaaattccaaactgatttacccctcatatgtgccatctccaattttagatgataattagattttccaagaaaataatgctatattcatgactagacatcagagagtaatgtctataaaaatgaccctccaagttcattagttcattacaagtccaaatagttgtctatatatggtgttggtgatttcagaatttctatcagataaatgtattgtgtggcataaagtatttaataaggcataaaattacttgaaatgttgctcattttagagatccacaaaagtgttttaatgaaaaggaaatatgagggtaaaaaaaaattgctaatcataattttctaacagaagttacgttaaagccaggcatcaaacccttgagaaaatggctataaaggaagaggaaagcaaccatggtttagagttatgagaggtttttactaggacttcaagaatctgacgattaaaaaaaaaaagtcttatctgctgcaattaataatgtgggtataaatgtcaccatacaatacataacaaggagaggaaaaaggctacagaacactcttacgacgtgtagcaaatttagagaataacagctagtatttactgagtgttttactacatgccaggcactattctaaatattttacatatattctcatttaatcctccctaatcctttgaggtggatacttccattattcccaataagcagatattccttaccagtaaggaaaccaaaggataaaatgtaatttactagaggtgaagcccaggtttaaactcaggcagtctggtgccaaagactgtacccttaactattatatgctgcctatgcagatcaatattagaaaagaaagactgaaagaaaacatgccaaagtatgtacagcagttaaggagtgagactgattaatttttgagctgttctgttttccaaactttctctatagagcatacgttcattctaattttttataactcaatctacctcaaggaattcaaaaaagcagtaggaaaactctaaaatatctaaagagactagcttaaattcaaagactgacaaaaataaacctagagataaagatgcaaggaatttaaatttatttaatcataaaaaagaaacactaatgcctaagcatattacagtgtatgaatgtatattatgctatagagaatgacaattgcgtatttgaaaatggactgaaaaaatgatatgctataaagaaataattcacatcagttttgatttgaaagctgtaagataatactacaaagcaggccatcaactttgttgtaagatttgtttgtgtaatctttcccactgaatttttaaacaaaaagagaaacatgtcaaagatagttcaggttcaattttgtttcggagggaatcatataaggaggaatgtttaattcactatagagccacaatggaaagacctgttattaacacgggtatcaaggaagtaatgacaaccaaatactcataattcgagggccagaattaatctgggaaattattcaacacaggttggtttactcctacagtaccatctggtctctacatcatactgtaccagcaagtagcaaccaaccaacatagaaacaggacaaataaatcaccgtaatagtaactataatgaatgatagtcccaatttcagttaaagtacaaagaactgggttgcaatataaaaagataatcttgtagtcacctttttgtctacaaaaaaaggagggcaggtattgggaatagaagagtaaggggaaatagtcacctgagcaacatagcacccatgatagcttctctcttcgctttaatactataactaaaataatcagtatgcacagcagctactttccagatttctagatattcaattttggtgtgtagttgcacctcctgtttgaaacatactatgtttttaaaatttagcagataccataaagatgcataatgtctgaaaaggaaaacccaaatactaaactgaaataaaatacagaaaatgatgattccaagaagtaagcaaaaaatttcatgaaccaactacattaagagcactaataaaaagggtagatatgataattattaggaatagtatttaatcttcctcctattttatttcctggaaaccagtctgatgctagttcaagtagaaaacacacaatgacataatgttttcagttttaaatattttaaaatgtttacagttgttttaataacaatttatttttcttttaaataaacattttttaagttaggtggttttttttaatgtcaaacattttaatcactcaattttgagtcaacaaacatttatagagcacctatatgagccaaatatgggctagagaataagagggaaaaagaaaagacatggtacttaccctcatggagattgcagtctagcagggaagaaagacatgaaacaaggacttacaacaatgttaagtgttatcaaatagaaggtatagggaaatcttgaaatatatagcaaaagggttctaaacacgttggggctgaggggtggatatctgggagtctgggaaaacttctctgaaaaactgacatttaaactaagacctgaaaaatgaacagccacagaatgctgatgtgagcgcagcatattccaggttgaggaaacagcatgtgcaatagcctgaggctggaaagagcatagcattcaagcaacatgaagaagtcaagattgacttgcacacagagtagagaaagggcaagtgtcaagagaagagactgagaaggtaggggagcggactatatagagtgctttctaagctaggttaggtattttggactaaattccagtaataacgggttgaagttttgggggagaaaagaatggagtaatatacatagtaagatttactttgggataactcattgcagttttctcttgaccacaatgagaatgaattggaaaggatataagtaaaagcaaaagctaactttgcaaaaaaatcaaagggttctgaaaacaaaatttcattttagaaaaaatttaatcagcttgacaccaaaattatcaacactttcccaaggaattaaatacctgatctcataagtatctggcactatataaaaacttgaaaagaacacaccatgtttcattgtttctagagttcaaatactgaggcaaaattcaaacacctgctattaccaaatcaacaaatggacagagctggcacattaacacataaagaatttcacagagaaggcaaaaaggtgctatataaatgtgacataaagttaaaagcataagatctgaggtacatgcatacatatacacacaaaaacagagatataatgtcattggttactgcttttctaagcttcagtttcctcattaataaagtaagatcagctgagtgtggtggctcacacctgtaatcctagcactttgggagaccgaggtggatcacttgaggtcacgagttcgagaccagcctggccaacatggtgaaaccccgtctctactaaaaatacaaaaattagccgggtgtggtggtgcatgcctgcagtctcagctacttgaggggctgaggcaggagaatggcttgaacctgggaggagcaagttggagtgagccgacattgcgccactgcacttcagcctgggcaacagagcgagactcaatctcaaaagaataaaattaaattaaaaatgtagggttatcttaaagagttgctatagaaaacagacgagacaaaatgttttgcaaattcaaaggtattttatactaacattgatatggactgtccctaaacaatcaaatcttcatgtgccataaaagttaatttaactgaacatagttttcttttactttttaaaagacttttgttggagcccaattttccccgaggcttccttatggagctgaacaaattattcctttgtttataaaaatatctattcagcctgatctgatcatggacttcccaggtccaaaagatgtctaagaaaacactgaatacgtaactttaaaggatccctgaagaaattcaaaataaaaagtcatgaccttatgagaaaataatatcataatttgcttcacctacacagatatgagtattcaacaaaatcaaacccaataatcactctggaaaaatatgtgatgcaaactaaaagggaaaatggctagtgattcctaattactactggaattgcttgccagatggtttatatgaagtggaggggatatccctcatcacatctataacctaaaaacaaatgttatcctattagttacagaagaaattaaaacacagctagctacaaaagcaacaatttaaactcacctaagggagtttcatttcttcaaagttttgcccccttttttttgtcaaccattaatttcaaaagaaatttaagcagaggaaaaaataaataaatatatattttgtacatctgatactttggcataatgaacattatttccctaaaaaaaaaaaaaatgctaatacaccttgcaagtctctcacagctaacctgtttttaagaggcaaaaaaaagggaggaaatagaaaggcaggggaggggctagggagaaaatagttaataaaacaacaaaacctgtgtcaaatagaaatatgaagatcattcagggaaaacactaaaaaacaaagaccaagacaaaaaagaatataactgagtcaacaatcattaaaccaaaaaaaaaaaagcgaagtataaaacctttataagactaaaattatgactagaaaaacagaaaatgagctctaaaagtaaaaatggttactgagaaacaataggttaaaataaatatatttaaataagtgatgagtagatatctaactaggaataaagtatggattgagtaagaaaatggaaagaaaaaacactgaatatagcatattagaaaaagataagtgaacaggaaaaaaaccccatatattatatcctataatttgctgaattatatgtaactgttaacttatatattaaaattatgtaatctcatatatttgtaatataaaaaaattctaataattatgtaagaaatatatgaggaaaaatataaacctaagagtctaagaaaaaaaaaccttgattgaataaatttaagaaattaccagctacagaaaattcaaacacaatattggaaatatggcaaaatatagcacaaggtgagaaagaagagtcttctctacaaagatttgcatctagctttaagtgactgtttgctgtctttattcaaactagtcttctcagagaatcacaaaattataaatgtagacaagatttaagactttttttttttttaaatgcaaaggcttcttagcattaattggatgtctgggtagtgaagctacttttcaaggcaaagttttttccttaccctcaaacatgtttaagaatcaggattctcaaaactccttatcctcacacaaaactgcagaacttaatagcaaacctccacagacaagtaaaataaaaatatggaaatacttaggcaaaacaccaaaaacgatgacaaatgaaagacctagagataaaaagtttactttgctaacatgtcaaatgtaagaaaaatgcaaacaaagcaatcagcagaaattgctttaatttaatgtattacaatctttttcacaagataaacatgcattaaaccaacttccaaatttaatcttaaaaacccctttaatgtatttaggtctcttctttcctatctccccttactcatgcacatttattactgaagtataagcaaatatagaataaactatatctgaaaacaggcataatgtgggtatggaggtaagagaaaggacaatactaaagattcgctaatacctttggaagtaaatgctgctatgccaagtacacactcacatctctcttccacaataaaagaatcacaagctagtaataacaacagatcagtgggatcttttgtctttgcttttgaaaacagtattaaaggaggttctagagcactggaaggcaggtgaaccactttgggtctcttgctgagactgagttctagttcaattttcacaacttacatcaaagaccaaaaggttcaaagtagttgggaattctaagcacataataaaataaaacaggataagaaaacactgagacaagctcaagtggcttctcaaattgtataggtatgtattttatattccaagtgtaatgaactgataactcagtctactcataaaccttaatttcttaaaaatcagtttctggtttggtgattttatcttctcttaccacaaagctattcttggatcaagcttctcttctttctaccaaccttcattccatttttgtaataccttacccacctttgaactttacttgtcccatcccagtgtatgaatttcatcaactacctactacttttcatttaatatttactaatgctgaagagttgggtctctaaagagcaataaaacataatgtctgtcctccaggacctaacagtatagtagaggaaacaaaggtaaataaatagccataaccatgtgataaaatcaatagtagagataagtaaaaccacttaattctgcccagtttaacatctgagttttaaaggataaacagatatttgtacaacgtacagaaaacagaggggcattccagggagaaataaaacatgtgaggagacaaaaggatgaaaaccaacatggctgattctaagaactgccaaagagttgtactaagctagagcacaaagtgcaggtgaggacatggcaggaaaagtgggaaaaggtcagtcttgtgtgacacattaaaatggttgacactatcattcagtcactctcaatgggggaaaggaggaggcatgacatggtaatatgtgcacttcaaaaagtttagagcagtgataattttgcccaggagatatttggcaatgtctggagactttttaaattgtgacaagggggtagtggggatgctactggcaattagcaagcagaggccagggatgctaataaacatccaacaatgcacaggaaagctgcctacatccaggaattatctggcccaaaatgtctcaacagtgccaaggttaaaaaaccctggttagaatctgactcctaggatacatacaggaatagactaggggcaggggctcaagtagcaattaagttctgggagagcagaggtcttgttcttgcttatccgcaactccagaccctagcccagtgccagatataattagagatatacaatatttactcagcgagtgaatgaataatgtagaactccaagcaagaagtaccaaggcctgaaatgtggctgtggcaatgataatggagaacaaattcaagaaagtagaataaacaggtattatttattaaacggacataagaaatgaacagtaaatctaaaataaaattatctccaggcttctggcttggagtaacaaggttcagagagtgtcaaaaattacattttaggtatttttaagtttgagatgaatgtgagatattcaagaggtccaacagtttccatatggtttgaaatatctaagaaaatccaattttgccacaagcttaacctaaaattatcctctgaactacagtaaggagtccaataaaaaattaacaaaccctatgtcaagtacggtatcctggactggatcctggaataagaaaacgtaattagtgggaaaacttagtgagacatgaataaggtctgctgtttagttaacagtaatgcaccaatattggcttagtagttctgagaaatagaccaggtaatgcaaaagtataacatttggggaaactgcatgaggggtatacaggagctctttaaaccatctgtgtaacttctcagtaaataatttcttactccaaaattaaaagtttatttaaagaaaaactactaccccaaatgtgtcaaaattttaatatttgggatctatattaaacgtttaaatctacgtacgtatcttaagaaaagctaaaatatcaagaattttttcttacctccactgatacttggtaataccacactgaccaaggaaagagcaaagaaaacttttaaaggtagccagacagaaaatggcaaagactcaacaaaagaagaaataagtcagtactacaaacattcatatatatttttttcaatcttctgaaccatactgtaacagcttgaaataatgcattgtaccactatgcaggcaactattctggcaagaagactgatgaattatttctccccaacaccatcctgttcattactttatagcgataataaaacaaaatatataccatggtgaactctagcaaaacacagacactaacctatgactatgcaagttgagtcattttggtcagtctagcttctctgcagtgctttcaaaaaatatatgtaaatacaatttttaaaagtaggcaaaatgggcaatccactacaggtgtttcttaaaataaagcaaagattttcctgaaaatcacaatggaagagagagagaaaaaaattatcttaaactctaaaaataaaatagtctttcagaaaggtacatcaatcacctgcttggcaattaattctgttacctaaatgaatcaattacatttctatgcttggatgcaaaacccaaggtatttttgccatacgtatatatagggttctgtaccatctgttgtctccgacatccactacggatctcagaatgtatctcctgtgaataagggaagataaatgttcctctctccttgtcagcagtatttactaatctgaccatcaacgacgtggcctaaaatattatatttgattttataaatatttggttcccttttacaaaaaatgactaacaccaattttcttgagtagccaagtgttattattaataaattcagtttactgggaataaagcatagcataatggagtcaaacagtctgggttgaatcctggcttcacttctcactgcatgtgtgaacttggtcaagttaccaaaatctttccatgcttcagtctcctctgtaaaataagcataatagttcctacctatagaacatttaaggttttaaaagagtaaataaatagaaaatgcttagaacagtgtctggcatacaggaattactcacaaagcaaatgttatttaccatcaatcattcttacactttcattacctaccacaggcctgactgacaatgtactgaaagaacaagcataacgtgttctccttattatgtggatctatagtttttcaaagacaggataaacttttcctaaatgggaaaactcctataatattttatctttcccttcttgcaggaatcctattataccaccttagaatacttttctaagtacaaatataccctcgtctctcaaatttttttgttgttttaatggttgtttattatagcaagattatatattgaaattatttaaacaggacaatcttatgttttaaaaaaaatcatagatgattacccaccacgcagatatcacatacgttatctctgaaaagtaagtcagagcaaacaattcagaatacatcagagagtcaaaaacatgtaaaacatagaagaaaggataacgaagcagataaagtgtttaagtcccataggaaaggagagagaaaggaacacagaggttattttaagataatgactgagaatttttcaatgtcaataaaagacatttccagaatctaagcaagacaaatttttaaaaaattaacatcaagacatatcatactcaaactgcagaaaaccagaaagagaaaaaatcttaaaaacaattagagaaaaagggattgccttcaaagtagcaagttagatagtagatttaaacccaagtacatcaatgattatatgtaaatgactaaatgttccagataaaacacagattatcatattgtatttttaaaagccacttttatatttttaaaagacaagaaataaaaggaaacaaaaagttgaaagcaaaattatatatatatatatatatatatatatatatataaaaatgccaaacaaaagtcatataaacttacgatctgtataataagtatccagaaaaggaagaattaagtaagatttgaacaggtggaagaaagaagagtaacattcctggctaggcaggaacatgagagtggaaaagaagctatgattgaactttgaaggtctctgaaaattaggagtacacttagagaaagtacaacactattaaaatatcttaacctctttatactatcttcagcagaagagtgacacaacaaaggtagaactgaataaggttactattacagtgttacacactatactgggagggagagagagatagaagacaccataggcaagaagattagtttggatgatgctgtattaatccgggtaaggcaagggcctggtagtagcagatgtgaaaggcatgaacctaggaggcatctcaagaagacatacttgcatcagtactccctctttgaaactttagccaaaaaaagaaaaaaaaaaaaaagtactgtgctcttaaaaagataactacttttgtctcctaccattataactaatctgaattatatattgattctactaactcgacctaataatatataccttaatatggaactttcgtaaaaataaaattccaatgagctaattggcctacataaggtgcaatgtgcacaatccctcattatacatgactttttcactattaaaactactactaagaaaaaaaatctatttttttctttttgaatgcagatgtacataatatgaagttctctaataccacagtaactaaaaaaccttttcattttcaaaagctctttaccaagaggctattaactactagtgaactcaaacaactcaccaatgctgggtgggctaccatagttaactggtgactcaggtggtcttccacagtgcaacgcagccagagcagatcctttccacacaacatgcttgcagcctattaaacacatgtatttttatgcatacaaagaacacaaaaacaaaagtgagataaataatgtctaaatcctatgagaaaattttcatacttttatttgtgcgtagtaaagactgtcttccagctcctaataaagtttgtactccaggaacactttgaggaatttcttgttcaagaagaggtcctagtcgatgacatatttgcagcaagtgatcaggtgctaagtgtctgtaatacttcacctattatgtaaaagacaaatatagtaggtttcagtttatcattttaattttcaaaatctttgagcaacaataaaaaaattcatccaagtataaaatattttgttttgcgtctttgatgtaaagtaaatctccagaataattaagaattaagaactgatagtttgttattaaaaaatttaagaacacttaacatctatgctgaatttcataatttaccaaaaaccttacagagagaaaggcaaaattccaactgctgctttaaatatctttcaatcataaaataaagctacccactataaaaagtttgagcacttttggaacaacttcaaaatactgcttaatttataccggcatttgaaaccatgacatgaaatgctaatatttttattagtctttcagtaaataaacaatattcactatctaaataaaattatattggaaaaaaatactattgtattacttcataattagctatcaagttagaaaaaaatttccacagtagtatctagttcagctattctcaaagtatgttttgggaacccctggaggtccctcagataaaacttatgaataacactatattagttgtttttcactttttctcatgaatgcacggtggaggtttccacaggacacataacatgtgatgtcttaacagactgaatgcagaagcagataggaaaatgtctcctctattaagccagatattaaagattcacagaaatgtaaaacaatgccacgcttctcacaaatttgttttgtttgggaataattattataaaaatgttacttatattaaaataagattagtttattagtattatttaataggtctccaatatgttaaatgctaagtttctaatatggtaaataccaatagattataagctacacaaataaaggaacttcaatacattttagtaagtgtaaaggggtcctaagaccaagaaatttgaaaattgttcctcaagtttaccaagtaatggcaactttaacatgaattccttttgataagactgatgtggggggaggtgaggatttaaatcatctcattccatcttgcaatatctgctatactcttaactgcagaaatccatgaatgatatgtttttaaaggtagctaatacccatctaaactgaagccaataggagaaacctaccttactctttatcaaaatacactccttctttcacaaagataacatggagccatactgccaccaaataatctttggtaagattatttaaaacagcagtttggcatacagtaggtgataagccagtaaaatgagtatttttggaaaaaggagttctaatacagtttgtatcatatgaattatacatattacctccttgttttgcagtcaaaaagcacactgacattgaagtctctgaaaaatcctgagattatttcccaaactcatttagctacagaatcccttttttccctaataatatctatcatattcactcagaacatactttaggaaacactagtatgattagctaaattaaaaagcatttaaaagaaaacttaccaaaatgagtttttaaaatcgtatacttttctttaatcttccccaaaataatttactcaaaaataaaatttagaagtctagaatacttgtaaggttgcttccagttctaagcttgcaaatgattattttaatgtgacttaattgatcaaaattccttttaaaaattttactttaaagaagatggaagttcattacttattaacttcagatgtgtgatgatcctgttttagtatcctctggcaaaatatattttcaggtagtgaaactgaaaatccttactgtaatattctatctttcaataaaatattatgaatccactctgactcaagctttctttggtgatttagaatgtttgaatttttcaaaatcaactttcattttaaagttagaagagatacttccagttcttaaattccttgtgctttctctggcttttgagactttatacaagctgatgcctctgctggcaatcttgtcttacctgctcacctctacacctcattctccttcatgtctcagtctatgtctcactcactgccttccatgacctatttacaccacctgtgcccctttttggacactttgtgttcccacagcacattatactcctcgaatgtcccttcatccctctagcactgtgtagtacttaccatattaattgttcttaatatattttgatacttagacttttaagaatctaatgacagctacgatcttcttccctgcaaaaatacacaagccctaccattatgtgccctgttttgggggttcataagactcatgcactatactaaaattgccagtttacttgtctgtgtcctgcataaggagagattgggccatgtttacctctgtctacccaatacctaatgcagtacttatagttaagtgcttaataaagttctagttggatatatgaagatttaagaatatgcagaaacgactgacttccccactctcaaaaaacccaaaacattttgttagcacctatcccagcacataaaaatagatgcagtaaaatttttttacatggtataattctttattctaatagttttggggtgagtttgtttgttttgagacggagtctccctctgtcacccaggacggagtacaatggcgggatctcggctcactgcaacctccgcctcccaggttcaagtgattctcctgcctcgcctcccgagtagctggaattacaggcgccccaccacctaacctggctaatttttgtatttttagtagagacggggttttgccatgttgaccaggctggtctcgaacttctgacctcaagtaattcgccccctaggcctcccaagtgctgggattacaggcatgagccacagcgtccggccaattctaacagttttaaaacactttttaaagaaagccttagaacatgtcttcaaacaaattttagacaaaacagattaaagtaaaggtactcagaaagtatcttacttaagtggcatcagggaacacatatctcaatgcttgactctctacttgcttcctcttagcagtccctgaggtaccaccatgaaagggtctctggaaagaacagtggaaacagactaacaaacaatgctattatcctctctttcccaagaatcctcttccccacccctcattttctcagcagatgacctaacctactttacaggagaaccttttttaaagctgaagctttgtttcttccctatcaaatctgtaaacctatctacacctgtaccaattctgtctcttattaacacaactgtccctcttaataaatatttaagatccaatccctctacttatgctttgaatcctaacttctctgactgcacggaaatcctacactataaattagacctccacattctagtatgtacactaacttcctctcaaccaactccttcccatctacatgtaaacatgctcattttgcatccactttaggaaaaaacaaaaatcctgcctaactgctattatccaacacctccccacccaaatatcctggctacccctttcccgttctcttcctctccggaaacatgtttttaaaagaatggtccatagtccatctaatttcgtatcccatcctctcctcaatctactccacactagctcccacactcatgactttacaaaaaatagctcttatcaaaagttcaccttcaattatcaataactccaataagcatttttaaatctataccttatttaatgtcccataatatttcacctaactgttgaacacttcctcatttgcaaactatcttctcttaacttttgtgacactccttggcttgctttcttcctctccaaccattccttctcatttttcttttaggcttatactcctctatatagccattaaatagtgaagttccttaagatcctaggtaccagagtccagttccagatcctcttttcttctcactatatactctctctttggacaattgttataacaatgattgccaaatttctatttatagcatagacttgcatagcaaactttcttaaaacagctctctagagccccaaagcatctgaaactcaacatatgcaaaactgaattgatggatcctcatgaaaacattccccactaaagtgttccctacctgggtggatgtcaaccccatttatccaaccttggaagccagaaaccaaggagctgcattttgcacacagttcattccttcctccttcccatcatattcccaatatccaagcagtcaccaagttcaacttaatttttccttcctatttttaaatccatctacctgtatctccataacagtccaaataatctttgcaataaatgcatacctttcccatatgcactcatgccccattcaaatctattctctatactgcaattagaatgttctttccaaaatacatattggatcaagtcacccctacttaaaacacttctgatgctttcctcactctttggataaagatccaaatccttaacttggtctatcagcccagaaatgcatgtatggtcacttcttatttttctagcttcacctggcacattcccagtccctttccccaactctcacttttcacgtttcaaacacatggccttctttcaggttatttacgcataatctctctctcctgtcagactttatcatatagcatacactctgcatttccccacacttccttgcccagacaactcttccatgtgtctcagatcttccctctaatgtaacttcctttgtttttctatactctacacaatccaagtctgttttcttggttgcacacttcctgtgactttcctttattaactcaatatattggcttgtgattatacatttaaaagtgtaatttaatgtttttctacttcaccattaactacaaaagagcaggggccatgaatctttttgctcttaactataccacacacacacacacacacacacacacacacacacacacacagaataaacaaaaatattttttaaaataaaacaatcttttctactttttctaaacattctttataaacatattaatcatattcatattcttcataaatattaatccattatttacagatatacatatgtgattttcagttttcaacttagtaagaaccccatatctttaatataaacttaagcttttaatttaaattagcttttatttcactggtaaataattaaaagacacatttaaaataatataataataaaatctcttactatattgtatatatgtggtttctcagtaatctgccatacaatattatttcaggggaaaaataacccctcaagatccccaatttctgatatacgagttactttctgtgaccctaagtgctttcaaattcttaacattcaagacataaaaagtatgaccagattataaagtcagtgtgataaattatactaatatagctaacacatattggctgcacactgaatgccaggccctatggtaagtgtggtaagttttacatggaactactcataactctgagaggtatatactatcattattcccattctataaaaaaattatagaatttatttaaaaagatattgagaccttcccaagttcaaacacagcacataagagagtcaaaccatagcaatctaactctggaccctacaattcatactatcacacaaatgacctattacctcaaatatgtgtatatatcaatgtgcaagatataagcaagtcatacaacagacattttgaatagttttcaacagacattaaactgagccagaaaaagagaaacatttcacagttcacttgcactactaaggaaactagcataaaagcataaattcctataggtaaaagggaacactttaaaaaattctaagggtaaaagtagaagataaaactacaatatttataagattatactgctctaaacccttaatttaaaattagaaagtaaaaacagattaaagagttaataccaaatttgttactattttttaaatttccccaagaatgcccatgtatcagtagtgctcaaaactttttaaactcatccaccctggttttgtttttgtttgttttttaagggggcagggggatctcgttctgttaccaaggctagggggcacagtcacagctcattgcagcctcaaattcctgggctcaaacgatcctccgacctcagcctccagagcagctgagactataggtgcgttacatcacacctaatttttattttatttttttagatacagcatctctctatgctgcccaggctagtctggaactcctggcctcaagtgatcctcctgcctcagcctctcaagtagctaggattacatgtatgagccaccatgcctagctctcatccctttttgatgaacaaaacattttctctcctccaataagatgcaagaatgggccctatggatgcaaatcctgatgccatcccattgagattcacacctctactggctaaaccaggaggctagtcagagctttttcaaacttatgtcccttccacctccgttctcagttgagttgcttgctatgggaacaacaatctttggctaactgtccatccattttaactctttttcatagttaaaatttgaattagccaaaggtatcctttttttaaaatatcatgttatattatttagagtgcaagtcagcaaacatttgtaaccatcatatggtaaatatttgaggctttaagaataatatactatctccattgcatgttcttattttttaaaaacaattctttaaaaatgcaataacaatgcttagcttaatggccttccaaaaacagatctcctgcacaatttacccacagcagccattagtttaccaccccctgatcgaaagaatgattcatggttctagcaacgtttccatcagcaagaacaaaagaattctgtgaaacagcagcaactctgctcattccttgtccatcttggcccaagtcataaatctgtatcatcccttttccagatcacatatataaagtatttctaaaattatccattgcaatttgaacaaaagggtcatcttctgagaatgtgaattacagtatggaagaaacaagaaactgagctctttgagaggtttattatcccgcatttttggctaaaacaacattttctgatagtatgctacttcacataaacagccatccttcccagccaaaaaattttgccattatctcagtggaaggtattgttaaaaggcaagtgttatcagtaggaagaaaatacaatggatctggaagctgctgcattctaccagacttacagtcactaagaactctactcagtttttaaagaaatggtcagtagtttaaaatgcgaaatctcactgggcatggtagctcatgcctgtaatcccagcactttgggaggctgaggctgatggatcacttgaggtcaggagttcaagaccagccctgccagcatggtgaaaccccgtctctaccaaaaatacaaaaattagccggacgtggtggcacgtgcctgtagtcccagatactcaggaggctgaagcaggagaattgcctgaaccctggagacagaggttgcagtgagccaagatcatgccactgcacaccagcctgagtgacagggcaagactttgtctcaaaaacaaagcgaaataaacaaaaaaccccacgaaatctcaagtcacacagcctgggtttaaagttgctaaggctgagggtgggaaaaacatttactctttctaagcctcagtgtcctcatctgtatgactggtatcacaacagtcaccaccttggaaagtagtcataaattattaaattaaaccatatgaagcagttagtactgtacctaacagttggctgttactttaattacattctatattacatattgacccttcgtgttgaacaaaatctttgtaaatctaaataggaaaaaaatttgagcatgtttttaacaaccttttcaaagtgatgttagatcaatatattcattccaattatttgatttagctgtgaagacctagaagtgcccttctttcctctagtatataattattatgaaaataagaccctgctgtttggcaccagtatcatttgtctaactaggctttcacttcatccttatgttctgaacacctaattggttctaaaaaagtcagtttttcagatcttctcaaatctattctctcacagttttgtcagatataaaaggctagttacacttctgctctctattatgtttttgagttctaaaaggccagtttattggaatatagttcttatttgtacactactgtagcatataaacaaaattatagaaatacaatatattgtatatacattaattatattttattatttataatatataatatacaatatataaatataaatttatagaaatactatgtgataattactgtgtaaggcctatattaaaatcccagcccctaaaattctaaaaacatccaagcttacaattataaatctgttactaacagtgtgggttaaattatatctataatctttaaagaaagaatgcattttctgattttttaactgtaatagcttctccacagaaaaacagagtacttactttgtgctgagtgttcccaaaatttatttatcttcataagcacataagcttaactactctccctatttcatacataaagaaaactgaagttcaaaggcagcagtttgtctgagggcacacatttggaaaataggagaaatagtaatcaaacccaggtctcctgattccaagtttactgatatttttattatgttacagcttccttattagaactttagtttttctccccatcgacacgtagatttgattaaaaacttaatagaacccatataagtcagtacaagtcaagtcctctaacctgggtaactattctcagaaggacccttagatgcctattatttctttataattataataaaattaatatagaaccttattaagtgtaaaaatcttgatggtctatttgctcaagtaattgtgaataaacaagcttcaaagaatatgtcatattcagaatttacttaactgttaagaattcatttagataataattcagtttacattatcaatacaaataccaacacaaatttgtcatttaaagaaaatgcaatactataagaaaaacaaacaaaaaaagaaaatgcaatactacgcttccaaattttattcatcataaaccaattacatcttgctaaaaaaaagagactctattcagaattgaggtttccataaaccaaagtagggatgctccataaaaaataatttaaaatacaacaaaatgacaacatttaactgcttaaaataacaaattttcaagttttgatgtttaagtcgtcatatgtgctaatttgtgtaattttaaaattctctttaaagcattattagtaaaacgttaaactcaaatctaggaatctgatgaaaagttactgtgtattaatttaaggacgaaacatcctttaactgcttatactaaggccaatgtaaataatcttgaatgaccagtttcatttttaatgtttcagtttcaagcacagtactcaaaataacacaattcttatacaatgacagcaaagttgtttcagacaacggatgtttcactaagttgcctagaatttagtgtctctacacccaaaaactaaaccagagtcaaacacaaggtatgtatttcttgcattatactataacctttcccaagacaagttacatgcctttttctattcatgacagagaccaaatagaccatgacacatgaccatgggtcagctggagtccagaacacagtcatcctccaatcttcatgggggaatggttccaggaaaccccacaccaaaatcaaaatccaggatgctctaattcctcatataaactggcacagtattttcgtataacctttacactcctcctgcacactttaaatcatctctagattacttataatatctaatacaacgtaaatgttatgtaaataattgttacaatgtattttaaaaattttttatgtatttttttctaaactattttgatctacagttgaattcacagatgtgacacccgcagataaggaaggcctgctgtacatgaattcttcctgtgtctcttctgcaaaaaaacctgccatagccatctgtactgtcatggataacctagggcttacctataccacacatctcattctccttacaatttagttttaatataagaagaagctgcattcgactatgttgccacctaaattttcttatcaaaaactcctcatccaaccatccttttcttacctgtgatgcctaagctcaaatacggctgtttttgagtgtgtaaggtaaataaaggaggacctgcctcacaactgatgattgccttctctgtagtaacttgtcaacttacattcatcactattcaataagataacagtttgtgatttttcagtaccctgctttatcaaattccatcaagaaaaaacatgttatcattttcaccaatttgttataaaatacttacactcctctatattccataggtccaataacattattcactgcaaagtaatttattaactcacaatttcctcataatttcattgacatagcttctcaaaaaataatagaaactgagaatgttcacatttaaaatgtctttctaaaattttaaatttgattcctataatagcctttgccacttttagatataaccacaaaagcaacttaaatatcctaaaattagctgttaaaaatttttttctaagtaaaagtgcattaaaataggaagttattttaaataagtatgtttggtattctctggcaactaaaggctacttagcttagtattacagatatttttctatgaattctgaaatttatacaaggaaactactagtaagaatgaaactaacctatttagtattacttgttcagagtaagttgtacaagatacattttatttgttacaattctgtagtgacatggtaaatacccacaaaacttgagaaagggaagaatgactgtcaattggttttaacttaagctgaagctgcattagactattattattattattttcttattattcatccacaactttccagattatgaaaaaaaaaaagaaaaccaaaaactaacttacaaagaaattctgaatcaactaaaaaactgaaatcggttaggatttattttacctaaagctaaagtctttcttgaattctgtttaaaatatatataaacagattgacaaaacagaagtcaagacatctcttctctgacagtttccaaaaagaatacaaactattgtgatggttgtaaatctcacaaataactgtatagaaggaggcagacgccatagccaaattctcttcatatattcctaaatgtcatttgatgattacccaaagaaaaaaagctttgcatattctctaatccattcaggatatgtcaaccacccaaattatttcttaatgttcagtgttccataaaaacaagtcacagtagaaatcagtagcacaatttttccagaggtctatccacaatgccttaaccatgatttatttttggtattaaacatttttcctttattttttagacctgtttcatccttgagtctataaagtaattatagtatcttccgaagattttttttttcttttcttgagacagggtatcactgtgtcacccacgctggagggcagtggcatggggcacagcttattgcagcttcaacctccctgggctcaggcgatcctctcacctcagccttcggggtaattgggactacaggcatgcgccactacacctggctaatttttttttttagtttttgtagagatggggttttccattttgtccaggctggtctccaactcatggcctcccaaagtactgggattacaggcatgagccaccacacctggactcttctgaagttttaatgtggcctttttatactgcagattctatgttcagaaagtatatacatacttgtgcctggaataaaaatgtaaatgcttttcttaaagaaaactttattataaagctctaagcagaacaacctttactaccactactaaagttctgataatagcaaaccaaaacccttacatagtacttgattcatgccagttattactcacagaccacacaaagtagaagctattattagcccattttacagaatggaaaacagactccctcaatacgattctgcctcacaacaatcaaataaaaaccattatggtttgggaaagggacattccagctcccgctttatgctcatgccgctttacaactatttaagaatattaacaatatagtaagtattgcatttaaaaagtttgtaaatgcctcaaattttaaaaaatggtataagcatcaatagaataattctatatgtaagaaagaatgaagaatgctccttcagcctctcagcaacatattcacacctaacattttattcatcatataacccttcacgcctaacatattttattcatcattataacgaccatatgagattaaagctgttttagagactaccacgcaaagcttctatttcattgatactctactaaaaaaggaaatagtaactctactacaactacacacatttactttattacatgttcacccaaccccaaaaaattataataatcaagtgttgaaactatgttatcttcaatatagaatgggaatccctacttctaaaacatttaatatgatatcttttttttttccctgaaagttgtctctaggttttataccttaactttcacattaatcagcacacactaaataaatgtatacctaagatatatacttaaataaatcctatccatcattcctattcatctctgaatttgagaccaacaataatgaaaactagtacttaaactatgatggaaatcatggtaattttggggcattttacaacgtagttagtgtctcaaatcatctttgcaacaagaaatgatattaccaccaaagaatggcactatgaaaagcatttatataattttgtaacctatgtgatttctacttttctgtgttttggaaaactaagctctaagaatgaaataaagcttagttcttaaatacaatgtactgctatttctagttcaaaatcacagattttcagattgaaaaaatttcaatccacttatttttcaaatgagataactgggacaaagagaaattccatgacttgcccaagattacctacagtttaactgtcagcggggcttaaaaccacaatccacatctcctgactcccaatcctttcacttaaaacaaacaagcaaacaaacaaaaaagatttctaataaagtggaataattttaagaaaggcaagtatcactattttacaaggaaaaaattaaatcattttaacagattggcaaaacatgaactagttcttggggggaaaaaagagaagtcttacaagaaaaaatgtaatcaagagagtgccaaattcggtaaaatgcttgaaaattctgcctctagatctcgtaaatatgcaatcatcattaagtgacaactagaaagcagacttaataaactaactagattcactattcaaactaagaaataaacaaatgacaaagctttcctttcgtccaaaaaaagttttttattctacagtttaagaattctgatacttggaaaaagtgccccttttctttaaaataaatctcatattttaaaaaatgtaaaatctaattaaacgtataccatagtaccaaaaacaacttttagcttcctatccaattccatttactttgttaaaaatgttttaaatcttaaggtagatggtgataatcagtcatgttttataccagagacagaaacaaccataagatacgaccatttcctttctcaatcacacttgaaatgaacgcatcaattttaacctgcaaacttttaaaactgctcttaaaattctactttcctcttgattaaaattcaaccattgcgattgtaactagactaactacagatgatcagtgactatttttaaattcacatctacaaatattacaccccattttaagcagcaataatttgaggtttcctagaaatttcaatgcgatgtgatatatgagttctcccatttaaaatattgctcagtttattagttaatacaacaaatcatttccaggtagagtagaaactaatgactcaacaagtaattttcaaatcaatgttaaataaattcaactcgatatacaacaacgtaaaactttttaagtcagaataattaaaatagaaaatactgtacaagagactttgcatgtgctgacttagatattaaacagcgagatcaactattgaacaaaaaaatccagtgttccaaatgtttttagacctaactaaatctcaactaaaaaggtaaaataaagttaactcacacacctagatatacagtttgatggatgagaaagcacctcaaatggtaccttgcatccagtagatatagagtaagcaatatgctgaatgaatgaaaagagaaaacgagtcaaagaactccaagttctaataagatttctaaactgtctgatgagtatgccaacgttcctgttctagtaaggagaaaactccaagcaagaaaaaccacttccattcaaaataggtgaattttgagcataatacatagatagaaagaatgcttactgtatcttaaatctgcgatgcagactagggatagaaattcactttactaataattcctccccccaccctccccccaaaaattaaattaactcaaaatcaaaattgatagctcatttttactgaaaaaaaaaacaaaaaaacaaaatgatattcctacgaggattagccattaccataatttagccagataacattaagctgcttcatttaaaaaatgtaacattaccaaaagattaagaaaatgcagcattcctcagtgacttaaggtttgtgggtttttaagagatgcacagatgtaaaagcagatgcaaagacgagttttgtaaaacctgccccatcttaaaaatggagtattataatctttgcgataattttttcaaatatcaaggaagacatgtaaattcactgaagacttctatcaagtatttgtaaacctaaaaattaatttcaaattagtaaatcttggagtttacttccagctccattcactttggccaagaattgaatgaaagtaacccaaatcactccttgaaaattaacacacgttcagtgtgaaaatgaatacactaatacactgttaaatctccattagatgtattaaacctcagtacccttgcttatttcaacagccttgagcggttatcaacatcttatattaaaccacaagagatttatacacaaaagttaggaaatacactacataccaaaaaaagcgccattataatcatgtcctgctttcacctcacaaaagacactcattctaagctcgctgaaacttcctagtcattagagaagttctgatgaagtaacattagtaatcataactatctcaaaacagttacaaaagcctcataaaatcaacacactacataaatttcaaaggcttggtgggtccggtgcgactgctttaactgccccacacacatattcacacaacgaacctgtatcagtttaggagaaagtgttacagaaaatatagctcctttaaagtaacttccaatcacaatactgaagtgataaatccacttctgaaaagcaatttttaaagattcctaaaatactcattttgacaacccacaaaattaaggtttttaagctattaaacaaaatatgtcccaatataaacacaactttcataggccaagttccatcccacagtaaatatgtggacaaaaatcaaaactcttcagtgtactccaataataatttttaattaaacgagaggcataccataagaattaaaaaaagcctactaaacttctggttttagggaattacaggctttacacttctgcaaagatgtgttttagtaaatgcaagacgaagcactgaccaactatcatcacacatcagaatccttccaacaaaaaacccaggactgaatttaaggaaaacaaaataaacgacagagggggaaaaaataatgtcttgccacggtaccgcagcggctggatagcctgcttgtgaaatgctaatgccacttcggagcagttagtcaccagctattgtgtagggcaggagaaagccgagccggccgcgcgtgcagagcgagcaagcgaacgagcgagcgcgctctccctcttcgcgccgctcccgccgccgccgactctcgcgcgcccccgcgcccgcacggacgcgcgcgccggcccctcctcctccggccttgcactgcacaacactcatgacgtatctttatttctagcacattaacaaaatatcacaaataaattgtccgcagcccctgcggccccgaagtacgagtacccccggccactggcccccgcagaccccgcgccggcctcccaaccctccccatggcctttggagctttcacgttctagggccaagtttttgtctctgtaaaaaattgcgggaaattcaatttttattcgactcagggaaaagtttctttgctctgcgacgtgaatgtctcaccagattctggtaggtcctgggatgctccttcccggtccagtcggtgcgccggggcagcagctgcggggagaggacaccccgtgagcccgcccccggccctacccgccgtcccctccccgccgcccggcccgcggtccccgagccccggcccgcctccgcgggcagggcggcagggccagaggcgcccgcgccggggtaccgcgggccgcgccgcttacctccttctcggccacctcgcggatcagcacctgcaacaacaaagcggggagagctgagccccgcgccccgggccgcggtccgccgtgctcgcctccctcccccacgcccgcgagcgcgagcgccggcccggcccgcgccgcgcgccgccgtacctgagccgcctgctgacagggtccatcttccaggaaccgggcgatgaggaagtagagctctgcgcgggagagagggacggggagacacacaggctgagcggtcgggcggcggggggcgggggaccgcgggcggaatcgcccggtgccagcggccccggcagccccccgacttacccgatcgcagctccgagaggcctttcctctcacaagacatgtttatgggtcacttcagggccgccgacgggacaccccgccgccgaggggaagcggggacggtgccgccgcctgccctatagctgtcagtgtgtgttcacgagccgagcttcggctccaccattcaagcaacggcggcggaggcggaggaggaggaggaggaaacaacaactctcaggcagcgactacggccgtggccgcctccgccgcggatccctccgccgcagaaaggagtccgccgccttcgcggcccagggctcggccccggctctggcccgcgcccccgccccccggcgctaaaaaaggagtgcctccgacccctcgtccccagcgctccgcacgcggcacagtgagacccccacccgctcctccccgcagggcgtgcgatttatttatttatttccagtcggagaagatgtcggagcccaagccgccggttggctggaaggcgctttctctgtggaggccgatagtggcagggagggggccggggacggttccgcggagggatctgacgcacacggagccgcagcacaggctctattcagcggcgctggctggagctgagatggaagttagtttctatgtagcagaaatatgaaacaaatgaagcaaaactgcccagagaggggaaatgccccaaggatgggtctcactcacgcgcgtacacagacacacacgcagagagcactctcacgctgggcaagctcgggatcgcgctacccttcccgagttgaatgatagtgtttggtttctgtctcttgccatgtgcatgtgtataaatgctgcggattggcatctgtgtaagtcttgtcctgcgttatttctgcagcctatgcaagtgttgtgtaatttattggagtgctgtatattgcaatagaggtttgggctgctttttgttaagcacttgcgttttgcaaacccgttatttgctgaagccacctctgcatatttcttttattactgccattgcctttggcgtacgttttttaaatgttttttattgttaaacgggcaaagcgaactcttgatttgtacttcagatactctttttccttattacaaaaaggctagtgatggctaattaggtatttggaattaaagaaccttaaagcttttttaagtgtttacgagaagggagaatgtaaacctgagggaaaggaaaggacgctaatattcatgtctaactgatctggaggtaatttagtgacagatcgataacctgcctaaggatattgaaagagtatactacagtttagccaaggtgaatagtgattaaataatttaaataatctgtgtatcttgcagttgacttcgtcatgctaattaatggcttctaatttgagatgtaaaccattcctgtttacagttaatcacgggaagacttcttgaaaactgacgaaaaggagaaaaaaaaatctttcgtaaattagtatgtaattaccgattttatatgctaaatcatacatctgtgttttgctgatgaggataagggccttgtttttaaaaaaacgaatatgggtgaaattaatggaaacaatagaaaaagccatttgttagaaaacaaggacaccaaatgatatttatctccagatgatttaagcactttccaaaaagacttgagagttcaatattttttaaggattgcattttaaagggaatttggatagtcgttcttttgttaacatttaacaaaagattctccttaaaaatgttagataataaactgcattttatgggtctggtttaaaaaggttatttgtggggaaaggaccaacaagctgtattgtggttttctagattgtttcctcaagccttgtaacctcctagctccttacattcctagtgggaaatacttgctgcaaatgccttgggctgcactgtaagcccaagtgtgctgcaccagtgtgatgccctatactaaaacatccagaaatcatcatacatatgaggaagaagaaataaagcctcaaaccctttggaataataggatataaaattgccttttgtaactgaatcttaaaaatggaaggttaccatgacttgtcctattgcaacctggttatcagaataacttattttttttaagatagctattctcaaatactgaacatatttgcatctttaaagacactttattctattcaattataggtaaagtagcctatttctaggtggttaggcttgaaaagatagactgaaaagataggaaattttgtatgcctttttgcaaattgtatttacttctaagaccgatgctgttttagcttaacttttaaaaaagtgttcttcaaataattgtaatattttacacgatcttgaagttcttcaaataaacagagtttagaaactaaaaattatagtgggattttctggttttgaaggcttggaa

SEQ ID NO: 70 - DNA, 2837 bp, Homo sapiens
cgagattggctgtggaagaactaactgaaaatggtttgacattagaagaatggttgccatcaacatggattacagataccattccccgaagatgtccatttgtgccacagatgggtgatgaggtttattatttccgacaaggacatgaagcctatgtcgaaatggcccggaaaaataaaatatatagtatcaatcccaaaaaacaaccatggcataaaatggagctacgggaacaagaacttatgaaaatagttggcataaagtatgaagtgggattacctaccctttgctgccttaaacttgcttttctagatcctgatactggtaaactgactggtggatcatttaccatgaaataccatgatatgcctgacgtcatagattttctagtcttgagacaacaatttgatgatgcaaaatacaggcgatggaatataggtgaccgcttcaggtctgtcatagatgatgcctggtggtttggaacaatcgaaagccaggaacctcttcaacttgagtaccctgatagtctgtttcaatgctacaatgtttgctgggacaatggagatacagaaaagatgagtccttgggatatggagcttatacctaataatgctgtatttcctgaagaactaggtaccagtgttcctttaactgatggtgagtgcagatcactaatctataaacctcttgatggagaatggggtaccaatcccagggatgaagaatgtgaaagaattgtggcaggaataaaccagttgatgacactagatattgcctcagcatttgtggcccccgtggatctgcaagcctatcccatgtattgcacagtagtggcatatccaacggatctaagtacaattaaacaaagactggaaaacaggttttacaggcgggtttcttccctaatgtgggaagttcgatatatagagcataatacacgaacatttaatgagcctggaagccctattgtgaaatctgctaaattcgtgactgatcttcttctacattttataaaggatcagacttgttataacataattccactttataattcaatgaagaagaaagttttgtctgattctgaggatgaagagaaagatgttgatgtgccaggaacttctactcgaaaaaggaaggaccatcagcgtagaagaagattacgtaatagagcccagtcttacgatattcaagcatggaagaaccagtgtgaagaattgttaaatctcatatttcaatgtgaagattcagagcctttccgtcagccggtagatctccttgaatatccagactacagagacatcattgacactccaatggattttgctaccgttagagaaactttagaggctgggaattatgagtcaccaatggagttatgtaaagatgtcagacttattttcagtaattccaaagcatatacaccaagcaaaagatcaaggatttacagcatgagtttgcgcctgtctgctttctttgaagaacacattagttcagttttatcagattataaatctgctcttcgttttcataaaagaaataccataaccaaaaggaggaagaaaagaaacagaagcagctctgtttccagtagtgctgcatcaagccctgaaaggaaaaaaaggatcttaaaaccccagctaaaatcagaaagctctacctctgcattctctacacctacacgatcaataccgccaagacacaatgctgctcagataaacggtaaaacagaatctagttctgtggttcgaaccagaagcaaccgagtggttgtagatccagttgtcactgagcaaccatctacttcttcagctgcaaagacttttattacaaaagctaatgcatctgcaataccagggaaaacaatactagagaattctgtgaaacattccaaagctttgaatactctttccagtcctggtcaatccagttttagtcatggcactaggaataattctgcaaaagaaaacatggaaaaggaaaagccagtcaaacgtaaaatgaagtcatctgtactcccaaaggcgtccactctttcaaagtcatcagctgtcattgagcaaggagattgtaagaacaacgctcttgtaccaggaaccattcaagtaaatggccatggaggacagccatcaaaacttgtgaagaggggacctggaaggaaacctaaagtagaagttaataccaatagtggtgaaattatacacaagaaaaggggtagaaagcccaaaaagctacagtatgcaaagccagaagatttagagcaaaataatgtgcatcccatcagagatgaagtacttccttcttcaacatgcaattttctttctgaaactaataatgtaaaggaagatttgttacagaaaaagaatcgtggaggtaggaagcccaaaaggaagatgaagacacaaaaattagatgcagatctcctagtccctgcaagtgtcaaagtgttaaggagaagtaacccgaaaaaaatagatgatcctatagatgaggaagaagagtttgaagaactcaaaggctctgaaccccacatgagaactagaaatcaaggtcgaaggacagctttctataatgaggatgactctgaagaggagcaaaggcagctgttgttcgaagacacctctttaacttttggaacttctagtagaggacgagtccgaaagttgactgaaaaagcaaaagctaatttaattggttggtaacttgtaccaaaatattttacttcaaaatctataaagcaggtacagttaaggaataagtaggactaaggcttctgcttccttgctgctgtggtggagtagggaatgttatgatttgatttgcaaaaaaaaaaaaaaaag

SEQ ID NO: 71 - protein, 898 aa, Homo sapiens
RLAVEELTENGLTLEEWLPSTWITDTIPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQLEYPDSLFQCYNVCWDNGDTEKMSPWDMELIPNNAVFPEELGTSVPLTDGECRSLIYKPLDGEWGTNPRDEECERIVAGINQLMTLDIASAFVAPVDLQAYPMYCTVVAYPTDLSTIKQRLENRFYRRVSSLMWEVRYIEHNTRTFNEPGSPIVKSAKFVTDLLLHFIKDQTCYNIIPLYNSMKKKVLSDSEDEEKDVDVPGTSTRKRKDHQRRRRLRNRAQSYDIQAWKNQCEELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTITKRRKKRNRSSSVSSSAASSPERKKRILKPQLKSESSTSAFSTPTRSIPPRHNAAQINGKTESSSVVRTRSNRVVVDPVVTEQPSTSSAAKTFITKANASAIPGKTILENSVKHSKALNTLSSPGQSSFSHGTRNNSAKENMEKEKPVKRKMKSSVLPKASTLSKSSAVIEQGDCKNNALVPGTIQVNGHGGQPSKLVKRGPGRKPKVEVNTNSGEIIHKKRGRKPKKLQYAKPEDLEQNNVHPIRDEVLPSSTCNFLSETNNVKEDLLQKKNRGGRKPKRKMKTQKLDADLLVPASVKVLRRSNPKKIDDPIDEEEEFEELKGSEPHMRTRNQGRRTAFYNEDDSEEEQRQLLFEDTSLTFGTSSRGRVRKLTEKAKANLIGW

SEQ ID NO: 72 - DNA, 3892 bp, Mus musculus
gggggggggggctagaagagtttttagttttgtctgttaggatgtcttttgagagttttgtaaagaatatacgttttgcttttgtctctagccctccatcagtgattaggaaaagctgaataactttcgtcacttctgctgcttttctagtaaaaggttttaatactggagagtaaaatttttgcacagatttatttccttgtgtttgaagatagtactaatgctgttgcatgctttctcagagattggctgtaggagaactaactgagaatggcctaacgttagaagagtggttgccttcagcttggattacagacacacttcccaggagatgtccatttgtgccacagatgggtgatgaggtttattattttcgacaagggcatgaagcatatgttgagatggcccggaaaaataaaatttatagtatcaatcctaaaaagcagccatggcataagatggaactaagggaacaagaactaatgaaaattgttggtataaagtatgaagtggggttgcctaccctttgctgccttaaacttgcttttctagatcctgatactggcaaactgaccggtggatcatttaccatgaaataccatgatatgcctgacgtcatagattttctagtcttgagacaacaatttgatgatgcaaagtatagacgatggaatataggtgaccgcttcagatctgtcatagatgatgcctggtggtttggaacaattgaaagtcaagagcctcttcaacctgagtaccctgatagtttgtttcagtgttataatgtatgttgggacaatggagatacagaaaagatgagtccttgggatatggaattaatacctaataatgctgtctttccagaagaactgggtaccagtgttcctttaactgatgttgaatgtaggtcgctaatttataaacctcttgatggagattggggagccaatcccagggatgaagaatgtgaaagaattgttggaggaataaatcagctgatgacactagatattgcgtctgcatttgttgcccctgtggaccttcaagcttatcccatgtattgcactgtggtggcctatccaacggatctaagtacaattaaacaaagactggagaacaggttttacaggcgcttttcatcactaatgtgggaagttcgatatatagaacataatacacgaacattcaatgagccaggaagcccaattgtgaaatctgctaaatttgtgactgatcttctcctgcattttataaaggatcagacttgttataacataattccactttacaactcaatgaagaagaaagttttgtctgactctgaggaagaagagaaagatgctgatgttccagggacttctaccagaaagcgcaaggatcatcaacctagaagaaggttacgcaacagagctcagtcttacgatattcaggcatggaagaaacaatgtcaagaattactgaatctcatatttcaatgtgaagactcagaaccttttcgacagccagtggatcttcttgaatatccagactaccgagacatcattgacactccaatggactttgccactgttagagagactttagaggctgggaattatgagtcacccatggagttatgtaaagatgtcaggctcattttcagtaattctaaagcatacacaccaagcaagagatcaaggatttacagcatgagtttacgcctgtctgctttctttgaagaacatattagttcagttttgtcagattataaatctgctcttcgttttcataaaagaaacaccataagcaagaagaggaagaagcgaaacaggagcagctccctgtccagcagtgctgcctcaagccctgaaaggaaaaaaaggatcttaaaaccccagctaaagtcagaagtatctacctctccattctccatacctacaagatcagtactaccaagacataatgctgcacaaatgaatggtaaaccagaatccagttctgtggttcgaactaggagcaaccgtgtagctgtagatccagttgtcaccgagcagccctctacatcatcagccacaaaagcttttgtttcaaaaactaatacatctgccatgccaggaaaagcaatgctagagaattctgtgagacattccaaagccttgagcacactttccagccctgatccgctcacattcagccatgctacaaagaataattctgcaaaagaaaacatggaaaaggaaaagcctgtcaaacgtaaaatgaagtcttctgtgttttcaaaagcatctccacttccaaagtcagccgcagtcatagagcaaggagagtgtaagaacaatgttcttataccaggaaccattcaagtaaatggccatggaggacaaccatcaaaactcgtgaagagaggacctgggaggaagcccaaggtagaagttaacaccagcagtggtgaagtgacacacaagaaaagaggtagaaagcccaagaatctgcagtgtgcaaagcaggaaaactctgagcaaaataacatgcatcccatcagggctgacgtgcttccttcttcaacatgcaacttcctttctgaaactaatgctgtcaaggaggatttgttacagaaaaagagtcgtggaggcagaaaacccaaaaggaagatgaaaactcacaacctagattcagaactcatagttcctacaaatgttaaagtgttaaggagaagtaaccggaaaaaaacagatgatcctatagatgaggaagaggagtttgaagaactcaaaggctctgagcctcacatgagaactagaaatcagggtcgaaggacaactttctataatgaggatgactccgaggaagaacagagacagctgttgttcgaggacacctccttgacatttggaacttctagtagaggacgagtccgaaagttgactgaaaaagcaaaggctaatttaattggttggtaacttgaagcaaaatattgcattttaaaaaatctgtaacgcaggtacagttaaggagtaagtagaactaaggtctctgcttccttgctgctatgacggattagggaatgttacaatttgacttgggaaaatggacaaaaacacatttagaagataatttacatctttgaatgaaaaaaatctatatacatatatatttcaaatgtttgctatttattgcccttaggtaggttattcggttccacattcatttcatttgctgtttgaaattgaggacctgttataaattctggtttatttatggaagagacagctctgctacactattaagaaacatagtattcctagagataaagtatgttccctcttaaattgagttatttttgaccaagtgaggtacatttttactgatagcagaaggcatgccctaggaagagagatgttacaaagagtagcagtacattaagaatggcttcctctaaagataactttccagttcccaccatttggtatcctgaaaagtgttgtgaactgtaggtgttcaattacagaatatctagaggaagcttttgttttactccatttctgccaaacttaggagaaaaatgtattgatgcaaaggaaacatatccacattggaaaacatttgactgtctaatttttcagaccttgattcttatatcagtcactctatctctgtttattgtgccaaagactgagaatcagtgcagtggaaagcctgtttttgactgtcaggacagcatacacttttcagtactggaaaagctatatattctaaagagcaagttattacaaaattatgctgagttatatcctttttttggtactaaatgtaggaaaataatgcactggtgggtcctttgacagagatatcttagagaaaaaaaaaaaaaaggaattcgatatcaagcttatcgataccgtcgacctcgagg

SEQ ID NO: 73 - protein, 902 aa, Mus musculus; identical to SEQ ID NO: 5

SEQ ID NO: 74 - DNA, 3264 bp, Homo sapiens
cggatcttggagaatccaaaaagcaacagacaaatcaacacaattatcgtacaagatctgcattggaagagactcctagaccctcagaagagatagaaaatggcagtagttcttcagatgaaggcgaagtagttgctgtcagtggtggaacatccgaagaagaagagagagcatggcacagtgatggcagttctagtgactactccagtgattactctgactggacagcagatgcaggaattaatctgcagccaccaaagaaagttcctaagaataaaaccaagaaagcagaaagcagttcagatgaagaagaagaatctgaaaaacagaagcaaaaacagattaaaaagggaaaagaaaaagcaaatgaagaaaaagatggaccaatatcaccaaagaaaaagaaagcccaaagaaagaaaacaaaagagattggctgtggaagaactaactgaaaatggtttgacattagaagaatggttgccatcaacatggattacagataccattccccgaagatgtccatttgtgccacagatgggtgatgaggtttattatttccgacaaggacatgaagcctatgtcgaaatggcccggaaaaataaaatatatagtatcaatcccaaaaaacaaccatggcataaaatggagctacgggaacaagaacttatgaaaatagttggcataaagtatgaagtgggattacctaccctttgctgccttaaacttgcttttctagatcctgatactggtaaactgactggtggatcatttaccatgaaataccatgatatgcctgacgtcatagattttctagtcttgagacaacaatttgatgatgcaaaatacaggcgatggaatataggtgaccgcttcaggtctgtcatagatgatgcctggtggtttggaacaatcgaaagccaggaacctcttcaacttgagtaccctgatagtctgtttcaatgctacaatgtttgctgggacaatggagatacagaaaagatgagtccttgggatatggagcttatacctaataatgctgtatttcctgaagaactaggtaccagtgttcctttaactgatggtgagtgcagatcactaatctataaacctcttgatggagaatggggtaccaatcccagggatgaagaatgtgaaagaattgtggcaggaataaaccagttgatgacactagatattgcctcagcatttgtggcccccgtggatctgcaagcctatcccatgtattgcacagtagtggcatatccaacggatctaagtacaattaaacaaagactggaaaacaggttttacaggcgggtttcttccctaatgtgggaagttcgatatatagagcataatacacgaacatttaatgagcctggaagccctattgtgaaatctgctaaattcgtgactgatcttcttctacattttataaaggatcagacttgttataacataattccactttataattcaatgaagaagaaagttttgtctgattctgaggatgaagagaaagatgttgatgtgccaggaacttctactcgaaaaaggaaggaccatcagcgtagaagaagattacgtaatagagcccagtcttacgatattcaagcatggaagaaccagtgtgaagaattgttaaatctcatatttcaatgtgaagattcagagcctttccgtcagccggtagatctccttgaatatccagactacagagacatcattgacactccaatggattttgctaccgttagagaaactttagaggctgggaattatgagtcaccaatggagttatgtaaagatgtcagacttattttcagtaattccaaagcatatacaccaagcaaaagatcaaggatttacagcatgagtttgcgcctgtctgctttctttgaagaacacattagttcagttttatcagattataaatctgctcttcgttttcataaaagaaataccataaccaaaaggaggaagaaaagaaacagaagcagctctgtttccagtagtgctgcatcaagccctgaaaggaaaaaaaggatcttaaaaccccagctaaaatcagaaagctctacctctgcattctctacacctacacgatcaataccgccaagacacaatgctgctcagataaacggtaaaacagaatctagttctgtggttcgaaccagaagcaaccgagtggttgtagatccagttgtcactgagcaaccatctacttcttcagctgcaaagacttttattacaaaagctaatgcatctgcaataccagggaaaacaatactagagaattctgtgaaacattccaaagctttgaatactctttccagtcctggtcaatccagttttagtcatggcactaggaataattctgcaaaagaaaacatggaaaaggaaaagccagtcaaacgtaaaatgaagtcatctgtactcccaaaggcgtccactctttcaaagtcatcagctgtcattgagcaaggagattgtaagaacaacgctcttgtaccaggaaccattcaagtaaatggccatggaggacagccatcaaaacttgtgaagaggggacctggaaggaaacctaaagtagaagttaataccaatagtggtgaaattatacacaagaaaaggggtagaaagcccaaaaagctacagtatgcaaagccagaagatttagagcaaaataatgtgcatcccatcagagatgaagtacttccttcttcaacatgcaattttctttctgaaactaataatgtaaaggaagatttgttacagaaaaagaatcgtggaggtaggaagcccaaaaggaagatgaagacacaaaaattagatgcagatctcctagtccctgcaagtgtcaaagtgttaaggagaagtaacccgaaaaaaatagatgatcctatagatgaggaagaagagtttgaagaactcaaaggctctgaaccccacatgagaactagaaatcaaggtcgaaggacagctttctataatgaggatgactctgaagaggagcaaaggcagctgttgttcgaagacacctctttaacttttggaacttctagtagaggacgagtccgaaagttgactgaaaaagcaaaagctaatttaattggttggtaacttgtaccaaaatattttacttcaaaatctataaagcaggtacagttaaggaataagtaggactaaggcttctgcttccttgctgctgtggtggagtagggaatgttatgatttgatttgcaaaaaaaaaaaaaaaag

SEQ ID NO: 75 - protein, 1039 aa, Homo sapiens
RILENPKSNRQINTIIVQDLHWKRLLDPQKRKMAVVLQMKAKLLSVVEHPKKKREHGTVMAVLVTTPVITLTGQQMQELICSHQRKFLRIKPRKQKAVQMKKKNLKNRSKNRLKREKKKQMKKKMDQYHQRKRKPKERKQKRLAVEELTENGLTLEEWLPSTWITDTIPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQLEYPDSLFQCYNVCWDNGDTEKMSPWDMELIPNNAVFPEELGTSVPLTDGECRSLIYKPLDGEWGTNPRDEECERIVAGINQLMTLDIASAFVAPVDLQAYPMYCTVVAYPTDLSTIKQRLENRFYRRVSSLMWEVRYIEHNTRTFNEPGSPIVKSAKFVTDLLLHFIKDQTCYNIIPLYNSMKKKVLSDSEDEEKDVDVPGTSTRKRKDHQRRRRLRNRAQSYDIQAWKNQCEELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTITKRRKKRNRSSSVSSSAASSPERKKRILKPQLKSESSTSAFSTPTRSIPPRHNAAQINGKTESSSVVRTRSNRVVVDPVVTEQPSTSSAAKTFITKANASAIPGKTILENSVKHSKALNTLSSPGQSSFSHGTRNNSAKENMEKEKPVKRKMKSSVLPKASTLSKSSAVIEQGDCKNNALVPGTIQVNGHGGQPSKLVKRGPGRKPKVEVNTNSGEIIHKKRGRKPKKLQYAKPEDLEQNNVHPIRDEVLPSSTCNFLSETNNVKEDLLQKKNRGGRKPKRKMKTQKLDADLLVPASVKVLRRSNPKKIDDPIDEEEEFEELKGSEPHMRTRNQGRRTAFYNEDDSEEEQRQLLFEDTSLTFGTSSRGRVRKLTEKAKANLIGW

SEQ ID NO: 76 - DNA, 3860 bp, Mus musculus
ggacagcagatgctggaattaacttgcagccaccaaagcccgttcctcctaagcataaaaccaagaaaccagaaagtagttcagatgaagaagaagaatctgaaaaccagaagcaaaaacatattaaaaaggaaagaaaaaaagcaaatgaagaaaaagatggaccaacatcaccaaagaaaaaaaaagcccaaagaaagaaaacaaaagagattggctgtaggagaactaactgagaatggcctaacgttagaagagtggttgccttcagcttggattacagacacacttcccaggagatgtccatttgtgccacagatgggtgatgaggtttattattttcgacaagggcatgaagcatatgttgagatggcccggaaaaataaaatttatagtatcaatcctaaaaagcagccatggcataagatggaactaagggaacaagaactaatgaaaattgttggtataaagtatgaagtggggttgcctaccctttgctgccttaaacttgcttttctagatcctgatactggcaaactgaccggtggatcatttaccatgaaataccatgatatgcctgacgtcatagattttctagtcttgagacaacaatttgatgatgcaaagtatagacgatggaatataggtgaccgcttcagatctgtcatagatgatgcctggtggtttggaacaattgaaagtcaagagcctcttcaacctgagtaccctgatagtttgtttcagtgttataatgtatgttgggacaatggagatacagaaaagatgagtccttgggatatggaattaatacctaataatgctgtctttccagaagaactgggtaccagtgttcctttaactgatgttgaatgtaggtcgctaatttataaacctcttgatggagattggggagccaatcccagggatgaagaatgtgaaagaattgttggaggaataaatcagctgatgacactagatattgcgtctgcatttgttgcccctgtggaccttcaagcttatcccatgtattgcactgtggtggcctatccaacggatctaagtacaattaaacaaagactggagaacaggttttacaggcgcttttcatcactaatgtgggaagttcgatatatagaacataatacacgaacattcaatgagccaggaagcccaattgtgaaatctgctaaatttgtgactgatcttctcctgcattttataaaggatcagacttgttataacataattccactttacaactcaatgaagaagaaagttttgtctgactctgaggaagaagagaaagatgctgatgttccagggacttctaccagaaagcgcaaggatcatcaacctagaagaaggttacgcaacagagctcagtcttacgatattcaggcatggaagaaacaatgtcaagaattactgaatctcatatttcaatgtgaagactcagaaccttttcgacagccagtggatcttcttgaatatccagactaccgagacatcattgacactccaatggactttgccactgttagagagactttagaggctgggaattatgagtcacccatggagttatgtaaagatgtcaggctcattttcagtaattctaaagcatacacaccaagcaagagatcaaggatttacagcatgagtttacgcctgtctgctttctttgaagaacatattagttcagttttgtcagattataaatctgctcttcgttttcataaaagaaacaccataagcaagaagaggaagaagcgaaacaggagcagctccctgtccagcagtgctgcctcaagccctgaaaggaaaaaaaggatcttaaaaccccagctaaagtcagaagtatctacctctccattctccatacctacaagatcagtactaccaagacataatgctgcacaaatgaatggtaaaccagaatccagttctgtggttcgaactaggagcaaccgtgtagctgtagatccagttgtcaccgagcagccctctacatcatcagccacaaaagcttttgtttcaaaaactaatacatctgccatgccaggaaaagcaatgctagagaattctgtgagacattccaaagccttgagcacactttccagccctgatccgctcacattcagccatgctacaaagaataattctgcaaaagaaaacatggaaaaggaaaagcctgtcaaacgtaaaatgaagtcttctgtgttttcaaaagcatctccacttccaaagtcagccgcagtcatagagcaaggagagtgtaagaacaatgttcttataccaggaaccattcaagtaaatggccatggaggacaaccatcaaaactcgtgaagagaggacctgggaggaagcccaaggtagaagttaacaccagcagtggtgaagtgacacacaagaaaagaggtagaaagcccaagaatctgcagtgtgcaaagcaggaaaactctgagcaaaataacatgcatcccatcagggctgacgtgcttccttcttcaacatgcaacttcctttctgaaactaatgctgtcaaggaggatttgttacagaaaaagagtcgtggaggcagaaaacccaaaaggaagatgaaaactcacaacctagattcagaactcatagttcctacaaatgttaaagtgttaaggagaagtaaccggaaaaaaacagatgatcctatagatgaggaagaggagtttgaagaactcaaaggctctgagcctcacatgagaactagaaatcagggtcgaaggacaactttctataatgaggatgactccgaggaagaacagagacagctgttgttcgaggacacctccttgacatttggaacttctagtagaggacgagtccgaaagttgactgaaaaagcaaaggctaatttaattggttggtaacttgaagcaaaatattgcattttaaaaaatctgtaacgcaggtacagttaaggagtaagtagaactaaggtctctgcttccttgctgctatgacggattagggaatgttacaatttgacttgggaaaatggacaaaaacacatttagaagataatttacatctttgaatgaaaaaaatctatatacatatatatttcaaatgtttgctatttattgcccttaggtaggttattcggttccacattcatttcatttgctgtttgaaattgaggacctgttataaattctggtttatttatggaagagacagctctgctacactattaagaaacatagtattcctagagataaagtatgttccctcttaaattgagttatttttgaccaagtgaggtacatttttactgatagcagaaggcatgccctaggaagagagatgttacaaagagtagcagtacattaagaatggcttcctctaaagataactttccagttcccaccatttggtatcctgaaaagtgttgtgaactgtaggtgttcaattacagaatatctagaggaagcttttgttttactccatttctgccaaacttaggagaaaaatgtattgatgcaaaggaaacatatccacattggaaaacatttgactgtctaatttttcagaccttgattcttatatcagtcactctatctctgtttattgtgccaaagactgagaatcagtgcagtggaaagcctgtttttgactgtcaggacagcatacacttttcagtactggaaaagctatatattctaaagagcaagttattacaaaattatgctgagttatatcctttttttggtactaaatgtaggaaaataatgcactggtgggtcctttgacagagatatcttagagaaaaaaaaaaaaaaggaattcgatatcaagcttatcgataccgtcgacctcgagg

SEQ ID NO: 77 - protein, 968 aa, Mus musculus
GQQMLELTCSHQSPFLLSIKPRNQKVVQMKKKNLKTRSKNILKRKEKKQMKKKMDQHHQRKKKPKERKQKRLAVGELTENGLTLEEWLPSAWITDTLPRRCPFVPQMGDEVYYFRQGHEAYVEMARKNKIYSINPKKQPWHKMELREQELMKIVGIKYEVGLPTLCCLKLAFLDPDTGKLTGGSFTMKYHDMPDVIDFLVLRQQFDDAKYRRWNIGDRFRSVIDDAWWFGTIESQEPLQPEYPDSLFQCYNVCWDNGDTEKMSPWDMELIPNNAVFPEELGTSVPLTDVECRSLIYKPLDGDWGANPRDEECERIVGGINQLMTLDIASAFVAPVDLQAYPMYCTVVAYPTDLSTIKQRLENRFYRRFSSLMWEVRYIEHNTRTFNEPGSPIVKSAKFVTDLLLHFIKDQTCYNIIPLYNSMKKKVLSDSEEEEKDADVPGTSTRKRKDHQPRRRLRNRAQSYDIQAWKKQCQELLNLIFQCEDSEPFRQPVDLLEYPDYRDIIDTPMDFATVRETLEAGNYESPMELCKDVRLIFSNSKAYTPSKRSRIYSMSLRLSAFFEEHISSVLSDYKSALRFHKRNTISKKRKKRNRSSSLSSSAASSPERKKRILKPQLKSEVSTSPFSIPTRSVLPRHNAAQMNGKPESSSVVRTRSNRVAVDPVVTEQPSTSSATKAFVSKTNTSAMPGKAMLENSVRHSKALSTLSSPDPLTFSHATKNNSAKENMEKEKPVKRKMKSSVFSKASPLPKSAAVIEQGECKNNVLIPGTIQVNGHGGQPSKLVKRGPGRKPKVEVNTSSGEVTHKKRGRKPKNLQCAKQENSEQNNMHPIRADVLPSSTCNFLSETNAVKEDLLQKKSRGGRKPKRKMKTHNLDSELIVPTNVKVLRRSNRKKTDDPIDEEEEFEELKGSEPHMRTRNQGRRTTFYNEDDSEEEQRQLLFEDTSLTFGTSSRGRVRKLTEKAKANLIGW